(19) United States
(12) Patent Application Publication
(10) Pub. No.: US 2011/0311550 A1
(43) Pub. Date: Dec. 22, 2011

(54) AGENTS FOR HCV TREATMENT

(75) Inventors: Mansun Law, San Diego, CA (US); Dennis R. Burton, La Jolla, CA (US)

(73) Assignee: The Scripps Research Institute, La Jolla, CA (US)

(21) Appl. No.: 12/998,486

(22) PCT Filed: Oct. 23, 2009
(86) PCT No.: PCT/US2009/005786

§ 371 Date: Jul. 18, 2011

Related U.S. Application Data
(60) Provisional application No. 61/197,292, filed on Oct. 24, 2008.
(60) Provisional application No. 61/200,347, filed on Nov. 26, 2008.

(51) Int. Cl. A61K 39/42; C12N 15/13; A61P 31/14; C12N 1/21; C12N 5/10; C07K 16/10; C12Q 1/70; C12N 15/63

(52) U.S. Cl. 424/161.1; 435/5; 536/23.53; 435/320.1; 435/252.33; 435/339; 530/389.4; 530/387.3

(57) ABSTRACT

Provided are polypeptides, including antibodies and fragments thereof, useful for preventing or treating new or recurring infection of hepatitis C virus, as well as methods of preventing or treating new or recurring hepatitis C viral infection. Also provided are modified E1 and E2 polypeptides.

### RELATED APPLICATIONS

Benefit of priority is claimed to U.S. Provisional Application Ser. No. 61/197,292, to Mansun Law and Dennis Burton, entitled “AGENTS FOR HCV TREATMENT,” filed Oct. 24, 2008, and U.S. Provisional Application Ser. No. 61/200,347, to Mansun Law and Dennis Burton, entitled “AGENTS FOR HCV TREATMENT,” filed Nov. 26, 2008.

This application is related to International Application No PCT/US02/02303, to Toshiaki Maruyama, Ian Jones, Dennis Burton, and Robert Fox, entitled “IMMUNOPOLYPEPTIDES TO HEPATITIS C VIRUS,” filed Jan. 25, 2002; U.S. application Ser. No. 12/290,017, to Mansun Law, Toshiaki Maruyama, Dennis Burton, Jonathan Ball and Norman Kneteman, entitled “HCV NEUTRALIZING EPITOPES,” filed Oct. 24, 2008; and International Application No. PCT/US09/05785, to Mansun Law and Dennis Burton, entitled “AGENTS FOR HCV TREATMENT,” filed Oct. 23, 2009, the disclosures of which are specifically incorporated by reference herein in their entireties.

### FIELD OF THE INVENTION

Provided are polypeptides, including antibodies and fragments thereof, useful for preventing or treating new or recurring infection of hepatitis C virus, as well as methods of preventing or treating new or recurring hepatitis C viral infection. Also provided are modified E1 and E2 polypeptides.

### BACKGROUND

It is estimated that hepatitis C virus (HCV) infects about 2-3% of the world population, i.e. 120 to 170 million people worldwide. HCV infection predisposes the patient to chronic liver cirrhosis, cancer and liver failure. About 85% of individuals initially infected with HCV become chronically infected. Once established, chronic HCV infection causes an inflammation of the liver, and this can progress to scarring and eventually, liver cirrhosis. Some patients with cirrhosis will go on to develop liver failure or liver cancer. In the United States and Western Europe, the complications of chronic hepatitis and cirrhosis are the most common reasons for liver transplantation. In addition, liver disease caused by HCV is the leading cause of death in patients co-infected with human immunodeficiency virus. Given the large number of infected people worldwide, HCV infection can be a burden on health care systems worldwide.

Accordingly, there is a need for therapeutic agents and methods for the treatment of new or recurring hepatitis C viral infections.

### SUMMARY

Provided herein are polypeptides, including antibodies and fragments thereof, that can bind to hepatitis C virus. Thus, provided herein are anti-hepatitis C virus polypeptides and antibodies. Included among the polypeptides provided herein are broadly neutralizing antibodies that can neutralize genetically diverse HCV isolates, targeting different non-overlapping conserved viral epitopes, thereby protecting against hepatitis C virus (HCV) infection. In some examples, the antibodies bind to conformational HCV E1E2 neutralizing epitopes. In other examples, the antibodies bind to conformational E2 neutralizing epitopes. Also provided are antibodies that bind to the same epitope as any of the antibodies provided herein. Also provided are nucleic acids encoding the polypeptides and antibodies, as well as compositions, such as pharmaceutical compositions, containing one or more anti-HCV polypeptides, such as one or more anti-HCV antibodies, or nucleic acids encoding the polypeptides. Such polypeptides, antibodies, nucleic acid molecules and compositions can be used to prevent or treat HCV infection or reduce HCV replication. In one example, a combination of the polypeptides, such as antibodies, including E1E2- and/or E2-specific neutralizing antibodies, can be used to prevent new HCV infections, treat chronic HCV infection, as well as prevent recurrent HCV infection, e.g. following liver transplantation. An antibody combination, for example, can include at least one antibody provided herein that binds specifically with a neutralizing epitope on HCV E1E2 complex and at least one antibody that binds specifically with a neutralizing epitope on HCV E2 polypeptide. Such antibody combinations can be particularly useful for blocking viral entry during the infection process. Thus, also provided are methods of preventing, reducing or treating new or recurring HCV infections or HCV replication.

Provided herein are polypeptides containing at least one CDR sequence that contains a sequence of amino acids selected from among any set forth in any of SEQ ID NOS: 725-834, 985 and 1090-1133; or a sequence having at least 70%, 80%, 90%, 91%, 92%, 93%, 94%, 95%, 96%, 97%, 98%, 99% or more sequence identity with a sequence of amino acids set forth in any of SEQ ID NOS: 725-834 and 1090-1133, and that, when in an antibody or antigen-binding fragment thereof, the antibody or antigen-binding fragment thereof selectively binds to E1, E1/E2 complex and/or to an HCV virion. In some examples, the polypeptide has two CDR sequences, such as a first CDR (CDR1) with an amino acid sequence set forth in any of SEQ ID NOS: 725-741, 778-796, 985, 1090-1091 and 1101-1113, and a second CDR (CDR2) with an amino acid sequence set forth in any of SEQ ID NOS: 742-759, 797-815, 1092-1096 and 1114-1121. In other examples, the polypeptide has three CDR sequences. In such instances, the CDR1 sequence can have an amino acid sequence set forth in any of SEQ ID NOS: 725-741, 778-796, 985, 1090-1091 or 1101-1113; the CDR2 sequence can have an amino acid sequence set forth in any of SEQ ID NOS: 742-759, 797-815, 1092-1096 and 1114-1121; and the third CDR (CDR3) sequence can have an amino acid sequence set forth in any of SEQ ID NOS: 760-777, 816-834, 1097-1100 and 1122-1133. The CDR1 can be the first CDR from the N-terminal of the polypeptide, followed by the CDR2 and then the CDR3.

The polypeptides provided herein can contain a framework sequence with a sequence of amino acids set forth in any of SEQ ID NOS:835-982 and 1134-1205. In some examples, the polypeptide contains two framework sequences, wherein the first framework (framework-1) sequence, from the N-terminus of the polypeptide, has a sequence of amino acids set forth in any of SEQ ID NOS: 835-852, 907-925, 1134-1143 and 1165-1173, and the second framework (framework-2) sequence, from the N-terminus of the polypeptide, has a sequence of amino acids set forth in any of SEQ ID NOS: 853-870, 926-944, 1144-1147, and 1174-1181. Also provided are polypeptides that contain three framework regions. For example, the polypeptide can contain a framework-1 sequence, from the N-terminus of the polypeptide, with a sequence of amino acids set forth in any of SEQ ID NOS: 835-852, 907-925, 1134-1143 and 1165-1173; a framework-2 sequence, from the N-terminus of the polypeptide, with a sequence of amino acids set forth in any of SEQ ID NOS: 853-870, 926-944, 1144-1147, and 1174-1181; and a third framework sequence (framework-3), from the N-terminus of the polypeptide, with a sequence of amino acids set forth in any of SEQ ID NOS: 871-888, 945-963, 1148-1156 and 1182-1193. In further examples, the polypeptide contains a fourth framework sequence (framework-4), from the N-terminus of the polypeptide, having a sequence of amino acids set forth in any of SEQ ID NOS: 889-906, 964-982, 1157-1164, and 1194-1205.

Thus, in some examples, the polypeptide contains three framework sequences and three CDR sequences, wherein the arrangement of the CDR and framework sequences, from the N-terminus of the polypeptide, is framework-1, CDR1, framework-2, CDR2, and framework-3, and wherein the CDR and framework sequences are joined by peptide bonds. In other examples, the polypeptide contains four framework sequences and three CDR sequences, wherein the arrangement of the CDR and framework sequences, from the N-terminus of the polypeptide, is framework-1, CDR1, framework-2, CDR2, framework-3, CDR3, and framework-4, and wherein the CDR and framework sequences are joined by peptide bonds. The first CDR sequence can have a sequence of amino acids set forth in any of SEQ ID NOS: 725-741, 778-796, 985, 1090-1091 and 1101-1113; the second CDR sequence can have a sequence of amino acids set forth in any of SEQ ID NOS: 742-759, 797-815, 1092-1096 and 1114-1121; and the third CDR sequence can have a sequence of amino acids set forth in any of SEQ ID NOS: 725-741, 778-796, 1090-1091 or 1101-1113.

Provided herein are polypeptides that have a sequence of amino acids set forth in any of SEQ ID NOS: 986-1023, 1062-1089 and 1365. In some examples, the polypeptide is an antibody light chain or antibody heavy chain. Thus, provided herein are antibodies or antigen-binding fragments thereof that contain any of the polypeptides described above. In some examples, the antibody or antigen-binding fragment contains a first and a second polypeptide, which can be covalently linked by at least one disulfide bond. The antibody or antigen-binding fragments can be single-chain Fv (scFv), Fab, Fab′, F(ab′)₂, Fv, dsFv, diabody, Fd or Fd′ fragments. In some examples, the antibody or antigen-binding fragment thereof is an IgG, IgA, IgM, IgD or IgE.

In particular examples, the antibody or antigen-binding fragment contain one or more CDR(s) on one polypeptide with a sequence of amino acids set forth in any of SEQ ID NOS: 725-777 and 1090-1091, and one or more CDR(s) on the other polypeptide with a sequence of amino acids set forth in any of SEQ ID NOS: 778-834 and 1101-1113. In one example, the antibody or antigen-binding fragment has a polypeptide with a sequence of amino acids set forth in any of SEQ ID NOS: 986-1004 and 1062-1075 and another polypeptide with a sequence of amino acids set forth in any of 1005-1023, 1076-1089 and 1365. For example, provided are antibodies or antigen-binding fragments wherein one polypeptide has an amino acid sequence set forth in SEQ ID NO: 989, and the other polypeptide has an amino acid sequence set forth in SEQ ID NO: 1008; or one polypeptide has an amino acid sequence set forth in SEQ ID NO: 993, and the other polypeptide has an amino acid sequence set forth in SEQ ID NO: 1012, or one polypeptide has an amino acid sequence set forth in SEQ ID NO: 999, and the other polypeptide has an amino acid sequence set forth in SEQ ID NO: 1018, or one polypeptide has an amino acid sequence set forth in SEQ ID NO: 1004, and the other polypeptide has an amino acid sequence set forth in SEQ ID NO: 1023.

In some aspects, the antibodies or antigen-binding fragments thereof selectively binds to the E1E2 complex of at least one hepatitis C virus. These antibodies can selectively bind to the E1E2 complex of at least one hepatitis C virus and prevent its binding to CD81. In a particular example, the antibody or antigen-binding fragments provided herein selectively binds to an epitope comprising one or more of SEQ ID NOS: 694, 695 and 696. The antibodies or antigen-binding fragments can selectively binds to the E1E2 complex of at least one hepatitis C virus and neutralizes at least one hepatitis C virus, such as HCVpp-H77, HCVpp-J6, or any combination thereof. In some examples, the antibodies neutralizes a hepatitis C virus from genotype 1, 2, 3, 4, 5 or 6 or any subtype thereof, or any combination thereof.

The antibodies or antigen-binding fragments provided herein can prevent or reduce hepatitis C viral replication in a human liver-chimeric mouse when the antibody is administered to the mouse. The antibody can be administered to the mouse before the mouse is infected with hepatitis C virus. The hepatitis C virus can be genotype 1a and the mouse can be infected by intrajugular venous injection of hepatitis C virus. In some examples, the antibodies prevent or reduce hepatitis C viral replication in a human liver-chimeric mouse when administered to the mouse at about 200 mg/kg, about 24 hours before the mouse is infected with the hepatitis C virus. The hepatitis C viral replication can be indicated by the level of hepatitis C viral RNA in the serum of the human liver-chimeric mouse. In some examples, hepatitis C viral replication is reduced by any amount relative to a control human liver-chimeric mouse that has not been administered the antibody. In a particular example, about 200 mg/kg of the antibody is injected intraperitoneally about 24 hours prior to intrajugular injection of about 2×10⁵ copies of genotype 1a hepatitis C viral RNA and prevents hepatitis C viral replication, and wherein prevention of hepatitis C viral replication is indicated by the absence of serum hepatitis C viral RNA determined by quantitative polymerase chain reaction at six days post intrajugular injection of the hepatitis C viral RNA.

Provided are antibody or antigen-binding fragments thereof, wherein the antibody or antigen-binding fragment thereof selectively binds to the hepatitis C virus (HCV) E1E2 complex; the antibody or antigen-binding fragment thereof does not bind to a purified E2 polypeptide that is not in an E1E2 complex; and the antibody or antigen-binding fragment thereof does not bind to a linear epitope on the HCV E1 polypeptide. Such antibodies can neutralize at least one isolate of the hepatitis C virus, such as a plurality of hepatitis C virus isolates, such as at least 2, 3, 4, 5 or 6 isolates of hepatitis C virus. The antibodies can neutralize a hepatitis C virus from genotype 1, 2, 3, 4, 5 or 6 or a subtype thereof, and can neutralize hepatitis C virus from two or more genotypes.

In some examples, the antibody or antigen-binding fragment thereof can compete with an anti-HCV antibody, such as an anti-HCV antibody comprising a heavy chain with a sequence of amino acids set forth in SEQ ID NO:989 and a light chain with a sequence of amino acids set forth in SEQ ID NO:1008; or an anti-HCV antibody comprising a heavy chain with a sequence of amino acids set forth in SEQ ID NO:1004 and a light chain with a sequence of amino acids set forth in SEQ ID NO:1023; wherein at least 70% of the binding of the anti-HCV antibody is inhibited in a competition assay by the antibody or antigen-binding fragment thereof under saturating conditions.

Where the antibody is an antigen-binding fragment thereof, the fragment can be a single-chain Fv (scFv), Fab, Fab′, F(ab′)₂, Fv, dsFv, diabody, Fd, or a Fd′ fragment. The antibody or antigen-binding fragment thereof contain V_{H} CDRs and/or V_{L} CDRs, such as, for example, a variable heavy chain (V_{H}) complementary determining region 1 (CDR1) with a sequence of amino acids set forth in any of SEQ ID NOS:725-741, 983 and 1090-1091; a V_{H} complementary determining region 2 (CDR2) with a sequence of amino acids set forth in any of SEQ ID NOS:742-759 and 1092-1096; a V_{H} complementary determining region 3 (CDR3) with a sequence of amino acids set forth in any of SEQ ID NOS:760-777 and 1094-1110; a variable light chain (V_{L}) CDR1 with a sequence of amino acids set forth in any of SEQ ID NOS:778-796 and 1101-1113; a V_{L} CDR2 with a sequence of amino acids set forth in any of SEQ ID NOS:797-815 and 1114-1121; and/or a V_{L} CDR3 with a sequence of amino acids set forth in any of SEQ ID NOS:816-834 and 1122-1133. Thus, in some aspects, the antibody or antigen-binding fragment thereof contains a heavy chain, wherein the heavy chain comprises a sequence of amino acids set forth in any of SEQ ID NOS:986-1004 or 1062-1075 and/or contains a light chain, wherein the light chain contains a sequence of amino acids set forth in any of SEQ ID NOS:1005-1023 or 1076-1089.

Provided herein are antibodies or antigen-binding fragments thereof that selectively binds to the same epitope as that which is selectively bound by any of the antibodies or antigen-binding fragments thereof described above.

Also provided are compositions, containing an effective amount of the antibody or antigen-binding fragment and a pharmaceutically acceptable carrier. In some examples, the composition also contains an effective amount of at least one additional anti-HCV antibody or antigen-binding fragment, such as any described above. For example, the composition can contain first antibody that selectively binds to an epitope on the hepatitis C virus E2 polypeptide, and a second antibody that selectively binds to the hepatitis C virus E1E2 complex. The epitope on the hepatitis C virus E2 polypeptide can contain three discontinuous amino acid regions of the hepatitis C virus E2 polypeptide, the discontinuous amino acid regions having the sequence: TAGLVGLLTPGAKQNIQLINTNGSWHINS (SEQ ID NO: 694); GWLAGLFYQHKF (SEQ ID NO: 695), and GAPTYSWGANDTDVFVLN (SEQ ID NO: 696); or QLINTNGSWHINS (SEQ ID NO: 1062); GWLAGLFYQHKF (SEQ ID NO: 695), and GAPTYSWGANDTDVFVLN (SEQ ID NO: 696).

The compositions also can contain an antiviral agent, such as an agent used to treat a mammal infected with a human immunodeficiency virus, a biological stabilizer, such as an anti-coagulant, a preservative, a protease inhibitor, or any combination thereof. The anticoagulant can be citrate, ethylene diamine tetraacetic acid, heparin, oxalate, fluoride or any combination thereof; the preservative can be boric acid, sodium formate or sodium borate; and the protease inhibitor can be dipeptidyl peptidase IV inhibitor. The compositions also can contain a sample from the body of a mammal, wherein one antibody in the composition is present in an amount that is at least about 150 mg of antibody per kg of the sample or at least about 0.15 mg/mL of the sample. In some examples, the sample is a blood product, such as plasma, platelet, leukocytes or stem cell.

Provided are methods for detecting hepatitis C virus in a sample. Such methods include the steps of contacting the sample with an antibody or antigen-binding fragment provided herein, and determining whether the antibody binds specifically to the sample, wherein the binding of the antibody to the sample indicates that the sample contains a hepatitis C virus. In some examples, determining is effected by detecting complexes of the antibody with a HCV or envelope protein thereof in the sample. The sample can be from a mammal, such as a body fluid or tissue. In some instances, the mammal is a human. The sample can be a fluid sample.

Also provided are methods for treating or preventing hepatitis C viral infection in a mammal susceptible to infection by a hepatitis C virus, in which an effective amount of an antibody or antigen-binding fragment described above, or a composition described above, is administered to a mammal. In some examples, at least two antibodies or antigen binding fragments thereof are administered. The antibodies can recognize non-overlapping epitopes.

In the methods herein the antibody or composition can be administered prior to exposure to a hepatitis C virus, such as within 3, 7 or 14 days of exposure to a hepatitis C virus. In other examples, the antibody or composition is administered after exposure to a hepatitis C virus. The antibody or composition administered to the mammal can be administered by intravenous or intraparenteral injection. Typically, the mammal is a human. The methods also can include administering to the mammal a second dose of the antibody or composition at a selected time after the first administration of the antibody or composition. In some examples, the mammal has recurrent hepatitis C viral infection, and/or the mammal is a liver transplantee. In the latter instances, the antibody or composition can be administered at one or more select time points after the transplant, or before and after the transplant. In some examples, the antibody is administered daily for seven days after the transplant; administered every 2 weeks from weeks 2-12 after the transplant, or is administered monthly from twelve weeks after the transplant and onwards. Further, the liver transplant can be infused with the antibody or composition.

In particular examples, the human also is infected with a human immunodeficiency virus. In such examples, the methods also can contain a step of administering an agent used to treat infection by a human immunodeficiency virus.

Provided are uses of an antibody or antigen-binding fragment thereof described above or a composition described above, in the preparation of a medicament for treatment or prevention of hepatitis C virus infection. The antibodies or antigen-binding fragments thereof or compositions provided herein can be used to treat or prevent hepatitis C virus infection. In some examples, the hepatitis C virus infection is a recurrent infection. In other examples, the hepatitis C virus infection is a new infection. Further, the subject can be a liver transplantee.

Provided are articles containing an antibody or antigen-binding fragment thereof or a composition described above and instructions for use of the antibody or composition to prevent or treat infection of a mammal or mammalian cell with a hepatitis C virus. The article can include a vessel for collecting a body fluid. In some examples, at least two antibodies or antigen-binding fragments thereof are included. Such antibodies can recognize non-overlapping epitopes. In some examples, at least one of the antibodies recognizes a conformational epitope of the hepatitis C viral E2 protein that comprises amino acids 396-424; amino acids 436-447, and amino acids 523-540, such as one where the sequence of amino acids 396-424 is TAGLVGLLTPGAKQNIQLINTNGSWHINS (SEQ ID NO: 694); the sequence of amino acids 436-447 is GWLAGLFYQHKF (SEQ ID NO: 695), and the sequence of amino acids 523-540 is GAPTYSWGANDTDVFVLN (SEQ ID NO: 696). In some aspects, at least one of the antibodies recognizes a conformational epitope of the hepatitis C viral E2 protein that comprises amino acids 412-424; amino acids 436-447, and amino acids 523-540. In some examples, the sequence of amino acids 412-424 is QLINTNGSWHINS (SEQ ID NO: 1062); the sequence of amino acids 436-447 is GWLAGLFYQHKF (SEQ ID NO: 695), and the sequence of amino acids 523-540 is GAPTYSWGANDTDVFVLN (SEQ ID NO: 696).

The vessel can be a collection bag, tube, capillary tube or syringe, and can be evacuated. The article further can contain a biological stabilizer, such as an anti-coagulant, preservative, protease inhibitor, or any combination thereof. The anti-coagulant can be citrate, ethylene diamine tetraacetic acid, heparin, oxalate, fluoride or any combination thereof; the preservative can be boric acid, sodium formate or sodium borate; and the protease inhibitor can be dipeptidyl peptidase IV inhibitor. In some examples, the antibody and/or stabilizer is freeze dried.

Provided are isolated nucleic acid molecules that encodes a polypeptide or antibody or antigen-binding fragment described above. In some examples, the isolated nucleic molecules contain a sequence of nucleic acids set forth in any of SEQ ID NOS: 1024-1042 or 1043-1061. The nucleic acid molecules can be operably linked to an expression control sequence, such as a viral, phage, bacterial, or mammalian promoter. Also provided are expression vectors containing the nucleic acid molecule, and cells containing the expression vector. In some examples, the cell is a bacterial or mammalian cell, such as Chinese hamster ovary cell or 293 cell.

Provided are modified E1 polypeptides containing a modification in an E1 polypeptide or variant thereof, wherein the modification is at a position corresponding to position 293, 295, 314, or 316 in an E1 polypeptide with a sequence set forth in SEQ ID NO:1377, with numbering corresponding to the HCV polyprotein set forth in SEQ ID NO:607, or in corresponding residues in a E1 polypeptide. The modification can be an amino acid replacement with any amino acid. In some examples, the modification is selected from among F293A, P295A, T314A and H316A. The modified E1 polypeptides can further contain a further modification at another position in the E1 polypeptide. For example, the modified E1 polypeptide can contain 2, 3, 4, 5, 6 or more modifications. In some examples, the unmodified E1 polypeptide has a sequence of amino acids set forth in any of SEQ ID NOS:1377-1361. In further examples, the modified E1 polypeptide has a sequence of amino acids set forth in any of SEQ ID NOS:1206-1285.

Provided are E1E2 complexes, containing a modified E1 polypeptide described herein. Also provided are hepatitis C viruses, containing any of the modified E1 polypeptides provided herein. In some examples, such the infectivity of the virus is increased compared to the infectivity of a hepatitis C virus that contains an unmodified E1 polypeptide. The hepatitis C virus can be used, for example, in an assay. Thus, also provided are assays, containing the steps of a) contacting the hepatitis C virus of any of claims 147-149 with an anti-viral agent; and b) contacting cells with the hepatitis C virus.

Typically, the infectivity f the hepatitis C virus is assessed, an the neutralization of the hepatitis C virus by the anti-viral agent is assessed. The anti-viral agent can be a polypeptide, a peptide, an anti-sense molecule and a small molecule. In some examples, the anti-viral agent is an anti-HCV antibody or fragment thereof. In the assays provided herein, step a) can be performed before step b); step b) can be performed before step a); or steps a) and b) can performed simultaneously. Typically, the cell is a mammalian cell.

Also provided are nucleic acid molecules that encodes the modified E1 polypeptide; vectors that contain the nucleic acid molecule; and cells that contain the vector. In some examples, the cell is a bacterial cell, a yeast cell or a eukaryotic cell, such as a mammalian cell, such as one selected from among baby hamster kidney cells (BHK-21), 293 cells, Huh7 cells and CHO cells.

Any feature or combination of features described herein are included within the scope provided that the features included in any such combination are not mutually inconsistent, as will be apparent from the context, this specification and the knowledge of one of ordinary skill in the art. Other features and advantages will be apparent from the following detailed description.

### BRIEF DESCRIPTION OF THE FIGURES

FIG. 1A-C illustrate properties of anti-HCV E2 Fabs isolated as described herein. FIG. 1A illustrates the specificity of anti-E2 Fabs. The binding of Fabs to GST-E1E2 complex and E2 is compared. The GST-E1E2 fusion protein was captured by a goat anti-GST antibody while soluble E2 and ovalbumin were coated directly onto ELISA plates. Fabs were added to the antigens and subsequently detected with phosphatase-conjugated goat anti-human F(ab)′2 IgG. Recombinant Fabs were produced in cleared lysate of E. coli transformed with the corresponding phagemids. FIG. 1B illustrates competition between MAb AR3A and anti-E2 Fabs. Vaccinia-expressed E1E2 was captured onto ELISA wells by lectin and preincubated with saturating concentration of soluble Fabs before the addition of MAb AR3A. Binding of MAb AR3A was detected with a goat anti-human IgG Fc antibody and the % reduction of binding compared to that in the absence of a Fab is shown. Lightly-shaded bars indicate that Fabs bind E2 better than E1E2; while bars of medium shading indicate that Fabs bind E1E2 better than E2. FIG. 1C illustrates the inhibition of anti-E2 Fab binding to E1E2 by mouse. MAb H53. E1E2 was captured onto ELISA wells in the same manner as shown for FIG. 1B and was pre-incubated with a saturating concentration of MAb H53 before the addition of soluble Fabs. Binding of Fabs was detected with a goat anti-human IgG F(ab)′2 antibody and the % reduction of binding compared to that without MAb H53 is shown. Lightly-shaded bars indicate that Fabs bind E2 better than E1E2; while bars of medium shading indicate that Fabs bind E1E2 better than E2.

FIG. 2 shows neutralization of HCVpp by human Fabs. Infectivity in Relative Light Units (RLU) is shown for infection of pseudotype virus particles generated with viral Env gene from murine leukemia virus (MLV) (FIG. 2A), H77 (GT 1a) (FIG. 2B), OH8 (GT 1b) (FIG. 2C), CON1 (GT 1b) (FIG. 2D) or J6 (GT 2a) (FIG. 2E) in the presence of 10 μg/mL Fabs. AR1-Fabs: B2, D1 & E; AR2-Fabs: F & G; AR3-Fabs: C1, J2, J3 & L4. Control, anti-HIV gp120 Fab b12; Empty, background infectivity from pseudotype virus generated without Env gene. Dotted lines indicate HCVpp infectivity in the absence of any antibody. Error bars represent SEM calculated from three experiments performed in the same manner.

FIG. 3 is a schematic diagram of E2 regions important for binding of AR3-specific antibodies. E2 (residues 384-746) is a transmembrane glycoprotein, and a truncated form of E2 (residues 384-661) can be expressed as a soluble protein that retains its ability to bind cell lines expressing HCV receptors and CD81-LEL (Michalak, J. P. et al. J. Gen. Virol. 78, 2299-2306 (1997)). The regions that were investigated by antibody competition (light boxes) and alanine mutagenesis (dark boxes) form the AR3 discontinuous epitopes (dotted boxes). The dotted boxes correspond to (1) amino acids 396-424 having the sequence TAGLVGLLTPGAKQNIQLINTNGSWHINS (SEQ ID NO: 694), (2) amino acids 436-447 having the sequence GWLAGLFYQHKF (SEQ ID NO: 695), and (3) amino acids 523-540 having the sequence GAPTYSWGANDTDVFVLN (SEQ ID NO: 696). The crucial residues in these regions are S424, G523, P525, G530, D535, V538 and N540. The locations of the N-linked glycans are indicated by branched forks. The hypervariable regions (see Troesch, M. et al. Virology 352, 357-367 (2006)) and the transmembrane regions are indicated by the designation HVRs and TM.

FIG. 4 illustrates the levels of human MAb in human liver-chimeric mice 24 hours post-administration. Human liver-chimeric mice (n=6) were injected intraperitoneally with a dose of 200 mg/kg of the isotype control mAb b6, AR3A or AR3B and blood samples were collected at 24 hours before challenging with a genotype 1a HCV-infected human serum KP (100 μL) by intrajugular venous injection. Intravenous administration of human serum is the most reliable way to assure delivery of human serum but a stressful procedure: 5 of 18 treated mice did not recover after the procedure. Human IgG in mouse sera were quantified as in FIG. 4. Filled bars, mice that died after intrajugular injection of KP serum; Open bars, mice that survived the procedure and were used in the protection experiments. The mean serum human IgG levels±s.d. in the surviving mice of group b6, AR3A and AR3B are 2.5±0.3, 3.1±0.5 and 2.6±0.3 mg/mL, respectively. Note that decay of the human MAbs following virus challenge, which may be an explanation for the infection of several antibody-treated mice at later time points, could not be determined as in FIG. 5 because the infected human serum contains normal human antibodies interfering with IgG quantification. In a control experiment using transplanted mice with low levels of human chimerism, human IgG antibodies were readily detected in the mice challenged with 100 μL of the infected serum (n=5, Day 1 mean±s.d. mouse serum human IgG concentration: 2.6±0.5 mg/mL) (data not shown).

FIG. 5 demonstrates passive antibody protection against an HCV population. Human liver-chimeric mice (n=6) injected intraperitoneally with 200 mg/kg of the isotype control mAb b6 (left), mAb AR3A (middle) or AR3B (right), were challenged 24 hours later by intrajugular venous injection of genotype 1a HCV-infected human serum (˜2×10⁵ HCV RNA copies). One or two mice per group did not recover from anesthesia after intrajugular injection. Data shown are serum viral load in mice quantified by real-time TaqMan PCR. Owing to morbidity, mice N680 and N672 were killed on days 41 and 45, respectively. IU, international units; ID, identification number; i.p., intraperitoneally; i.v., intravenously. Results indicate the absence of serum HCV RNA 6 days after viral challenge in mice injected with mAb AR3A and mAb AR3B.

FIG. 6 is a sequence comparison of a viral quasispecies population in the HCV genotype 1a-infected human serum. Partial E2 amino acid sequences (residues 384-622) of a total of 40 clones (represented by KP S9 (SEQ ID NO: 701), KP R14 (SEQ ID NO: 702), KP S6 (SEQ ID NO: 703), KP S18 (SEQ ID NO: 704), KP S16 (SEQ ID NO: 705), KP R8 (SEQ ID NO: 706), KP S20 (SEQ ID NO: 707), KP S4 (SEQ ID NO: 708), KP R3 (SEQ ID NO: 709), KP S3 (SEQ ID NO: 710), KP S12 (SEQ ID NO: 711), KP S15 (SEQ ID NO: 712), KP S5 (SEQ ID NO: 713), KP R7 (SEQ ID NO: 714), KP R11 (SEQ ID NO: 715), KP R1 (SEQ ID NO: 716), KP R12 (SEQ ID NO: 717), KP S7 (SEQ ID NO: 718), KP R15 (SEQ ID NO: 719), KP R18 (SEQ ID NO: 720), KP S11 (SEQ ID NO: 721) and KP R20 (SEQ ID NO: 722)) randomly selected from two independent RT-PCR cloning are shown. The top sequence, clone KP S9, represents the consensus and dominant sequence in this infectious serum. The periods indicate regions of amino acid sequence identity. The frequency of each clone is bracketed. Hypervariable regions (HVRs) are within the dashed-line boxes. Regions important for binding of AR3-antibodies are within the solid-line boxes. The corresponding sequences of isolates H77 (SEQ ID NO: 723) and UKN1b12.16 (SEQ ID NO: 724), sharing 87% and 75% amino acid identity with KP S9, respectively, are shown for comparison.

FIG. 7 illustrates the results of an analysis of the binding of full-length IgGs of anti-HCV E1E2 antibodies to E1E2 complexes containing E1 polypeptides with N196A, N209A, N234A, N305A or N325A mutations.

FIG. 8 illustrates the sensitivity of HCVpp containing the E1 mutations F293A, P295A T314A or H316A to neutralization by anti-HCV antibodies C1 IgG, G IgG and N4 IgG. Den3 IgG is an isotype control antibody specific for Dengue virus NS2.

### DETAILED DESCRIPTION

### Outline

A. Definitions

B. Hepatitis C Virus
E1 and E2

C. Anti-HCV Polypeptides and Antibodies
1. Polypeptides
2. Anti-HCV antibodies
   a. General structure of antibodies
      i. Structural and Functional Domains of Antibodies
      ii. Antibody Fragments
   b. Exemplary Anti-HCV antibodies
   c. Additional modifications of anti-HCV antibodies
      i. Modifications to reduce immunogenicity
      ii. Fc modifications
      iii. Pegylation
      iv. Conjugation of a detectable moiety
      v. Conjugation of a therapeutic moiety
      vi. Modifications to improve binding specificity
3. Methods For Producing Anti-HCV Polypeptides and antibodies
   a. Nucleic acids
   b. Vectors
   c. Cell Expression Systems
      i. Prokaryotic Expression
      ii. Yeast Cells
      iii. Insect cells
      iv. Mammalian cells
      v. Plants
   d. Purification
   e. Additional methods for producing antibodies
4. Assessing anti-HCV antibodies
   a. Binding assays
   b. In vitro assays for analyzing virus neutralization effects of antibodies
   c. In vivo Animal models
5. Uses for anti-HCV Polypeptides and Antibodies
6. Pharmaceutical Compositions
7. Miscellaneous Compositions and Articles of Manufacture

D. Modified E1 and E2 polypeptide
1. Exemplary E1 modifications
2. Exemplary E2 modifications
3. Production of the modified E1 and E2 polypeptides
   a. Nucleic acids encoding modified E1 and/or E2
   b. Vectors and Expression systems
   c. Purification and assessment
4. Uses for the modified E1 and E2 polypeptides.
5. Articles of manufacture and kits

E. EXAMPLES

### DEFINITIONS

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

As used herein, the term “hepatitis C virus,” “HCV,” or “HCVs” includes different viral genotypes, subtypes, quasispecies and isolates. It includes any noncytopathic RNA virus that has a single and positive-stranded RNA genome belonging to the Hepacivirus genus of the Flaviviridae family. The term includes different isolates of HCV such as, without limitation, those having polyprotein sequences and accession numbers shown above, as well as any others in the NCBI database. Examples of different genotypes encompassed by this term include, without limitation, genotype 1, 2, 3, 4, 5 and 6, as described in Simmonds et al. (Hepatology 42:962-973). Reference to HCV also includes those of any additional genotypes that are established. Examples of different subtypes include, without limitation, 1a, 1b, 1c, 2a, 2b, 2c, 2i, 2k, 3a, 3b, 3k, 4a, 4d, 4f, 5a, 6a, 6b, 6c, 6d, 6e, 6f, 6g, 6h, 6i, 6j, 6k, 6l, 6m, 6n, 6o, 6q, 6p and 6t. The term also includes cell culture HCVs (HCVcc) and pseudotype HCVs (HCVpp), as well as HCV quasispecies. Various HCVs are described by Simmonds P. in Genetic diversity and evolution of hepatitis C virus—15 years on, J Gen Virol 85:3173-3188 (2004) and Simmonds et al. in Consensus proposals for a unified system of nomenclature of hepatitis C virus genotypes, Hepatology 42:962-973 (2005). HCV nucleotide sequences are known in the art. For example, see Viral Bioinfomatics Research Center (hcvdb.org) and the Hepatitis C Virus database (hcv.lanl.gov/).

As used herein, the term “polypeptide” refers to a polymer composed of three or more naturally-occurring or non-naturally occurring amino acid residues of the L or D configuration, regardless of post-translational modification. The terms “polypeptide” and “protein” are used interchangeably herein.

As used herein, a “peptide” refers to a polypeptide that is from 2 to about or 40 amino acids in length.

As used herein, an “amino acid” is an organic compound containing an amino group and a carboxylic acid group. A polypeptide contains two or more amino acids. For purposes herein, amino acids contained in the antibodies provided include the twenty naturally-occurring amino acids (Table 1), non-natural amino acids, and amino acid analogs (e.g., amino acids wherein the α-carbon has a side chain). As used herein, the amino acids, which occur in the various amino acid sequences of polypeptides appearing herein, are identified according to their well-known, three-letter or one-letter abbreviations (see Table 1). The nucleotides, which occur in the various nucleic acid molecules and fragments, are designated with the standard single-letter designations used routinely in the art.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3557-59 (1968) and adopted at 37 C.F.R. §§1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glutamic Acid and/or |
| | | Glutamine |
| W | Trp | Tryptophan |
| R | Arg | Arginine |
| D | Asp | Aspartic acid |
| N | Asn | Asparagine |
| B | Asx | Aspartic Acid and/or |
| | | Asparagine |
| C | Cys | Cysteine |
| X | Xaa | Unknown or other |

All sequences of amino acid residues represented herein by a formula have a left to right orientation in the conventional direction of amino-terminus to carboxyl-terminus. In addition, the phrase “amino acid residue” is defined to include the amino acids listed in the Table of Correspondence (Table 1), modified, non-natural and unusual amino acids. Furthermore, a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al., Molecular Biology of the Gene, 4th Edition, 1987, The Benjamin/Cummings Pub. co., p. 224).

Such substitutions can be made in accordance with those set forth in Table 2 as follows:

TABLE 2

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative or non-conservative substitutions.

As used herein, “naturally occurring amino acids” refer to the 20 L-amino acids that occur in polypeptides.

As used herein, the term “non-natural amino acid” refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally occurring amino acids and include, but are not limited to, the D-isostereomers of amino acids. Exemplary non-natural amino acids are known to those of skill in the art, and include, but are not limited to, 2-Aminoadipic acid (Aad), 3-Aminoadipic acid (Baad), β-alanine/β-Amino-propionic acid (Bala), 2-Aminobutyric acid (Abu), 4-Aminobutyric acid/piperidinic acid (4Abu), 6-Aminocaproic acid (Acp), 2-Aminoheptanoic acid (Ahe), 2-Aminoisobutyric acid (Aib), 3-Aminoisobutyric acid (Baib), 2-Aminopimelic acid (Apm), 2,4-Diaminobutyric acid (Dbu), Desmosine (Des), 2,2′-Diaminopimelic acid (Dpm), 2,3-Diaminopropionic acid (Dpr), N-Ethylglycine (EtGly), N-Ethylasparagine (EtAsn), Hydroxylysine (Hyl), allo-Hydroxylysine (Ahyl), 3-Hydroxyproline (3Hyp), 4-Hydroxyproline (4Hyp), Isodesmosine (Ide), allo-Isoleucine (Aile), N-Methylglycine, sarcosine (MeGly), N-Methylisoleucine (MeIle), 6-N-Methyllysine (MeLys), N-Methylvaline (MeVal), Norvaline (Nva), Norleucine (Nle), Ornithine (Orn).

As used herein, “antibody” refers to a full-length immunoglobulin molecule or an immunologically-active fragment of an immunoglobulin molecule such as the Fab or F(ab′)₂ fragment generated by, for example, cleavage of the antibody with an enzyme such as pepsin or co-expression of an antibody light chain and an antibody heavy chain in bacteria, yeast, insect cell or mammalian cell. The antibodies provided herein can be generated using one or more polypeptides provided herein that form a structure resembling a Fab, bivalent F(ab′)₂, IgG, IgD, IgA, IgE or IgM and is capable of selectively binding to HCV or an HCV antigen. Typically, the antibodies provided herein are anti-HCV antibodies that immunoreacts with epitopes on the E2 glycoprotein or the E1E2 complex of HCV, i.e. selectively binds with these epitopes on the HCV. In some instances, the anti-HCV antibodies neutralize the virus.

The term “antibody” is used in the broadest sense, and specifically covers monoclonal antibodies (including full length monoclonal antibodies), polyclonal antibodies, multispecific antibodies (e.g., bispecific antibodies), and antibody fragments (e.g., Fab, F(ab′)2, Fd and Fv) so long as they exhibit the desired biological activity.

The term “variable” in the context of variable region of antibodies, refers to the fact that certain portions of the variable regions differ extensively in sequence among antibodies and are used in the binding and specificity of each particular antibody for its particular antigen. The variability is concentrated in three segments (a triplet) called complementarity determining regions (CDRs) also known as hypervariable regions both in the light chain and the heavy chain variable regions. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342: 877).

The more highly conserved portions of variable regions are called the framework (FR). The variable domains of native heavy and light chains each comprise three FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies (see Kabat et al.) The constant regions are not involved directly in binding an antibody to an antigen, but exhibit various effector function, such as participation of the antibody in antibody-dependent cellular toxicity.

A “species-dependent antibody,” e.g., a mammalian anti-human IgE antibody, is an antibody which has a stronger binding affinity for an antigen from a first mammalian species than it has for a homologue of that antigen from a second mammalian species. Normally, the species-dependent antibody “bind specifically” to a human antigen (i.e., has a binding affinity (Kd) value of no more than about 1×10⁻⁷ M, preferably no more than about 1×10⁻⁸ and most preferably no more than about 1×10⁻⁹ M) but has a binding affinity for a homologue of the antigen from a second non-human mammalian species which is at least about 50 fold, or at least about 500 fold, or at least about 1000 fold, weaker than its binding affinity for the human antigen. The species-dependent antibody can be of any of the various types of antibodies as defined above, but preferably is a humanized or human antibody.

The term “antibody variation” refers to an amino acid sequence variant of an antibody wherein one or more of the amino acid residues have been modified. Such mutant necessarily have less than 100% sequence identity or similarity with the amino acid sequence having at least 75% amino acid sequence identity or similarity with the amino acid sequence of either the heavy or light chain variable domain of the antibody, more preferably at least 80%, more preferably at least 85%, more preferably at least 90%, and most preferably at least 95%.

The term “antibody fragment” refers to a portion of a full-length antibody, generally the antigen binding or variable region. Examples of antibody fragments include Fab, Fab′, F(ab′)2, Fd and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)2 fragment that has two antigen binding fragments which are capable of crosslinking antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, refers to Fv, F(ab) and F(ab′)2 and Fd fragments.

As used herein, an antigen-binding fragment refers to an antibody fragment that contains an antigen-binding portion that binds to the same antigen as the antibody from which the antibody fragment is derived. An antigen-binding fragment, as used herein, includes any antibody fragment that when inserted into an antibody framework (such as by replacing a corresponding region) results in an antibody that immunospecifically binds (i.e. exhibits Ka of at least or at least about 10⁷-10⁸ M⁻¹) to the antigen. Antigen-binding fragments include, antibody fragments, such as Fab fragments, Fab′ fragments, F(ab′)2 fragments, Fv fragments, disulfide-linked Fvs (dsFv), Fd fragments, Fd′ fragments, single-chain Fvs (scFv), single-chain Fabs (scFab), and also includes other fragments, such as CDR-containing fragments, and polypeptides that immunospecifically bind to an antigen or that when inserted into an antibody framework results in an antibody that immunospecifically binds to the antigen.

An “Fv” fragment is the minimum antibody fragment which contains a complete antigen recognition and binding site. This region consists of a dimer of one heavy and one light chain variable domain in a tight, non-covalent association (VH-VL dimer). It is in this configuration that the three CDRs of each variable region interact to define an antigen binding site on the surface of the VH-VL dimer. Collectively, the six CDRs confer antigen binding specificity to the antibody. However, even a single variable region (or half of an Fv comprising only three CDRs specific for an antigen) has the ability to recognize and bind antigen, although at a lower affinity than the entire binding site.

The Fab fragment (also designated as F(ab)) also contains the constant region of the light chain and the first constant region (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant regions have a free thiol group. F(ab′) fragments are produced by cleavage of the disulfide bond at the hinge cysteines of the F(ab′)2 pepsin digestion product. Additional chemical couplings of antibody fragments are known to those of ordinary skill in the art.

The light chains of antibodies (immunoglobulin) from any vertebrate species can be assigned to one of two clearly distinct types, called kappa (κ) and lambda (λ), based on the amino acid sequences of their constant domain.

Depending on the amino acid sequences of the constant domain of their heavy chains, “immunoglobulins” can be assigned to different classes. There are at least five (5) major classes of immunoglobulins: IgA, IgD, IgE, IgG and IgM, and several of these may be further divided into subclasses (isotypes), e.g. IgG-1, IgG-2, IgG-3 and IgG4; IgA-1 and IgA-2. The heavy chains constant domains that correspond to the different classes of immunoglobulins are called α, δ, ε, γ and μ, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies composed of the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In additional to their specificity, the monoclonal antibodies are advantageous in that they are synthesized by hybridoma or phage infected bacterial culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, Nature 256, 495 (1975), or may be made by recombinant methods, e.g., as described in U.S. Pat. No. 4,816,567. The monoclonal antibodies also can be isolated from phage antibody libraries using the techniques described in Clackson et al. Nature 352: 624-628 (1991), as well as in Marks et al., J. Mol. Biol. 222: 581-597 (1991).

The antibody subclasses including monoclonal antibodies, fragments and single chains thereof include “chimeric” forms in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567); Morrison et al. Proc. Natl. Acad. Sci. 81, 6851-6855 (1984).

The antibody subclasses also include fully human forms in which the entire sequence is derived from human immunoglobulins (recipient antibody) including the complementary determining region (CDR) of the polypeptide In some instances, Fv framework residues of the human immunoglobulin are replaced by corresponding non-human residues. Furthermore, a polypeptide can include residues which are found neither in a human immunoglobulin nor in a non-human mammalian sequence.

“Single-chain Fv” or “scFv” antibody fragments include the VH and VL regions of an antibody, wherein these regions are present in a single polypeptide chain. Generally, the Fv polypeptide further includes an polypeptide linker between the VH and VL regions which enables the scFv to form the desired structure for antigen binding. For a review of sFv see Pluckthun in The Pharmacology of Monoclonal Antibodies, vol. 113, Rosenburg and Moore eds. Springer-Verlag, New York, pp. 269-315 (1994).

The term “diabodies” refers to a small antibody fragments with two antigen-binding sites, which fragments comprise a heavy chain variable region (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Holliger et al., Proc. Natl. Acad. Sci. USA 90: 6444-6448 (1993).

The phrase “functional fragment or analog” of an antibody is a compound having qualitative biological activity in common with a full-length antibody. For example, a functional fragment or analog of an anti-IgG antibody is one which can bind to an IgG immunoglobulin in such a manner so as to prevent or substantially reduce the ability of such molecule from having the ability to bind to the high affinity receptor, Fc gamma receptor.

The term “variants” refers to substitutional, insertional and/or deletional variants. “Substitutional” variants are those that have at least one amino acid residue in a native sequence removed and a different amino acid inserted in its place at the same position. The substitutions may be single, where only one amino acid in the molecule as been substituted, or they may be multiple, where two or more amino acids have been substituted in the same molecule. “Insertional” variants are those with one or more amino acids inserted immediately adjacent to an amino acid at a particular position in a native sequence. Immediately adjacent to an amino acid means connected to either the alpha-carboxyl or alpha-amino functional group of the amino acid. “Deletional” variants are those with one or more amino acids in the native amino acid sequence removed. Ordinarily, deletional variants will have one or two amino acids deleted in a particular region of the molecule.

As used herein, the term “bind selectively” or “selectively binds,” in reference to a polypeptide or an antibody provided herein, means that the polypeptide or antibody binds with a selected epitope without substantially binding to another epitope. Typically, an antibody or fragment thereof that selectively binds to a selected epitope specifically binds to the epitope, such as with an affinity constant Ka of about or 1×10⁷M⁻¹ or 1×10⁸M⁻¹ or greater, as defined below.

As used herein, “specifically binds” or “immunospecifically binds,” with respect to an antibody or antigen-binding fragment thereof are used interchangeably herein and refer to the ability of the antibody or antigen-binding fragment to form one or more noncovalent bonds with a cognate antigen, by noncovalent interactions between the antibody combining site(s) of the antibody and the antigen. The antigen can be an isolated antigen or presented in a virus. Typically, an antibody that specifically binds to a virus antigen or virus is one that binds to the virus antigen (or to the antigen in the virus or to the virus) with an affinity constant Ka of about or 1×10⁷M⁻¹ or 1×10⁸M⁻¹ or greater (or a dissociation constant (IQ) of 1×10⁻⁷ M or 1×10⁻⁸ M or less). Affinity constants can be determined by standard kinetic methodology for antibody reactions, for example, immunoassays, surface plasmon resonance (SPR) (Rich and Myszka (2000) Curr. Opin. Biotechnol 11:54; Englebienne (1998) Analyst. 123:1599), isothermal titration calorimetry (ITC) or other kinetic interaction assays known in the art (see, e.g., Paul, ed., Fundamental Immunology, 2nd ed., Raven Press, New York, pages 332-336 (1989); see also U.S. Pat. No. 7,229,619 for a description of exemplary SPR and ITC methods for calculating the binding affinity of anti-HCV antibodies). Instrumentation and methods for real time detection and monitoring of binding rates are known and are commercially available (e.g., BiaCore 2000, Biacore AB, Upsala, Sweden and GE Healthcare Life Sciences; Malmqvist (2000) Biochem. Soc. Trans. 27:335). An antibody that specifically binds to a virus antigen (or virus) can bind to other peptides, polypeptides, or proteins or viruses with equal or lower binding affinity. Typically, an antibody or antigen-binding fragment thereof provided herein that binds specifically to a HCV E2 protein does not cross-react with other antigens or cross reacts with substantially (at least 10-100 fold) lower affinity for such antigens. Similarly, an antibody or antigen-binding fragment thereof provided herein that binds specifically to a HCV E1E2 complex does not cross-react with other antigens or cross reacts with substantially (at least 10-100 fold) lower affinity for such antigens. An antibody or antigen-binding fragment thereof provided herein that binds selectively to E2 typically also binds to the E1E2 complex. Antibodies or antigen-binding fragments that selectively binds to a particular virus antigen (e.g. a HCV E2 or E1E2) can be identified, for example, by immunoassays, such as radioimmunoassays (RIA), enzyme-linked immunosorbent assays (ELISAs), surface plasmon resonance, or other techniques known to those of skill in the art. An antibody or antigen-binding fragment thereof that specifically binds to an epitope on a HCV E2 or E1E2 typically is one that binds to the epitope (presented in the protein or virus) with a higher binding affinity than to any cross-reactive epitope as determined using experimental techniques, such as, but not limited to, immunoassays, surface plasmon resonance, or other techniques known to those of skill in the art. Specifically binding to an isolated HCV protein (i.e., a recombinantly produced protein), such as HCV E1E2, does not necessarily mean that the antibody will exhibit the same immunospecific binding and/or neutralization of the virus. Such measurements and properties are distinct. The affinity for the antibody or antigen-binding fragments for the virus or the antigen as presented in the virus can be determined. For purposes herein, when describing an affinity or related term, the target, such as the isolated protein or the virus, will be identified.

As used herein, the term “epitope” refers to any antigenic determinant on an antigen to which the paratope of an antibody binds, and is made up of one or more segments of amino acids. An epitope can be a linear or conformational epitope, and can be continuous or discontinuous. Typically, linear epitopes are continuous, i.e. made up of one continuous stretch of amino acids. Conformational epitopes can be discontinuous i.e. made up of two or more discontinuous segments of amino acids that come together to form an epitope when the antigen is folded. Methods for determining whether antibodies binds to the same epitope are known in the art. Epitopes can be defined or mapped by standard methods well known in art. For example, epitopes can be mapped using assays, such as ELISA assays, utilizing peptide libraries or site-directed mutagenesis of the antigen (such as alanine-scanning of the antigen).

As used herein, “binds to the same epitope” with reference to two or more antibodies means that the antibodies compete for binding to an antigen and bind to the same, overlapping or encompassing continuous or discontinuous segments of amino acids. Those of skill in the art understand that the phrase “binds to the same epitope” does not necessarily mean that the antibodies bind to exactly the same amino acids. The precise amino acids to which the antibodies bind can differ. For example, a first antibody can bind to a segment of amino acids that is completely encompassed by the segment of amino acids bound by a second antibody. In another example, a first antibody binds one or more segments of amino acids that significantly overlap the one or more segments bound by the second antibody. For the purposes herein, such antibodies are considered to “bind to the same epitope.”

Antibody competition assays can be used to determine whether an antibody “binds to the same epitope” as another antibody. Such assays are well known in the art and are described herein (see. e.g. Examples 1-4). Typically, competition of 70% or more, such as 70%, 71%, 72%, 73%, 74%, 75%, 80%, 85%, 90%, 95% or more, of an antibody known to interact with the epitope by a second antibody under conditions in which the second antibody is in excess and the first saturates all sites, is indicative that the antibodies “bind to the same epitope.” To assess the level of competition between two antibodies, for example, radioimmunoassays or assays using other labels for the antibodies, such as biotin (see, e.g., Example 1) can be used. For example, an HCV antigen, such as the E1E2 complex, can be incubated with a saturating amount of a first anti-HCV antibody or antigen-binding fragment thereof conjugated to a labeled compound (e.g., ³H, ¹²⁵I or biotin) in the presence of the same amount of a second unlabeled anti-HCV antibody. The amount of labeled antibody that is bound to the antigen in the presence of the unlabeled blocking antibody is then assessed and compared to binding in the absence of the unlabeled blocking antibody. Competition is determined by the percentage change in binding signals in the presence of the unlabeled blocking antibody compared to the absence of the blocking antibody. Thus, if there is a 70% inhibition of binding of the labeled antibody in the presence of the blocking antibody compared to binding in the absence of the blocking antibody, then there is competition between the two antibodies of 70%. Thus, reference to competition between a first and second antibody of 70% or more, such as 70%, 71%, 72%, 73%, 74%, 75%, 80%, 85%, 90%, 95% or more, means that the first antibody inhibits binding of the second antibody (or vice versa) to the antigen by 70%, 71%, 72%, 73%, 74%, 75%, 80%, 85%, 90%, 95% or more (compared to binding of the antigen by the second antibody in the absence of the first antibody). Thus, inhibition of binding of a first antibody to an antigen by a second antibody of 70%, 71%, 72%, 73%, 74%, 75%, 80%, 85%, 90%, 95% or more indicates that the two antibodies bind to the same epitope.

The term “neutralize,” as used herein in reference to a polypeptide or antibody described herein, means that the polypeptide or antibody can prevent or reduce HCV infection or replication. Neutralization can be assessed using methods well known in art and described herein, such as the neutralization of binding assays described in the examples, below, and by assessing HCV replication in in vivo animal models following administration of the polypeptide or antibody being tested. In some examples, binding or replication is reduced by 10%, 20%, 30%, 40%, 50%, 60%, 70%, 80%, 90%, 95%, 96%, 97% or more.

The term “reduce,” as used herein in reference to HCV infection or replication, means a decrease in the amount of HCV ribose nucleic acids (RNA) in a sample. HCV infection or replication is indicated by the amount of HCV RNA detected in a sample from a source (cell culture or animal) that has been exposed to HCV and is susceptible to HCV infection. Whether a polypeptide or antibody will bind selectively to HCV and neutralize it can be determined using methods known in the art, as well as the methods described herein, including determining the level of HCV RNA or detecting reduction of signals from a reporter gene encoded by the virus such as, for example, the relative light unit (RLU) for luciferase or the mean fluorescence intensity (MFI) of green fluorescent protein (GFP).

As used herein, the term “prevent,” “preventing” or “prevention” refers to use in a prophylactic manner, i.e. prevention of the occurrence of symptoms and/or their underlying cause. The terms “treat,” “treating” and “treatment,” as used herein, refer to reducing the severity and/or frequency of symptoms, eliminating the symptoms and/or underlying cause or improving or remediating damage associated with the infection. The term “reduce” or “reduction” means a decrease in any amount, for example, a decrease of 5%, 10%, 20%, 40%, 50%, 60%, 70% or more than 70%.

As used herein, the term “nucleic acid” refers to a polymer of deoxynucleic ribose nucleic acids (DNA), as well as ribose nucleic acids (RNA). The term includes linear molecules, as well as covalently closed circular molecules. It includes single stranded molecules, as well as double stranded molecules.

As used herein, the wild-type form of a polypeptide or nucleic acid molecule is a form encoded by a gene or by a coding sequence encoded by the gene. Typically, a wild-type form of a gene, or molecule encoded thereby, does not contain mutations or other modifications that alter function or structure. The term wild-type also encompasses forms with allelic variation as occurs among and between species. As used herein, a predominant form of a polypeptide or nucleic acid molecule refers to a form of the molecule that is the major form produced from a gene.

As used herein, “species variants” refer to variants in polypeptides among different species, including different mammalian species, such as mouse and human, and species of microorganisms, such as viruses and bacteria.

The term “isolated,” as used herein with reference to a nucleic acid molecule, means that the nucleic acid molecule is free of unrelated nucleic acid sequences, i.e. nucleic acid sequences encoding other genes or those involved in the expression of such other genes, that flank it's 5′ and 3′ ends in the naturally-occurring genome of the organism from which the nucleic acid is derived. Accordingly, an “isolated nucleic acid” has a structure that is different from that of any naturally occurring nucleic acid or to that of any fragment of a naturally occurring genomic nucleic acid spanning more than three separate genes. Thus, the term “isolated nucleic acid molecule” includes, for example, (1) a DNA molecule that has the sequence of part of a naturally occurring genomic DNA molecule, but is not flanked by both of the coding sequences that flank that part of the molecule in the genome of the organism in which it naturally occurs; (2) a nucleic acid incorporated into a vector or into the genomic DNA of a prokaryote or eukaryote in a manner such that the resulting molecule is not identical to any naturally-occurring vector or genomic DNA; (3) a separate molecule such as a cDNA, a genomic fragment, a fragment produced by polymerase chain reaction (PCR), or a restriction fragment; and (4) a recombinant nucleotide sequence that is part of a hybrid gene, i.e. a gene encoding a fusion protein. Specifically excluded from this definition are nucleic acids present in mixtures of (1) DNA molecules, (2) transfected cells, and (3) cell clones, e.g., as these occur in a DNA library such as a cDNA or genomic DNA library.

As used herein, the term “operably linked” means that a nucleic acid and an expression control sequence is positioned in such a way that the expression control sequence directs expression of the nucleic acid when the appropriate molecules such as transcriptional activator proteins are bound to the expression control sequence.

As used herein, the phrase “expression control sequence” means a nucleic acid sequence sufficient to direct transcription of another nucleic acid sequence that is operably linked to the expression control sequence to produce an RNA transcript when appropriate molecules such as transcriptional activator proteins are bound to the expression control sequence.

As used herein, the phrase “expression vector” means a nucleic acid molecule capable of transporting and/or allowing for the expression of another nucleic acid to which it has been linked. The product of that expression is referred to as a messenger ribose nucleic acid (mRNA) transcript. Thus, expression vectors contain appropriate expression control sequences that may direct expression of a nucleic acid that is operably linked to the expression control sequence to produce a transcript. Typically, the nucleic acid encoding the polypeptide is operably linked to an expression control sequence such as a promoter, e.g. a phage, viral, bacterial or mammalian promoter.

The phrase “substantially identical” with respect to an antibody or polypeptide sequence means an antibody or polypeptide sequence exhibiting at least 70%, preferably 80%, more preferably 90% and most preferably 95% sequence identity to the reference antibody or polypeptide sequence. The term with respect to a nucleic acid sequence means a sequence of nucleotides exhibiting at least about 85%, preferably 90%, more preferably 95% and most preferably 97% sequence identity to the reference nucleic acid sequence. For polypeptides, the length of the comparison sequences will generally be at least 25 amino acids. For nucleic acids, the length will generally be at least 75 nucleotides.

The term “identity” or “homology” means the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions shall be construed as reducing identity or homology. Methods and computer programs for the alignment are well known in the art. Sequence identity may be measured using sequence analysis software (e.g., Sequence Analysis Software Package, Genetics Computer Group, University of Wisconsin Biotechnology Center, 1710 University Ave., Madison, Wis. 53705). This software matches similar sequences by assigning degrees of homology to various substitutions, deletions, and other modifications.

The terms “cell”, “cell line” and “cell culture” are used interchangeably, and all such designations include progeny. It is also understood that all progeny may not be precisely identical in DNA content, due to deliberate or inadvertent mutations. Mutant progeny that have the same function or biological property, as screened for in the originally transformed cell, are included.

The “host cells” generally are prokaryotic or eukaryotic hosts.

“Transformation” means introducing DNA into an organism so that the DNA is replicable, either as an extrachromosomal element or by chromosomal integration.

“Transfection” refers to the taking up of an expression vector by a host cell whether or not any coding sequences are in fact expressed.

The terms “transfected host cell” and “transformed” refer to the introduction of DNA into a cell. The cell is termed “host cell” and it may be either prokaryotic or eukaryotic. Typical prokaryotic host cells include various strains of E. coli. Typical eukaryotic host cells are mammalian, such as Chinese hamster ovary or cells of human origin. The introduced DNA sequence may be from the same species as the host cell of a different species from the host cell, or it may be a hybrid DNA sequence, containing some foreign and some homologous DNA.

The terms “replicable expression vector” and “expression vector” refer to a piece of DNA, usually double-stranded, which may have inserted into it a piece of foreign DNA. Foreign DNA is defined as heterologous DNA, which is DNA not naturally found in the host cell. The vector is used to transport the foreign or heterologous DNA into a suitable host cell. Once in the host cell, the vector can replicate independently of the host chromosomal DNA and several copies of the vector and its inserted (foreign) DNA may be generated.

The term “vector” means a DNA construct containing a DNA sequence which is operably linked to a suitable control sequence capable of effecting the expression of the DNA in a suitable host. Such control sequences include a promoter to effect transcription, an optional operator sequence to control such transcription, a sequence encoding suitable mRNA ribosome binding sites, and sequences which control the termination of transcription and translation. The vector may be a plasmid, a phage particle, or simply a potential genomic insert. Once transformed into a suitable host, the vector may replicate and function independently of the host genome, or may in some instances, integrate into the genome itself. “Phage” and “vector” sometimes are used interchangeably herein, as the phage is the form of vector. However, the term vector is intended to include such other form of vectors which serve equivalent function as and which are, or become, known in the art. Typical expression vectors for bacterial expression and mammalian cell culture expression, for example, are based on pRK5 (EP 307,247), pSV16B (WO 91/08291) and pVL1392 (Pharmingen).

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “nucleic acid” refers to at least two linked nucleotides or nucleotide derivatives, including a deoxyribonucleic acid (DNA) and a ribonucleic acid (RNA), joined together, typically by phosphodiester linkages. Also included in the term “nucleic acid” are analogs of nucleic acids such as peptide nucleic acid (PNA), phosphorothioate DNA, and other such analogs and derivatives or combinations thereof. Nucleic acids also include DNA and RNA derivatives containing, for example, a nucleotide analog or a “backbone” bond other than a phosphodiester bond, for example, a phosphotriester bond, a phosphoramidate bond, a phosphorothioate bond, a thioester bond, or a peptide bond (peptide nucleic acid). The term also includes, as equivalents, derivatives, variants and analogs of either RNA or DNA made from nucleotide analogs, single (sense or antisense) and double-stranded nucleic acids. Deoxyribonucleotides include deoxyadenosine, deoxycytidine, deoxyguanosine and deoxythymidine. For RNA, the uracil base is uridine.

Nucleic acids can contain nucleotide analogs, including, for example, mass modified nucleotides, which allow for mass differentiation of nucleic acid molecules; nucleotides containing a detectable label such as a fluorescent, radioactive, luminescent or chemiluminescent label, which allow for detection of a nucleic acid molecule; or nucleotides containing a reactive group such as biotin or a thiol group, which facilitates immobilization of a nucleic acid molecule to a solid support. A nucleic acid also can contain one or more backbone bonds that are selectively cleavable, for example, chemically, enzymatically or photolytically cleavable. For example, a nucleic acid can include one or more deoxyribonucleotides, followed by one or more ribonucleotides, which can be followed by one or more deoxyribonucleotides, such a sequence being cleavable at the ribonucleotide sequence by base hydrolysis. A nucleic acid also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well-known methods (see, for example, Weiler et al. (1997) Nucleic Acids Res. 25:2792-2799).

As used herein, the terms “polynucleotide” and “nucleic acid molecule” refer to an oligomer or polymer containing at least two linked nucleotides or nucleotide derivatives, including a deoxyribonucleic acid (DNA) and a ribonucleic acid (RNA), joined together, typically by phosphodiester linkages. Polynucleotides also include DNA and RNA derivatives containing, for example, a nucleotide analog or a “backbone” bond other than a phosphodiester bond, for example, a phosphotriester bond, a phosphoramidate bond, a phosphorothioate bond, a thioester bond, or a peptide bond (peptide nucleic acid). Polynucleotides (nucleic acid molecules), include single-stranded and/or double-stranded polynucleotides, such as deoxyribonucleic acid (DNA), and ribonucleic acid (RNA) as well as analogs or derivatives of either RNA or DNA. The term also includes, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine. Polynucleotides can contain nucleotide analogs, including, for example, mass modified nucleotides, which allow for mass differentiation of polynucleotides; nucleotides containing a detectable label such as a fluorescent, radioactive, luminescent or chemiluminescent label, which allow for detection of a polynucleotide; or nucleotides containing a reactive group such as biotin or a thiol group, which facilitates immobilization of a polynucleotide to a solid support. A polynucleotide also can contain one or more backbone bonds that are selectively cleavable, for example, chemically, enzymatically or photolytically cleavable. For example, a polynucleotide can include one or more deoxyribonucleotides, followed by one or more ribonucleotides, which can be followed by one or more deoxyribonucleotides, such a sequence being cleavable at the ribonucleotide sequence by base hydrolysis. A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well-known methods (see, for example, Weiler et al. (1997) Nucleic Acids Res. 25:2792-2799). Exemplary of the nucleic acid molecules (polynucleotides) provided herein are oligonucleotides, including synthetic oligonucleotides, oligonucleotide duplexes, primers, including fill-in primers, and oligonucleotide duplex cassettes.

An “isolated” nucleotide is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguishable from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

A nucleotide is “operably linked” when it is placed into a functional relationship with another nucleic acid sequence. This can be a gene and a regulatory sequence(s) which are connected in such a way as to permit gene expression when the appropriate molecules (e.g., transcriptional activator proteins) are bound to the regulatory sequences(s). For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, “E1” refers to an envelope glycoprotein on hepatitis C virus. The E1 polypeptide is located in the HCV polyprotein immediately after the core protein at amino acid residues corresponding to residues 192-383 of the HCV genotype 1a, H77 isolate polyprotein set forth in SEQ ID NO:607. Exemplary amino acid sequences of E1 polypeptides are set forth in SEQ ID NOS:1377-1361. Reference to an E1 polypeptide includes isolated E1 polypeptides and E1 polypeptides in an E1E2 complex. Reference to an E1 polypeptide also includes truncated forms thereof, such as soluble forms that are truncated at their C-terminal to remove the transmembrane domain, that retain a property, such as a binding property, and variants, such as those from different HCV isolates and quasispecies, including polypeptides that have at least 40%, 45%, 50%, 55%, 65%, 70%, 75%, 80%, 85%, 90%, 95%, 96%, 97%, 98%, 99% or more sequence identity to the E1 polypeptide from HCV genotype 1a, isolate H77, set forth in SEQ ID NO: 1337. Included are modified E1 polypeptides, such as those set forth in any of SEQ ID NOS: 1206-1284 and variants thereof.

E1 polypeptides include synthetic molecules prepared by translation of nucleic acids, proteins generated by chemical synthesis, such as syntheses that include ligation of shorter polypeptides, through recombinant methods, proteins isolated from HCV-infected human and non-human tissue and cells, chimeric E1 polypeptides and modified forms thereof. E1 polypeptides also include fragments or portions of E1 that are of sufficient length or include appropriate regions to retain at least one property of a full-length E1 polypeptide, such as the ability to form a complex with E2 that, when on a viral particle, can bind to host cells, or the ability to bind to anti-E1 or E1E2 antibodies. E1 polypeptides also include those that contain chemical or posttranslational modifications and those that do not contain chemical or posttranslational modifications. Such modifications include, but are not limited to, pegylation, albumination, glycosylation, farnysylation, carboxylation, hydroxylation, phosphorylation, and other polypeptide modifications known in the art.

As used herein, “E2” refers to an envelope glycoprotein on hepatitis C virus. The E2 polypeptide is located in the HCV polyprotein immediately after the E1 polypeptide at amino acid residues corresponding to residues 384-746 of the HCV genotype 1a, H77 isolate polyprotein set forth in SEQ ID NO:607. Exemplary amino acid sequences of E2 polypeptides are set forth in SEQ ID NOS:1312-1336. Reference to an E2 polypeptide includes isolated E2 polypeptides and E2 polypeptides in an E1E2 complex. Reference to an E2 polypeptide also includes truncated forms thereof, such as soluble forms that are truncated at their C-terminal to remove the transmembrane domain, that retain a property, such as a binding property, and variants, such as those from different HCV isolates and quasispecies, including polypeptides that have at least 40%, 45%, 50%, 55%, 65%, 70%, 75%, 80%, 85%, 90%, 95%, 96%, 97%, 98%, 99% or more sequence identity to the E2 polypeptide from HCV genotype 1a, isolate H77, set forth in SEQ ID NO: 1312. Included are modified E2 polypeptides, such as those set forth in any of SEQ ID NOS: 1286-1311 and variants thereof.

E2 polypeptides include synthetic molecules prepared by translation of nucleic acids, proteins generated by chemical synthesis, such as syntheses that include ligation of shorter polypeptides, through recombinant methods, proteins isolated from HCV-infected human and non-human tissue and cells, chimeric E2 polypeptides and modified forms thereof. E2 polypeptides also include fragments or portions of E2 that are of sufficient length or include appropriate regions to retain at least one property of a full-length E2 polypeptide, such as the ability to bind to anti-E2 antibodies. E2 polypeptides also include those that contain chemical or posttranslational modifications and those that do not contain chemical or posttranslational modifications. Such modifications include, but are not limited to, pegylation, albumination, glycosylation, farnysylation, carboxylation, hydroxylation, phosphorylation, and other polypeptide modifications known in the art.

Reference to an amino acid position in any of the mature, processed HCV proteins, such as E1 or E2, is made with numbering relative to the full length HCV polypeptide, such as that set forth in SEQ ID NO:607, and not with reference to the mature polypeptide. Thus, for example, reference to amino acid position 192 in the E1 polypeptide from HCV genotype 1a, isolate H77 corresponds to position 1 of the mature E1 polypeptide set forth in SEQ ID NO:1337. Similarly, reference to amino acid position 425 in the E2 polypeptide from HCV genotype 1a, isolate H77 corresponds to position 42 of the mature E2 polypeptide set forth in SEQ ID NO:1312.

As used herein, corresponding residues refers to residues that occur at aligned loci. Related or variant polypeptides are aligned by any method known to those of skill in the art. Such methods typically maximize matches, and include methods such as using manual alignments and by using the numerous alignment programs available (for example, BLASTP) and others known to those of skill in the art. By aligning the sequences of polypeptides, one skilled in the art can identify corresponding residues, using conserved and identical amino acid residues as guides. For example, by aligning the sequences of E2 polypeptides from different HCV isolates, one of skill in the art can identify corresponding residues, using conserved and identical amino acid residues as guides. Corresponding positions also can be based on structural alignments, for example by using computer simulated alignments of protein structure. In other instances, corresponding regions can be identified.

As used herein, a “modification” is in reference to modification of a sequence of amino acids of a polypeptide or a sequence of nucleotides in a nucleic acid molecule and includes deletions, insertions, and replacements of amino acids and nucleotides, respectively. Methods of modifying a polypeptide are routine to those of skill in the art, such as by using recombinant DNA methodologies.

As used herein, “deletion,” when referring to a nucleic acid or polypeptide sequence, refers to the deletion of one or more nucleotides or amino acids compared to a sequence, such as a target polynucleotide or polypeptide or a native or wild-type sequence.

As used herein, “insertion” when referring to a nucleic acid or amino acid sequence, describes the inclusion of one or more additional nucleotides or amino acids, within a target, native, wild-type or other related sequence. Thus, a nucleic acid molecule that contains one or more insertions compared to a wild-type sequence, contains one or more additional nucleotides within the linear length of the sequence. As used herein, “additions,” to nucleic acid and amino acid sequences describe addition of nucleotides or amino acids onto either termini compared to another sequence.

As used herein, “substitution” refers to the replacing of one or more nucleotides or amino acids in a native, target, wild-type or other nucleic acid or polypeptide sequence with an alternative nucleotide or amino acid, without changing the length (as described in numbers of residues) of the molecule. Thus, one or more substitutions in a molecule does not change the number of amino acid residues or nucleotides of the molecule. Substitution mutations compared to a particular polypeptide can be expressed in terms of the number of the amino acid residue along the length of the polypeptide sequence. For example, a modified polypeptide having a modification in the amino acid at the 19^{th} position of the amino acid sequence that is a substitution of Isoleucine (Ile; I) for cysteine (Cys; C) can be expressed as I19C, Ile19C, or simply C19, to indicate that the amino acid at the modified 19^{th} position is a cysteine. In this example, the molecule having the substitution has a modification at Ile 19 of the unmodified polypeptide.

“Treatment” refers to both therapeutic treatment and prophylactic or preventative measures. Those in need of treatment include those already with the disorder as well as those in which the disorder is to be prevented.

A “disorder” is any condition that would benefit from treatment with the polypeptide. This includes chronic and acute disorders or diseases including those pathological conditions which predispose the mammal to the disorder in question.

“Mammal” for purposes of treatment refers to any animal classified as a mammal, including human, domestic and farm animals, nonhuman primates, and zoo, sports, or pet animals, such as dogs, horses, cats, cows, etc.

The word “label” when used herein refers to a detectable compound or composition which is conjugated directly or indirectly to the antibody. The label may itself be detectable (e.g., radioisotope labels or fluorescent labels) or, in the case of an enzymatic label, may catalyze chemical alteration of a substrate compound or composition which is detectable.

As used herein, “solid phase” means a non-aqueous matrix to which the antibody can adhere. Example of solid phases encompassed herein include those formed partially or entirely of glass (e.g. controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g. an affinity chromatography column). This term also includes a discontinuous solid phase of discrete particles, such as those described in U.S. Pat. No. 4,275,149.

As used herein, “affinity maturation using phage display” (AMPD) refers to a process described in Lowman et al., Biochemistry 30(45): 10832-10838 (1991), see also Hawkins et al., J. Mol. Biol. 226, 889-896 (1992). While not strictly limited to the following description, this process can be described briefly as: several hypervariable region sites (e.g. 6-7 sites) are mutated to generate all possible amino acid substitutions at each site. The antibody mutants thus generated are displayed in a monovalent fashion from filamentous phage particles as fusions to the gene III product of M13 packaged within each particle. The phage expressing the various mutants can be cycled through rounds of binding selection, followed by isolation and sequencing of those mutants which display specific immuno-binding, preferably high affinity binding. The method is also described in WO 92/09690, issued Jun. 11, 1992. A modified procedure involving pooled affinity display is described in Cunningham, B. C. et al., EMBO J. 13(11), 2508-2515 (1994).

As used herein, the term “phage library” refers to the phage library used in the affinity maturation process described above and in Hawkins et al., J. Mol. Biol. 226: 889-896 (1992), and in Lowman et al., Biochemistry 30(45): 10832-10838 (1991). Each library includes a variable region (e.g. 6-7 sites) for which all possible amino acid substitutions are generated. The antibody mutants thus generated are displayed in a monovalent fashion from filamentous phage particles as fusions to the gene III product of M13 packaged within each particle and expressed on the exterior of the phage.

As used herein, “high affinity” means an affinity constant (Kd) of at least 10⁻⁷ M, and especially preferably at least 10⁻¹⁰ M under physiological conditions.

By “pharmaceutically acceptable” it is meant a carrier, diluent, excipient, and/or salt that is compatible with the other ingredients of the formulation, and not deleterious to the recipient thereof, for example, a buffered aqueous, oil or organic medium containing optional stabilizing agents and adjuvants for stimulation of immune binding.

### B. HEPATITIS C VIRUS

Hepatitis C virus (HCV) is a noncytopathic, positive-stranded RNA virus that causes acute and chronic hepatitis and hepatocellular carcinoma (Hoofnagle, J. H. (2002) Hepatology 36, S21-29). The hepatocyte is the primary target cell, although various lymphoid populations, especially B cells and dendritic cells also can be infected at lower levels (Kanto et al. (1999) J. Immunol. 162, 5584-5591; Auffermann-Gretzinger et al. (2001) Blood 97, 3171-3176; Hiasa et al. (1998) Biochem. Biophys. Res. Commun. 249, 90-95). A striking feature of HCV infection is its tendency towards chronicity with at least 70% of acute infections progressing to persistence (Hoofnagle, J. H. (2002) Hepatology 36, S21-29). HCV chronicity is often associated with significant liver disease, including chronic active hepatitis, cirrhosis and hepatocellular carcinoma (Alter, H. J. & Seeff, L. B. (2000) Semin. Liver Dis. 20, 17-35). With over 170 million people currently infected, HCV represents a growing public health concern.

The single stranded HCV RNA genome contains a 9.6 kb positive strand RNA genome composed of a 5′ non coding region (NCR), a single open reading frame (ORF) encoding a large polyprotein, and a 3′-NCR. The polyprotein has about 3010-3033 amino acids (Q.-L. Choo, et al. Proc. Natl. Acad. Sci. USA 88, 2451-2455 (1991); N. Kato et al., Proc. Natl. Acad. Sci. USA 87, 9524-9528 (1990); A. Takamizawa et al., J. Virol. 65, 1105-1113 (1991)). Nucleic acid and amino acid sequences for different isolates of HCV can be found in the art, for example, in the National Center for Biotechnology Information (NCBI) database.

An example of an HCV subtype 1a is strain H77. Its polyprotein sequence (Genbank ID AAB66324) is as follows:

| (SEQ ID NO: 607) | |
|---|---|
| 1 | MSTNPKPQRK TKRNTNRRPQ DVKFPGGGQI VGGVYLLPRR GPRLGVRATR |
| | KTSERSQPRG |
| 61 | RRQPIPKARR PEGRTWAQPG YPWPLYGNEG CGWAGWLLSP RGSRPSWGPT |
| | DPRRRSRNLG |
| 121 | KVIDTLTCGF ADLMGYIPLV GAPLGGAARA LAHGVRVLED GVNYATGNLP |
| | GCSFSIFLLA |
| 181 | LLSCLTVPAS AYQVRNSSGL YHVTNDCPNS SIVYEAADAI LHTPGCVPCV |
| | REGNASRCWV |
| 241 | AVTPTVATRD GKLPTTQLRR HIDLLVGSAT LCSALYVGDL CGSVFLVGQL |
| | FTFSPRRHWT |
| 301 | TQDCNCSIYP GHITGHRMAW DMMMNWSPTA ALVVAQLLRI PQAIMDMIAG |
| | AHWGVLAGIA |
| 361 | YFSMVGNWAK VLVVLLLFAG VDAETHVTGG SAGRTTAGLV GLLTPGAKQN |
| | IQLINTNGSW |
| 421 | HINSTALNCN ESLNTGWLAG LFYQHKFNSS GCPERLASCR RLTDFAQGWG |
| | PISYANGSGL |
| 481 | DERPYCWHYP PRPCGIVPAK SVCGPVYCFT PSPVVVGTTD RSGAPTYSWG |
| | ANDTDVFVLN |
| 541 | NTRPPLGNWF GCTWMNSTGF TKVCGAPPCV IGGVGNNTLL CPTDCFRKHP |
| | EATYSRCGSG |
| 601 | PWITPRCMVD YPYRLWHYPC TINYTIFKVR MYVGGVEHRL EAACNWTRGE |
| | RCDLEDRDRS |
| 661 | ELSPLLLSTT QWQVLPCSFT TLPALSTGLI HLHQNIVDVQ YLYGVGSSIA |
| | SWAIKWEYVV |
| 721 | LLFLLLADAR VCSCLWMMLL ISQAEAALEN LVILNAASLA GTHGLVSFLV |
| | FFCFAWYLKG |
| 781 | RWVPGAVYAF YGMWPLLLLL LALPQRAYAL DTEVAASCGG VVLVGLMALT |
| | LSPYYKRYIS |
| 841 | WCMWWLQYFL TRVEAQLHVW VPPLNVRGGR DAVILLMCVV HPTLVFDITK |
| | LLLAIFGPLW |
| 901 | ILQASLLKVP YFVRVQGLLR ICALARKIAG GHYVQMAIIK LGALTGTYVY |
| | NHLTPLRDWA |
| 961 | HNGLRDLAVA VEPVVFSRME TKLITWGADT AACGDIINGL PVSARRGQEI |
| | LLGPADGMVS |
| 1021 | KGWRLLAPIT AYAQQTRGLL GCIITSLTGR DKNQVEGEVQ IVSTATQTFL |
| | ATCINGVCWT |
| 1081 | VYHGAGTRTI ASPKGPVIQM YTNVDQDLVG WPAPQGSRSL TPCTCGSSDL |
| | YLVTRHADVI |
| 1141 | PVRRRGDSRG SLLSPRPISY LKGSSGGPLL CPAGHAVGLF RAAVCTRGVA |
| | KAVDFIPVEN |
| 1201 | LETTMRSPVF TDNSSPPAVP QSFQVAHLHA PTGSGKSTKV PAAYAAQGYK |
| | VLVLNPSVAA |
| 1261 | TLGFGAYMSK AHGVDPNIRT GVRTITTGSP ITYSTYGKFL ADGGCSGGAY |
| | DIIICDECHS |
| 1321 | TDATSILGIG TVLDQAETAG ARLVVLATAT PPGSVTVSHP NIEEVALSTT |
| | GEIPFYGKAI |
| 1381 | PLEVIKGGRH LIFCHSKKKC DELAAKLVAL GINAVAYYRG LDVSVIPTSG |
| | DVVVVSTDAL |
| 1441 | MTGFTGDFDS VIDCNTCVTQ TVDFSLDPTF TIETTTLPQD AVSRTQRRGR |
| | TGRGKPGIYR |
| 1501 | FVAPGERPSG MFDSSVLCEC YDAGCAWYEL TPAETTVRLR AYMNTPGLPV |
| | CQDHLEFWEG |
| 1561 | VFTGLTHIDA HFLSQTKQSG ENFPYLVAYQ ATVCARAQAP PPSWDQMWKC |
| | LIRLKPTLHG |
| 1621 | PTPLLYRLGA VQNEVTLTHP ITKYIMTCMS ADLEVVTSTW VLVGGVLAAL |
| | AAYCLSTGCV |
| 1681 | VIVGRIVLSG KPAIIPDREV LYQEFDEMEE CSQHLPYIEQ GMMLAEQFKQ |
| | KALGLLQTAS |
| 1741 | RQAEVITPAV QTNWQKLEVF WAKHMWNFIS GIQYLAGLST LPGNPAIASL |
| | MAFTAAVTSP |
| 1801 | LTTGQTLLFN ILGGWVAAQL AAPGAATAFV GAGLAGAAIG SVGLGKVLVD |
| | ILAGYGAGVA |
| 1861 | GALVAFKIMS GEVPSTEDLV NLLPAILSPG ALVVGVVCAA ILRRHVGPGE |
| | GAVQWMNRLI |
| 1921 | AFASRGNHVS PTHYVPESDA AARVTAILSS LTVTQLLRRL HQWISSECTT |
| | PCSGSWLRDI |
| 1981 | WDWICEVLSD FKTWLKAKLM PQLPGIPFVS CQRGYRGVWR GDGIMHTRCH |
| | CGAEITGHVK |
| 2041 | NGTMRIVGPR TCRNMWSGTF PINAYTTGPC TPLPAPNYKF ALWRVSAEEY |
| | VEIRRVGDFH |
| 2101 | YVSGMTTDNL KCPCQIPSPE FFTELDGVRL HRFAPPCKPL LREEVSFRVG |
| | LHEYPVGSQL |
| 2161 | PCEPEPDVAV LTSMLTDPSH ITAEAAGRRL ARGSPPSMAS SSASQLSAPS |
| | LKATCTANHD |
| 2221 | SPDAELIEAN LLWRQEMGGN ITRVESENKV VILDSFDPLV AEEDEREVSV |
| | PAEILRKSRR |
| 2281 | FARALPVWAR PDYNPPLVET WKKPDYEPPV VHGCPLPPPR SPPVPPPRKK |
| | RTVVLTESTL |
| 2341 | STALAELATK SFGSSSTSGI TGDNTTTSSE PAPSGCPPDS DVESYSSMPP |
| | LEGEPGDPDL |
| 2401 | SDGSWSTVSS GADTEDVVCC SMSYSWTGAL VTPCAAEEQK LPINALSNSL |
| | LRHHNLVYST |
| 2461 | TSRSACQRQK KVTFDRLQVL DSHYQDVLKE VKAAASKVKA NLLSVEEACS |
| | LTPPHSAKSK |
| 2521 | FGYGAKDVRC HARKAVAHIN SVWKDLLEDS VTPIDTTIMA KNEVFCVQPE |
| | KGGRKPARLI |
| 2581 | VFPDLGVRVC EKMALYDVVS KLPLAVMGSS YGFQYSPGQR VEFLVQAWKS |
| | KKTPMGFSYD |
| 2641 | TRCFDSTVTE SDIRTEEAIY QCCDLDPQAR VAIKSLTERL YVGGPLTNSR |
| | GENCGYRRCR |
| 2701 | ASGVLTTSCG NTLTCYIKAR AACRAAGLQD CTMLVCGDDL VVICESAGVQ |
| | EDAASLRAFT |
| 2761 | EAMTRYSAPP GDPPQPEYDL ELITSCSSNV SVAHDGAGKR VYYLTRDPTT |
| | PLARAAWETA |
| 2821 | RHTPVNSWLG NIIMFAPTLW ARMILMTHFF SVLIARDQLE QALNCEIYGA |
| | CYSIEPLDLP |
| 2881 | PIIQRLHGLS AFSLHSYSPG EINRVAACLR KLGVPPLRAW RHRAHCVRAR |
| | LLSRGGRAAI |
| 2941 | CGKYLFNWAV RTKLKLTPIA AAGRLDLSGW FTAGYSGGDI YHSVSHARPR |
| | WFWFCLLLLA |
| 3001 | AGVGIYLLPN R |

An example of an HCV subtype 1b is strain HCV-L2, which can be found in the NCBI database as accession number U01214 (gi 437107). Its polyprotein sequence (AAA75355) is as follows:

| (SEQ ID NO: 608) | |
|---|---|
| 1 | MSTNPKPQRK TKRNTNRRPQ DVKFPGGGQI VGGVYLLPRR GPRLGVRATR |
| | KTSERSQPRG |
| 61 | RRQPIPKARQ PEGRAWAQPG YPWPLYANEG LGWAGWLLSP RGSRPSWGPT |
| | DPRRRSRNLG |
| 121 | KVIDTPTCGF ADLMGYIPLV GAPLGGVARA LAHGVRVLED SVNYATGNLP |
| | GCSFSIFLLA |
| 181 | LLSCLTVPAS AYEVRNVSGI YHVTNDCSNS SIVYEAADLI MHTPGCVPCV |
| | REANSSRCWV |
| 241 | ALTPTLAARD SSIPTATIRR HVDLLVGAAA FCSAMYVGDL CGSVFLVSQL |
| | FTFSPRLHQT |
| 301 | VQDCNCSIYP GHLTGHRMAW DMMMNWSPTA ALVVSQLLRI PQAIVDMVAG |
| | AHWGVLAGLA |
| 361 | YYPMVGNWAK VLIVMLLFAG VDGTTVTMGG TVARTTYGFT GLFRPGASQK |
| | IQLINTNGSW |
| 421 | HINRTALNCN DSLNTGFLAA LFYTHRFNAS GCPERMASCQ SIDKFVQGWG |
| | PITYAENGSS |
| 481 | DQRPYCWHYA PRRCGIVPAS QVCGPVYCFT PSPVVVGTTD RSGAPTYSWG |
| | ENETDVLLLN |
| 541 | NTRPPQGNWF GCTWMSSTGF TKTCGGPPCN IGGAGNNTLT CPTDCFRKHP |
| | EATYTKCGSG |
| 601 | PWLTPRCLVD YPYRLWHYPC TVNFTTFKVR MYVGGVEHRL IAACNWTRGE |
| | RCNLEDRDRS |
| 661 | ELSPLLLSTT EWQILPCSYT TLPALSTGLI HLHQNIVDVQ YLYGIGSAVV |
| | SFVIKWEYVL |
| 721 | LFFLLLADAR VCACLWMILL IAQAEAALEN LVVLNAASVA GAHGILSFLV |
| | FFCAAWYIKG |
| 781 | RLVPGAAYAS YGVWPLLLLL LALPPRAYAM DQGMAASSGG TVLVGLMLLT |
| | LSPYYKVVLA |
| 841 | RLIWWLQYFI TRAEAHLQVW VPPLNVRGGR DAVILLTCAV YPELVFDITK |
| | LLLAIFGPLM |
| 901 | VLQAGIIKMP YFVRAQGLIR ACMLVRKVAG GHYVQMAFMK LAALTGTYVY |
| | DHLTPLRDWA |
| 961 | HTGLRDLAVA VEPVVFSDME TKIITWGADT AECGDIILGY RSSARRGREI |
| | LLGPADSLEG |
| 1021 | QGWRLLAPIT AYAQQTRGLL GCIITSLTGR DKNQVEGEVQ VVSTATQSFL |
| | ATCVNGVCWT |
| 1081 | VFHGAGSKTL AGPKGPITQM YTNVDQDLVG WQAAPGMRSL TPCTCGSSDL |
| | YLVTRHADVI |
| 1141 | PVRRRGDGRG SLLSPRPVSY LKGSSGGPLL WPSGHAVGIF RAAVCTRGVA |
| | KAVDFVPVES |
| 1201 | METTMRSPVF TDNSSPPAVP QTFQVAHLHA PTGSGKSTKV PAAYAAQGYK |
| | VLVLNPSVAA |
| 1261 | TLGFGAYMSK AHGTDPNIRT GARTITTGAP ITYSTYGKFF ADGGCSGGAY |
| | DIIICDECHS |
| 1321 | TDSTTILGIG TVLDRAETAG ARLVVLATAT PPGSTTVPHP NIEEVALPNT |
| | GEIPFYGRAI |
| 1381 | PIEFIKGGRH LIFCPSKKKC DELAAKLSAL GINAVAYYRG LDVSVIPTSG |
| | DVVVVATDAL |
| 1441 | MTGYTGDFDS VIDCNTCVTQ TVDFSLDPTF TIETTTVPQD AVSRTQRRGR |
| | TGRGRGGIYR |
| 1501 | FVTPGERPSG MFDSSVLCEC YDAGCAWYEL TPAETTVRLR AYLNTPGLPV |
| | CQDHLEFWES |
| 1561 | VFTGLNHIDA HFLSQTKQAG DNFPYLVAYQ ATVCARAQAP PPSWDQMWKC |
| | LIWLKPVLHG |
| 1621 | PTPLLYRLGA VQNEITLTHP ITKLIMASMS ADLEVVTSTW VLVGGVLAAL |
| | AAYCLTTGSV |
| 1681 | VIVGRIILSG RPAVIPDREV LYREFDEMEE CASHLPYIEQ GVQLAEQFKQ |
| | KALGLLQTAT |
| 1741 | KQAEAAAPVV ESKWRALETF WAKHMWNFIS GIQYLAALST LPGNPAIASL |
| | MAFTASITSP |
| 1801 | LTTQNTLLFN ILGGWVAAQL APASAASAFV GAGSAGAAIG TIGLGKVLVD |
| | ILAGYGAGVA |
| 1861 | GALVAFKVMS GEMPSTEDLV NLLPAILSPG ALVVGVVCAA ILRRHVGPGE |
| | GAVQWMNRLI |
| 1921 | AFASRGNHDS PTHYVPESDA AARVTQILSS LTITQLLKRL HQWINEDCST |
| | PCSGSWLRDV |
| 1981 | WDWICTVLTD FKTWLQSKLL PRLPGVPFFS CQRGYKGVWR GDGIMQTTCP |
| | CGAQITGHVK |
| 2041 | NGSMRIVGPK TCSNTWHGTF PINAYTTGPC TPAPTPNYSR ALWRVAAEEY |
| | VEVTRVGDFH |
| 2101 | YVTGMTTDNV KCPCQVPAPE FFTEVDGVRL HRYAPACKTL LREEVTFQVG |
| | LNQYLVGSQL |
| 2161 | PCEPEPDVAV LTSMLTDPSH ITAETAKRRL ARGSPPSLAS SSASQLSAPS |
| | LKATCTTHHD |
| 2221 | SPDADLIEAN LLWRQEMGGN ITRVESESKV VILDSFDPLR AEEGEGEVSV |
| | AAEILRKSKK |
| 2281 | FPPALPEWAR PDYNPPLLES WKDPDYVPPV VHGCPLPPAK APPIPPPRRK |
| | RTVVLTESTV |
| 2341 | SSALAELAVK TFGSSESSAV DSGTATAPPD QVSDNGDKGS DAESYSSMPP |
| | LEGEPGDPDL |
| 2401 | SDGSWSTVSE EASEDVVCCS MSYSWTGALI TPCAAEESKL PINALSNSLL |
| | RHHNMVYATT |
| 2461 | SRSAGLRQKK VTFDRLQVLD DHYRDVLKEM KAKASTVKAK LLSVEEACKL |
| | TPPHSAKSKF |
| 2521 | GYGAKDVRNL SSRAVNHIRS VWKDLLEDTE TPIDTTIMAK SEVFCVQPEK |
| | GGRKPARLIV |
| 2581 | FPDLGVRVCE KMALYDVVST LPQAVMGPSY GFQYSPGQRV EFLVNAWKSK |
| | KCPMGFSYDT |
| 2641 | RCFDSTVTES DIRTEESIYQ CCDLAPEAKQ AIKSLTERLY IGGPLTNSKG |
| | QNCGYRRCRA |
| 2701 | SVVLTTSCGN TLTCYLKASA ACRAAKLQDC TMLVNGDDLV VICESAGTQE |
| | DAANLRAFTE |
| 2761 | AMTRYSAPPG DPPQPEYDLE LITSCSSNVS VAHDASGKRV YYLTRDPTTP |
| | LARAAWETAR |
| 2821 | HTPVNSWLGN IIMYAPTLWA RMILMTHFFS ILLAQEQLEK ALECQIYGAC |
| | YSIEPLDLPQ |
| 2881 | IIERLHGLSA FSLHSYSPGE INRVASCLRK LGVPPLRVWR HRARRVRAKL |
| | LSQGGRAATC |
| 2941 | GKYLFNWAVR TKLKLTPIPA ASRLDLSSWF VAGYSGGDIY HSVSHARPRW |
| | FMLCLLLLSV |
| 3001 | GVGIYLLPNR |

An example of an HCV subtype 1c strain HC-G9 can be found in the NCBI database as accession number D14853 (gi 464177). The polyprotein sequence (BAA03581.1) is as follows:

| (SEQ ID NO: 609) | |
|---|---|
| 1 | MSTNPKPQRK TKRNTNRRPQ DVKFPGGGQI VGGVYLLPRR GPRVGVRATR |
| | KTSERSQPRG |
| 61 | RRQPIPKARR PEGRSWAQPG YPWPLYGNEG CGWAGWLLSP RGSRPSWGPS |
| | DPRRRSRNLG |
| 121 | KVIDTLTCGF ADLMGYIPLV GAPLGGAARA LAHGVRVLED GVNYATGNLP |
| | GCSFSIFLLA |
| 181 | LLSCLTVPAS AVGVRNSSGV YHVTNDCPNA SVVYETENLI MHLPGCVPYV |
| | REGNASRCWV |
| 241 | SLSPTVAARD SRVPVSEVRR RVDSIVGAAA FCSAMYVGDL CGSIFLVGQI |
| | FTFSPRHHWT |
| 301 | TQDCNCSIYP GHVTGHRMAW DMMMNWSPTG ALVVAQLLRI PQAIVDMIAG |
| | AHWGVLAGLA |
| 361 | YYSMVGNWAK VVVVLLLFAG VDAETRVTGG AAGHTAFGFA SFLAPGAKQK |
| | IQLINTNGSW |
| 421 | HINRTALNCN ESLDTGWLAG LLYYHKFNSS GCPERMASCQ PLTAFDQGWG |
| | PITHEGNASD |
| 481 | DQRPYCWHYA LRPCGIVPAK KVCGPVYCFT PSPVVVGTTD RAGVPTYRWG |
| | ANETDVLLLN |
| 541 | NSRPPMGNWF GCTWMNSSGF TKTCGAPACN IGGSGNNTLL CPTDCFRKHP |
| | DATYSRCGSG |
| 601 | PWLTPRCLVD YPYRLWHYPC TVNYTIFKIR MFVGGVEHRL DAACNWTRGE |
| | RCDLDDRDRA |
| 661 | ELSPLLLSTT QWQVLPCSFT TLPALSTGLI HLHQNIVDVQ YLYGLSSAVT |
| | SWVIKWEYVV |
| 721 | LLFLLLADAR ICACLWMMLL ISQVEAALEN LIVLNAASLV GTHGIVPFFI |
| | FFCAAWYLKG |
| 781 | KWAPGLAYSV YGMWPLLLLL LALPQRAYAL DQELAASCGA TVFICLAVLT |
| | LSPYYKQYMA |
| 841 | RGIWWLQYML TRAEALLQVW VPPLNARGGR DGVVLLTCVL HPHLLFEITK |
| | IMLAILGPLW |
| 901 | ILQASLLKVP YFVRAHGLIR LCMLVRKTAG GQYVQMALLK LGAFAGTYIY |
| | NHLSPLQDWA |
| 961 | HSGLRDLAVA TEPVIFSRME IKTITWGADT AACGDIINGL PVSARRGREV |
| | LLGPADALTD |
| 1021 | KGWRLLAPIT AYAQQTRGLL GCIITSLTGR DKNQVEGEVQ IVSTATQTFL |
| | ATCVNGVCWT |
| 1081 | VYHGAGSRTI ASASGPVIQM YTNVDQDLVG WPAPQGARSL TPCTCGASDL |
| | YLVTRHADVI |
| 1141 | PVRRRGDNRG SLLSPRPISY LKGSSGGPLL CPMGHAVGIF RAAVCTRGVA |
| | KAVDFVPVES |
| 1201 | LETTMRSPVF TDNSSPPTVP QSYQVAHLHA PTGSGKSTKV PAAYAAQGYK |
| | VLVLNPSVAA |
| 1261 | TLGFGAYMSK AHGIDPNVRT GVRTITTGSP ITHSTYGKFL ADGGCSGGAY |
| | DIIICDECHS |
| 1321 | VDATSILGIG TVLDQAETAG VRLTILATAT PPGSVTVPHS NIEEVALSTE |
| | GEIPFYGKAI |
| 1381 | PLNYIKGGRH LIFCHSKKKC DELAAKLVGL GVNAVAFYRG LDVSVIPTTG |
| | DVVVVATDAL |
| 1441 | MTGYTGDFDS VIDCNTCVVQ TVDFSLDPTF SIETSTVPQD AVSRSQRRGR |
| | TGRGKHGIYR |
| 1501 | YVSPGERPSG MFDSVVLCEC YDAGCAWYEL TPAETTVRLR AYLNTPGLPV |
| | CQDHLEFWES |
| 1561 | VFTGLTHIDA HFLSQTKQSG ENFPYLVAYQ ATVCARAKAP PPSWDQMWKC |
| | LIRLKPTLTG |
| 1621 | ATPLLYRLGG VQNEITLTHP ITKYIMACMS ADLEVVTSTW VLVGGVLAAL |
| | AAYCLSTGSV |
| 1681 | VIVGRIILSG KPAVIPDREV LYREFDEMEE CAAHIPYLEQ GMHLAEQFKQ |
| | KALGLLQTAS |
| 1741 | KQAETITPAV HTNWQKLESF WAKHMWNFVS GIQYLAGLST LPGNPAIASL |
| | MSFTAAVTSP |
| 1801 | LTTQQTLLFN ILGGWVAAQL AAPAAATAFV GAGITGAVIG SVGLGKVLVD |
| | ILAGYGAGVA |
| 1861 | GALVAFKIMS GEAPTAEDLV NLLPAILSPG ALVVGVVCAA ILRRHVGPGE |
| | GAVQWMNRLI |
| 1921 | AFASRGNHVS PTHYVPESDA SVRVTHILTS LTVTQLLKRL HVWISSDCTA |
| | PCAGSWLKDV |
| 1981 | WDWICEVLSD FKSWLKAKLM PQLPGIPFVS CQRGYRGVWR GEGIMHARCP |
| | CGADITGHVK |
| 2041 | NGSMRIVGPK TCSNTWRGSF PINAHTTGPC TPSPAPNYTF ALWRVSAEEY |
| | VEVRRLGDFH |
| 2101 | YITGVTTDKI KCPCQVPSPE FFTEVDGVRL HRYAPPCKPL LRDEVTFSIG |
| | LNEYLVGSQL |
| 2161 | PCEPEPDVAV LTSMLTDPSH ITAETAARRL NRGSPPSLAS SSASQLSAPS |
| | LKATCTTHHD |
| 2221 | SPDADLITAN LLWRQEMGGN ITRVESENKI VILDSFDPLV AEEDDREISV |
| | PAEILLKSKK |
| 2281 | FPPAMPIWAR PDYNPPLVEP WKRPDYEPPL VHGCPLPPPK PTPVPPPRRK |
| | RTVVLDESTV |
| 2341 | SSALAELATK TFGSSTTSGV TSGEAAESSP APSCDGELDS EAESYSSMPP |
| | LEGEPGDPDL |
| 2401 | SDGSWSTVSS DGGTEDVVCC SMSYSWTGAL ITPCAAEETK LPINALSNSL |
| | LRHHNLVYST |
| 2461 | TSRSAGQRQK KVTFDRLQVL DDHYRDVLKE AKAKASTVKA KLLSVEEACS |
| | LTPPHSARSK |
| 2521 | FGYGAKDVRS HSSKAIRHIN SVWQDLLEDN TTPIDTTIMA KNEVFCVKPE |
| | KGGRKPARLI |
| 2581 | VYPDLGVRVC EKRALYDVVK QLPIAVMGTS YGFQYSPAQR VDFLLNAWKS |
| | KKNPMGFSYD |
| 2641 | TRCFDSTVTE ADIRTEEDLY QSCDLVPEAR AAIRSLTERL YIGGPLTNSK |
| | GQNCGYRRCR |
| 2701 | ASGVLTTSCG NTITCYLKAS AACRAAKLRD CTMLVCGDDL VVICESAGVQ |
| | EDAANLRAFT |
| 2761 | EAMTRYSAPP GDPPQPEYDL ELITSCSSNV SVAHDGAGKR VYYLTRDPET |
| | PLARAAWETA |
| 2821 | RHTPVNSWLG NIIMFAPTLW VRMVLMTHFF SILIAQEHLE KALDCEIYGA |
| | VHSVQPLDLP |
| 2881 | EIIQRLHGLS AFSLHSYSPG EINRVAACLR KLGVPPLRAW RHRAHCVRAT |
| | LLSQGGRAAI |
| 2941 | CGKYLFNWAV KTKLKLTPLP SASQLDLSNW FTGGYSGGDI YHSVSHVRPR |
| | WFFWCLLLLS |
| 3001 | VGVGIYLLPN R |

Other HCV polyprotein sequences are known in the art, see for example, the web sites http://www.hcvdb.org/viruses.asp; http://www.ncbi.nlm.nih.gov/ and http://hcv.lanl.gov/content/sequence/LOCATE/locate.html. Additional examples include a Taiwan isolate of hepatitis C virus available in the NCBI database at accession number P29846 (gi: 266821). Other examples of HCV polyprotein sequences include those at the NCBI accession number AF009606, AY734971, AJ238799, AY545953, AY734974, AB047639, AF177036, AY734977, AY734982, AY734984, AY734987, EF427672, and AY736194.

The HCV polyprotein that is generated by translation of the HCV ORF is processed to generate at least ten proteins termed C, E1, E2, p7, NS2, NS3, NS4A, NS4B, NS5A and NS5B (see e.g. Moradpour et al., (Nature Review (2007) 5:453-463). The C protein constitutes the nucleocapsid; E1 and E2 are transmembrane envelope glycoproteins; p7 is a membrane spanning protein of unknown function; and the various non-structural (NS) proteins have replication functions (Bartenschlager and Lohmann, 2000; Op De Beeck et al., 2001). The polyprotein precursor is co- and post-translationally processed by cellular and viral proteases into the mature structural and non-structural proteins. The structural proteins, including the core protein (corresponding to amino acid residues 1-191 of the H77 polyprotein set forth in SEQ ID NO:607), and the envelope glycoproteins E1 (corresponding to amino acid residues 192-383 of SEQ ID NO:607) and E2 (corresponding to amino acid residues 384-746 of SEQ ID NO:607) as well as p7 (corresponding to amino acid residues 747-809 of SEQ ID NO:607) are processed by the endoplasmic reticulum (ER) signal peptidases. The non-structural proteins, including NS2 (corresponding to amino acid residues 810-1026 of SEQ ID NO: 607), NS3 (corresponding to amino acid residues 1027-1657 of SEQ ID NO: 607), NS4A (corresponding to amino acid residues 1658-1711 of SEQ ID NO: 607), NS4B (corresponding to amino acid residues 1712-1972 of SEQ ID NO: 607); NS5A (corresponding to amino acid residues 1973-2420 of SEQ ID NO: 607) and NS5B (corresponding to amino acid residues 2421-3011 of SEQ ID NO: 607), are processed by two viral proteases, the NS2-3 protease and the NS3-4A serine protease.

E1 and E2

HCV entry into host cells requires attachment of the viral particle to the cell surface, followed by fusion of the viral envelope with the cellular membrane. The HCV envelope glycoproteins, E1 and E2, are thought to be involved in mediating virus entry into susceptible target cells. The E2 glycoprotein in particular is believed to be responsible for target cell binding. Thus, these polypeptides are targets for anti-viral agents, including anti-HCV antibodies.

As noted above, E1 and E2 are produced as part of the HCV polyprotein and then processed by cellular proteases before forming a non-covalent E1E2 complex, which is retained in the ER. Both E1 and E2 are heavily glycosylated. For example, the E1 polypeptide of H77 contains four N-linked glycans at positions corresponding to position 196, 209, 234 and 305 corresponding to the polyprotein set forth in SEQ ID NO: 607, while the E2 polypeptide contains 11 N-linked glycans at positions corresponding to position 417, 423, 430, 448, 476, 532, 540, 556, 576, 623 and 645 corresponding to the polyprotein set forth in SEQ ID NO: 607 (reference to an amino acid position of any protein of HCV, including E1 and E2, is made with reference to the position in the polyprotein, not to the mature protein).

Both E1 and E2 contain C-terminal transmembrane domains, which are involved in heterodimerization. The C-terminal region of E1, approximately corresponding to amino acids 331-383, is mainly responsible for membrane association and for inducing changes in membrane permeability. E1 also has a conserved internal hydrophobic region spanning amino acids 262-291 that could be involved in membrane permeabilizing activity (Ciccaglione et al., (2001) 82:2243-2250). The C-terminal region of E2 approximately corresponds to amino acids 715-746. Mutagenesis of the residues in the transmembrane domain indicate that these residues are critical for E1E2 heterodimerization and membrane retention, and viral entry. (Ciczora et al., (2005) J Gen Virol. 86:2793-2798)

Studies indicate that the E1E2 complex is involved in virus attachment to the host cell and internalization of the virus through clathrin-dependent endocytosis and fusion with host membrane. Further, the E1E2 complex binds to human LDLR, CD81 and SCARB1/SR-BI receptors, although these interactions have not been shown to be sufficient for infection (see e.g. Hsu et al., (2003) PNAS 100:7271-7276). While the precise mechanism of HCV binding and entry is not known, both E1 and E2 are targets of neutralizing antibodies. Development of polypeptides that are immunoreactive with the E1 or E2 polypeptide is believed to provide an especially effective regimen for preventing or treating HCV infection. For example, polypeptides that are immunoreactive with the E1 or E2 polypeptide can be used prophylactically in an HCV vaccine to protect against a new infection or prevent recurrent infection, for example, following liver transplantation. Polypeptides that are immunoreactive with the E1 or E2 polypeptide can be particularly useful in situations where the sera of a patient do not exhibit an immunoresponse to a viral challenge but the patient nevertheless carries the virus.

### C. ANTI-HCV POLYPEPTIDES AND ANTIBODIES

Provided herein are polypeptides that, either alone or in combination with other polypeptides, can bind to HCV. Included among the polypeptides provided herein are antibodies and fragments thereof that bind to HCV. In some instances, the antibodies and fragments thereof selectively bind E2, including E2 when expressed as a recombinant protein alone or in the E1E2 complex. In other instances, the antibodies and fragments thereof selectively bind the E1E2 complex but do not bind isolated E2 alone or a linear epitope of E1. Such antibodies can bind, for example, a conformational epitope in the E1E2 complex containing residues from E1, or a conformational epitope in the E1E2 complex, wherein the epitope is made up of residues from both the E1 and E2 polypeptides. Also provided are antibodies that bind to the same epitope as any of the antibodies described herein.

1. Polypeptides

The polypeptides provided herein that, either alone or in combination with other polypeptides, bind to HCV have an amino acid sequence that incorporates at least one complementarity-determining region (CDR) amino acid sequence set forth in the Tables 10, 11, 18, 19, 30 or 31, including those set forth in SEQ ID NOS: 78-308, 725-834 and 1090-1133, or a CDR having at least 70%, 80%, 90%, 91%, 92%, 93%, 94%, 95%, 96%, 97%, 98%, 99% or more sequence identity with a sequence of amino acids set forth in any of SEQ ID NOS: 78-308, 725-834 and 1090-1133. Typically, the polypeptides have at least one CDR with a sequence set forth in any of SEQ ID NOS: 725-834 or 1090-1133. In some examples, the polypeptides have two or three CDR sequences (a CDR triplet). Each CDR can have any of the CDR sequences described herein. In instances where the polypeptide contains a CDR triplet, the CDR triplet typically includes one CDR1 sequence, one CDR2 sequence and one CDR3 sequence described herein. For example, the first CDR can have any one of the CDR1 sequences set forth in SEQ ID NOS: 78-108; 171-205; 725-741, 778-796, 1090, 1091 and 1101-1113; the second CDR has any one of the CDR2 sequences set forth in SEQ ID NOS: 109-139; 206-240; 742-759, 797-815, 1092-1096 and 1114-1121; and the third CDR has any one of the CDR3 sequences set forth in SEQ ID NOS: 140-170; 241-275; 760-777, 816-834, 1097-1100 and 1122-1133. These sequences also are set forth in Tables 10, 11, 18, 19, 30 and 31.

The CDRs in a triplet can all be light chain CDR sequences, heavy chain CDR sequences or any combination of three CDR sequences described herein. Exemplary light chain CDR sequences include those set forth in SEQ ID NOS: 171-275, 291-308, 778-834 and 1101-1133. Exemplary heavy chain CDR sequences include those set forth in SEQ ID NOS: 78-170, 276-290, 725-777 and 1090-1100.

The sequences of the three CDRs in a polypeptide can be selected from the CDR sequences of one antibody group, i.e. one of the 22 different antibody groups A, B, C, D, E, F, G, H, I, J, K, L, M, N, O, P, Q, R, S, T, U, and V set forth in Tables 3 and 4. The antibody groups are based on heavy chain sequence identity. In some examples, a polypeptide containing three CDRs contains three CDRs from a single chain Fab fragment set forth in Tables 3 and 4 (SEQ ID NOS: 1-66, 986-1023 and 1062-1089). For example, a polypeptide provided herein can contain the heavy chain CDR1, CDR2 and CDR3 from the Fab heavy chain of any one of the Fabs set forth in Table 3, below. In another example, the polypeptide contains the CDR1, CDR2 and CDR3 from a Fab light chain set forth in Table 4.

When a polypeptide has two or more CDRs, for example, a CDR triplet, the CDRs can be appropriately spaced and held in place by spacer sequences such that in three-dimensional space, the CDRs and spacer sequences form a globular immunoglobulin domain in which the CDRs come together to form an antigen-binding surface. Thus, included among the polypeptides provided herein are those that have spacer amino acid sequences between each CDR sequence. These spacer sequences can resemble those in a human or other mammalian antibody, i.e. can be of similar lengths as that between the CDR sequences of a human or other mammalian antibody. In some examples, the CDRs and spacer sequences are arranged so that the resulting polypeptide resembles the variable region of a single chain of an antibody.

Although any spacer peptide sequence, such as an amino acid sequence having little or no ionic or lipophilic side chains, can be used, typically, a spacer sequence is a mammalian antibody variable region framework sequence, which are well known in the art and can readily be identified by one of skill in the art. For example, human framework sequences are provided, for example, in IMGT®, the international ImMunoGeneTics information system (http://imgt.cines.fr/) and Kabat et al., Sequences of proteins of immunological interest edn 5th: National Institutes of Health Publication No. 91-3242 (1991), and the National Center for Biotechnology Information (NCBI) genebank. In some examples, the polypeptides provided herein that, alone or in combination with another polypeptide, bind HCV, contain any one or more of the CDR or framework sequences described herein, including any combination of CDR and framework sequences described herein. When a polypeptide has two or three CDRs, the CDRs can be linked by human framework sequences. In some examples, the human framework sequences are consensus framework sequences of a human immunoglobulin, such as an IgG. In other examples, the framework sequences linking the two or three CDRs are any of those set forth in Tables 10, 11, 18, 19, 30 or 31, including any with a sequence of amino acids set forth in any of SEQ ID NOS: 309-572; 573-587; 593-610 and 835-982, 1134-1205. Any framework sequence can be used to link two or more CDRs.

In some examples, when a polypeptide has two or more, e.g. three, CDRs, and flanking framework sequences, the first CDR and first framework sequences is any combination of CDR 1 and framework 1 sequences described herein. The second CDR and framework sequences can be any combination of CDR2 and framework 2 sequences described herein. Similarly, the third CDR and framework sequences can be any combination of CDR3 and framework3 sequences described herein. For example, the polypeptides provided herein include those that have a combination of CDR and framework sequences from within the same antibody group disclosed herein, i.e. from within any one of groups A-V, but from different antibodies. Alternatively, a polypeptide can have a combination of CDR and framework sequences such as present in one of the Fab fragment light or heavy chains shown below. As such, included in the polypeptides provided herein are light chains and heavy chains of an antigen-binding fragment (Fab) of an antibody. Table 3 sets forth the heavy and light chains of exemplary Fabs described herein. These Fabs, as described in further detail below and in the Examples, selectively bind E2 and/or the E1E2 complex.

TABLE 3
Sequences of Fab Heavy Chain

| | | |
|---|---|---|
| | SEQ | |
| Fab | ID NO | Heavy Chain Polypeptide Sequence |

| | | |
|---|---|---|
| A | 1 | LEQSGAEVKKPGSSVKVSCKASGGTFSSFVINWVRQAPGQGLEW |
| | | VGGIFQAPGPEREWLRDINPISGTINYAQRFQGRVTMTADESMT |
| | | TVYMELSSLRSEDTAMYYCARENKFRYCRGGSCYSGAFDMWGQG |
| | | TMVTVSSAS |
| B1 | 2 | LEQSGAEVKKPGSSVKVSCRASGSPFSSYTITWVRQAPGQGLEW |
| | | MGGIILMTGKANYAQKFQGRVTITADRSTTTAYMEMSSLTSDDT |
| | | AIYYCARDPYVYAGDDVWSLSRWGQGTLVIVSSAS |
| B2 | 3 | LEQSGAEVKKPGSSVKVSCRASGSPYSSYTITWVRQAPGQGLEW |
| | | MGGIILMTGKANYAQKFQGRVTITADRATATAYMEMSSLTSDDT |
| | | AIYYCARDPYVYAGDDVRSLSRWGQGTPVIVSSAS |
| B3 | 4 | LEQSGAEVKKPGSSVKVSCRASGSPYSSYTITWVRQAPGQGLEW |
| | | MGGIILMTGKANYAQKFQGRVTITADRATATAYMEMSSLTSDDT |
| | | AIYYCARDPYVYAGDDVWSLSRWGQGTPVIVSSAS |
| B4 | 1062 | EVQLLEQSGAEVKKPGSSVKVSCRASGSPFSSYTITWVRQAPGQ |
| | | GLEWMGGIILMTGKANYAQKFQGRVTITADRSTTTAYMEMSSPT |
| | | SDDTAIYYCARDPYVYAGDDVWSLSRWGQGTLVIVSP |
| B5 | 1063 | EVQLLESGAEVKKPGSSVKVSCRASGSPYSSYTITWVRQAPGQG |
| | | LEWMGGIILMTGKANYAQKFQGRVTITADRATATAYMEMSSLTS |
| | | DDTAIYYCARDPYVYAGDDVWSLSRWGQGTPVIVSSAS |
| B6a & | 1064 | EVQLLEQSGAEVKKPGSSVKVSCRASGSPYSSYTITWVRQAPGQ |
| B6b & | | GLEWMGGIILMTGKANYAQKFQGRVTITADRATATAYMEMSSLT |
| B6c | | SDDTAIYYCARDPYVYAGDDVWSLSRWGQGTPVTVSSAS |
| B7 | 1065 | EVQLLEQSGAEVKKPGSSVKVSCRASGSPYSSYTITWVRQAPGQ |
| | | GLEWMGGIILMTGKANYAQKFQGRVTITADRATATAYMEMSSLT |
| | | SDDTAIYYCARDPYVYAGDDVWSLSRWGQGTPVIVSSAS |
| C1 & C1* | 5 | LEQSGAEVKTPGSSVRVSCRPPGGNFNSYSINWVRQAPGHGLEW |
| | | VGTFIPMFGTSKYAQKFQGRVTITADGSSGTAYMDLNSLRSDDT |
| | | AFYYCVRPETPRYCSGGFCYGEFDNWGQGTLVTVSSAS |
| C2 & C2* | 6 | LEQSGAEVKKPGSSVRVSCRAPGGTFNSYSVNWVRQAPGHGLEW |
| | | VGTLIPMFGTSSYAQKFQGRVTITADGSSGTAYMELNSLRSDDT |
| | | AVYYCVRPETPRYCRGGFCYGEFDNWGQGTLVTVSSAS |
| C3 | 7 | LEQSGAEVKEPGSSVRVSCRAPGGTFNSYSINWVRQAPGHGLEW |
| | | VGTLIPMFGTSNYAQKFQGRVTITADGSSGTAYMELNSLRSDDT |
| | | AVYYCVRPETPRYCSGGVCYGEFDNWGQGTLVTVSSAS |
| C4 | 8 | LESGAEVKKPGSSVRVSCRPPGGTFNSYSINWVRQAPGHGLEWV |
| | | GTIIPMFGTSKYAQKLQGRVTITADGSSGTAYMELNSLRSDDTA |
| | | VYYCVRPETPRYCSGGFCYGEFDNWGQGTLVTVSSAS |
| C5 | 9 | LEQSGAEVKKPGSSVRVSCRAPGGTLNSYSINWVRQAPGHGLEW |
| | | VGTLIPMFGTSNYAQKFQGRVTITADGSSGTAYMELNSLRSDDT |
| | | AVYYCVRPETPRYCSGGFCYGEFDNWGQGTLVTVSSAS |
| C6 | 10 | LEQSGAEVKKPGSSVRVSCRPPGGTFNSYSINWVRQAPGHGLEW |
| | | VGTIIPMFGTSKYAQKLQGRVTITADGSSGTAYMELNSLRSDDT |
| | | AVYYCVRPETPRYCSGGFCYGEFDNWGQGTLVTVSSAS |
| D1 | 11 | LESGGGLVQPGGSLRLSCEASGYYFSSFAMSWVRQTPGKGLEWV |
| | | SSIAGGTLGRTSYRDSVKGRFTISRDNSKNTVFLHMNNLRPEDT |
| | | AVYYCAKDPLLFAGGPNWFDHWGQGTLVTVSSAS |
| D2 | 12 | LEQSGGGLVQPGGSLRLSCEASGYYFSSFAMSWVRQTPGKGLEW |
| | | VSSIAGGTLGRTSYRDSVKGRFTISRDNSKNTVFLHMNNLRPED |
| | | TAVYYCAKDPLLFAGGPNWFDHWGQGTLVTVSSAS |
| D3 | 13 | LESGGGLVQPGGSLRLSCEASGYYFSSFAMSWVRQTPGKGLEWV |
| | | SSIAGGTLGRTSYRDSVKGRFTISRDNSKNTMFLHMNNLRPEDT |
| | | AVYYCAKDPLLFAGGPNWFDHWGQGTLVTVSSAS |
| D4 | 14 | LESGGGLVQPGGSLRLSCEASGYYFSSFAMSWVRQTPGKGLEWV |
| | | SSIAGGTLGRTSYRDSVKGRFTISRDNSKNTVFLHMSNLRPEDT |
| | | AVYYCAKDPLLFAGGPNWFDHWGQGTLVTVSSAS |
| E | 15 | LEQSGAELKKPGSSVKVSCKPSDGTFRAYTLSWVRQAPGQTLEW |
| | | MGRIMPTVGITNYAQKFQGRVTISADMSTATAYMELSSLRSDDT |
| | | AIYYCAKGPYVGLGEGFSEWGQGTLVTVSSAS |
| E2 | 1066 | EVGLLEQSGAELKKPGSSVKVSCKPSDGTFRAYTLSWVRQAPGQ |
| | | TLEWLGRIMPTVGITNYAQKFQGRVTISADMSTATAYMQLSSLR |
| | | PDDTAIYYCAKGPYVGLGEGFSEWGQGTLVTVSS |
| E3 | 1067 | EVGLLEQSGAELKKPGSSVKVSCKPSDGTFRAYTLSWVRQAPGQ |
| | | ALEWMGRIMPTVGITNYARKFQGRVTISADMSTATAYMELSSLR |
| | | SDDTAIYYCAKGPYVGLGEGFSEWGQGTLVTVSS |
| E4 | 1068 | EVGLLEQSGAELKKPGSSVKVSCKPSDGTFRAYTLSWVRQAPGQ |
| | | TLEWMGRIMPTVGITNYAQKFQGRVTISADMSTATAYMELSSLR |
| | | SDDTAIYYCGKGPYVGLGEGFSEWGQGTLVTVSS |
| F | 16 | LEQSGNEVKKPGASVKVSCRAYGYNFGSERLSWVRQAPGQGLEW |
| | | MGWISAYNGGINYSQKFQGRFTMTTDTSTRTGYMELRNLRSDDT |
| | | AVYYCARGGGTEWGQGTLVIVSSDE |
| G | 17 | LEQSGAEMKKPGASLKVSCKTSGYTFDDYGVTWVRQAPGQGLEW |
| | | MGWISAYSGNTFYARKFQGRVTMTTDPSTRTAYMELRSLRSDDT |
| | | AVYFCARDRGLAINGVVFPYFGLDVWGQGTTVTVSSAS |
| H1, H1* | 18 | LEQSGAEVKKPGSSVKVSCEASGGTFDNYSLNWVRQAPGQGLEW |
| & H1** | | IGGVVPLFGTTKYAQKFQGRVTISDDKSTGTGHMELRSLRSEDT |
| | | AVYYCVHCVTPRHCGGGFCYGEFDYWGQGTLVTVSSAS |
| H2 | 19 | LEQSGAEVKKPGSSVKVSCETSGGTFDNYALNWVRQAPGQGLEW |
| | | IGGVVPLFGTTKYAQKFQGRVTISDDKSTGTGHMELRSLRSEDT |
| | | AVYYCVHCVTPRHCGGGFCYGEFDYWGQGTLVTVSSAS |
| H3 | 20 | LEQSGAEVKKPGSSVKVSCETSGGTLDNYALNWVRQAPGQGLEW |
| | | IGGVVPLFGTTRNAQKFQGRVTISDDKSTGTGHMELRSLRSEDT |
| | | AVYYCVHCVTPRYCGGGFCYGEFDYWGQGTLVTVSSAS |
| I | 21 | LESGGGLVQPGRSLRLSCKASGFNFAQYTMNWVRQAPGKGLEWI |
| | | GLIRTTAYDAATHYAASVEGRFTISRDDSKSTAYLQINGLKTED |
| | | TAVYYCARPHGPGLSLGIYSAEYFDEWGQGTLVTVSSAS |
| J1 | 22 | LEQSGPEVKKPGSSVKVSCKGSGDRFNDPVTWVRQAPGQGLEWI |
| | | GGIIPAFGATKYAQKFQGRVVISADASTDTAYMELSSLRSEDTA |
| | | VYYCAKVGVRGIILVGGLAMNWLDPWGQGTLVTVSAAS |
| J2 | 23 | LEQSGPEVKKPGSSVKVSCKDSGDTFNEPVTWVRQAPGQGLEWI |
| | | GGIIPAFGVTKYAQKFQGRVIISADASTATAYLELSSLRSEDTA |
| | | VYYCAKVGLRGIVMVGGLAMNWLDPWGQGTQVTVSSAS |
| J3 & | 24 | LEQSGPEVKKPGSSVKVSCKGSGDTFNDPVTWVRQAPGQGLEWI |
| J3* | | GGIIPLFGAAKYAQKFQGRVTISADASALTTYMEMSSLRPEDTA |
| | | VYYCAKVGLRGITLVGGLAMNWLDPWGQGTLITVSSAS |
| J4 | 25 | LEQSGAEVKKPGSSVRVSCEVSGDTFREPVSWVRQAPGQGFEWI |
| | | GGIIPMFGATHYAQKLQGRITISADQSTNTVYMELRSLRSDDTA |
| | | VYYCAKVGLRGINMVGGLAMNWFDPWGQGTLVTVSSAS |
| J5 | 1069 | EVQLLEQSGAEVKKPGSSVTVSCKASGGTVSSYPITWVRQAPGQ |
| | | GLEWMGGTIPVFGAPKYAPKFQGRVTITADESTSTAYMELRSLR |
| | | SDDTAMYYCAIVGMRGITLVGGLAMNWLDPWGQGTLVIVSS |
| K | 26 | LEQSGPGLVKPGRPFSLTCAISGDSVSSDSAAWNWVRQSPSRGL |
| | | EWLGRTFYRSKWYYDYTVSVKSRITINSDTSKNQFSLHLNSVTP |
| | | EDTAVYYCVRDFYIGPTRDVYYGMDVWGQGTTVTVSSAS |
| L1 | 27 | LEQSGAEVKKPGSSVKVSCKASGDTFRSYVITWARQAPGQGLEW |
| | | MGAIIPFFGTTNLAQKFQGRVTITADESTQTVYMDLSSLRSDDT |
| | | AVYYCAKAGDLSVGGVLAGGVPHLRHFDP WGQGTLVTVSSAS |
| L2 | 28 | LEQSGAEVKMPGSSVKVSCKASGDTFRSSVITWARQAPGQGLEW |
| | | MGAIIPFFGTTNLAQKFQGRVTITADESTKTVYMDLSSLRSDDT |
| | | AVYYCAKAGDLSVGGVLAGGVPHLRHFDPWGQGTLVTVSSAS |
| L3 | 29 | LEQSGAEVKKPGSSVKVSCKASGDTFRSYVITWARQAPGQGLEW |
| | | MGAIIPFFGTTNLAQKFQGRVTITADESTKTVYMDLSSLTSDDT |
| | | AVYYCAKAGDLSVGGVLAGGVPHLRHFDPWGQGTLVTVSSAS |
| L4 | 30 | LEQSGAEVKKPGSSVKVSCKASGDTFRSYVITWARQAPGQGLEW |
| | | MGAIIPFFGTTNLAQKFQGRVTITADESTKTVYMDLSSLRSDDT |
| | | AVYYCAKAGDLSVGGVLAGGVPHLRHFDPWGQGTLVTVSSAS |
| L5 | 1070 | EVQLLEQSGTEVKKPGSSVKVSCKVPGDTFRSYVITWVRQAPGQ |
| | | GLEWLGGILPFFGTTNLAQKFQGRVTLTADESTTTAYMELSSLR |
| | | AEDTAVYYCAKAGDLAFGGVIAGGVPHLSHFDPWGQGTLVTVSS |
| L6 | 1071 | EVQLLEQSGTEVKKPGSSVKVSCKVPGDTFRSYVITWVRQSPGQ |
| | | GLEWLGGILPFFGTTNLAQKFQGRVTLTADESTTTAYMELSSLR |
| | | SEDTAVYYCAKAGDLAFGGVIAGGVPHLSHFDPWGQGTLVTVSA |
| L7 | 1072 | EVQLLEQSGAEVKKPGSSVKVTCKVPGDTFRSYVITWVRQAPGQ |
| | | GLEWLGGILPFFGTTNLAQKFQGRVTLTADESTTTAYMELSSLR |
| | | AEDTAVYYCAKAGDLAFGGVIAGGVPHLSHFDPWGQGTLVTVSS |
| M | 31 | LEQSGAEVKKPGASVKVSCKASGYTFTNYAITWVRQAPGQGLEW |
| | | MGWISGDSTNTYYGQKFQGRVTMTTDTSTSTAYMELTSLTSEDT |
| | | AVYYCARESLYMIAFGRVIWPPLDYWGQGTLVTISSAS |
| N1 | 986 | EVQLLEQSGPEVKKPGDSLRISCKMSGDSLVTTWIGWVRQKPGQ |
| | | GLEWMGIINPGDSSTNIYPGDSATRYGPSFQGQVTISIDKSTST |
| | | AYLQWNAVKPSDTGIYYCARHVPVPISGTFLWREREMHDFGYFD |
| | | DWGQGTLVIVSS |
| N2 | 987 | EVQLLEQSGPEVKKPGDSLRISCKMSGDSLVTTWIGWVRQKPGQ |
| | | GLEWMGIINPGDSSTNIYPGDSATRYGPSFQGQVTISIDKSTST |
| | | AYLQWNTVKPSDTGIYYCARHVPVPISGTFLWREREMHDFGYFD |
| | | DWGQGTLVIVSS |
| N3 | 988 | EVQLLEQSGAEVKKPGDSLRISCKMSGDSLVWIGWVRQKPGQGL |
| | | EWMGIINPGDSATNIYPGDSDTRYGPSFQGQVTISIDKSTSTAY |
| | | LQWNAVKASDTGIYYCARHVPVPISGTFLWREREMHDLGYFDDW |
| | | GQGTLVIVSS |
| N4 | 989 | EVQLLEQSGPEVKKPGDSLRISCKMSGDSLVTTWIGWVRQKPGQ |
| | | GLEWMGIINPGDSSTNIYPGDSATRYGPSFQGQVTISIDKSTST |
| | | AYLQWNNVKASDTGIYYCARHVPVPISGTFLWREREMHDFGYFD |
| | | DWGQGTLVIVSS |
| O1 | 990 | EVQLLEQSGAEVKKAGESVRLSCKASGYRFGDYWIAWVRQLPGR |
| | | APEWMGIIYPDDSDTKYSPSFQGQVTISADKSIRTTFLDWGSLK |
| | | ASDTAIYYCARGCLGAKCYYPHYYYGLDVWGQGTTVIVSS |
| P1 | 991 | EVQLLESGGGVVQPGGSLRLSCAASGFTFTSFTMHWVRQAPGKG |
| | | LEWVALISHDGSNKDYADSVRGRFTVSRDNSKKMVYLQMSSLRP |
| | | DDAAVYYCARGGPAYYTYSDTLTGYHNVVGDYWGQGTLVTVSS |
| P1a | 992 | EVQLLESGGGVVQPGGSLRLSCAASGFTFTSFTMHWVRQAPGKG |
| | | LEWVALISHDGSNKDYADSVRGRFTVSRDNSKKMVYLQMSSLRP |
| | | DDAAVYYCARGGPAYYTYSDTLTGYHNVVGDYWGQGTLVTVSS |
| P2 | 993 | EVQLLESGGGVVQPGKSLRLSCAVSGFTLNTFAMHWVRQVPGKG |
| | | LEWVALTSHDGSRQDYADSVRGRFTISRDNSKSMVFLLMNSLRA |
| | | EDTAVYYCVRGGPAYYTYNDVLTGYAYVVGDFWGQGTLVTVSS |
| P3 | 994 | EVQLLESGGGVVQPGKSLTVSCAASGFTFSTFTMHWVRQAPGKW |
| | | LEWVAVISHDGGTEHYADSVTGRFTISRDNSKNTLHLQMNSLRP |
| | | EDTAVYFCARGGPAYYLYNDVLTGYYNVVGDFWGQGTLVTVSS |
| P4 | 995 | EVQLLESGGGVVQPGKSLTVSCAASGFTFSTFTMHWVRQAPGKG |
| | | LEWVAVISHDGGTEHYADSVTGRFTISRDNSKNTLHLQMNSLRP |
| | | EDTAVYFCARGGPAYYLYNDVLTGYYNVVGDFWGQGTLVTVSS |
| P5 | 996 | EVQLLESGGGVVQPGKSLTVSCAASGFTFSTFTMHWVRQAPGKW |
| | | LEWVAVISHDGGTEHYADSVTGRFTISRDNSKNTLHMQMNSLRL |
| | | EDTAVYFCARGGPAYYLYNDVLTGYYNVVGDFWGQGTLVTVSS |
| P6 | 997 | EVQLLEQSGAEVRKPGASVKVSCKASGYTFTNNGLNWVRQAPGQ |
| | | GLEWMGWISPYNGDTDFAHKFQGRISMTTDTSTNTAYMELRSLR |
| | | SDDTAVYYCARDRNSAGGTWLFRDPPPGSTFFDSWGQGSLVTVS |
| | | S |
| Q1 | 998 | EVQLLEQSGAEVKKPGASVKVSCKASGYTFTNNGLNWVRQAPGQ |
| | | GLEWMGWISPYNGDTDFAHKFQGRISMTTDTSTNTAYMELRSLR |
| | | SDDTAVYYCARDRNSAGGTWLFRDPPPGSTFFDSWGQGSLVTVS |
| | | S |
| Q2 | 999 | EVQLLEQSGAEVKKPGASVKVSCKASGYTFSIYGVAWVRQAPGQ |
| | | GLEWMGWISPQNGDTHSPQKFQGRLTMTTDTSTSTAYMELRSLR |
| | | SDDTAVYFCARDYGVNFGGGSEHNLDYWGRGTRVTVSS |
| R1 | 1000 | EVQLLEQSGAEVKKPGTSVKVSCTASGYIFTSFGISWVRQAPGQ |
| | | GLEWMGRIDTYNGKTNYAQKLQGRVTMTTDTYTSTAYMELRSLT |
| | | SDDTAVYYCARDDCRSSTCYLAQHNWQAYYHDSWGQGTLVTVSS |
| S1 | 1001 | EVQLLESGGGLVQPGGSLRLSCAASGFTFSRFWMHWVRQAPGKG |
| | | LVWVARINSDGSSTTYADSVKGRFTISRDNAKNTLYLQMNSLRD |
| | | EDTAVYFCARGGDSSSPYYYPMDVWGQGTTVAVSS |
| S2 | 1073 | EVQLLEQSGAEVKKPGASVKVSCTASGYIFTSFGISWVRQAPGQ |
| | | GLEWMGRIDTYNGKTNYAQKLQGRVTMTTDTYTSTAYMELRSLT |
| | | SDDTAVYYCARDDCRSSTCYLAQHNWQAYYHDSWGQGTLVTVSS |
| T1 | 1002 | EVQLLEQSGPEVKRPGTSVKMSCKISGGASITQAMSWVRQAPGQ |
| | | GLEWMGGITPIFGTVNYAQKILGRVTITADEDTVSLELSSLKSE |
| | | DTAVYYCAREVNLKTWNLAHPNVFDVWGQGTMLTVSS |
| U1 | 1003 | EVQLLEQSGAEVQKPGASVKVSCKPSGYIFTNFGISWVRQAPGQ |
| | | GLEWMAWINTYNGKTTYAQSLQGRVTLTTDPYTNTVFMELRSLR |
| | | SDDTAVYYCARENEGEYVWGHFRSDYWGQGTLVTVSS |
| U2 | 1074 | EVQLLEQSGAEVKRPGSSVKLSCKNSGGTFITQAMSWVRQAPGQ |
| | | GLEWMGGIIPVFGTVNYAQNILDRATATADEDTFSLELRGLRLE |
| | | DTAVYYCAREVNLKTWNLARPDVFDIWGPGTLVTVSS |
| U3 | 1075 | EVQLLEQSGAEVKRPGSSVKISCKNSGGTFITQAMSWVRQAPGQ |
| | | GLEWMGGIIPIFGTVNYAQKILGRVTITADEDTVSMELSSLGFE |
| | | DTAVYYCAREVNLKSWNLAHPDVFDIWGQGTLVTVSS |
| V1 | 1004 | EVQLLEQSGPEVKKPGDSLRISCKMSGDSLVTTWIGWVRQKPGQ |
| | | GLEWMGIINPGDSSTNIYPGDSATRYGPSFQGQVTISIDKSTST |
| | | AYLQWNAVKPSDTGIYYCARHVPVPISGTFLWREREMHDFGYFD |
| | | DWGQGTLVIVSS |

TABLE 4
Sequences of Fab Light Chain

| | | |
|---|---|---|
| | (SEQ ID | |
| Fab | NO) | Light Chain Polypeptide Sequences |

| | | |
|---|---|---|
| A | 32 | ELTQSPATLSVSPGESATLSCRASQSVSDNLAWYQQKPGQAPRLLI |
| | | YGASSRAPAIPGRFSGSGSGTDFTLTISRLEPEDLAVYHCQQYGAS |
| | | PWTFGQGTKVEIKRTVAAPSVFIFPPSDEQLKSGTASVV |
| B1 | 33 | ELTLTQSPGTLSLSPGERATLSCRASQSVSNSYLAWYQQKPGQAPR |
| | | LLIYGASSRATGIPDRFSGSGSGTDFTLTISRLEPEDFAVYYCQQY |
| | | GSSPQTFGQGTKVEIKRTVAAPSVFIFPPSDEQLKSGTASVV |
| B2 | 34 | ELTQSPDSLAVSLGERATINCKSSQSVLYSSNNKNYLAWYQQKPGQ |
| | | PPQLLIYWASTRESGVPDRFSGSGSGTDFTLTISSLQAEDVAVYFC |
| | | QQYYSTPFTFGPGTKVEIKRTVAAPSVFIFPPSDEQLKSGTASVV |
| B3 | 35 | ELVMTQSPGTLSLSPGERATLSCRASQRVGSSYLAWYQQKPGQAPR |
| | | LLVYGASSRATGIPDRFSGSGSGTDFTLTISRLQPEDFAVYYCQQY |
| | | GTTFGQGTRVDIKRTVAAPSVSIFPPSDEQLKSGTASVV |
| B4 | 1076 | ELTLTQSPGTLSLSPGKRATLSCRASQSVSGSYLAWYQQKPGQAPR |
| | | LLIYGASNRATGIPHRFSGSGSGTDFTLTISRLEPEDFAVYYCQQY |
| | | GSSPPTFGQGTRVDIKRT |
| B5 | 1077 | ELTQSPLSLPVIIGQPASISCSSSESLVDSDGNTYLHWFQQRPGQS |
| | | PRRLIYKVSNRDAGVPDRFSGSGSGTDFTLKISRVEAEDVAVYYCM |
| | | QATHWPPITFGPGTRLEVKRT |
| B6a | 1078 | LTQSPGTLSLSPGERATLSCRASQSVASNYVAWYQQKPGQAPRLLI |
| | | YGTSYRATGIPGRFSGSGSGTDFTLTISGLEPEDFAVYYCQQYGSS |
| | | PQTFGQGTKVEIKRT |
| B6b | 1082 | ELTQSPGTLSLSPGERATLSCRASQSVSRGNLAWYQQKRGQPPRLL |
| | | IYGASYRATGIPDRFSGSGSGTDFTLTITKLEPEDFAVYYCQQYGH |
| | | SLAFGQGTKVEIKGT |
| B6c | 1365 | ELTQSPGTLSLSPGERATLSCRASQSVSSSYLAWYQQKPGQAPRLL |
| | | IYGASSRATGIPDRFSGSGSGTDFTLTISRLEPEDFAVYYCQQYGS |
| | | SPQTFGQGTKVEIKRT |
| B7 | 1079 | ELTQSPLSLPVTLGQPASISCRSSQSLVYSDGNTYLNWFQQRPGQS |
| | | PRRLIYKVSNRDSGVPDRFSGSGSGTDFTLKISRVEAEDVGVYYCM |
| | | QGTHWPPTFGQGTKVDIKRT |
| C1 | 36 | ELTLTQSPGTLSLSPGERATLSCRASQSVSGNYLAWYQQKPGQAPR |
| | | LLIYGASNRATGIPHRFSGSGSGTDFTLTISRLEPEDFAVYYCQQY |
| | | GSSPTFGQGTRVDIKRTVAAPSVFIFPPSDEQLKSGTASV |
| C1* | 37 | ELTQSPSTLSLSPGEGATLSCRPSQSVSRNYLAWYQQKPGQAPRLL |
| | | IYGASTRATGIPDRFSGSGSGTNFTLTISRLEPEDFAVYFCQHYGN |
| | | SPPYTFGQGTKLEIKRTVAAPSVFIFPP |
| C2 | 38 | ELTQSPGTLSLSPGERAALSCRASQSISTNYLAWYQQKPGQAPRLL |
| | | IYGTSNRATGIPDRFSGTGSGTDFSLTISRLEPEDSAVYYCQQYGT |
| | | SPFTFGPGTKVDIKRTVAAPSVFIFPPS |
| C2* | 39 | ELTQSPGTLSLSPGERATLSCRASQSVSSSYLAWYQQKPGQAPRLL |
| | | IYGASSRATGIPDRFSGSGSGTDFTLTISRLEPEDFAVYYCQQYGS |
| | | SPQTFGQGTKVEIKRTVAAPSVFIFPPSDEQLKSGTASVV |
| C3 | 40 | ELTQSPGTLSLSPGERATLSCRASQSVSSSYLAWYQQKPGQAPRLL |
| | | IYGASSRATGIPDRFSGSGSGTDFTLTISGLEPEDFAVYYCQQYGS |
| | | SPLTFGGGTKVEIKRTVAAPSVFIFPPSD |
| C4 | 41 | ELTQSPGTLSLSPGERATLSCRASQSVSSSYLAWYQQKPGQAPRLL |
| | | IYGASSRATGIPDRFSGSGSGTDFTLTISRLEPEDFAVYYCQHYGS |
| | | SSYTFGQGTRVEIKRTVAAPSVFIFPPSDEQLKSGTASVV |
| C5 | 42 | ELTQSPATLYVSPGERATLSCRASQSVPDNHLAWYQQKPGQTPRLL |
| | | IYGASKRATGIPDRFSGSGSGTDFTLTISRLEPEDFAVYYCQQYGS |
| | | SPQTFGQGTKVEIKRTVAAPSVFIFPPSDEQLKSGTASVV |
| C6 | 43 | ELTQSPGTLSVSPGEAATLSCRASQSVSSNLAWYQQKPGQAPRLLI |
| | | YGASTRATGIPARFSGSGSGTDFTLTISRLEPEDFAVYYCQQYGGS |
| | | PPYTFGQGTKLEIKRTVAAPSVFIFPPSDEQLKSGTASVV |
| D1 | 44 | ELTQSPGTLSLSPGERATLSCRASQSVSSSYLAWYQQKPGQAPRLL |
| | | IYGASSRATGIPDRFSGSGSGTDFTLTISRLEPEDFAVYYCQQYGS |
| | | SPQTFGQGTKVEIKRTVAAPSVFIFPPSDEQLKSGTASV |
| D2 | 45 | ELTQSPATLSVSPGERATLSCRASQTISDNLAWYQQKPGQAPRLLI |
| | | YGASSRATGIPDRFSGSGSGTDFTLTISRLEPEDFAVYYCQQYGSS |
| | | PQTFGQGTKVEIKRTVAAPSVFIFPPSD |
| D3 | 46 | ELTLTQSPGTLSLSPGERATLSCRASQTVSSSYLAWYQQKPGQAPR |
| | | LLIYGASSRATGIPDRFSGSGSGTDFTLTISRLEPEDFAVYYCQQY |
| | | GSSPQTFGQGTKVEIKRTVAAPSVFIFPPSDEQLKSGTA |
| D4 | 47 | ELVMTQSPGTLSLSPGERATLSCRASQSVSSSYLAWYQQKPGQAPR |
| | | LLIYGASSRATGIPDRFSGSGSGTDFTLTITRLEPEDFAVYYCQQY |
| | | GSSPQTFGQGTKVQIKRTVAAPSVFIFPPSDEQLKSGTA |
| E | 48 | ELVLTQSPLSLPVTLGQPASISCRSTQSLVYSDGNTYLNWFHQRAG |
| | | QPPRRLIYKVSNRDSGVPERFSGSGSGTDFTLKISRVEAEDVGIYY |
| | | CMQGAHWPPTFGGGTKVEINRTVAAPSVFIFPPSDEQLKSGTAS |
| E2 | 1080 | ELTQSPLSLPVTLGQPASISCRSSQSLVYSDGNTYLIWFQQRPGQS |
| | | PRRLIYNVSNRDSGVPDRFSGSGSGTDFTLKISRVEAEDLGVYYCM |
| | | QGAHWPPTFGQGTRLEIKGT |
| E3 | 1081 | ELVLTQSPLSLPVTLGQPASISCRSTQSLVYSDGNTYLNWFHQRPG |
| | | QPPRRLIYKVSNRDSGVPERFSGSGSGTDFALKISRVEADDVGIYY |
| | | CMQGAHWPPTFGGGTKVEISRT |
| F | 49 | ELQMTQSPSFLSASVGDRVTITCRASQGISSYLAWYQQKPGKAPKL |
| | | LISSVSTLQSGVSSRFSGSGSGTGFTLTISSLQSEDSATYYCEQLN |
| | | SFPYTFGQGTKLEIKRTVAAPSVFIFPPSD |
| G | 50 | ELTQSPVSLPVTPGEPASISCRSSQSLLHSNGNHYLDWYLQKPGQS |
| | | PQLLMYLGSNRASGVPDRFSGSGSGTDFTLKISRVEAEDVGVYYCM |
| | | QGLQTPWTFGQGTKVEIKRTVAAPSVFIFPPSD |
| H1 | 51 | ELTLTQSPGTLSLSPGERATLSCRASQSISSSYLAWYQQKPGQAPR |
| | | LLIYGASRRATGIPDRFSGSGSGTDFTLTISRLEPEDFAVYYCQQY |
| | | GSSPLTFGGGTKVEIKRTVAAPSVFIFPPSD |
| H1* | 52 | ELTQSPATLSVSPGERATLSCRASRGISSNLAWYQQKPGQAPRLLI |
| | | YGASSRATGIPDRFSGSGSGTDFTLTISRLEPEDFAVYYCQQYGSS |
| | | PQTFGQGTEVEIKRTVAAPSVFIFPPSDEQ |
| H1** | 53 | ELTLTQSPGTLSLSPGERATLSCRASQSVSSDSLAWYQQKPGQAPR |
| | | LLIYGASSRATGIPDRFSGSGSGTDFTLTISRLEPEDLGVYYCQQY |
| | | GPSPPGYTFGQGTKLEIKRTVAAPSVFIFPPSDEQLKSGTASVV |
| H2 | 54 | ELTLTQSPGTLSLSPGERGTLSCRASQSVSSSYLAWYQQKPGQAPR |
| | | LLIYGASSRATGIPDRFSGSGSGTDFTLTISRLEPEDFAVYYCQQY |
| | | GSSPQTFGQGTKVEIKRTVAAPSVFIFPPSDEQLKSGTASVV |
| H3 | 55 | ELTQSPATLSVSPGERATLSCRASQSVSSNLAWYQQKPGQAPRLLI |
| | | YGASTRATGIPARFSGSGSGTEFTLTVSRLEPEDSAVYFCQQYYRS |
| | | PLTFGGGTKVEIKRTVAAPSVFIFPPSDEQLKSGTASVV |
| I | 56 | ELTLTQSPATLSVSPGERATLFCRANQSVGRNLAWYQQKPGQAPRL |
| | | LIYGISTRTTTTPTRFSGSGSGTDFTLTISRLQSEDFAVYYCQQYN |
| | | KWPPWTFGQGTKLEIKRTVAAPSVFVFPPS |
| J1 | 57 | ELTQSPGTLSLSPGERATLSCRASQSVSSSYLAWYQQKPGQAPRLL |
| | | IYGASSRATGIPDRFSGSGSGTDFTLTISRLEPEDFAVYYCQQYGS |
| | | SPQTFGQGTKVEIKRTVAAPSVFIFPPSDEQLKSGTASVV |
| J2 | 58 | ELTQSPGTLSLSPGERATLSCRASQSVSSSYLAWYQQKPGQAPRLL |
| | | IYGASSRATGIPDRFSGSGSGTDFTLTISGLEPEDFAVYYCQQYGS |
| | | SPQTFGQGTKVEIKRTVAAPSVFIFPPSDEQLKSGTASV |
| J3 | 59 | EFTLTQSPGTLSLSPGERGTLSCRASQSVSSSYLAWYQQKPGQAPR |
| | | LLIYGASSRATGIPDRFSGSGSGTDFTLTISRLEPEDFAVYYCQQY |
| | | GSSPQTFGQGTKVEIKRTVAAPSVFIFPPSDEQLKSGTASVV |
| J3* | 60 | ELTLTQSPGTLSLSPGERATLSCRASQSVSSSHLAWYQQKPGQAPR |
| | | LLIYGASSRATGIPDRFSGSGSGTDFTLTISRLEPEDFAVYYCQQY |
| | | GSSPQTFGQGTEVEIKRTVAAPSVFIFPPSDEQLKSGTASVV |
| J4 | 61 | ELTQSPGTLSLSPGERATLSCRASQSVSSNSLAWYQQKPGLAPRLL |
| | | IYGASSRATGIPERFSGSGAGTGFTLTISTLEPEDFAIYYCQQYGG |
| | | SPPRFTFGPGTKVDIRRTVAAPSVFIFPPSDEQLKSGTASVV |
| J5 | 1083 | ELTQSPGTLSLSPGERATLSCRASQSVSSNYLAWYQQKPGQAPRLL |
| | | IYGASSRATGIPDRFSGSGSGTDFTLSISRLEPEDFAVYYCQQYGS |
| | | SPLTFGGGTKVEIKRT |
| K | 62 | ELTQSPGTLSLSPGERATLSCRASQSVSSSYLAWYQQKPGQAPRLL |
| | | IYGASSRATGIPDRFSGSGSGTDFTLTISRLEPEDFAVYYCQQYGS |
| | | SPQTFGQGTKVEIKRTVAAPSVFIFPPSDEQLKSGTASVV |
| L1 & | 63 | ELTQSPGTLSLSPGERATLSCRASQSITSRYLAWYQQKPGQAPRLL |
| L2 | | IYGASSRATGIPDRFSGSGSGTDFTLTISRLEPEDFAVYYCQQYGD |
| | | SPVGFGPGTKVEIKRTVAAPSVFIFPPSDEQLKSGTASVV |
| L3 | 64 | ELVMTQSPATLSLSPGERATLSCRASQSVGSYLAWYQQKPGQAPRL |
| | | LIYDASNRATGIPARFSGSGSGTDFTLTISSLEPEDFAVYFCQQYG |
| | | SSPLTFGQGTKVEIKRTVAAPSVFIFPPSDEQLKSGTASVV |
| L4 | 65 | ELTQSPGTLSLSPGERATLSCRAGQTVASNSLAWYQHKPGQAPRLL |
| | | IYGASIRASGIPDRFSGSGSGTDFTLTISRLEPEDFAVYYCQQYGL |
| | | SPSTFGQGTRLEIKRTVAAPSVFIFPPSDEQLKSGTASVV |
| L5 | 1084 | ELTQSPGTLSLSPGERATLSCRASQSVRSNYLAWYQQKPGQAPRLL |
| | | IYGASSRATGIPDRFSGSGSGTDFTLTISRLEPEDFAMYYCQQYGS |
| | | SMCSFGQGTKVEIKRT |
| L6 | 1085 | ELTQSPGTLSLSPGERATLSCRASQSVSSSYLAWYQQKPGQAPRLL |
| | | IYGASSRATGIPDRFSGSGSGTDFTLTISRLEPEDFAVYYCQQYGS |
| | | SVTFGPGTKVDIKRT |
| L7 | 1086 | ELTQSPGTLSLSPGERATLSCRASQSVPSSYLGWYQQKPGQAPRLL |
| | | IYGASSRATGIPERFSGSGSGTDFTLTISRLEPEDFAVYYCQQYGS |
| | | SLSFGQGTKVDLKRT |
| M | 66 | ELTLTQSPGTLSLSPGERATLSCRASQSIRSSYLAWYQQKPGQAPR |
| | | LLIYAAASRATGIPDRFSGSGSGTDFTLTISRLEPEDFAVYFCHHY |
| | | GGSPRTFGQGTKVEIKRTVAAPSVFIFPPSDEQLKSGT |
| N1 | 1005 | ELTQSPGTLSLSPGERATLSCRASQSVSSSYLAWYQQKPGQAPRLL |
| | | IYGASNRAAGIPDRFSGSGSGADFTLTISRLEPEDFAVYYCQQYGS |
| | | SLITFGQGTRLEIKRT |
| N2 | 1006 | ELTQSPGTLSLFPGERATLSCRASQSILGRYLAWYQQKGGRAPRLL |
| | | IFGASKRATGIPDRFSGSGSGTDFTLTIGRLEPEDFAVYYCQHYGS |
| | | SITFGQGTRLDIKRT |
| N3 | 1007 | ELTQSPGTLSLSPGERATLSCRASQSVSSSYLAWYQQKPGQAPRLL |
| | | IYGASSRATGIPDRFSGSGSGTDFTLTISRLEPEDFAVYYCQQYGS |
| | | SLTFGGGTKVEIKRT |
| N4 | 1008 | ELTLTQSPGTLSLSPGERATLSCRASQSVSNNYLAWYQQKPGQAPR |
| | | LLIYGASSRATGIPDRFSGSGSGTGFTLIISRLEPEDFAVYYCQQY |
| | | GSSSITFGQGTRLEIKRT |
| O1 | 1009 | ELTQSPSSLSASVGDRVTITCRATQGIDNYLNWYQQKPGKPPRLLI |
| | | YGASSLQSGVPSRFSGGGSGTHFTLTITNLQPEDFATYYCQQSYST |
| | | PPETFGQGTKVEIKRT |
| P1 | 1010 | ELTQSPGTLSLSPGERATLSCRASQVRGFLAWFQQKPGQAPRLLIY |
| | | GASNRAPGIPDRFSGSGSGTDFTLTISRLEPEDFAVYYCQQYGDSP |
| | | PITFGQGTRLEIKRT |
| P1a | 1011 | ELTQSPGTLSLSPGERATLSCRTSQSVSSTYLAWYQQKPGQPPRLL |
| | | IYGASNRAPGIPDRFSGSGSGTDFTLTISRLEPEDFAVYYCQQYGD |
| | | SPPITFGQGTRLDIKRT |
| P2 | 1012 | ELVLTQSPGTLSLSPGERATLSCRASQSVSSSYLAWYQQKPGQAPR |
| | | LLIYGASSRATGIPDRFSGSGSGTDFTLTISRLEPEDFAVYYCQQY |
| | | GDSPPFFGPGTKVDIKRT |
| P3 | 1013 | ELTQSPGTLSLSPGERATLSCRASQSVSSTYLAWYQQKPGQAPRLL |
| | | IYGASSRATGIPDRFSGSGSGTDFTLTISRLEPEDFAVYYCQQYGS |
| | | SPLTFGGGTKVEIKRT |
| P4 | 1014 | ELTQSPGTLSLSPGERATLSCRASQSVSSSNLAWFQHKSGRAPRLL |
| | | IYGASNRAPDIPDRFSGSGSGTDFTLSISRLEPEDFAVYYCQRYGD |
| | | SPPITFGQGTRLEIKRT |
| P5 | 1015 | ELTQSPASLSLSPGERATLSCRASQSVGTYFAWYQQKPGQAPRLLI |
| | | YGASNRATGIPDRFSGSGSGTDFTLTVSRLEPEDFAVYYCQQYGSS |
| | | PTFGQGTKVEIKRT |
| P6 | 1016 | ELIQSPGTLFLSSGERATLSCRASQSVSSSNLAWFQHKSGRAPRLL |
| | | IYGASNRAPDIPDRFSGSGSGTDFTISISRLEPEDFAVYYCQRYGD |
| | | SPPITFGQGTRLEIKRT |
| Q1 | 1017 | ELTQSPGTLSLSPGERATLSCRASQSVSSSYLAWYQQKPGQAPRLL |
| | | IYGASSRATGIPDRFSGSGSGTDFTLTISRLEPEDFAVYYCQQYGS |
| | | SPQTFGQGTKVEIKRT |
| Q2 | 1018 | ELTQSPASLSLSPGGSATLACRASRGVNSNLAWYHQKPGQAPRLLI |
| | | YSASTRATGIPGRFSGSGFGTEFTLTINNLQSDDFGVYYCQQYDDT |
| | | PQITFGQGTRLDIKRL |
| R1 | 1019 | ELTQSPLSLPVTPGEPASISCRSSYSLLHINGYKYLDWYLQRPGQS |
| | | PQLLIYLGSNRAPGVPDRFSGSGSGTSFTLKISRVEAEDVGVYYCM |
| | | QSLQAPWTFGQGTKVEMKRT |
| S1 | 1020 | ELVLTQSPGTLSLSPGERATLSCRASQSVSSSYLAWYQQKPGQAPR |
| | | LLIYGASSRATGIPDRFSGSGSGTDFTLTISRLEPEDFAVYYCQQY |
| | | GSSPQTFGQGTKVEIKRT |
| S2 | 1087 | ELTQSPATLSLSPGERATLSCRASQSVSSRFLAWYQQKPGQAPRLL |
| | | IYGASSRATGIPDRFSGSGSGTDFTLTISRLEPEDFAVYYCQQYGS |
| | | SPQTFGQGTKVEIKRT |
| T1 | 1021 | ELTQSPGTLSLSPGERATLSCRASQSVSSSYLAWYQQKPGQAPRLL |
| | | IYGASSRATGIPDRFSGSGSGTDFTLTISRLEPEDFAVYYCQQYGS |
| | | SPQTFGQGTKVEIKRT |
| U1 | 1022 | ELTQSPSSLSASVGDRVTITCRASQSISSFLNWYQQKPGKAPKLLI |
| | | YAASSLQSGVPPRFSGSGSGTDFTLTISSLQPEDFATYYCQQSYST |
| | | PRTFGQGTKLEIKRT |
| U2 | 1088 | ELTQSPSSLSASIGDRVTIPCRASQSILNHLNWYQQKPGQPPKLLI |
| | | FAASNLQSGVPSRFSGSGSGTDFTLTISSLQTEDFATYYCQQSYST |
| | | PRTFGQGTKVEVKRT |
| U3 | 1089 | ELTQSPSSLSASVGDRVTITCRASQSIRSYLNWYQQKPGKAPNLLI |
| | | YSASNLQSGVPSRFRGSGSGTDFTLTISSLQPEDFATYYCQQSYRT |
| | | PRTFGQGTKVESKRT |
| V1 | 1023 | ELTQSPSSLSASVGDRVTITCQASQDISNFLNWYQRRPGKAPNLLI |
| | | YDATHLETGVPSRFSGSGFGTHFTLTINSLQPEDIGTYYCQHFDDV |
| | | PSFTFGPGTKVDLKRT |

Thus, the polypeptides provided herein that, alone or in combination with other polypeptides, bind HCV, purified E2 and/or the E1/E2 complex, can have an amino acid sequence that resembles a mammalian antibody light or heavy chain. For example, a polypeptide can have additional amino acid residues C-terminal to the CDR and framework sequences. The additional residues form a sequence resembling that of the constant region of the light or heavy chain of a human or other mammalian antibody. Mammalian antibody constant regions are known in the art. Examples of mammalian constant region sequences are described in Kabat et al., Sequences of proteins of immunological interest edn 5th: National Institutes of Health Publication No. 91-3242 (1991).

Thus, the C-terminal segment of a polypeptide provided herein can have one or more than one immunoglobulin domains as is typically present in the light and heavy chain constant regions of human or other mammalian antibodies. The constant region of a mammalian antibody light chain typically has one immunoglobulin domain, while the constant region of a mammalian antibody heavy chain typically has three or four immunoglobulin domains. The polypeptide also can have one or more than one cysteine residues that allow for formation of intra-chain disulfide bond between amino acid residues within a polypeptide of the invention or for formation of inter-chain disulfide bonds between two polypeptides of the invention. Further, the polypeptide can have a region that resembles the hinge region of a mammalian antibody heavy chain. The hinge region, when present in a polypeptide provided herein, is located between the first and second immunoglobulin domains and can have from 10 to over 60 amino acid residues. A portion of the hinge region can adopt a random and flexible conformation allowing for molecular motion.

In instances where the constant region is included in the polypeptides that, alone or in combination with other polypeptides, bind HCV, the constant region can have an amino acid sequence of a constant region of any of the immunoglobulin classes. The constant region can be from the light chain or heavy chain, including any known in the art. Exemplary Fab′ human consensus constant region sequences include, for example, those provided within the genebank of the National Center for Biotechnology Information. Typically, the polypeptide contains a constant region from an IgG immunoglobulin, such IgG1, IgG2, IgG3 or IgG4. For example, a polypeptide that, alone or in combination with other polypeptides, binds HCV, can contain a CDR with a sequence set forth in any of SEQ ID NOS: 78-308, 725-834 and 1090-1133 and an IgG1 constant region, such as, for example, a light chain kappa with a sequence set forth in SEQ ID NO:1364, or a heavy chain constant region. Exemplary of heavy chain constant regions are CH1, CH2, CH3 and hinge regions. For example, a polypeptide can contain the IgG1 CH1 region set forth in SEQ ID NO:1362, or the IgG1 hinge-CH2-CH3 region set forth in SEQ ID NO:1363.

In instances where the polypeptide has a CDR triplet spaced with antibody framework sequences, it can have a structure like the variable region of an antibody and can mimic, or in certain examples is, the variable region of a human or other mammalian antibody. If the polypeptide is composed of CDR triplets and framework sequences from the light or heavy chain groups as discussed above, the polypeptide is a variable region of a human monoclonal antibody. If the appropriate constant region sequence is added, the polypeptide has a structure mimicking, or is, a complete single light or heavy chain of a human or other mammalian antibody. For example, with addition of the constant region sequences from a Fab, a Fab′, or an IgG, IgE, IgA, IgD or IgM antibody, to a polypeptide of the invention, the polypeptide is a heavy or light single chain Fab or Fab′ fragment or a full-length single chain IgG, IgE, IgA, IgD or IgM molecule.

2. Anti-HCV Antibodies

Included among the polypeptides provided herein are antibodies and fragments thereof. For example, any two or more polypeptides described above can associate through covalent and non-covalent interactions to form a structure resembling a mammalian antibody. Covalent interactions are in the form of disulfide bonds, and non-covalent interactions are hydrophobic interactions. The two polypeptides can be any combination of polypeptides, e.g. two polypeptides that resemble mammalian antibody light chains, two polypeptides that resemble mammalian antibody heavy chains, two mixed CDR chains, or a polypeptide that resembles a mammalian antibody light chain and one that resembles a mammalian antibody heavy chain. For example, when a polypeptide described above that resembles a light chain and polypeptide described above that resembles a heavy chain polypeptide associate, the resulting complex can have a construction like that of a Fab or a Fab′ fragment. Two Fab′ fragments can associate to form a F(ab′)₂ fragment. When the F(ab)ₓ fragments are attached to the appropriate number of constant region Fc domain of a human or other mammalian immunoglobulin, it is a complete human or other mammalian monoclonal antibody. Thus, a polypeptide that resembles a mammalian antibody light chain can associate with a polypeptide that resembles a mammalian antibody heavy chain to form a monovalent antigen-binding fragment (Fab). In addition, two such dimers can associate to form a bivalent F(ab′)₂ or to form a structure resembling a mammalian IgG, IgE, IgA, IgD or IgM antibody.

Each polypeptide in an antibody can have three CDRs, i.e. a CDR triplet, with framework sequences as discussed above. The three CDRs in one single polypeptide chain can be selected from the light CDR group, and the three CDRs in the other polypeptide chain can be selected from the heavy CDR group. Each polypeptide can have matched triplets and framework regions as discussed above. For example, CDR1 sequences with Framework 1 sequences, CDR2 sequences with Framework 2 sequences, and CDR3 sequences with Framework 3 sequences. Additional examples include light chain CDR1 and light chain Framework 1 sequences or heavy chain CDR1 and heavy chain Framework 1 sequences; light chain CDR2 and light chain Framework 2 sequences or heavy chain CDR2 and heavy chain Framework 2 sequences; and light chain CDR3 with light chain Framework 3 sequences or heavy chain CDR3 and heavy chain Framework 3 sequences.

In the antibodies provided herein, one polypeptide can have a light chain constant region, and the other polypeptide can have a heavy chain constant region such that the two polypeptides associate to form an antigen-binding fragment (Fab). Two Fabs also can associate through inter-chain disulfide bonding between the heavy chain constant regions to form a bivalent Fab, i.e. F(ab′)₂. Thus, in a Fab or a F(ab′)₂ provided herein, one polypeptide can have light chain CDR, framework, and constant region sequences described herein and one polypeptide can have heavy chain CDR, framework, and constant region sequences as described herein.

The antibodies provided herein can contain any one or more of the polypeptides described above. Thus, the antibodies provided herein contain one or more CDRs set forth in SEQ ID NOS:78-308, 725-834 and 1090-1133. In particular examples, the antibodies contain one or more CDRs with a sequence of amino acids set forth in any of SEQ ID NOS: 725-834 and 1090-1133. Included among such antibodies are full length antibodies, or antigen-binding fragments thereof, including, for example, scFv, Fab, Fab′, F(ab′)₂, Fv, dsFv, diabody, Fd, or Fd′ fragments. The antibodies or antigen-binding fragments thereof selectively bind to HCV. In some examples, the antibodies or antigen-binding fragments thereof bind to E2. For example, the antibodies can bind to a discontinuous conformational epitope on E2 within amino acid residues 396-424, 436-447 and 535-540. In other examples, the antibodies or antigen-binding fragments thereof selectively bind to the E1E2 complex, but do not bind to a linear epitope of E1 or to an isolated E2 polypeptide. Also included are antibodies that bind to the same epitope as any of the antibodies described herein.

a. General Structure of Antibodies

Native antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable region (VH) followed by a number of constant regions. Each light chain has a variable region at one end (VL) and a constant region at its other end. The constant region of the light chain is aligned with the first constant region of the heavy chain, and the light chain variable region is aligned with the variable region of the heavy chain. The variable region of either chain has a triplet of hypervariable or complementarity determining regions (CDR's) spaced within a framework sequence as explained below. The framework and constant regions of the antibody have highly conserved amino acid sequences such that a species consensus sequence may typically be available for the framework and constant regions. Particular amino acid residues are believed to form an interface between the light and heavy chain variable regions (Chothia et al., J. Mol. Biol. 186, 651-63, 1985); Novotny and Haber, Proc. Natl. Acad. Sci. USA 82 4592-4596 (1985). Antibodies are produced naturally by B cells in membrane-bound and secreted forms. Antibodies specifically recognize and bind antigen epitopes through cognate interactions. Antibody binding to cognate antigens can initiate multiple effector functions, which cause neutralization and clearance of toxins, pathogens and other infectious agents.

Diversity in antibody specificity arises naturally due to recombination events during B cell development. Through these events, various combinations of multiple antibody V, D and J gene segments, which encode variable regions of antibody molecules, are joined with constant region genes to generate a natural antibody repertoire with large numbers of diverse antibodies. A human antibody repertoire contains more than 10¹⁰ different antigen specificities and thus theoretically can specifically recognize any foreign antigen. Antibodies include such naturally produced antibodies, as well as synthetically, i.e. recombinantly, produced antibodies, such as antibody fragments, including the anti-HCV antibodies or antigen-binding fragments provided herein.

In folded antibody polypeptides, binding specificity is conferred by antigen-binding site domains, which contain portions of heavy and/or light chain variable region domains. Other domains on the antibody molecule serve effector functions by participating in events such as signal transduction and interaction with other cells, polypeptides and biomolecules. These effector functions cause neutralization and/or clearance of the infecting agent recognized by the antibody. Domains of antibody polypeptides can be varied according to the methods herein to alter specific properties.

i. Structural and Functional Domains of Antibodies

Full-length antibodies contain multiple chains, domains and regions. A full length conventional antibody contains two heavy chains and two light chains, each of which contains a plurality of immunoglobulin (Ig) domains. An Ig domain is characterized by a structure called the Ig fold, which contains two beta-pleated sheets, each containing anti-parallel beta strands connected by loops. The two beta sheets in the Ig fold are sandwiched together by hydrophobic interactions and a conserved intra-chain disulfide bond. The Ig domains in the antibody chains are variable (V) and constant (C) region domains. Each heavy chain is linked to a light chain by a disulfide bond, and the two heavy chains are linked to each other by disulfide bonds. Linkage of the heavy chains is mediated by a flexible region of the heavy chain, known as the hinge region.

Each full-length conventional antibody light chain contains one variable region domain (V_{L}) and one constant region domain (C_{L}). Each full-length conventional heavy chain contains one variable region domain (V_{H}) and three or four constant region domains (C_{H}) and, in some cases, hinge region. Owing to recombination events discussed above, nucleic acid sequences encoding the variable region domains differ among antibodies and confer antigen-specificity to a particular antibody. The constant regions, on the other hand, are encoded by sequences that are more conserved among antibodies. These domains confer functional properties to antibodies, for example, the ability to interact with cells of the immune system and serum proteins in order to cause clearance of infectious agents. Different classes of antibodies, for example IgM, IgD, IgG, IgE and IgA, have different constant regions, allowing them to serve distinct effector functions.

Each variable region domain contains three portions called complementarity determining regions (CDRs) or hypervariable (HV) regions, which are encoded by highly variable nucleic acid sequences. The CDRs are located within the loops connecting the beta sheets of the variable region Ig domain. Together, the three heavy chain CDRs (CDR1, CDR2 and CDR3) and three light chain CDRs (CDR1, CDR2 and CDR3) make up a conventional antigen-binding site (antibody combining site) of the antibody, which physically interacts with cognate antigen and provides the specificity of the antibody. A whole antibody contains two identical antibody combining sites, each made up of CDRs from one heavy and one light chain. Because they are contained within the loops connecting the beta strands, the three CDRs are non-contiguous along the linear amino acid sequence of the variable region. Upon folding of the antibody polypeptide, the CDR loops are in close proximity, making up the antigen combining site. The beta sheets of the variable region domains form the framework regions (FRs), which contain more conserved sequences that are important for other properties of the antibody, for example, stability.

ii. Antibody Fragments

Antibodies provided herein include antibody fragments, which are derivatives of full-length antibodies that contain less than the full sequence of the full-length antibodies but retain at least a portion of the specific binding abilities of the full-length antibody. The antibody fragments also can include antigen-binding portions of an antibody that can be inserted into an antibody framework (e.g., chimeric antibodies) in order to retain the binding affinity of the parent antibody. Examples of antibody fragments include, but are not limited to, Fab, Fab′, F(ab′)2, single-chain Fvs (scFv), Fv, dsFv, diabody, Fd and Fd′ fragments, and other fragments, including modified fragments (see, for example, Methods in Molecular Biology, Vol 207: Recombinant Antibodies for Cancer Therapy Methods and Protocols (2003); Chapter 1; p 3-25, Kipriyanov). Antibody fragments can include multiple chains linked together, such as by disulfide bridges and can be produced recombinantly. Antibody fragments also can contain synthetic linkers, such as peptide linkers, to link two or more domains. Methods for generating antigen-binding fragments are well-known in the art and can be used to modify any antibody provided herein. Fragments of antibody molecules can be generated, such as for example, by enzymatic cleavage. For example, upon protease cleavage by papain, a dimer of the heavy chain constant regions, the Fc domain, is cleaved from the two Fab regions (i.e. the portions containing the variable regions).

Single chain antibodies can be recombinantly engineered by joining a heavy chain variable region (V_{H}) and light chain variable region (V_{L}) of a specific antibody. The particular nucleic acid sequences for the variable regions can be cloned by standard molecular biology methods, such as, for example, by polymerase chain reaction (PCR) and other recombination nucleic acid technologies. Methods for producing sFvs are described, for example, by Whitlow and Filpula (1991) Methods, 2: 97-105; Bird et al. (1988) Science 242:423-426; Pack et al. (1993) Bio/Technology 11:1271-77; and U.S. Pat. Nos. 4,946,778, 5,840,300, 5,667,988, 5,658,727, 5,258,498). Single chain antibodies also can be identified by screening single chain antibody libraries for binding to a target antigen. Methods for the construction and screening of such libraries are well-known in the art.

b. Exemplary Anti-HCV Antibodies

Provided herein are anti-HCV antibodies and antigen-binding fragments thereof that bind to HCV. Typically, the antibodies neutralize a hepatitis C virus from at least one genotype, such as genotype 1, 2, 3, 4, 5 or 6, or a subtype thereof. In some instances, the antibody or antigen-binding fragment thereof neutralize hepatitis C virus from 2, 3, 4, 5, 6 or more genotypes or subtypes thereof. The anti-HCV antibodies provided herein contain one or more polypeptides described above. Thus, the antibodies provided herein contain one or more CDRs set forth in SEQ ID NOS:78-308, 725-834 and 1090-1133. In particular examples, the antibodies contain one or more CDRs with a sequence of amino acids set forth in any of SEQ ID NOS: 725-834 and 1090-1133.

The anti-HCV antibodies or antigen-binding fragment thereof provided include monoclonal antibodies, multispecific antibodies, bispecific antibodies, human antibodies, humanized antibodies, camelised antibodies, chimeric antibodies, single-chain Fvs (scFv), single chain antibodies, single domain antibodies, Fab fragments, F(ab′) fragments, disulfide-linked Fvs (sdFv), and anti-idiotypic (anti-Id) antibodies, intrabodies, or antigen-binding fragments of any of the above. In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The anti-HCV antibodies or antigen-binding fragments thereof provided herein can contain any constant region known in the art, such as any human constant region known in the art, including, but not limited to, human light chain kappa (κ), human light chain lambda (λ), the constant region of IgG1, the constant region of IgG2, the constant region of IgG3 or the constant region of IgG4.

Exemplary of the anti-HCV antibodies provided herein include the Fab fragments set forth in Tables 3 and 4, including A, B1, B2, B3, B4, B5, B6a, B6b, B6c, B7, C1, C1*, C2, C2*, C3, C4, C5, C6, D1, D2, D3, D4, E, E2, E3, E4, F, G, H1, H1*, H1**, H2, H3, I, J1, J2, J3, J3*, J4, J5, K, L1, L2, L3, L4, L5, L6, L7, M, N1, N2, N3, N4, O1, P1, P1a, P2, P3, P4, P5, P6, Q1, Q2, R1, S1, S2, T1, U1, U2, U3 and V1 Fabs. Thus, exemplary of the anti-HCV antibodies provided herein are those with a Fab heavy chain with a sequence of amino acids set forth in any of SEQ ID NOS:1-31, 987-1004 and 1062-1075, and a Fab light chain with a sequence of amino acids set forth in any of SEQ ID NOS: 32-77, 1005-1023, 1076-1089 and 1365. The heavy chain of Fab E4, with a sequence set forth in SEQ ID NO:1068, can be combined with the light chain of any of the other Fabs provided herein to generate an anti-HCV Fab. In particular examples, the heavy chain of Fab E4 is combined with the light chain of Fab E (SEQ ID NO:48), Fab E2 (SEQ ID NO:1080) or Fab E3 (SEQ ID NO:1081) to generate an anti-HCV Fab. These Fabs were isolated from a phage display library made from the bone marrow of a patient with chronic HCV infection (genotype 1a) and Sjögren's Syndrome (see Examples 1-5), by panning against E1E2 complex.

Many of the Fabs exhibit robust neutralizing activity, as determined by neutralizing of binding (NOB) assays (e.g. 50% at 0.5˜1.0 μg/mL; see, e.g., Examples 2 and 3). The Fabs and IgGs made from the Fabs were characterized and found to bind conformational E2 epitopes or conformational epitopes on the E1E2 complex. For example, Fabs B1, B2, B3, D1, D2, D3, D4 and E bind the antigenic region (AR) 1 on E2. The antibodies bind better to E2 alone than the E1E2 complex, as do Fabs F and G, which bind AR2. Fabs A, C1, C2, C3, C4, C5, C6, H1, H2, H3, I, J1, J2, J3, J4, L1, L2, L3, L4 and M bind AR3. These antibodies bind better to the E1E2 complex than to E2. The discontinuous epitopes in AR3 are formed by at least three segments between amino acids 396-424, 436-447 and 523-540; the first and third segments also contribute to the CD81-binding domain of E2.

Fab U1 competes strongly with AR3-binding antibodies, and partially with ARIA, which is the full length IgG1 of Fab B2. Fabs N1, N2, N3, N4, O1, P1, P1a, P2, P3, P4, P5, P6, Q1, Q2, R1, S1, T1, and V1 do not significantly compete with any of the AR1-, AR2 or AR3-binding antibodies, indicating that they bind to a different epitope on E1 or E2. Many of these Fabs cross-react with E1E2 from different HCV isolates and genotypes. IgG1 antibodies generated from the N4 and V1 Fabs selectively bind folded E1E2, but do not bind denatured E1E2, nor E1E2 containing modifications in the E1 polypeptide that disrupt the correct folding of the E1 protein within the E1E2 complex. Thus, it appears that N4 and V1 selectively bind a conformational epitope presented on the E1E2 complex. Correct folding of the E1/E2 complex appears to be critical for the binding of both N4 and V1 (see Example 4). The N4 IgG and V1 IgG (and fragments thereof) recognize two distinct discontinuous epitopes on the E1/E2 complex, that are different from the epitopes located at antigenic regions (AR) 1, 2 and 3 described herein (see Example 1) and elsewhere (Law et al., (2008) Nature Med. 14:25-27).

Also included in the anti-HCV antibodies provided herein are those that bind to the same epitope as any of the antibodies described above. For example, also included are antibodies and antigen-binding fragments that bind to the same epitope as any of Fabs N1, N2, N3, N4, O1, P1, P1a, P2, P3, P4, P5, P6, Q1, Q2, R1, S1, T1, and V1. In a particular example, the antibodies bind to the same epitope as Fab N4, Q2, or V1 (or the full length IgGs thereof). Whether an antibody binds to the same epitope as another antibody can readily be determined by one of skill in the art. For example, epitopes can be mapped, such as by mutation of the antigen. In other example, competition assays, such as a competition ELISA, can be performed, such as one described in Example 1. Briefly, for example, saturating concentrations of the blocking antibody (the antibody being tested for competition) are added to the captured antigen, such as E1E2 complex, and incubated before an equal amount of the biotinylated antibody (e.g. biotinylated N1, N2, N3, N4, O1, P1, P1a, P2, P3, P4, P5, P6, Q1, Q2, R1, S1, T1, or V1) is added. The amount of biotinylated antibody that binds is then assessed and compared to the binding in the absence of the blocking antibody. Competition is determined by the percentage change in binding signals in the presence of the blocking antibody compared to the absence of the blocking antibody. Competition of 70% or more (i.e. the amount of biotinylated antibody bound to the antigen in the presence of the blocking antibody is 30% or less of the amount of the biotinylated antibody bound to the antigen in the absence of the antibody) typically is indicative that the antibodies bind to the same epitope. Thus, if competition of 70%, 71%, 72%, 73%, 74%, 75%, 80%, 85%, 90%, 95% or 100% is observed between an anti-HCV antibody and, for example, N4 IgG, then the anti-HCV antibody can be considered to bind to the same epitope as N4 IgG.

The antibodies provided herein include fragments of A, B1, B2, B3, B4, B5, B6a, B6b, B6c, B7, C1, C1*, C2, C2*, C3, C4, C5, C6, D1, D2, D3, D4, E, E2, E3, E4, F, G, H1, H1*, H1**, H2, H3, I, J1, J2, J3, J3*, J4, J5, K, L1, L2, L3, L4, L5, L6, L7, M, N1, N2, N3, N4, O1, P1, P1a, P2, P3, P4, P5, P6, Q1, Q2, R1, S1, S2, T1, U1, U2, U3 and V1 Fabs, that selectively bind E2 or E1E2, and full length antibodies, including full length IgG1 antibodies, of these Fabs. Such antibodies include chimeric antibodies, single-chain Fvs (scFv), single chain antibodies, single domain antibodies, Fab fragments, F(ab′) fragments, disulfide-linked Fvs (sdFv), anti-idiotypic (anti-Id) antibodies, intrabodies, or antigen-binding fragments of any of the above.

The anti-HCV antibodies provided herein can neutralize hepatitis C virus. For example, the antibodies can neutralize 10%, 15%, 20%, 25%, 30%, 35%, 40%, 45%, 50%, 55%, 60%, 65%, 70%, 75%, 80%, 85%, 90%, 95% or more of the virus. Further, the antibodies can neutralize one or more than one genotype, such as 1, 2, 3, 4, 5 or 6 genotypes. The antibodies or antigen-binding fragments thereof can neutralize hepatitis C virus from any one or more of genotypes 1, 2, 3, 4, 5 or 6, or any subtype thereof.

c. Additional Modifications of Anti-HCV Antibodies

The anti-HCV antibodies or antigen-binding fragments thereof provided herein can be further modified. Modifications of an anti-HCV antibody or antigen-binding fragment can improve one or more properties of the antibody, including, but not limited to, decreasing the immunogenicity of the antibody or antigen-binding fragment, improving the half-life of the antibody or antigen-binding fragment, such as reducing the susceptibility to proteolysis and/or reducing susceptibility to oxidation, and altering or improving of the binding properties of the antibody or antigen-binding fragment. Exemplary modifications include, but are not limited to, modifications of the primary amino acid sequence of the anti-HCV antibody or antigen-binding fragment thereof and alteration of the post-translational modification of the anti-HCV antibody or antigen-binding fragment thereof. Exemplary post-translational modifications include, for example, glycosylation, acetylation, pegylation, phosphorylation, amidation, derivatization with protecting/blocking group, proteolytic cleavage, linkage to a cellular ligand or other protein. Other exemplary modifications include attachment of one or more heterologous peptides to the anti-HCV antibody or antigen-binding fragment to alter or improve one or more properties of the antibody or antigen-binding fragment thereof.

Generally, the modifications do not result in increased immunogenicity of the antibody or antigen-binding fragment thereof or significantly negatively affect the binding of the antibody or antigen-binding fragment thereof to HCV. Methods of assessing the binding of the modified antibodies or antigen-binding fragments thereof to a HCV protein are provided herein and known in the art. For example, modified antibodies or antigen-binding fragments thereof can be assayed for binding to E1, E2 or E1E2 by methods such as, but not limited to, ELISA, surface plasmon resonance (SPR), or through in vitro neutralization assays.

Provided herein are methods of improving the half-life of any of the anti-HCV antibodies or antigen-binding fragments provided. Increasing the half-life of the anti-HCV antibodies or antigen-binding fragments thereof provided can increase the therapeutic effectiveness of the anti-HCV antibodies or antigen-binding fragments thereof and allow for less frequent administration of the antibodies or antigen-binding fragments thereof for prophylaxis and/or treatment, such as preventing or treating a HCV infection, preventing, treating, and/or alleviating of one or more symptoms of a HCV infection, or reducing the duration of a HCV infection.

Modification of the anti-HCV antibodies or antigen-binding fragments thereof produced herein can include one or more amino acid substitutions, deletions or additions, either from natural mutation or human manipulation from the parent antibody from which it was derived. Methods for modification of polypeptides, such as antibodies, are known in the art and can be employed for the modification of any antibody or antigen-binding fragment provided herein. In some examples, the pharmacokinetic properties of the anti-HCV antibodies or antigen-binding fragments thereof provided can be enhanced through Fc modifications by techniques known to those skilled in the art. Standard techniques known to those skilled in the art can be used to introduce mutations in the nucleotide molecule encoding an antibody or an antigen-binding fragment provided herein in order to produce a polypeptide with one or more amino acid substitutions. Exemplary techniques for introducing mutations include, but are not limited to, site-directed mutagenesis and PCR-mediated mutagenesis.

The anti-HCV antibodies and fragments thereof provided herein can be modified by the attachment of a heterologous peptide to facilitate purification. Generally such peptides are expressed as a fusion protein containing the antibody fused to the peptide at the C- or N-terminus of the antibody or antigen-binding fragment thereof. Exemplary peptides commonly used for purification include, but are not limited to, hexa-histidine peptides, hemagglutinin (HA) peptides, and flag tag peptides (see e.g., Wilson et al. (1984) Cell 37:767; Witzgall et al. (1994) Anal Biochem 223:2, 291-8). The fusion does not necessarily need to be direct, but can occur through a linker peptide. In some examples, the linker peptide contains a protease cleavage site which allows for removal of the purification peptide following purification by cleavage with a protease that specifically recognizes the protease cleavage site.

The anti-HCV antibodies and fragments thereof provided herein also can be modified by the attachment of a heterologous polypeptide that targets the antibody or antigen-binding fragment to a particular cell type, either in vitro or in vivo. In some examples an anti-HCV antibody or antigen-binding fragment thereof provided herein can be targeted to a particular cell type by fusing or conjugating the antibody or antigen-binding fragment thereof to an antibody specific for a particular cell surface receptor or other polypeptide that interacts with a specific cell receptor.

In some examples, an anti-HCV antibody or antigen-binding fragment thereof provided herein can be targeted to a target cell surface and/or taken up by the target cell by fusing or conjugating the antibody or antigen-binding fragment thereof to a peptide that binds to cell surface glycoproteins, such as a protein transduction domain (e.g. a TAT peptide). Exemplary protein transduction domains include, but are not limited to, PTDs derived from proteins such as human immunodeficiency virus 1 (HIV-1) TAT (Ruben et al. (1989) J. Virol. 63:1-8), the herpes virus tegument protein VP22 (Elliott and O'Hare (1997) Cell 88:223-233), the homeotic protein of Drosophila melanogaster Antennapedia (Antp) protein (Penetratin PTD; Derossi et al. (1996) J. Biol. Chem. 271:18188-18193), the protegrin 1 (PG-1) anti-microbial peptide SynB (e.g., SynB1, SynB3, and Syn B4; Kokryakov et al. (1993) FEBS Lett. 327:231-236) and basic fibroblast growth factor Pans (1994) FASEB J. 8:841-847). PTDs also include synthetic PTDs, such as, but not limited to, polyarginine peptides (Futaki et al. (2003) J. Mol. Recognit. 16:260-264; Suzuki et al. (2001) J. Biol. Chem. 276:5836-5840), transportan (Pooga et al. (1988) FASEB J. 12:67-77; Pooga et al. (2001) FASEB J. 15:1451-1453), MAP (Oehlke et al. (1998) Biochim. Biophys. Acta. 1414:127-139), KALA (Wyman et al. (1997) Biochemistry 36:3008-3017) and other cationic peptides, such as, for example, various β-cationic peptides (Akkarawongsa et al. (2008) Antimicrob. Agents and Chemother. 52(6):2120-2129).

The anti-HCV antibodies and fragments thereof provided herein can be modified by the attachment of diagnostic and/or therapeutic moiety to the antibody or antigen-binding fragment thereof. The anti-HCV antibodies and fragments thereof provided herein can be modified by the covalent attachment of any type of molecule, such as a diagnostic or therapeutic molecule, to the antibody or antigen-binding fragment thereof such that covalent attachment does not prevent the antibody or antigen-binding fragment thereof from binding to its corresponding epitope. For example, an anti-HCV antibody or antigen-binding fragment thereof provided herein can be further modified by covalent attachment of a molecule such that the covalent attachment does not prevent the antibody or antigen-binding fragment thereof from binding to HCV. In some examples, the antibodies or antigen-binding fragments thereof can be recombinantly fused to a heterologous polypeptide at the N terminus or C terminus or chemically conjugated, including covalent and non-covalent conjugation, to a heterologous polypeptide or other composition. For example, the heterologous polypeptide or composition can be a diagnostic polypeptide or other diagnostic moiety or a therapeutic polypeptide or other therapeutic moiety. Exemplary diagnostic and therapeutic moieties include, but are not limited to, drugs, radionuclides, toxins, fluorescent molecules (see, e.g. International PCT Publication Nos. WO 92/08495; WO 91/14438; WO 89/12624; U.S. Pat. No. 5,314,995; and EP 396,387). Diagnostic polypeptides or diagnostic moieties can be used, for example, as labels for in vivo or in vitro detection. Therapeutic polypeptides or therapeutic moieties can be used, for example, for therapy of a viral infection, such as HCV infection, or for treatment of one or more symptoms of a viral infection.

Additional fusion proteins of the anti-HCV antibodies or antigen-binding fragments thereof provided herein can be generated through the techniques of gene-shuffling, motif-shuffling, exon-shuffling, and/or codon-shuffling (collectively referred to as “DNA shuffling”). DNA shuffling can be employed to alter the activities of anti-HCV antibodies or antigen-binding fragments thereof provided herein, for example, to produce antibodies or antigen-binding fragments thereof with higher affinities and lower dissociation rates (see, generally, U.S. Pat. Nos. 5,605,793; 5,811,238; 5,830,721; 5,834,252; and 5,837,458, and Patten et al. (1997) Curr. Opinion Biotechnol. 8:724-33; Harayama (1998) Trends Biotechnol. 16(2):76-82; Hansson et al., (1999) J. Mol. Biol. 287:265-76; and Lorenzo and Blasco (1998) Biotechniques 24(2):308-13).

The anti-HCV antibodies or antigen-binding fragments thereof can also be attached to solid supports, which are useful for immunoassays or purification of the target antigen. Exemplary solid supports include, but are not limited to, glass, cellulose, polyacrylamide, nylon, polystyrene, polyvinyl chloride or polypropylene.

i. Modifications to Reduce Immunogenicity

In some examples, the antibodies or antigen-binding fragments thereof provided herein can be further modified to reduce the immunogenicity in a subject, such as a human subject. For example, one or more amino acids in the antibody or antigen-binding fragment thereof can be modified to alter potential epitopes for human T-cells in order to eliminate or reduce the immunogenicity of the antibody or antigen-binding fragment thereof when exposed to the immune system of the subject. Exemplary modifications include substitutions, deletions and insertion of one or more amino acids, which eliminate or reduce the immunogenicity of the antibody or antigen-binding fragment thereof. Generally, such modifications do not alter the binding specificity of the antibody or antigen-binding fragment thereof for its respective antigen. Reducing the immunogenicity of the antibody or antigen-binding fragment thereof can improve one or more properties of the antibody or antigen-binding fragment thereof, such as, for example, improving the therapeutic efficacy of the antibody or antigen-binding fragment thereof and/or increasing the half-life of the antibody or antigen-binding fragment thereof in vivo.

ii. Fc Modifications

The anti-HCV antibodies or antigen-binding fragments thereof provided herein can contain wild-type or modified Fc region. As described elsewhere herein, a Fc region can be linked to an anti-HCV antigen-binding fragment provided herein. In some examples, the Fc region can be modified to alter one or more properties of the Fc polypeptide. For example, the Fc region can be modified to alter (i.e. more or less) effector functions compared to the effector function of an Fc region of a wild-type immunoglobulin heavy chain. The Fc regions of an antibody interacts with a number of Fc receptors, and ligands, imparting an array of important functional capabilities referred to as effector functions. Fc effector functions include, for example, Fc receptor binding, complement fixation, and T cell depleting activity (see e.g., U.S. Pat. No. 6,136,310). Methods of assaying T cell depleting activity, Fc effector function, and antibody stability are known in the art. For example, the Fc region of an IgG molecule interacts with the FcγRs. These receptors are expressed in a variety of immune cells, including for example, monocytes, macrophages, neutrophils, dendritic cells, eosinophils, mast cells, platelets, B cells, large granular lymphocytes, Langerhans' cells, natural killer (NK) cells, and γδ T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. Recognition of and lysis of bound antibody on target cells by cytotoxic cells that express FcγRs is referred to as antibody dependent cell-mediated cytotoxicity (ADCC). Other Fc receptors for various antibody isotypes include FcεRs (IgE), FcαRs (IgA), and FcμRs (IgM).

Thus, a modified Fc domain can have altered affinity, including but not limited to, increased or low or no affinity for the Fc receptor. For example, the different IgG subclasses have different affinities for the FcγRs, with IgG1 and IgG3 typically binding substantially better to the receptors than IgG2 and IgG4. In addition, different FcγRs mediate different effector functions. FcγR1, FcγRIIa/c, and FcγRIIIa are positive regulators of immune complex triggered activation, characterized by having an intracellular domain that has an immunoreceptor tyrosine-based activation motif (ITAM). FcγRIIb, however, has an immunoreceptor tyrosine-based inhibition motif (ITIM) and is therefore inhibitory. Thus, altering the affinity of an Fc region for a receptor can modulate the effector functions induced by the Fc domain.

In one example, an Fc region is used that is modified for optimized binding to certain FcγRs to better mediate effector functions, such as for example, antibody-dependent cellular cytotoxicity, ADCC. Such modified Fc regions can contain modifications at one or more of amino acid residues (according to the Kabat numbering scheme, Kabat et al. (1991) Sequences of Proteins of Immunological Interest, U.S. Department of Health and Human Services), including, but not limited to, amino acid positions 249, 252, 259, 262, 268, 271, 273, 277, 280, 281, 285, 287, 296, 300, 317, 323, 343, 345, 346, 349, 351, 352, 353, and 424. For example, modifications in an Fc region can be made corresponding to any one or more of G20S, G20A, S23D, S23E, S23N, S23Q, S23T, K30H, K30Y, D33Y, R39Y, E42Y, T44H, V48I, S51E, H52D, E56Y, E56I, E56H, K58E, G65D, E67L, E67H, S82A, S82D, S88T, S108G, S108I, K110T, K110E, K110D, A111D, A114Y, A114L, A114I, I116D, I116E, I116N, I116Q, E117Y, E117A, K118T, K118F, K118A, and P180L of the exemplary Fc sequence set forth in SEQ ID NO:1605, or combinations thereof. A modified Fc region containing these mutations can have enhanced binding to an FcR such as, for example, the activating receptor FcγIIIa and/or can have reduced binding to the inhibitory receptor FcγRIIb (see e.g., US 2006/0024298). Fc regions modified to have increased binding to FcRs can be more effective in facilitating the destruction of viral (e.g. HCV) infected cells in patients.

In some examples, the antibodies or antigen-binding fragments provided herein can be further modified to improve the interaction of the antibody or antigen-binding fragment thereof with the FcRn receptor in order to increase the in vivo half-life and pharmacokinetics of the antibody or antigen-binding fragment thereof (see, e.g. U.S. Pat. No. 7,217,797, U.S Pat. Pub. Nos. 2006/0198840 and 2008/0287657). FcRn is the neonatal FcR, the binding of which recycles endocytosed antibody or antigen-binding fragment thereof from the endosomes back to the bloodstream. This process, coupled with preclusion of kidney filtration due to the large size of the full length molecule, results in favorable antibody serum half-lives ranging from one to three weeks. Binding of Fc to FcRn also plays a role in antibody transport.

Exemplary modifications of the Fc region include but are not limited to, mutation of the Fc region described in U.S. Pat. No. 7,217,797; U.S Pat. Pub. Nos. 2006/0198840, 2006/0024298 and 2008/0287657, and International Patent Pub. No. WO 2005/063816, such as mutations at one or more of amino acid residues (Kabat numbering, Kabat et al. (1991)) 251-256, 285-90, 308-314, in the C_{H}2 domain and/or amino acids residues 385-389, and 428-436 in the C_{H}3 domain of the Fc heavy chain constant region, where the modification alters Fc receptor binding affinity and/or serum half-life relative to unmodified antibody or antigen-binding fragment thereof. In some examples, the Fc region is modified at one or more of amino acid positions 250, 251, 252, 254, 255, 256, 263, 308, 309, 311, 312 and 314 in the C_{H}2 domain and/or amino acid positions 385, 386, 387, 389, 428, 433, 434, 436, and 459 in the C_{H}3 domain of the Fc heavy chain constant region. Such modifications correspond to amino acids Gly21, Pro22, Ser23, Phe25, Leu26, Phe27, Thr34, Pro75, Arg76, Glu78, Gln79, and Asn81 in the C_{H}2 domain and amino acids Gln146, Val147, Ser148, Thr150, Ser184, Gly 186, Ser187, Phe189, and Met212 in the C_{H}3 domain in an exemplary Fc sequence set forth in SEQ ID NO:1605. In some examples, the modification is at one or more surface-exposed residues, and the modification is a substitution with a residue of similar charge, polarity or hydrophobicity to the residue being substituted.

In particular examples, a Fc heavy chain constant region is modified at one or more of amino acid positions 251, 252, 254, 255, and 256 (Kabat numbering), where position 251 is substituted with Leu or Arg, position 252 is substituted with Tyr, Phe, Ser, Trp or Thr, position 254 is substituted with Thr or Ser, position 255 is substituted with Leu, Gly, Ile or Arg, and/or position 256 is substituted with Ser, Arg, Gln, Glu, Asp, Ala, Asp or Thr. In some examples, a Fc heavy chain constant region is modified at one or more of amino acid positions 308, 309, 311, 312, and 314, where position 308 is substituted with Thr or Ile, position 309 is substituted with Pro, position 311 is substituted with serine or Glu, position 312 is substituted with. Asp, and/or position 314 is substituted with Leu. In some examples, a Fc heavy chain constant region is modified at one or more of amino acid positions 428, 433, 434, and 436, where position 428 is substituted with Met, Thr, Leu, Phe, or Ser, position 433 is substituted with Lys, Arg, Ser, Ile, Pro, Gln, or His, position 434 is substituted with Phe, Tyr, or His, and/or position 436 is substituted with His, Asn, Asp, Thr, Lys, Met, or Thr. In some examples, a Fc heavy chain constant region is modified at one or more of amino acid positions 263 and 459, where position 263 is substituted with Gln or Glu and/or position 459 is substituted with Leu or Phe.

In some examples, a Fc heavy chain constant region can be modified to enhance binding to the complement protein C1q. In addition to interacting with FcRs, Fc also interact with the complement protein C1q to mediate complement dependent cytotoxicity (CDC). C1q forms a complex with the serine proteases C1r and C1s to form the C1 complex. C1q is capable of binding six antibodies, although binding to two IgGs is sufficient to activate the complement cascade. Similar to Fc interaction with FcRs, different IgG subclasses have different affinity for C1q, with IgG1 and IgG3 typically binding substantially better than IgG2 and IgG4. Thus, a modified Fc having increased binding to C1q can mediate enhanced CDC, and can enhance destruction of viral (e.g., HCV) infected cells. Exemplary modifications in an Fc region that increase binding to C1q include, but are not limited to, amino acid modifications at positions 345 and 253 (Kabat numbering).

In another example, a variety of Fc mutants with substitutions to reduce or ablate binding with FcγRs also are known. Such muteins are useful in instances where there is a need for reduced or eliminated effector function mediated by Fc. This is often the case where antagonism, but not killing of the cells bearing a target antigen is desired. Exemplary of such an Fc is an Fc mutein described in U.S. Pat. No. 5,457,035, which is modified at amino acid positions 248, 249 and 251 (Kabat numbering). In an exemplary Fc sequence set forth in SEQ ID NO:1605, amino acid 19 is modified from Leu to Ala, amino acid 20 is modified from Leu to Glu, and amino acid 22 is modified from Gly to Ala. Similar mutations can be made in any Fc sequence such as, for example, the exemplary Fc sequence. This mutein exhibits reduced affinity for Fc receptors.

The antibodies or antigen-binding fragments thereof provided herein can be engineered to contain modified Fc regions. For example, methods for fusing or conjugating polypeptides to the constant regions of antibodies (i.e. making Fc fusion proteins) are known in the art and described in, for example, U.S. Pat. Nos. 5,336,603, 5,622,929, 5,359,046, 5,349,053, 5,447,851, 5,723,125, 5,783,181, 5,908,626, 5,844,095, and 5,112,946; EP 307,434; EP 367,166; EP 394,827; PCT publications WO 91/06570, WO 96/04388, WO 96/22024, WO 97/34631, and WO 99/04813; Ashkenazi et al. (1991) Proc. Natl. Acad. Sci. USA 88:10535-10539; Traunecker et al. (1988) Nature 331:84-86; Zheng et al. (1995) J. Immunol. 154:5590-5600; and Vil et al. (1992) Proc. Natl. Acad. Sci. USA 89:11337-11341 (1992) and described elsewhere herein. In some examples, a modified Fc region having one or more modifications that increases the FcRn binding affinity and/or improves half-life can be fused to an anti-HCV antibody or antigen-binding fragment thereof provided herein.

iii. Pegylation

The anti-HCV antibodies or antigen-binding fragments thereof provided herein can be conjugated to polymer molecules such as high molecular weight polyethylene glycol (PEG) to increase half-life and/or improve their pharmacokinetic profiles. Conjugation can be carried out by techniques known to those skilled in the art. Conjugation of therapeutic antibodies with PEG has been shown to enhance pharmacodynamics while not interfering with function (see, e.g., Deckert et al., Int. J. Cancer 87: 382-390, 2000; Knight et al., Platelets 15: 409-418, 2004; Leong et al., Cytokine 16: 106-119, 2001; and Yang et al., Protein Eng. 16: 761-770, 2003). PEG can be attached to the antibodies or antigen-binding fragments with or without a multifunctional linker either through site-specific conjugation of the PEG to the N- or C-terminus of the antibodies or antigen-binding fragments or via epsilon-amino groups present on lysine residues. Linear or branched polymer derivatization that results in minimal loss of biological activity can be used. The degree of conjugation can be monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography. PEG-derivatized antibodies or antigen-binding fragments thereof can be tested for binding activity to HCV antigens as well as for in vivo efficacy using methods known to those skilled in the art, for example, by immunoassays described herein.

iv. Conjugation of a Detectable Moiety

In some examples, the anti-HCV antibodies and antibody fragments provided herein can be further modified by conjugation to a detectable moiety. The detectable moieties can be detected directly or indirectly. Depending on the detectable moiety selected, the detectable moiety can be detected in vivo and/or in vitro. The detectable moieties can be employed, for example, in diagnostic methods for detecting exposure to HCV or localization of HCV or binding assays for determining the binding affinity of the anti-HCV antibody or antigen-binding fragment thereof for HCV. The detectable moieties also can be employed in methods of preparation of the anti-HCV antibodies, such as, for example, purification of the antibody or antigen-binding fragment thereof. Typically, detectable moieties are selected such that conjugation of the detectable moiety does not interfere with the binding of the antibody or antigen-binding fragment thereof to the target epitope. Generally, the choice of the detectable moiety depends on sensitivity required, ease of conjugation with the compound, stability requirements, available instrumentation, and disposal provisions. One of skill in the art is familiar with labels and can identify a detectable label suitable for and compatible with the assay employed. Methods of labeling antibodies with detectable moieties are known in the art and include, for example, recombinant and chemical methods.

The detectable moiety can be any material having a detectable physical or chemical property. Such detectable labels have been well-developed in the field of immunoassays and, in general, most any label useful in such methods can be applied in the methods provided. Thus, a label is any composition detectable by spectroscopic, photochemical, biochemical, immunochemical, electrical, optical or chemical means. Useful labels include, but are not limited to, fluorescent dyes (e.g., fluorescein isothiocyanate, Texas red, rhodamine, and the like), radiolabels (e.g., ³H, ¹²⁵I, ³⁵S, ¹⁴C, or ³²P), in particular, gamma and positron emitting radioisotopes (e.g., ¹⁵⁷Gd, ⁵⁵Mn, ¹⁶²Dy, ⁵²Cr, and ⁵⁶Fe), metallic ions (e.g., ¹¹¹In, ⁹⁷Ru, ⁶⁷Ga, ⁶⁸Ga, ⁷²As, ⁸⁹Zr, and ²⁰¹Ti), enzymes (e.g., horse radish peroxidase, alkaline phosphatase and others commonly used in an ELISA), electron transfer agents (e.g., including metal binding proteins and compounds); luminescent and chemiluminescent labels (e.g., luciferin and 2,3-dihydrophthalazinediones, e.g., luminol), magnetic beads (e.g., DYNABEADS™), and colorimetric labels such as colloidal gold or colored glass or plastic beads (e.g., polystyrene, polypropylene, latex, etc.). For a review of various labeling or signal producing systems that can be used, see e.g. U.S. Pat. No. 4,391,904.

v. Conjugation of a Therapeutic Moiety

In some examples, anti-HCV antibodies and antigen-binding fragments provided herein can be further modified by conjugation to a therapeutic moiety. Exemplary therapeutic moieties include, but are not limited to, a cytotoxin (e.g., a cytostatic or cytocidal agent), a therapeutic agent (such as a cytokine or interferon) or a radioactive metal ion (e.g., alpha-emitters). Exemplary cytotoxins or cytotoxic agents include, but are not limited to, any agent that is detrimental to cells, such as, but not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Exemplary therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), anti-mitotic agents (e.g., vincristine and vinblastine), and antivirals, such as, but not limited to, nucleoside analogs, such as zidovudine, acyclovir, gangcyclovir, vidarabine, idoxuridine, trifluridine, and ribavirin; foscamet, amantadine, rimantadine, saquinavir, indinavir, ritonavir, and alpha-interferons.

In some examples, the anti-HCV antibodies and antibody fragments provided herein can be further modified by conjugation to a therapeutic moiety that is a therapeutic polypeptide. Exemplary therapeutic polypeptides include, but are not limited to, a toxin, such as abrin, ricin A, pseudomonas exotoxin, or diphtheria toxin; or an immunostimulatory agent, such as a cytokine, such as, but not limited to, an interferon (e.g., IFN-α, β, γ, ω), a lymphokine, a hematopoietic growth factor, such as, for example, GM-CSF (granulocyte macrophage colony stimulating factor), Interleukin-2 (IL-2), Interleukin-3 (IL-3), Interleukin-4 (IL-4), Interleukin-7 (IL-7), Interleukin-10 (IL-10), Interleukin-12 (IL-12), Interleukin-14 (IL-14), and Tumor Necrosis Factor (TNF).

vi. Modifications to Improve Binding Specificity

The binding specificity of the anti-HCV antibodies and antibody fragments provided can be altered or improved by techniques, such as phage display. Methods for phage display generally involve the use of a filamentous phage (phagemid) surface expression vector system for cloning and expressing antibody species of the library. Various phagemid cloning systems to produce combinatorial libraries have been described by others. See, for example the preparation of combinatorial antibody libraries on phagemids as described by Kang, et al., Proc. Natl. Acad. Sci., USA, 88:4363-4366 (1991); Barbas, et al., Proc. Natl. Acad. Sci., USA, 88:7978-7982 (1991); Zebedee, et al., Proc. Natl. Acad. Sci., USA, 89:3175-3179 (1992); Kang, et al., Proc. Natl. Acad. Sci., USA, 88:11120-11123 (1991); Barbas, et al., Proc. Natl. Acad. Sci., USA, 89:4457-4461 (1992); and Gram, et al., Proc. Natl. Acad. Sci., USA, 89:3576-3580 (1992), which references are hereby incorporated by reference.

In particular examples, DNA sequences encoding V_{H} and V_{L} domains are amplified from animal cDNA libraries (e.g., human or murine cDNA libraries of lymphoid tissues). The DNA encoding the V_{H} and V_{L} domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the V_{H} and V_{L} domains are usually recombinantly fused to either the phage gene III or gene VIII. Phage expressing an antigen-binding domain that binds to a HCV antigen, for example, HCV F protein, can be selected or identified with antigen, e.g., using labeled antigen or antigen bound or captured to a solid surface or bead. Examples of phage display methods that can be used to make the antibodies by phage display include those disclosed, for example, in Brinkman et al. (1995) J. Immunol. Methods 182:41-50; Ames et al. (1995) J. Immunol. Methods 184:177-186; Kettleborough et al. (1994) Eur. J. Immunol. 24:952-958; Persic et al. (1997) Gene 187:9-18; Burton et al. (1994) Advances in Immunology 57:191-280; PCT application No. PCT/GB91/O1 134; PCT publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen-binding fragment, and expressed in any desired host, including mammalian cells, insect cells, plant cells, yeast, and bacteria, e.g., as described herein. Techniques to recombinantly produce Fab, Fab′ and F(ab′)2 fragments can also be employed using methods known in the art such as those disclosed in PCT publication No. WO 92/22324; Mullinax et al. (1992) BioTechniques 12(6):864-869; Sawai et al. (1995) AJRI 34:26-34; and Better et al. (1988) Science 240: 1041-1043.

The resulting phagemid library can be manipulated to increase and/or alter the immunospecificities of the antibodies or antibody fragment of the library to produce and subsequently identify additional antibodies with improved properties, such as increased binding to a target antigen. For example, either or both the H and L chain encoding DNA can be mutagenized in a complementarity determining region (CDR) of the variable region of the immunoglobulin polypeptide, and subsequently screened for desirable immunoreaction and neutralization capabilities. The resulting antibodies can then be screened in one or more of the assays described herein for determining neutralization capacity.

For some uses, including in vivo use of antibodies in humans and in vitro detection assays, human or chimeric antibodies are used. Completely human antibodies are particularly desirable for therapeutic treatment of human subjects. Human antibodies can be made by a variety of methods known in the art including phage display methods described above using antibody libraries derived from human immunoglobulin sequences or synthetic sequences homologous to human immunoglobulin sequences. See also U.S. Pat. Nos. 4,444,887 and 4,716,111; and PCT publications WO 98/46645, WO 98/50433, WO 98/24893, WO98/16654, WO 96/34096, WO 96/33735, and WO 91/10741; each of which is incorporated herein by reference in its entirety.

3. Methods for Producing Anti-HCV Polypeptides and Antibodies

The anti-HCV antibodies or antigen-binding fragments thereof provided herein can be generated by any suitable method known in the art for the preparation of antibodies, including chemical synthesis and recombinant expression techniques. Various combinations of host cells and vectors can be used to receive, maintain, reproduce and amplify nucleic acids (e.g. nucleic acids encoding antibodies such as the anti-HCV antibodies or antigen-binding fragments thereof provided), and to express polypeptides encoded by the nucleic acids. In general, the choice of host cell and vector depends on whether amplification, polypeptide expression, and/or display on a genetic package, such as a phage, is desired. Methods for transforming host cells are well known. Any known transformation method (e.g., transformation, transfection, infection, electroporation and sonoporation) can be used to transform the host cell with nucleic acids. Procedures for the production of antibodies, such as monoclonal antibodies and antibody fragments, such as, but not limited to, Fab fragments and single chain antibodies are well known in the art.

Monoclonal antibodies can be prepared using a wide variety of techniques known in the art including, but not limited to, the use of hybridoma, recombinant expression, phage display technologies or a combination thereof. For example, monoclonal antibodies can be produced using hybridoma techniques including those known in the art and taught for example in Harlow et al. Antibodies: A Laboratory Manual (Cold Spring Harbor Laboratory Press, 2nd ed. 1988); Hammerling, Monoclonal Antibodies and T-Cell Hybridomas 5630681 (Elsevier N.Y. 1981).

Polypeptides, such as any set forth herein, including the anti-HCV antibodies or fragments thereof provided herein, can be produced by any method known to those of skill in the art including in vivo and in vitro methods. Desired polypeptides can be expressed in any organism suitable to produce the required amounts and forms of the proteins, such as for example, needed for analysis, administration and treatment. Expression hosts include prokaryotic and eukaryotic organisms such as E. coli, yeast, plants, insect cells, mammalian cells, including human cell lines and transgenic animals (e.g., rabbits, mice, rats, and livestock, such as, but not limited to, goats, sheep, and cattle), including production in serum, milk and eggs. Expression hosts can differ in their protein production levels as well as the types of post-translational modifications that are present on the expressed proteins. The choice of expression host can be made based on these and other factors, such as regulatory and safety considerations, production costs and the need and methods for purification.

a. Nucleic Acids

Provided herein are isolated nucleic acid molecules encoding a polypeptide described above, that, alone or in combination with another polypeptide, can bind HCV. Thus, also provided are isolated nucleic acid molecules encoding an anti-HCV antibody or antigen-binding fragment thereof provided herein. In some examples, the isolated nucleic acid molecule encodes a Fab fragment that is selected from among A, B1, B2, B3, B4, B5, B6, B7, C1, C1*, C2, C2*, C3, C4, C5, C6, D1, D2, D3, D4, E, E2, E3, E4, F, G, H1, H1*, H1**, H2, H3, I, J1, J2, J3, J3*, J4, J5, K, L1, L2, L3, L4, L5, L6, L7, M, N1, N2, N3, N4, O1, P1, P1a, P2, P3, P4, P5, P6, Q1, Q2, R1, S1, S2, T1, U1, U2, U3 and V1 Fabs. In some examples, the isolated nucleic acid molecule encode a full length IgG of the A, B1, B2, B3, B4, B5, B6, B7, C1, C1*, C2, C2*, C3, C4, C5, C6, D1, D2, D3, D4, E, E2, E3, E4, F, G, H1, H1*, H1**, H2, H3, I, J1, J2, J3, J3*, J4, J5, K, L1, L2, L3, L4, L5, L6, L7, M, N1, N2, N3, N4, O1, P1, P1a, P2, P3, P4, P5, P6, Q1, Q2, R1, S1, S2, T1, U1, U2, U3 and V1 Fabs, or any antigen-binding fragment thereof. Exemplary of these nucleic acid molecules are those set forth in SEQ ID NOS: 611-641 and 1024-1042 that encode a Fab heavy chain, and those set forth in SEQ ID NOS: 642-677 and 1043-1061 that encode a Fab light chain (see Table 5 and 6).

TABLE 5
Nucleic Acids Encoding Fab Heavy Chains

| | SEQ ID | |
|---|---|---|
| Fab | NO | Nucleic Acids Encoding Fab Heavy Chains |
| A | 611 | CTCGAGCAGTCTGGGGCTGAGGTGAAGAAGCCTGGGTCCTCGGTGAAAGTC |
| | | TCCTGCAAGGCGTCTGGGGGCACCTTCAGCAGCTTTGTTATCAACTGGGTG |
| | | CGACAGGCCCCTGGACAAGGGCTAGAGTGGGTGGGAGGGATCTTCCAGGCC |
| | | CCCGGACCAGAGCGTGAGTGGCTGCGGGACATTAACCCAATCTCTGGGACG |
| | | ATAAACTACGCACAGAGGTTCCAGGGCAGAGTCACGATGACCGCGGACGAA |
| | | TCCATGACCACAGTCTACATGGAGCTGAGCAGTCTGAGATCTGAAGACACG |
| | | GCCATGTATTACTGTGCGAGAGAAAACAAATTCAGATACTGTCGTGGTGGT |
| | | AGTTGCTACTCTGGTGCTTTTGATATGTGGGGCCAGGGGACAATGGTCACC |
| | | GTCTCTTCAGCCTCC |
| B1 | 612 | CTCGAGCAGTCTGGGGCAGAGGTGAAGAAGCCGGGGTCCTCAGTGAAAGTC |
| | | TCCTGCAGGGCCTCTGGAAGCCCCTTTGGTAGTTACACGATCACTTGGGTG |
| | | CGACAGGCCCCTGGACAAGGACTTGAGTGGATGGGAGGAATCATCCTGATG |
| | | ACTGGTAAAGCGAACTACGCACAGAAGTTCCAGGGCAGAGTCACGATCACT |
| | | GCGGACAGATCAACGACCACAGCCTACATGGAAATGAGCAGCCTGACATCT |
| | | GACGACACGGCCATTTATTACTGTGCGAGAGATCCCTATGTATATGCAGGG |
| | | GATGACGTGTGGTCTTTGTCTCGGTGGGGCCAGGGAACCCTGGTTATCGTC |
| | | TCCTCAGCCTCC |
| B2 | 613 | CTCGAGCAGTCTGGGGCAGAGGTGAAGAAGCCGGGGTCCTCAGTGAAAGTG |
| | | TCCTGCAGGGCCTCTGGAAGCCCCTATAGTAGTTACACGATCACTTGGGTG |
| | | CGACAGGCCCCTGGACAAGGACTTGAGTGGATGGGAGGAATCATCCTGATG |
| | | ACTGGTAAAGCGAACTACGCACAGAAGTTCCAGGGCAGAGTCACCATCACT |
| | | GCGGACAGAGCAACGGCCACAGCCTACATGGAAATGAGCAGCCTGACATCT |
| | | GACGACACGGCCATATATTACTGTGCGAGAGATCCCTATGTTTATGCAGGG |
| | | GATGACGTGCGGTCTTTGTCTCGGTGGGGCCAGGGAACCCCGGTCATCGTC |
| | | TCCTCAGCCTCC |
| B3 | 614 | CTCGAGCAGTCTGGGGCAGAGGTGAAGAAGCCGGGGTCCTCAGTGAAAGTG |
| | | TCCTGCAGGGCCTCTGGAAGCCCCTATAGTAGTTACACGATCACTTGGGTG |
| | | CGACAGGCCCCTGGACAAGGACTTGAGTGGATGGGAGGAATCATCCTGATG |
| | | ACTGGTAAAGCGAACTACGCACAGAAGTTCCAGGGCAGAGTCACCATCACT |
| | | GCGGACAGAGCAACGGCCACAGCCTACATGGAAATGAGCAGCCTGACATCT |
| | | GACGACACGGCCATATATTACTGTGCGAGAGATCCCTATGTTTATGCAGGG |
| | | GATGACGTGTGGTCTTTGTCTCGGTGGGGCCAGGGAACCCCGGTCATCGTC |
| | | TCCTCAGCCTCC |
| C1 & C1* | 615 | CTCGAGCAGTCTGGGGCTGAGGTGAAGACGCCTGGGTCCTCGGTGAGGGTC |
| | | TCCTGCAGGCCTCCTGGAGGCAACTTCAACAGTTATAGTATAAACTGGGTC |
| | | CGACAGGCCCCTGGACACGGCCTTGAGTGGGTGGGGACTTTCATCCCTATG |
| | | TTTGGAACCTCAAAGTACGCGCAGAAGTTTCAGGGGAGAGTCACGATTACC |
| | | GCGGACGGGTCCTCGGGCACCGCTTACATGGACCTGAACAGCCTGAGATCT |
| | | GACGACACGGCCTTTTACTACTGTGTGCGTCCTGAAACGCCCAGATATTGT |
| | | AGTGGCGGTTTCTGCTATGGTGAGTTTGACAACTGGGGCCAGGGAACCCTG |
| | | GTCACCGTCTCCTCAGCCTCC |
| C2 & C2* | 616 | CTCGAGCAGTCTGGGGCTGAAGTGAAGAAGCCTGGGTCTTCGGTGAGAGTC |
| | | TCCTGCAGGGCTCCTGGTGGCACCTTCAACAGCTATAGTGTGAACTGGGTC |
| | | CGACAGGCCCCTGGGCACGGCCTTGAGTGGGTGGGGACGCTCATCCCTATG |
| | | TTTGGTACCTCAAGTTACGCGCAGAAGTTCCAGGGGAGAGTCACCATTACC |
| | | GCGGACGGGTCCTCGGGCACCGCCTACATGGAACTGAACAGCCTGAGATCT |
| | | GACGACACGGCCGTCTACTACTGTGTGCGACCTGAAACGCCCAGATATTGT |
| | | CGTGGCGGTTTCTGCTATGGTGAATTTGACAACTGGGGCCAGGGAACCCTG |
| | | GTCACCGTCTCCTCGGCCTCC |
| C3 | 617 | CTCGAGCAGTCTGGGGCTGAGGTGAAGGAGCCTGGGTCCTCGGTGAGGGTC |
| | | TCCTGCAGGGCTCCTGGAGGCACCTTCAACAGCTATAGTATAAATTGGGTC |
| | | CGACAGGCCCCTGGACACGGCCTTGAGTGGGTGGGGACGCTCATCCCTATG |
| | | TTTGGTACCTCAAACTACGCGCAGAAGTTCCAGGGGAGAGTCACCATTACC |
| | | GCGGACGGGTCCTCGGGCACCGCCTACATGGAGCTGAACAGCCTGAGATCT |
| | | GACGACACGGCCGTATACTACTGTGTGCGACCTGAAACGCCCAGATATTGT |
| | | AGTGGCGGTGTCTGCTATGGTGAATTTGACAACTGGGGCCAGGGAACCCTG |
| | | GTCACCGTCTCCTCAGCCTCC |
| C4 | 618 | CTCGAGTCTGGGGCTGAGGTGAAGAAGCCTGGGTCCTCGGTGAGGGTCTCC |
| | | TGCAGGCCTCCTGGAGGCACCTTCAATAGCTATAGTATAAACTGGGTCCGA |
| | | CAGGCCCCTGGACACGGCCTTGAGTGGGTGGGGACGATCATCCCTATGTTT |
| | | GGAACCTCAAAGTACGCGCAGAAGTTGCAGGGGAGAGTCACGATTACCGCG |
| | | GACGGGTCCTCGGGCACCGCTTACATGGAGCTGAACAGCCTGAGATCTGAC |
| | | GACACGGCCGTATATTACTGTGTGCGACCTGAAACGCCCAGATATTGTAGT |
| | | GGCGGTTTCTGCTATGGTGAATTTGACAACTGGGGCCAGGGAACCCTGGTC |
| | | ACCGTCTCCTCAGCCTCC |
| C5 | 619 | CTCGAGCAGTCTGGGGCTGAGGTGAAGAAGCCTGGGTCCTCGGTGAGGGTC |
| | | TCCTGCAGGGCTCCTGGAGGCACCCTCAACAGCTATAGTATAAACTGGGTC |
| | | CGACAGGCCCCTGGACACGGCCTTGAGTGGGTGGGGACGCTCATCCCTATG |
| | | TTTGGTACCTCAAACTACGCGCAGAAGTTCCAGGGGAGAGTCACCATTACC |
| | | GCGGACGGGTCCTCGGGCACCGCCTACATGGAGCTGAACAGCCTGAGATCT |
| | | GACGACACGGCCGTATACTACTGTGTGCGACCTGAAACGCCCAGATATTGT |
| | | AGTGGCGGTTTCTGCTATGGTGAATTTGACAACTGGGGCCAGGGAACCCTG |
| | | GTCACCGTCTCCTCAGCCTCC |
| C6 | 620 | CTCGAGCAGTCTGGGGCTGAGGTGAAGAAGCCTGGGTCCTCGGTGAGGGTC |
| | | TCCTGCAGGCCTCCGGGAGGCACCTTCAACAGTTATAGTATAAACTGGGTC |
| | | CGACAGGCCCCTGGACACGGCCTTGAGTGGGTGGGGACGATCATCCCTATG |
| | | TTTGGAACCTCAAAATACGCGCAGAAGTTGCAGGGGAGAGTCACGATTACC |
| | | GCGGACGGGTCCTCGGGCACCGCTTACATGGAGCTGAACAGCCTGAGATCT |
| | | GACGACACGGCCGTATACTACTGTGTGCGACCTGAAACGCCCAGATATTGT |
| | | AGTGGCGGTTTCTGCTATGGTGAATTTGACAATTGGGGCCAGGGAACCCTG |
| | | GTCACCGTCTCCTCAGCCTCC |
| D1 | 621 | CTCGAGTCTGGGGGAGGCTTGGTCCAGCCGGGGGGGTCCCTGAGACTCTCC |
| | | TGTGAAGCCTCTGGATATTATTTCAGTAGCTTTGCCATGAGTTGGGTCCGC |
| | | CAGACTCCAGGGAAGGGACTGGAGTGGGTCTCCAGTATTGCTGGTGGTACT |
| | | CTTGGAAGAACATCCTATAGAGACTCCGTGAAGGGCCGCTTCACCATCTCC |
| | | AGAGACAACTCCAAGAATACGGTGTTTCTCCACATGAACAACCTGAGACCC |
| | | GAGGACACGGCCGTCTATTATTGTGCGAAAGATCCACTTCTCTTCGCAGGA |
| | | GGACCTAATTGGTTCGACCACTGGGGCCAGGGAACCCTGGTCACCGTCTCC |
| | | TCAGCCTCC |
| D2 | 622 | CTCGAGCAGTCTGGGGGAGGCTTGGTCCAGCCGGGGGGGTCCCTGAGACTC |
| | | TCCTGTGAAGCCTCTGGATATTATTTCAGTAGCTTTGCCATGAGTTGGGTC |
| | | CGCCAGACTCCAGGGAAGGGACTGGAGTGGGTCTCCAGTATTGCTGGTGGT |
| | | ACTCTTGGAAGAACATCCTATAGAGACTCCGTGAAGGGCCGCTTCACCATC |
| | | TCCAGAGACAACTCCAAGAATACGGTGTTTCTCCACATGAACAACCTGAGA |
| | | CCCGAGGACACGGCCGTCTATTATTGTGCGAAAGATCCACTTCTCTTCGCA |
| | | GGAGGACCTAATTGGTTCGACCACTGGGGCCAGGGAACCCTGGTCACCGTC |
| | | TCCTCAGCCTCC |
| D3 | 623 | CTCGAGTCTGGGGGAGGCTTGGTCCAGCCGGGGGGGTCCCTGAGACTCTCC |
| | | TGTGAAGCCTCTGGATATTATTTCAGTAGCTTTGCCATGAGTTGGGTCCGC |
| | | CAGACTCCAGGGAAGGGACTGGAGTGGGTCTCCAGTATTGCTGGTGGTACT |
| | | CTTGGAAGAACATCCTATAGAGACTCCGTGAAGGGCCGCTTCACCATCTCC |
| | | AGAGACAACTCCAAGAATACAATGTTTCTCCACATGAACAACCTGAGACCC |
| | | GAGGACACGGCCGTCTATTATTGTGCGAAAGATCCACTTCTCTTCGCAGGA |
| | | GGACCTAATTGGTTCGACCACTGGGGCCAGGGAACCCTGGTCACCGTCTCC |
| | | TCAGCCTCC |
| D4 | 624 | CTCGAGTCTGGGGGAGGCTTGGTCCAGCCGGGGGGGTCCCTGAGACTCTCC |
| | | TGTGAAGCCTCTGGATATTATTTCAGTAGCTTTGCCATGAGTTGGGTCCGC |
| | | CAGACTCCAGGGAAGGGACTGGAGTGGGTCTCCAGTATTGCTGGTGGTACT |
| | | CTTGGAAGAACATCCTATAGAGACTCCGTGAAGGGCCGCTTCACCATCTCC |
| | | AGAGACAACTCCAAGAATACGGTGTTTCTCCACATGAGCAACCTGAGACCC |
| | | GAGGACACGGCCGTCTATTATTGTGCGAAAGATCCACTTCTCTTCGCAGGA |
| | | GGACCTAATTGGTTCGACCACTGGGGCCAGGGAACCCTGGTCACCGTCTCC |
| | | TCAGCCTCC |
| E | 625 | CTCGAGCAGTCTGGGGCTGAGCTGAAGAAGCCTGGGTCTTCGGTGAAGGTC |
| | | TCCTGCAAGCCCTCTGATGGCACCTTCAGGGCCTATACTCTCAGCTGGGTG |
| | | CGACAGGCCCCTGGACAAACGCTTGAGTGGATGGGCAGGATCATGCCTACT |
| | | GTTGGCATAACAAACTACGCACAGAAATTCCAGGGCAGAGTCACCATTTCC |
| | | GCGGACATGTCCACGGCCACAGCCTACATGGAACTGAGCAGCCTGCGATCT |
| | | GACGACACGGCCATATATTACTGTGCGAAAGGCCCGTACGTTGGCCTTGGG |
| | | GAAGGGTTCTCGGAGTGGGGCCAGGGAACTCTGGTCACCGTCTCCTCAGCC |
| | | TCC |
| F | 626 | CTCGAGCAGTCAGGAAATGAGGTGAAGAAGCCAGGGGCCTCAGTGAAAGTC |
| | | TCCTGCCGGGCTTATGGCTACAATTTTGGCAGCGAACGTCTCAGCTGGGTG |
| | | CGACAGGCCCCTGGACAAGGCCTTGAGTGGATGGGATGGATCAGCGCTTAC |
| | | AATGGTGGCATAAACTATTCACAGAAGTTCCAGGGCAGATTCACCATGACC |
| | | ACAGACACGTCCACGCGCACAGGCTACATGGAATTGAGGAACCTCAGATCT |
| | | GACGACACGGCCGTCTATTACTGTGCGAGAGGGGGGGGGACTGAGTGGGGC |
| | | CAGGGAACCCTGGTCATCGTCTCCTCAGATGAGTCCTCCTCAGCCTCC |
| G | 627 | CTCGAGCAGTCAGGAGCTGAGATGAAGAAGCCTGGGGCCTCATTGAAGGTC |
| | | TCCTGCAAGACTTCTGGTTACACGTTTGACGACTATGGTGTCACCTGGGTG |
| | | CGACAGGCCCCTGGACAAGGACTTGAGTGGATGGGCTGGATCAGCGCTTAC |
| | | AGTGGTAACACATTCTATGCACGGAAGTTCCAGGGCAGAGTCACCATGACC |
| | | ACAGACCCATCCACGCGCACTGCCTACATGGAGCTGAGGAGCCTGAGATCT |
| | | GACGACACGGCCGTGTATTTCTGTGCGAGAGATCCTGGTCTTGCGATTAAT |
| | | GGAGTGGTTTTCCCCTACTTCGGTTTGGACGTCTGGGGCCAAGGGACCACG |
| | | GTCACCGTCTCATCAGCCTCC |
| H1, H1* | 628 | CTCGAGCAGTCTGGGGCTGAGGTGAAGAAGCCTGGCTCCTCGGTGAAAGTC |
| & H1** | | TCCTGCGAGGCTTCTGGAGGCACCTTCGACAACTATTCTCTCAATTGGGTG |
| | | CGACAGGCCCCTGGACAAGGACTTGAGTGGATAGGAGGGGTCGTCCCTTTG |
| | | TTCGGTACAACAAAATACGCACAGAAGTTCCAGGGCAGAGTCACGATAAGC |
| | | GACGACAAATCGACGGGCACAGGACACATGGAGTTGAGAAGCCTGAGATCG |
| | | GAAGACACGGCCGTCTATTATTGTGTGAGATCAGTCACGCCCAGACATTGT |
| | | GGTGGTGGGTTTTGCTACGGTGAATTTGACTACTGGGGCCAGGGAACCCTG |
| | | GTCACCGTCTCCTCAGCCTCC |
| H2 | 629 | CTCGAGCAGTCTGGGGCTGAGGTGAAGAAGCCTGGCTCCTCGGTGAAAGTC |
| | | TCCTGCGAGACTTCTGGAGGGACTTTCGACAACTATGCTCTCAATTGGGTG |
| | | CGACAGGCCCCTGGACAAGGACTTGAGTGGATAGGAGGGGTCGTCCCTTTG |
| | | TTCGGTACAACAAAATACGCACAGAAGTTCCAGGGCAGAGTCACGATAAGC |
| | | GACGACAAATCGACGGGCACAGGACACATGGAGTTGAGAAGCCTGAGATCG |
| | | GAAGACACGGCCGTCTATTATTGTGTGAGATCAGTCACGCCCAGACATTGT |
| | | GGTGGTGGGTTTTGCTACGGTGAATTTGACTACTGGGGCCAGGGAACCCTG |
| | | GTCACCGTCTCCTCAGCCTCC |
| H3 | 630 | CTCGAGCAGTCTGGGGCTGAGGTGAAGAAGCCTGGCTCCTCGGTGAAAGTC |
| | | TCCTGCGAGACTTCTGGAGGGACCTTGGACAACTATGCTCTCAATTGGGTG |
| | | CGACAGGCCCCTGGACAAGGACTTGAGTGGATCGGAGGGGTCGTCCCTTTG |
| | | TTTGGTACAACAAGAAACGCACAGAAGTTCCAGGGCAGAGTCACGATAAGC |
| | | GACGACAAATCGACGGGCACAGGACACATGGAGTTGAGAAGCCTGCGATCG |
| | | GAAGACACGGCCGTTTATTATTGTGTGAGATCAGTCACGCCCAGATATTGT |
| | | GGTGGTGGGTTTTGCTACGGTGAATTTGACTACTGGGGCCAGGGAACCCTG |
| | | GTCACCGTCTCCTCAGCCTCC |
| I | 631 | CTCGAGTCTGGGGGAGGCTTGGTACAGCCAGGGCGGTCCCTGAGACTCTCC |
| | | TGCAAAGCGTCTGGCTTCAATTTTGCTCAGTATACTATGAACTGGGTCCGC |
| | | CAGGCTCCAGGGAAGGGACTGGAGTGGATCGGGCTCATTAGAACCACAGCT |
| | | TATGATGCGGCTACACATTATGCTGCGTCTGTGGAGGGCAGATTCACCATT |
| | | TCCAGAGATGATTCCAAAAGTACCGCCTATCTGCAGATAAACGGCCTGAAA |
| | | ACCGAGGACACAGCCGTCTATTACTGTGCTAGACCCCATGGACCCGGGTTA |
| | | AGTCTTGGCATTTACAGCGCTGAATACTTCGATGAGTGGGGCCAGGGCACC |
| | | CTGGTCACCGTCTCCTCAGCCTCC |
| J1 | 632 | CTCGAGCAGTCTGGGCCTGAGGTGAAGAAGCCTGGGTCCTCGGTGAAGGTC |
| | | TCCTGCAAGGGTTCTGGAGACAGGTTCAACGATCCTGTCACCTGGGTGCGA |
| | | CAGGCCCCTGGACAAGGCCTTGAGTGGATCGGAGGAATCATCCCTGCGTTT |
| | | GGTGCAACAAAGTATGCACAGAAGTTCCAGGGCAGAGTCGTCATTTCCGCG |
| | | GACGCATCCACGGACACGGCCTACATGGAACTGAGCAGCCTGAGATCTGAA |
| | | GACACGGCCGTCTATTATTGTGCGAAAGTAGGCGTGCGGGGCATTATTTTG |
| | | GTTGGGGGCCTGGCGATGAACTGGCTCGACCCCTGGGGCCAGGGAACCCTA |
| | | GTCACCGTCTCCGCAGCCTCC |
| J2 | 633 | CTCGAGCAGTCTGGGCCTGAGGTGAAGAAGCCTGGGTCGTCGGTGAAGGTC |
| | | TCCTGCAAGGATTCTGGAGACACCTTCAACGAACCTGTCACCTGGGTGCGA |
| | | CAGGCCCCTGGACAAGGCCTTGAGTGGATCGGAGGAATCATCCCTGCGTTT |
| | | GGTGTGACAAAGTACGCACAGAAATTCCAGGGCCGAGTCATCATTTCCGCG |
| | | GACGCATCTACGGCCACGGCCTATTTGGAGCTGAGCAGTCTGAGATCTGAA |
| | | GACACGGCCGTCTATTACTGTGCGAAAGTTGGCCTGCGGGGCATTGTAATG |
| | | GTTGGGGGCCTGGCGATGAACTGGCTCGACCCCTGGGGCCAGGGAACCCAA |
| | | GTCACCGTCTCCTCAGCCTCC |
| J3 & | 634 | CTCGAGCAGTCTGGGCCTGAGGTGAAGAAGCCTGGGTCCTCGGTGAAAGTC |
| J3* | | TCCTGCAAGGGTTCTGGAGACACCTTCAACGATCCTGTCACCTGGGTGCGA |
| | | CAGGCCCCTGGACAAGGCCTTGAGTGGATCGGAGGAATCATCCCTCTGTTT |
| | | GGTGCAGCAAAATACGCACAGAAGTTCCAGGGCAGAGTCACGATTTCCGCG |
| | | GACGCATCAGCGTTAACGACCTACATGGAGATGAGCAGCCTGAGACCGGAA |
| | | GACACGGCCGTCTATTATTGTGCGAAAGTGGGTCTGCGGGGCATTACTTTG |
| | | GTTGGGGGCCTGGCGATGAACTGGCTCGACCCCTGGGGCCAGGGAACCCTA |
| | | ATCACGGTCTCCTCAGCCTCC |
| J4 | 635 | CTCGAGCAGTCTGGGGCTGAGGTGAAGAAGCCTGGGTCCTCGGTGAGAGTC |
| | | TCCTGCGAGGTTTCTGGAGACACCTTCAGGGAGCCTGTCAGTTGGGTGCGA |
| | | CAGGCCCCTGGACAAGGATTTGAGTGGATCGGAGGAATCATCCCTATGTTT |
| | | GGCGCAACACATTACGCACAGAAGTTACAGGGCAGAATCACTATTTCTGCG |
| | | GACCAATCGACGAACACAGTCTACATGGAACTGAGGAGCCTGAGATCTGAC |
| | | GACACGGCAGTTTATTATTGTGCGAAAGTTGGACTGCGGGGCATTAATATG |
| | | GTTGGGGGCCTGGCGATGAACTGGTTCGACCCCTGGGGCCAGGGAACCCTA |
| | | GTCACCGTCTCCTCAGCCTCC |
| K | 636 | CTCGAGCAGTCAGGTCCAGGACTGGTGAAGCCTGGCAGACCCTTTTCACTC |
| | | ACCTGTGCCATCTCCGGGGACAGTGTCTCTAGCGACAGTGCTGCTTGGAAC |
| | | TGGGTCAGGCAGTCCCCGTCGAGAGGCCTTGAGTGGCTGGGAAGGACATTC |
| | | TACAGGTCCAAGTGGTATTATGATTATACAGTATCTGTGAAAAGTCGAATC |
| | | ACCATCAACTCAGACACATCCAAGAACCAGTTTTCCCTGCACCTCAACTCT |
| | | GTGACTCCCGAGGACACGGCTGTGTATTATTGTGTAAGAGATTTTTATATT |
| | | GGCCCAACCAGAGACGTCTACTACGGTATGGACGTCTGGGGCCAAGGGACC |
| | | ACGGTCACCGTCTCCTCAGCCTCC |
| L1 | 637 | CTCGAGCAGTCTGGGGCTGAGGTGAAGAAGCCTGGGTCGTCGGTGAAGGTC |
| | | TCCTGCAAGGCTTCTGGAGACACCTTCAGAAGTTATGTCATCACATGGGCG |
| | | CGACAGGCCCCTGGACAAGGGCTTGAGTGGATGGGAGCGATCATCCCTTTC |
| | | TTTGGAACAACAAACCTCGCACAGAAATTCCAGGGCAGAGTCACGATTACC |
| | | GCGGACGAATCAACGCAGACAGTCTACATGGACTTGAGCAGCCTGAGATCT |
| | | GACGACACGGCCGTTTATTATTGTGCGAAAGCCGGAGATCTTTCAGTTGGG |
| | | GGAGTTCTCGCCGGCGGGGTTCCGCACTTGCGACATTTTGACCCCTGGGGC |
| | | CAGGGAACCCTGGTCACCGTCTCCTCGGCCTCC |
| L2 | 638 | CTCGAGCAGTCTGGGGCTGAGGTGAAGATGCCCGGGTCGTCGGTGAAGGTC |
| | | TCCTGCAAGGCTTCTGGAGACACGTTCAGAAGTTCTGTTATCACATGGGCG |
| | | CGACAGGCCCCTGGACAGGGGCTTGAGTGGATGGGAGCGATCATCCCTTTC |
| | | TTTGGAACAACAAACCTCGCACAGAAGTTCCAGGGCAGAGTCACGATTACC |
| | | GCGGACGAATCAACGAAGACAGTCTACATGGACTTGAGCAGCCTGAGATCT |
| | | GATGACACGGCCGTTTATTATTGCGCGAAAGCCGGAGATCTTTCAGTTGGG |
| | | GGAGTTCTCGCCGGCGGGGTTCCGCACTTGCGACATTTCGACCCCTGGGGC |
| | | CAGGGAACCCTGGTCACCGTCTCCTCGGCCTCC |
| L3 | 639 | CTCGAGCAGTCTGGGGCTGAGGTGAAGAAGCCTGGGTCGTCGGTGAAGGTC |
| | | TCCTGCAAGGCTTCTGGAGACACCTTCAGAAGTTATGTTATCACATGGGCG |
| | | CGACAGGCCCCTGGACAAGGGCTTGAGTGGATGGGAGCGATCATCCCTTTC |
| | | TTTGGAACAACAAACCTCGCACAGAAGTTCCAGGGCAGAGTCACGATTACC |
| | | GCGGACGAATCAACGAAGACAGTCTACATGGACTTGAGCAGCCTGACATCT |
| | | GATGACACGGCCGTTTATTATTGTGCGAAAGCCGGAGATCTTTCAGTTGGG |
| | | GGAGTTCTCGCCGGCGGGGTTCCGCACTTGCGACATTTCGACCCCTGGGGC |
| | | CAGGGAACCCTGGTCACCGTCTCCTCGGCCTCC |
| L4 | 640 | CTCGAGCAGTCTGGGGCTGAGGTGAAGAAGCCTGGGTCGTCGGTGAAGGTC |
| | | TCCTGCAAGGCTTCTGGAGACACCTTCAGAAGTTATGTTATCACATGGGCG |
| | | CGACAGGCCCCTGGACAAGGGCTTGAGTGGATGGGAGCGATCATCCCTTTC |
| | | TTTGGAACAACAAACCTCGCACAGAAGTTCCAGGGCAGAGTCACGATTACC |
| | | GCGGACGAATCAACGAAGACAGTCTACATGGACTTGAGCAGCCTGAGATCT |
| | | GATGACACGGCCGTTTATTATTGTGCGAAAGCCGGAGATCTTTCAGTTGGG |
| | | GGAGTTCTCGCCGGCGGGGTTCCGCACTTGCGACATTTCGACCCCTGGGGC |
| | | CAGGGAACCCTGGTCACCGTCTCCTCGGCCTCC |
| M | 641 | CTGGAGCAGTCAGGAGCTGAGGTGAAGAAGCCTGGGGCCTCAGTGAAGGTC |
| | | TCCTGCAAGGCCTCTGGTTACACTTTTACTAACTATGCAATTACCTGGGTG |
| | | CGACAGGCCCCTGGACAAGGTCTTGAGTGGATGGGATGGATCAGCGGTGAC |
| | | AGCACTAACACATACTATGGTCAGAAGTTCCAGGGAAGAGTCACCATGACG |
| | | ACAGACACATCCACGAGCACAGCCTACATGGAGTTGACGAGCCTGACATCT |
| | | GAGGACACGGCCGTGTATTACTGTGCGAGAGAATCGCTCTATATGATTGCG |
| | | TTTGGGAGAGTTATATGGCCACCACTTGACTACTGGGGCCAGGGAACTCTG |
| | | GTCACCATCTCCTCTGCCTCC |
| N1 | 1024 | GAGGTGCAGCTGCTCGAGCAGTCTGGGCCAGAGGTGAAAAAGCCCGGGGAT |
| | | TCTCTGAGGATCTCCTGTAAGATGTCTGGAGACAGTTTAGTCACCACTTGG |
| | | ATCGGCTGGGTGCGCCAGAAGCCCGGGCAAGGCCTGGAGTGGATGGGGATC |
| | | ATCAATCCTGGTGACTCTTCTACCAACATCTATCCTGGTGACTCTGCCACG |
| | | CGATATGGCCCGTCCTTCCAAGGCCAGGTCACCATCTCAATCGACAAGTCA |
| | | ACCAGCACCGCCTACCTGCAGTGGAACGCTGTGAAGCCCTCGGACACCGGC |
| | | ATTTATTACTGTGCGAGACATGTCCCCGTACCAATCTCCGGGACTTTTCTT |
| | | TGGAGAGAGCGGGAAATGCATGATTTCGGCTACTTTGACGACTGGGGCCAG |
| | | GGAACCCTGGTCATCGTCTCCTCA |
| N2 | 1025 | GAGGTGCAGCTGCTCGAGCAGTCTGGGCCAGAGGTGAAAAAGCCCGGGGAT |
| | | TCTCTGAGGATCTCCTGTAAGATGTCTGGAGACAGTTTAGTCACCACTTGG |
| | | ATCGGCTGGGTGCGCCAGAAGCCCGGGCAAGGCCTGGAGTGGATGGGGATC |
| | | ATCAATCCTGGTGACTCTTCTACCAACATCTATCCTGGTGACTCTGCCACG |
| | | CGATATGGCCCGTCCTTCCAAGGCCAGGTCACCATCTCAATCGACAAGTCA |
| | | ACCAGCACCGCCTACCTGCAGTGGAACACTGTGAAGCCCTCGGACACCGGC |
| | | ATTTATTACTGTGCGAGACATGTCCCCGTACCAATCTCCGGGACTTTTCTT |
| | | TGGAGAGAGCGGGAAATGCATGATTTCGGCTACTTTGACGACTGGGGCCAG |
| | | GGAACCCTGGTCATCGTCTCCTCA |
| N3 | 1026 | GAGGTGCAGCTGCTCGAGCAGTCTGGGGCAGAGGTGAAGAAGCCCGGGGAT |
| | | TCTCTGAGGATCTCCTGTAAGATGTCTGGAGACAGTTTAGTCTGGATCGGC |
| | | TGGGTGCGCCAGAAGCCCGGGCAAGGCCTGGAGTGGATGGGGATCATCAAT |
| | | CCTGGTGACTCTGCTACCAACATCTATCCTGGCGACTCTGACACCCGATAT |
| | | GGCCCGTCCTTCCAAGGCCAGGTCACCATCTCAATCGACAAGTCCACCAGC |
| | | ACCGCCTACCTGCAGTGGAACGCTGTGAAGGCCTCGGACACCGGCATTTAT |
| | | TACTGTGCGAGACATGTCCCCGTACCAATCTCCGGGACTTTTCTTTGGAGA |
| | | GAGAGGGAAATGCATGATTTGGGCTACTTTGACGACTGGGGCCAGGGAACC |
| | | CTGGTCATCGTCTCCTCA |
| N4 | 1027 | GAGGTGCAGCTGCTCGAGCAGTCTGGGCCAGAGGTGAAAAAGCCCGGGGAT |
| | | TCTCTGAGGATCTCCTGTAAGATGTCTGGAGACAGTTTAGTCACCACTTGG |
| | | ATCGGCTGGGTGCGCCAGAAGCCCGGGCAAGGCCTGGAGTGGATGGGGATC |
| | | ATCAATCCTGGTGACTCTTCTACCAACATCTATCCTGGTGACTCTGCCACG |
| | | CGATATGGCCCGTCCTTCCAAGGCCAGGTCACCATCTCAATCGACAAGTCC |
| | | ACCAGCACCGCGTACCTGCAGTGGAACAATGTGAAGGCCTCGGACACCGGC |
| | | ATTTATTACTGTGCGAGACATGTCCCCGTACCAATCTCCGGGACTTTTCTT |
| | | TGGAGAGAGCGGGAAATGCATGATTTCGGCTACTTTGACGACTGGGGCCAG |
| | | GGAACCCTGGTCATCGTCTCCTCA |
| O1 | 1028 | GAGGTGCAGCTGCTCGAGCAGTCTGGGGCGGAGGTGAAAAAGGCCGGGGAG |
| | | TCTGTCAGACTCTCCTGTAAGGCTTCGGGATACAGGTTTGGCGACTACTGG |
| | | ATCGCCTGGGTGCGCCAGTTGCCCGGAAGAGCCCCGGAATGGATGGGGATC |
| | | ATCTATCCTGATGACTCTGATACCAAGTACAGCCCGTCCTTCCAAGGCCAG |
| | | GTCACCATCTCAGCCGACAAGTCCATCAGAACCACCTTCTTGGACTGGGGC |
| | | AGCCTGAAGGCCTCGGACACCGCCATCTATTACTGTGCGAGAGGCTGCCTT |
| | | GGTGCCAAGTGCTACTATCCTCACTACTATTACGGTTTGGACGTCTGGGGC |
| | | CAAGGGACCACGGTCATCGTCTCCTCA |
| P1 | 1029 | GAGGTGCAGCTGCTCGAGTCGGGGGGAGGCGTGGTCCAGCCTGGGGGGTCC |
| | | CTGAGACTCTCCTGTGCAGCCTCTGGATTCACATTCACTAGCTTTACTATG |
| | | CACTGGGTCCGCCAGGCTCCAGGCAAGGGGCTGGAGTGGGTGGCACTTATA |
| | | TCACATGATGGAAGCAATAAAGACTACGCGGACTCCGTGAGGGGCCGATTC |
| | | ACCGTCTCCAGAGACAATTCCAAGAAAATGGTTTATTTGCAGATGAGCAGC |
| | | CTGAGACCTGACGACGCGGCTGTCTATTACTGTGCGAGAGGGGGGCCCGCC |
| | | TATTATACTTATTCCGATACTTTGACTGGTTATCATAACGTCGTGGGGGAC |
| | | TACTGGGGCCAGGGAACCCTGGTCACCGTCTCCTCA |
| P1a | 1030 | GAGGTGCAGCTGCTCGAGTCGGGGGGAGGCGTGGTCCAGCCTGGGGGGTCC |
| | | CTGAGACTCTCCTGTGCAGCCTCTGGATTCACATTCACTAGCTTTACTATG |
| | | CACTGGGTCCGCCAGGCTCCAGGCAAGGGGCTGGAGTGGGTGGCACTTATA |
| | | TCACATGATGGAAGCAATAAAGACTACGCGGACTCCGTGAGGGGCCGATTC |
| | | ACCGTCTCCAGAGACAATTCCAAGAAAATGGTTTATTTGCAGATGAGCAGC |
| | | CTGAGACCTGACGACGCGGCTGTCTATTACTGTGCGAGAGGGGGGCCCGCC |
| | | TATTATACTTATTCCGATACTTTGACTGGTTATCATAACGTCGTGGGGGAC |
| | | TACTGGGGCCAGGGAACCCTGGTCACCGTCTCCTCA |
| P2 | 1031 | GAGGTGCAGCTGCTCGAGTCAGGGGGAGGCGTGGTCCAGCCTGGGAGGTCC |
| | | CTGAGACTCTCCTGTGCAGCCTCTGGATTCACTCTTGATACGTTTACTATG |
| | | CACTGGGTCCGCCAGTCTCCAGGCAAGGGGCTGGAGTGGGTGGCCCTTATC |
| | | TCACATGATGCCAACAATAAAGACTACGCGGACTCCGTGAAGGGCCGATTC |
| | | ACCATCTCCAGAGACAATTCCAAGAAATTGGTGTATTTGCAGATGGACAGC |
| | | CTGAGATCTGAGGACACGGCTGTCTATTACTGTGCGAGAGGGGGGCCCGCC |
| | | TATTATCTGTATTCCGATGTTTTGACTGGTTTTCATAACGTCGTGGGGGAC |
| | | TATTGGGGCCAGGGAACCCTGGTCACCGTCTCCGCA |
| P3 | 1032 | GAGGTGCAGCTGCTCGAGTCTGGGGGAGGCGTGGTCCAGCCTGGGAAGTCC |
| | | CTGAGACTCTCCTGTGCAGTCTCTGGATTCACGTTGAATACCTTTGCTATG |
| | | CACTGGGTCCGCCAGGTCCCAGGCAAGGGGCTGGAGTGGGTGGCTCTGACC |
| | | TCACATGATGGAAGCCGACAAGACTACGCAGACTCCGTGAGGGGCCGATTC |
| | | ACCATCTCCAGAGACAACTCCAAGAGTATGGTGTTTCTGCTGATGAACAGC |
| | | CTGAGAGCCGAGGACACGGCTGTGTATTATTGTGTGAGAGGGGGGCCCGCA |
| | | TATTATACGTACAACGATGTTTTGACTGGTTACGCTTATGTCGTGGGGGAC |
| | | TTCTGGGGCCAGGGAACCCTGGTAACCGTCTCCTCA |
| P4 | 1033 | GAGGTGCAGCTGCTCGAGTCTGGGGGAGGCGTGGTCCAGCCTGGGAAGTCC |
| | | CTGACAGTCTCCTGTGCAGCCTCTGGATTCACCTTCAGTACCTTTACCATG |
| | | CACTGGGTCCGCCAGGCTCCAGGCAAGTGGCTGGAGTGGGTGGCAGTCATC |
| | | TCACATGATGGCGGCACTGAACACTACGCAGACTCCGTGACGGGCCGATTC |
| | | ACCATCTCCCGAGACAATTCCAAGAACACGCTGCATCTGCAAATGAACAGC |
| | | CTGAGACCTGAGGACACGGCAGTGTACTTTTGTGCGAGAGGGGGGCCCGCG |
| | | TATTATTTGTATAACGATGTTCTGACTGGTTATTATAACGTCGTGGGGGAC |
| | | TTTTGGGGCCAGGGAACCCTGGTCACCGTCTCCTCA |
| P5 | 1034 | GAGGTGCAGCTGCTCGAGTCTGGGGGAGGCGTGGTCCAGCCTGGGAAGTCC |
| | | CTGACAGTCTCCTGTGCAGCCTCTGGATTCACCTTCAGTACCTTTACCATG |
| | | CACTGGGTCCGCCAGGCTCCAGGCAAGGGGCTGGAGTGGGTGGCAGTCATC |
| | | TCACATGATGGCGGCACTGAACACTACGCAGACTCCGTGACGGGCCGATTC |
| | | ACCATCTCCCGAGACAATTCCAAGAACACGCTGCATCTGCAAATGAACAGC |
| | | CTGAGACCTGAGGACACGGCAGTGTACTTTTGTGCGAGAGGGGGGCCCGCG |
| | | TATTATTTGTATAACGATGTCCTGACTGGTTATTATAACGTCGTGGGGGAC |
| | | TTCTGGGGCCAGGGAACCCTGGTCACCGTCTCCTCG |
| P6 | 1035 | GAGGTGCAGCTGCTCGAGTCTGGGGGAGGCGTGGTCCAGCCTGGGAAGTCC |
| | | CTGACAGTCTCCTGTGCAGCCTCTGGATTCACCTTCAGTACCTTTACCATG |
| | | CACTGGGTCCGCCAGGCTCCAGGCAAGTGGTTGGAGTGGGTGGCAGTCATC |
| | | TCACATGATGGCGGCACTGAACACTACGCAGACTCCGTGACGGGCCGATTC |
| | | ACCATCTCCCGAGACAATTCCAAGAACACGCTGCATATGCAAATGAACAGC |
| | | CTGAGACTTGAGGACACGGCAGTGTACTTTTGTGCGAGAGGGGGGCCCGCG |
| | | TATTATTTGTATAACGATGTTCTGACTGGTTATTATAACGTCGTGGGGGAC |
| | | TTTTGGGGCCAGGGAACCCTGGTCACCGTCTCCTCA |
| Q1 | 1036 | GAGGTGCAGCTGCTCGAGCAGTCTGGGGCTGAGGTGAGGAAGCCTGGGGCC |
| | | TCCGTGAAGGTCTCCTGCAAGGCTTCTGGTTACACCTTTACCAACAATGGT |
| | | CTCAACTGGGTGCGACAGGCCCCTGGACAAGGGCTTGAGTGGATGGGGTGG |
| | | ATCAGCCCTTACAATGGAGACACAGACTTTGCACATAAGTTCCAGGGCCGA |
| | | ATCAGCATGACCACAGACACATCCACGAATACAGCCTACATGGAGTTGAGG |
| | | AGTCTGAGATCGGACGACACGGCCGTGTATTACTGTGCGAGAGATCGGAAT |
| | | TCAGCAGGTGGTACCTGGCTTTTTCGCGACCCTCCACCTGGCTCGACGTTT |
| | | TTTGATTCCTGGGGCCAGGGATCCCTGGTCACCGTCTCCTCA |
| Q2 | 1037 | GAGGTGCAGCTGCTCGAGCAGTCAGGAGCTGAGGTGAAGAAGCCTGGGGCC |
| | | TCCGTGAAGGTCTCCTGCAAGGCTTCTGGTTACACCTTTACCAACAATGGT |
| | | CTCAACTGGGTGCGACAGGCCCCTGGACAAGGGCTTGAGTGGATGGGGTGG |
| | | ATCAGCCCTTACAATGGGGACACAGACTTTGCACATAAGTTCCAGGGCCGA |
| | | ATCAGCATGACCACAGACACATCCACGAATACAGCCTACATGGAGTTGAGG |
| | | AGTCTGAGATCAGACGACACGGCCGTGTATTACTGTGCGAGAGATCGGAAT |
| | | TCAGCAGGTGGTACCTGGCTTTTTCGCGACCCTCCACCTGGCTCGACGTTT |
| | | TTTGATTCCTGGGGCCAGGGATCCCTGGTCACCGTCTCCTCA |
| R1 | 1038 | GAGGTGCAGCTGCTCGAGCAGTCTGGGGCTGAGGTGAAGAAGCCTGGGGCC |
| | | TCAGTGAAGGTCTCCTGCAAGGCTTCTGGTTACACCTTTAGCATCTATGGT |
| | | GTTGCCTGGGTGCGACAGGCCCCTGGACAGGGACTTGAGTGGATGGGATGG |
| | | ATCAGCCCCCAGAATGGTGACACACACTCTCCACAGAAGTTCCAGGGCAGA |
| | | CTCACAATGACCACAGACACCTCCACGAGCACAGCCTACATGGAGCTGAGG |
| | | AGCCTGAGATCTGACGACACGGCCGTATATTTTTGTGCGAGAGACTATGGA |
| | | GTGAACTTCGGGGGAGGGTCCGAACACAACCTAGACTACTGGGGCCGGGGA |
| | | ACCCGGGTCACCGTCTCCTCA |
| S1 | 1039 | GAGGTGCAGCTGCTCGAGCAGTCTGGGGCTGAGGTTAAGAAGCCTGGGACC |
| | | TCAGTGAAGGTCTCCTGCACGGCTTCTGGTTACATCTTTACCAGTTTTGGT |
| | | ATTAGCTGGGTGCGACAGGCCCCTGGACAAGGACTTGAGTGGATGGGACGG |
| | | ATCGACACTTACAATGGTAAGACGAACTATGCACAGAAGCTCCAGGGCAGA |
| | | GTCACCATGACCACAGACACATACACGAGCACAGCCTACATGGAACTGAGG |
| | | AGCCTGACATCTGACGACACGGCCGTGTATTACTGTGCGAGAGATGATTGT |
| | | AGGAGTTCCACTTGCTATCTGGCTCAACACAACTGGCAGGCATATTACCAT |
| | | GACTCCTGGGGCCAGGGAACCCTGGTCACCGTCTCCTCA |
| T1 | 1040 | GAGGTGCAGCTGCTCGAGTCTGGGGGAGGCTTAGTTCAGCCTGGGGGGTCC |
| | | CTGAGACTCTCCTGTGCAGCCTCTGGATTCACCTTCAGTAGGTTCTGGATG |
| | | CACTGGGTCCGCCAAGCTCCAGGGAAGGGGCTGGTGTGGGTCGCACGTATT |
| | | AATAGTGATGGGAGTAGCACAACCTACGCGGACTCCGTGAAGGGCCGATTC |
| | | ACCATCTCCAGAGACAACGCCAAGAACACGTTGTATCTGCAAATGAACAGT |
| | | CTGAGAGACGAGGACACGGCTGTGTATTTCTGTGCAAGAGGGGGGGACAGC |
| | | TCATCCCCCTACTACTACCCTATGGACGTCTGGGGCCAAGGGACCACGGTC |
| | | GCCGTCTCATCA |
| U1 | 1041 | GAGGTGCAGCTGCTCGAGCAGTCAGGGCCTGAGGTGAAGAGGCCTGGGACC |
| | | TCGGTGAAAATGTCCTGCAAGATTTCGGGAGGCGCCTCCATCACTCAAGCC |
| | | ATGAGTTGGGTCCGACAGGCCCCAGGACAAGGTCTTGAGTGGATGGGGGGC |
| | | ATCACCCCTATCTTTGGAACAGTAAACTACGCACAGAAGATCTTGGGCAGA |
| | | GTCACCATTACCGCGGACGAGGACACAGTCTCCTTAGAGCTGAGCAGTCTG |
| | | AAGTCTGAGGACACGGCCGTCTATTATTGTGCGAGAGAGGTCAATTTGAAA |
| | | ACCTGGAACCTCGCCCATCCCAATGTCTTTGATGTCTGGGGCCAAGGGACA |
| | | ATGCTCACTGTCTCTTCA |
| V1 | 1042 | GAGGTGCAGCTGCTCGAGCAGTCTGGGGCTGAGGTGCAGAAGCCTGGGGCC |
| | | TCAGTGAAAGTCTCCTGCAAGCCGTCTGGTTACATTTTTACCAATTTTGGT |
| | | ATCAGCTGGGTTCGACAGGCCCCTGGACAAGGGCTTGAGTGGATGGCATGG |
| | | ATCAACACTTACAATGGTAAAACAACCTATGCACAGAGTCTCCAGGGCAGA |
| | | GTCACCCTGACCACCGACCCATACACGAACACAGTCTTCATGGAACTGAGG |
| | | AGCCTGAGATCTGACGACACGGCCGTCTATTACTGTGCGAGAGAAAACGAG |
| | | GGTGAATATGTTTGGGGTCATTTTCGTTCCGACTACTGGGGCCAGGGAACC |
| | | CTGGTCACCGTCTCCTCA |

TABLE 6
Nucleic Acids Encoding Fab Light Chains

| | | |
|---|---|---|
| | SEQ ID | |
| Fab | NO | Nucleic Acids Encoding Fab Light Chains |

| | | |
|---|---|---|
| A | 642 | GAGCTCACACAGTCTCCAGCCACCCTGTCTGTGTCTCCTGGGGAAAGC |
| | | GCCACCCTCTCCTGCAGGGCCAGTCAGAGTGTTAGCGACAACTTAGCC |
| | | TGGTACCAGCAGAAACCTGGCCAGGCTCCCAGGCTCCTCATCTATGGT |
| | | GCATCCAGCAGGGCCCCTGCCATCCCAGGCAGGTTCAGTGGCAGTGGG |
| | | TCTGGGACAGACTTCACTCTCACCATCAGCAGACTGGAGCCTGAAGAT |
| | | CTTGCAGTGTATCACTGTCAGCAGTATGGTGCGTCACCTTGGACGTTC |
| | | GGCCAAGGGACCAAGGTGGAAATCAAACGAACTGTGGCTGCACCATCT |
| | | GTCTTCATCTTCCCGCCATCTGATGAGCAGTTGAAATCTGGAACTGCC |
| | | TCTGTTGTG |
| B1 | 643 | GAGCTCACACTCACGCAGTCTCCAGGCACCCTGTCTTTGTCTCCAGGG |
| | | GAAAGAGCCACCCTCTCCTGTAGGGCCAGTCAGAGTGTTAGCAACAGC |
| | | TACTTAGCCTGGTACCAGCAGAAACCTGGCCAGGCTCCCAGGCTCCTC |
| | | ATCTATGGTGCATCCAGCAGGGCCACTGGCATCCCAGACAGGTTCAGT |
| | | GGCAGTGGGTCTGGGACAGACTTCACTCTCACCATCAGCAGACTGGAG |
| | | CCTGAAGATTTTGCAGTGTATTACTGTCAACAGTATGGTAGCTCACCT |
| | | CAGACGTTCGGCCAAGGGACCAAGGTGGAAATCAAACGAACTGTGGCT |
| | | GCACCATCTGTCTTCATCTTCCCGCCATCTGATGAGCAGTTGAAATCT |
| | | GGAACTGCCTCTGTTGTG |
| B2 | 644 | GAGCTCACCCAGTCTCCAGACTCCCTGGCTGTGTCTCTGGGCGAGAGG |
| | | GCCACCATCAACTGCAAGTCCAGCCAGAGTGTTTTATACAGCTCCAAC |
| | | AATAAGAACTACTTAGCTTGGTACCAGCAGAAACCAGGGCAACCTCCT |
| | | CAGCTGCTCATTTACTGGGCATCTACCCGGGAATCCGGGGTCCCTGAC |
| | | CGATTCAGTGGCAGCGGGTCTGGGACAGATTTCACTCTCACCATCAGC |
| | | AGCCTGCAGGCTGAAGATGTGGCAGTTTATTTCTGTCAGCAATATTAT |
| | | AGTACTCCCTTCACTTTCGGCCCTGGGACCAAAGTGGAAATCAAACGA |
| | | ACTGTGGCTGCACCATCTGTCTTCATCTTCCCGCCATCTGATGAGCAG |
| | | TTGAAATCTGGAACTGCCTCTGTTGTG |
| B3 | 645 | GAGCTCGTGATGACACAGTCTCCAGGCACCCTGTCTTTGTCTCCAGGG |
| | | GAAAGAGCCACCCTCTCCTGCAGGGCCAGTCAGCGTGTTGGCAGCAGC |
| | | TACTTAGCCTGGTACCAGCAGAAACCTGGCCAGGCTCCCAGGCTCCTC |
| | | GTCTATGGTGCATCCAGCAGGGCCACTGGCATCCCAGACAGGTTCAGT |
| | | GGCAGTGGGTCTGGGACAGACTTCACTCTCACCATCAGCAGGCTGCAG |
| | | CCTGAAGATTTTGCAGTATATTACTGTCAGCAGTATGGTACGACGTTC |
| | | GGCCAAGGGACCAGGGTGGACATCAAACGAACTGTGGCTGCACCATCT |
| | | GTCTCCATCTTCCCGCCATCTGATGAGCAGTTGAAATCTGGAACTGCC |
| | | TCTGTTGTG |
| C1 | 646 | GAGCTCACACTCACGCAGTCTCCAGGCACCCTGTCTTTGTCTCCAGGG |
| | | AAAAGAGCCACCCTCTCCTGCAGGGCCAGTCAGAGTGTTAGCGGCAGC |
| | | TACTTAGCCTGGTACCAGCAGAAACCTGGCCAGGCTCCCAGGCTCCTC |
| | | ATCTATGGTGCATCCAACAGGGCCACTGGCATCCCACACAGGTTCAGT |
| | | GGCAGTGGGTCTGGGACAGACTTCACTCTCACCATCAGCAGACTGGAG |
| | | CCTGAGGATTTTGCAGTGTATTACTGTCAGCAGTATGGTTCCTCACCG |
| | | ACGTTCGGCCAGGGGACCAGGGTGGACATCAAACGAACTGTGGCTGCA |
| | | CCATCTGTCTTCATCTTCCCGCCATCTGATGAGCAGTTGAAATCTGGA |
| | | ACTGCCTCTGTTGTG |
| C1* | 647 | GAGCTCACGCAGTCTCCAAGCACCCTGTCTTTGTCTCCAGGGGAAGGA |
| | | GCCACCCTCTCCTGCAGGCCCAGTCAGAGTGTTAGTAGAAACTACCTA |
| | | GCCTGGTATCAGCAGAAACCTGGCCAGGCTCCCAGACTCCTCATCTAT |
| | | GGTGCGTCCACCAGGGCCACCGGCATCCCAGACAGGTTCAGTGGCAGT |
| | | GGGTCTGGGACAAACTTCACTCTCACCATCAGCAGACTGGAGCCTGAA |
| | | GATTTTGCAGTGTATTTCTGTCAGCACTATGGTAACTCACCTCCATAC |
| | | ACTTTTGGCCAGGGGACCAAGCTGGAGATCAAACGAACTGTGGCTGCA |
| | | CCATCTGTCTTCATCTTCCCGCCA |
| C2 | 648 | GAGCTCACGCAGTCTCCAGGCACCCTGTCTTTGTCTCCAGGGGAAAGA |
| | | GCCGCCCTCTCTTGCAGGGCCAGTCAGAGTATTAGCACCAACTACTTA |
| | | GCCTGGTACCAGCAGAAACCAGGCCAGGCTCCCAGGCTCCTCATCTAT |
| | | GGTACGTCCAACAGGGCCACTGGCATCCCAGACAGGTTCAGTGGCACT |
| | | GGGTCTGGGACAGACTTCAGTCTCACCATCAGCAGACTGGAGCCTGAA |
| | | GATTCTGCAGTGTATTACTGTCAGCAGTATGGTACCTCACCATTCACT |
| | | TTCGGCCCTGGGACCAAAGTGGATATCAAACGAACTGTGGCTGCACCA |
| | | TCTGTCTTCATCTTCCCGCCATCT |
| C2* | 649 | GAGCTCACACAGTCTCCAGGCACCCTGTCTTTGTCTCCAGGGGAAAGA |
| | | GCCACCCTCTCCTGCAGGGCCAGTCAGAGTGTTAGCAGCAGCTACTTA |
| | | GCCTGGTACCAGCAGAAACCTGGCCAGGCTCCCAGGCTCCTCATCTAT |
| | | GGTGCATCTAGCAGGGCCACTGGCATCCCAGACAGGTTCAGTGGCAGT |
| | | GGGTCTGGGACAGACTTCACTCTCACCATCAGCAGACTGGAGCCTGAA |
| | | GATTTTGCAGTGTATTACTGCCAACAGTATGGTAGCTCACCTCAGACG |
| | | TTCGGCCAAGGGACCAAGGTGGAAATCAAACGAACTGTGGCTGCACCA |
| | | TCTGTCTTCATCTTCCCGCCATCTGATGAGCAGTTGAAATCTGGAACT |
| | | GCCTCTGTTGTG |
| C3 | 650 | GAGCTCACGCAGTCTCCAGGCACCCTGTCTTTGTCTCCAGGGGAAAGA |
| | | GCCACCCTCTCCTGCAGGGCCAGTCAGAGTGTTAGCAGCAGCTACTTA |
| | | GCCTGGTACCAGCAGAAACCTGGCCAGGCTCCCAGGCTCCTCATCTAT |
| | | GGTGCATCCAGCAGGGCCACTGGCATCCCAGACAGGTTCAGTGGCAGT |
| | | GGGTCTGGGACAGACTTCACTCTCACCATCAGCGGACTGGAGCCTGAA |
| | | GATTTTGCAGTGTATTACTGTCAGCAGTATGGAAGCTCACCTCTCACT |
| | | TTCGGCGGAGGGACCAAGGTGGAGATCAAACGAACTGTGGCTGCACCA |
| | | TCTGTCTTCATCTTCCCGCCATCT |
| C4 | 651 | GAGCTCACGCAGTCTCCAGGCACCCTGTCTTTGTCTCCAGGGGAAAGA |
| | | GCCACCCTCTCCTGCAGGGCCAGTCAGAGTGTTAGCAGCAGCTACTTA |
| | | GCCTGGTACCAGCAGAAACCTGGCCAGGCTCCCAGGCTCCTCATCTAT |
| | | GGTGCATCCAGCAGGGCCACTGGCATCCCAGACAGGTTCAGTGGCAGT |
| | | GGGTCTGGGACAGACTTCACTCTCACCATCAGCAGACTGGAGCCTGAA |
| | | GATTTTGCAGTGTATTACTGTCAGCACTATGGAAGTTCATCGTACACT |
| | | TTCGGCCAGGGGACCAGGGTGGAGATCAAACGAACTGTGGCTGCACCA |
| | | TCTGTCTTCATCTTCCCGCCATCTGATGAGCAGTTGAAATCTGGAACT |
| | | GCCTCTGTTGTG |
| C5 | 652 | GAGCTCACGCAGTCTCCAGGCACCTTGTATGTGTCTCCTGGGGAAAGA |
| | | GCCACCCTCTCCTGCAGGGCCAGTCAGAGTGTTCCCGACAACCACTTA |
| | | GCCTGGTACCAGCAGAAACCTGGCCAGACTCCCAGGCTCCTCATCTAT |
| | | GGTGCATCCAAGAGGGCCACGGGCATCCCAGACAGGTTCAGTGGCAGT |
| | | GGGTCTGGGACAGACTTCACTCTCACCATCAGCAGACTGGAGCCTGAA |
| | | GATTTTGCAGTGTATTACTGTCAGCAGTATGGTAGCTCACCTCAGACG |
| | | TTCGGCCAAGGGACCAAGGTGGAAATCAAACGAACTGTGGCTGCACCA |
| | | TCTGTCTTCATCTTCCCGCCATCTGATGAGCAGTTGAAATCTGGAACT |
| | | GCCTCTGTTGTG |
| C6 | 653 | GAGCTCACACAGTCTCCAGCCACCCTGTCTGTGTCTCCAGGGGAAAGC |
| | | GCCACCCTCTCCTGCAGGGCCAGTCAGAGTGTTAGCAGCAACTTAGCC |
| | | TGGTACCAGCAGAAACCTGGCCAGGCTCCCAGGCTCCTCATCTATGGT |
| | | GCATCGACCAGGGCCACTGGTATCCCAGCCAGGTTCAGTGGCAGTGGG |
| | | TCTGGGACAGACTTCACTCTCACCATCAGCAGACTGGAGCCTGAAGAT |
| | | TTTGCAGTGTATTACTGTCAGCAGTATGGTGGCTCACCTCCGTACACT |
| | | TTTGGCCAGGGGACCAAGCTGGAGATCAAACGAACTGTGGCTGCACCA |
| | | TCTGTCTTCATCTTCCCGCCATCTGATGAGCAGTTGAAATCTGGAACT |
| | | GCCTCTGTTGTG |
| D1 | 654 | GAGCTCACGCAGTCTCCAGGCACCCTGTCTTTGTCTCCAGGGGAAAGA |
| | | GCCACCCTCTCCTGCAGGGCCAGTCAGAGTGTTAGCAGCAGCTACTTA |
| | | GCCTGGTACCAGCAGAAACCTGGCCAGGCTCCCAGGCTCCTCATCTAT |
| | | GGTGCATCCAGCAGGGCCACTGGCATCCCAGACAGGTTCAGTGGCAGT |
| | | GGGTCTGGGACAGACTTCACTCTCACCATCAGCAGACTGGAGCCTGAA |
| | | GATTTTGCAGTGTATTACTGTCAACAGTATGGTAGCTCACCTCAGACG |
| | | TTCGGCCAAGGGACCAAGGTGGAAATCAAACGAACTGTGGCTGCACCA |
| | | TCTGTCTTCATCTTCCCGCCATCTGATGAGCAGTTGAAATCG |
| D2 | 655 | GAGCTCACGCAGTCTCCAGCCACCCTGTCTGTGTCTCCAGGGGAAAGA |
| | | GCCACCCTCTCCTGCAGGGCCAGTCAGACTATTAGCGACAACTTAGCC |
| | | TGGTACCAGCAGAAACCTGGCCAGGCTCCCAGGCTCCTCATCTATGGT |
| | | GCATCCAGCAGGGCCACTGGCATCCCAGACAGGTTCAGTGGCAGTGGG |
| | | TCTGGGACAGACTTCACTCTCACCATCAGCAGACTGGAGCCTGAAGAT |
| | | TTTGCAGTATATTACTGTCAACAGTATGGTAGCTCACCTCAGACGTTC |
| | | GGCCAAGGGACCAAGGTGGAAATCAAACGAACTGTGGCTGCACCATCT |
| | | GTCTTCATCTTCCCGCCATCTGAT |
| D3 | 656 | GAGCTCACACTCACGCAGTCTCCAGGCACCCTGTCTTTGTCTCCAGGG |
| | | GAAAGAGCCACCCTCTCCTGCAGGGCCAGTCAGACTGTTAGCAGCAGC |
| | | TACTTAGCCTGGTACCAGCAGAAACCTGGCCAGGCTCCCAGGCTCCTC |
| | | ATCTATGGTGCATCCAGCAGGGCCACTGGCATCCCAGACAGGTTCAGT |
| | | GGCAGTGGGTCTGGGACAGACTTCACTCTCACCATCAGCAGACTGGAG |
| | | CCTGAAGATTTTGCAGTGTATTACTGTCAACAGTATGGTAGCTCACCT |
| | | CAGACGTTCGGCCAAGGGACCAAGGTGGAAATCAAACGAACTGTGGCT |
| | | GCACCATCTGTCTTCATCTTCCCGCCATCTGATGAGCAGTTGAAATCT |
| | | GGAACTGCT |
| D4 | 657 | GAGCTCGTGATGACACAGTCTCCAGGCACCCTGTCTTTGTCTCCAGGG |
| | | GAAAGAGCCACCCTCTCCTGCAGGGCCAGTCAGAGTGTTAGCAGCAGC |
| | | TACTTAGCCTGGTACCAGCAGAAACCTGGCCAGGCTCCCAGGCTCCTC |
| | | ATCTATGGTGCATCCAGCAGGGCCACTGGCATCCCAGACAGATTCAGT |
| | | GGCAGTGGGTCTGGGACAGACTTCACTCTCACCATCACCAGACTGGAG |
| | | CCTGAAGATTTTGCAGTGTATTACTGTCAACAGTATGGTAGCTCACCT |
| | | CAGACGTTCGGCCAAGGGACCAAGGTGCAAATCAAACGAACTGTGGCT |
| | | GCACCATCTGTCTTCATCTTCCCGCCATCTGATGAGCAGTTGAAATCT |
| | | GGAACTGCT |
| E | 658 | GAGCTCGTGTTGACGCAGTCTCCACTCTCCCTGCCCGTCACCCTTGGA |
| | | CAGCCGGCCTCCATCTCCTGCAGGTCTACTCAGAGCCTCGTTTACAGT |
| | | GATGGAAACACCTACTTGAATTGGTTTCACCAGAGGGCAGGCCAACCT |
| | | CCAAGGCGCCTAATTTATAAGGTCTCTAACCGGGACTCTGGGGTCCCA |
| | | GAGAGATTCAGCGGCAGTGGGTCAGGCACTGATTTCACACTGAAAATC |
| | | AGCAGGGTGGAGGCTGAGGATGTTGGCATTTATTACTGCATGCAAGGA |
| | | GCACACTGGCCTCCCACTTTCGGCGGAGGGACCAAGGTGGAGATCAAT |
| | | CGAACTGTGGCTGCACCATCTGTCTTCATCTTCCCGCCATCTGATGAG |
| | | CAGTTGAAATCTGGAACTGCT |
| F | 659 | GAGCTCCAGATGACCCAGTCTCCATCCTTCCTGTCCGCTTCTGTGGGA |
| | | GACAGAGTCACCATCACTTGCCGGGCCAGTCAGGGCATTAGCAGTTAT |
| | | TTAGCCTGGTATCAGCAAAAACCAGGGAAAGCCCCTAAGCTCCTGATC |
| | | TCTTCTGTATCCACTTTGCAAAGTGGGGTCTCATCAAGGTTCAGCGGC |
| | | AGTGGATCTGGGACAGGATTCACTCTCACAATCAGCAGCCTGCAGTCT |
| | | GAAGATTCTGCAACTTATTACTGTGAACAACTGAATAGTTTCCCGTAC |
| | | ACTTTTGGCCAGGGGACCAAGCTGGAGATCAAACGAACTGTGGCTGCA |
| | | CCATCTGTCTTCATCTTCCCGCCATCT |
| G | 660 | GAGCTCACTCAGTCTCCAGTCTCCCTGCCCGTCACCCCTGGAGAGCCG |
| | | GCCTCCATCTCCTGCAGGTCTAGTCAGAGCCTCCTGCATAGTAATGGA |
| | | AACCACTATTTGGATTGGTACCTGCAGAAGCCAGGGCAGTCTCCACAG |
| | | CTCCTGATGTATTTGGGTTCTAATCGGGCCTCCGGGGTCCCTGACAGG |
| | | TTCAGTGGCAGTGGATCAGGCACAGATTTTACACTGAAAATCAGCAGA |
| | | GTGGAGGCTGAGGATGTTGGGGTTTATTACTGCATGCAAGGTCTACAG |
| | | ACCCCGTGGACGTTCGGCCAAGGGACCAAGGTGGAAATCAAACGAACT |
| | | GTGGCTGCACCATCTGTCTTCATCTTCCCGCCATCTGAT |
| H1 | 661 | GAGCTCACACTCACGCAGTCTCCAGGCACCCTGTCTTTGTCTCCAGGG |
| | | GAAAGAGCCACCCTCTCCTGCAGGGCCAGTCAGAGTATTAGCAGCAGC |
| | | TACTTAGCCTGGTACCAGCAGAAACCTGGCCAGGCTCCCAGGCTCCTC |
| | | ATCTATGGTGCATCCAGAAGGGCCACTGGCATCCCAGACAGGTTCAGT |
| | | GGCAGCGGGTCTGGGACAGACTTCACTCTCACCATCAGCAGACTGGAG |
| | | CCTGAAGATTTTGCAGTGTATTACTGTCAGCAGTATGGTAGCTCACCG |
| | | CTCACTTTCGGCGGAGGGACCAAGGTGGAGATCAAACGAACTGTGGCT |
| | | GCACCATCTGTCTTCATCTTCCCGCCATCTGAT |
| H1* | 662 | GAGCTCACACAGTCTCCAGCCACCCTGTCCGTGTCTCCAGGGGAAAGA |
| | | GCCACCCTCTCCTGCAGGGCCAGTCGGGGTATTAGCAGCAACTTAGCC |
| | | TGGTACCAGCAGAAGCCTGGCCAGGCTCCCAGGCTCCTCATCTATGGT |
| | | GCATCCAGCAGGGCCACTGGCATCCCAGACAGGTTCAGTGGCAGTGGA |
| | | TCTGGGACAGACTTCACTCTCACCATCAGCAGACTGGAGCCTGAAGAT |
| | | TTTGCAGTGTATTACTGTCAACAGTATGGTAGCTCACCTCAGACGTTC |
| | | GGCCAAGGGACCGAGGTGGAAATCAAACGAACTGTGGCTGCACCATCT |
| | | GTCTTCATCTTCCCGCCATCTGATGAGCAG |
| H1** | 663 | GAGCTCACACTCACGCAGTCTCCAGGCACCCTGTCTTTGTCTCCAGGG |
| | | GAGAGAGCCACCCTCTCCTGCAGGGCCAGTCAGAGTGTTAGCAGCGAC |
| | | TCCTTAGCCTGGTACCAGCAGAAACCTGGCCAGGCTCCCAGGCTCCTC |
| | | ATCTATGGTGCCTCTAGCAGGGCCACTGGCATCCCAGACAGGTTCAGT |
| | | GGCAGTGGGTCTGGGACAGACTTCACTCTCACCATCAGCAGACTGGAG |
| | | CCTGAAGATCTTGGAGTGTATTACTGTCAGCAGTATGGTCCCTCACCT |
| | | CCGGGGTACACTTTTGGCCAGGGGACCAAGCTGGAGATCAAACGAACT |
| | | GTGGCTGCACCATCTGTCTTCATCTTCCCGCCATCTGATGAGCAGTTG |
| | | AAATCTGGAACTGCCTCTGTTGTG |
| H2 | 664 | GAGCTCACACTCACGCAGTCTCCGGGCACCCTGTCTTTGTCTCCAGGG |
| | | GAAAGAGGCACCCTCTCCTGCAGGGCCAGTCAGAGTGTTAGCAGCAGC |
| | | TACTTAGCCTGGTACCAGCAGAAACCTGGCCAGGCTCCCAGGCTCCTC |
| | | ATCTATGGTGCATCCAGCAGGGCCACTGGCATCCCAGACAGGTTCAGT |
| | | GGCAGTGGGTCTGGGACAGACTTCACTCTCACCATCAGCAGACTGGAG |
| | | CCTGAAGATTTTGCAGTGTATTACTGTCAGCAGTATGGTAGCTCACCT |
| | | CAGACGTTCGGCCAAGGGACCAAGGTGGAAATCAAACGAACTGTGGCT |
| | | GCACCATCTGTCTTCATCTTCCCGCCATCTGATGAGCAGTTGAAATCT |
| | | GGAACTGCCTCTGTTGTG |
| H3 | 665 | GAGCTCACACAGTCTCCAGCCACCCTGTCTGTGTCTCCAGGGGAAAGA |
| | | GCCACCCTCTCCTGCAGGGCCAGTCAGAGTGTTAGCAGCAACTTAGCC |
| | | TGGTACCAGCAGAAACCTGGCCAGGCTCCCAGGCTCCTCATCTATGGT |
| | | GCATCCACCAGGGCCACTGGTATCCCAGCCAGGTTCAGTGGCAGTGGG |
| | | TCTGGGACAGAGTTCACTCTCACCGTCAGCAGACTGGAGCCTGAAGAT |
| | | TCTGCAGTGTATTTCTGTCAGCAGTATTATAGGTCCCCACTCACTTTC |
| | | GGCGGAGGGACCAAGGTGGAGATCAAACGAACTGTGGCTGCACCATCT |
| | | GTCTTCATCTTCCCGCCATCTGATGAGCAGTTGAAATCTGGAACTGCC |
| | | TCTGTTGTG |
| I | 666 | GAGCTCACACTCACGCAGTCTCCCGCCACCCTGTCTGTTTCTCCAGGG |
| | | GAAAGAGCCACCCTTTTTTGTAGGGCCAATCAGAGTGTTGGCCGCAAC |
| | | TTAGCCTGGTACCAGCAGAAACCTGGCCAGGCTCCCAGGCTCCTCATT |
| | | TATGGTATATCCACCAGGACCACTACTACCCCAACCAGGTTCAGTGGC |
| | | AGTGGGTCTGGGACAGACTTCACTCTCACCATCAGCCGCCTGCAGTCT |
| | | GAAGATTTTGCAGTTTATTACTGTCAGCAGTATAACAAGTGGCCTCCG |
| | | TGGACGTTCGGCCAAGGGACCAAGTTGGAAATCAAACGAACTGTGGCT |
| | | GCACCATCTGTCTTCGTCTTCCCGCCATCT |
| J1 | 667 | GAGCTCGTGTTGACACAGTCTCCAGGCACCCTGTCTTTGTCTCCAGGG |
| | | GAAAGAGCCACCCTCTCCTGCAGGGCCAGTCAGAGTGTTAGCAGCAGC |
| | | TACTTAGCCTGGTACCAGCAGAAACCTGGCCAGGCTCCCAGGCTCCTC |
| | | ATCTATGGTGCATCCAGCAGGGCCACTGGCATCCCAGACAGGTTCAGT |
| | | GGCAGTGGGTCTGGGACAGACTTCGCTCTCACCATCACCAGACTGGAG |
| | | CCTGAAGATTTTGCAGTGTATTACTGTCAGCAGTATGGTAGCTCACCT |
| | | CAGACGTTCGGCCAAGGGACCAAGGTGGAAATCAAACGAACTGTGGCT |
| | | GCACCATCTGTCTTCATCTTCCCGCCATCTGATGAGCAGTTGAAATCT |
| | | GGAACTGTCTCTGTTGTG |
| J2 | 668 | GCGGCCGAGCTCACTCAGTCTCCAGGCACCCTGTCTTTGTCTCCAGGG |
| | | GAAAGAGCCACCCTCTCCTGCAGGGCCAGTCAGAGTGTTAGCAGCAGC |
| | | TACTTAGCCTGGTACCAGCAGAAACCTGGCCAGGCTCCCAGGCTCCTC |
| | | ATCTATGGTGCATCCAGCAGGGCCACTGGCATCCCAGACAGGTTCAGT |
| | | GGCAGTGGGTCTGGGACAGACTTCACTCTCACCATCAGCAGACTGGAG |
| | | CCTGAAGATTTTGCAGTGTATTACTGTCAGCAGTATGGTAGCTCACCT |
| | | CAGACGTTCGGCCAAGGGACCAAGGTGGAAATCAAACGAACTGTGGCT |
| | | GCACCATCTGTCTTCATCTTCCCGCCATCTGATGAGCAGTTGAAATCT |
| | | GGAACTGCCTCTGTTGTG |
| J3 | 669 | GAGCTCACGCAGTCTCCAGGCACCCTGTCTTTGTCTCCAGGGGAAAGA |
| | | GCCACCCTCTCCTGCAGGGCCAGTCAGAGTGTTAGCAGCAGCTACTTA |
| | | GCCTGGTACCAGCAGAAACCTGGCCAGGCTCCCAGGCTCCTCATCTAT |
| | | GGTGCATCCAGCAGGGCCACTGGCATCCCAGACAGGTTCAGTGGCAGT |
| | | GGGTCTGGGACAGACTTCACTCTCACCATCAGCGGACTGGAGCCTGAA |
| | | GATTTTGCAGTGTATTACTGTCAACAATATGGTAGCTCACCTCAGACG |
| | | TTCGGCCAAGGGACCAAGGTGGAAATCAAACGAACTGTGGCTGCACCA |
| | | TCTGTCTTCATCTTCCCGCCATCTGATGAGCAGTTGAAATCTGGAACT |
| | | GCCTCTGTT |
| J3* | 670 | GAGTTCACACTCACGCAGTCTCCAGGCACCCTGTCTTTGTCTCCAGGG |
| | | GAAAGAGGCACCCTCTCCTGCAGGGCCAGTCAGAGTGTTAGCAGCAGC |
| | | TACTTAGCCTGGTACCAGCAGAAACCTGGCCAGGCTCCCAGGCTCCTC |
| | | ATCTATGGTGCATCCAGCAGGGCCACTGGCATCCCAGACAGGTTCAGT |
| | | GGCAGTGGGTCTGGGACAGACTTCACTCTCACCATCAGCAGACTGGAG |
| | | CCTGAAGATTTTGCAGTGTATTACTGTCAGCAGTATGGTAGCTCACCT |
| | | CAGACGTTCGGCCAAGGGACCAAGGTGGAAATCAAACGAACTGTGGCT |
| | | GCACCATCTGTCTTCATCTTCCCGCCATCTGATGAGCAGTTGAAATCT |
| | | GGAACTGCCTCTGTTGTG |
| J4 | 671 | GAGCTCACACTCACGCAGTCTCCAGGCACCCTGTCTTTGTCTCCAGGG |
| | | GAAAGAGCCACCCTCTCCTGCAGGGCCAGTCAGAGTGTTAGCAGCAGC |
| | | CACTTAGCCTGGTACCAGCAGAAACCTGGCCAGGCTCCCAGGCTCCTC |
| | | ATCTATGGTGCATCCAGCAGGGCCACTGGCATCCCAGACAGGTTCAGT |
| | | GGCAGTGGGTCTGGGACAGACTTCACTCTCACCATCAGCAGACTGGAG |
| | | CCTGAAGATTTTGCAGTGTATTACTGTCAACAGTATGGTAGCTCACCT |
| | | CAGACGTTCGGCCAAGGGACCGAGGTGGAAATCAAACGAACTGTGGCT |
| | | GCACCGTCTGTCTTCATCTTCCCGCCATCTGATGAGCAGTTGAAATCT |
| | | GGAACTGCCTCTGTTGTG |
| K | 672 | GAGCTCACGCAGTCTCCAGGCACCCTGTCTTTGTCTCCAGGGGAAAGA |
| | | GCCACCCTTTCCTGCAGGGCCAGTCAGAGTGTTAGCAGCAACTCCTTA |
| | | GCCTGGTACCAACAGAAACCTGGCCTGGCTCCCAGGCTCCTCATCTAT |
| | | GGTGCATCCAGCAGGGCCACTGGCATCCCAGAGAGGTTCAGTGGCAGT |
| | | GGGGCTGGGACAGGCTTCACTCTCACCATCAGCACACTGGAGCCTGAA |
| | | GATTTTGCAATTTATTACTGTCAACAATATGGTGGCTCGCCTCCAAGA |
| | | TTCACTTTCGGCCCTGGGACCAAAGTGGATATCAGACGAACTGTGGCT |
| | | GCACCATCTGTCTTCATCTTCCCGCCATCTGATGAGCAGTTGAAATCT |
| | | GGAACTGCCTCTGTTGTG |
| L1 | 673 | GAGCTCACGCAGTCTCCAGGCACCCTGTCGTTGTCTCCAGGGGAAAGA |
| | | GCCACTCTCTCCTGCAGGGCCAGTCAGAGTATTACCAGCAGGTACTTA |
| | | GCCTGGTACCAGCAGAAACCTGGCCAGGCTCCCAGGCTCCTCATCTAT |
| | | GGTGCATCCAGCAGGGCCACTGGCATCCCAGACAGGTTCAGTGGCAGT |
| | | GGGTCTGGGACAGACTTCACTCTCACCATCAGCAGACTGGAGCCTGAA |
| | | GATTTTGCAGTGTATTACTGTCAGCAGTATGGTGACTCCGTCGGTTTC |
| | | GGCCCTGGGACCAAAGTGGAAATCAAACGAACTGTGGCTGCACCATCT |
| | | GTCTTCATCTTCCCGCCATCTGATGAGCAGTTGAAATCTGGAACTGCC |
| | | TCTGTTGTG |
| L2 | 674 | GAGCTCACGCAGTCTCCAGGCACCCTGTCGTTGTCTCCAGGGGAAAGA |
| | | GCCACTCTCTCCTGCAGGGCCAGTCAGAGTATTACCAGCAGGTACTTA |
| | | GCCTGGTACCAGCAGAAACCTGGCCAGGCTCCCAGGCTCCTCATCTAT |
| | | GGTGCATCCAGCAGGGCCACTGGCATCCCAGACAGGTTCAGTGGCAGT |
| | | GGGTCTGGGACAGACTTCACTCTCACCATCAGCAGACTGGAGCCTGAA |
| | | GATTTTGCAGTGTATTACTGTCAGCAGTATGGTGACTCCGTCGGTTTC |
| | | GGCCCTGGGACCAAAGTGGAAATCAAACGAACTGTGGCTGCACCATCT |
| | | GTCTTCATCTTCCCGCCATCTGATGAGCAGTTGAAATCTGGAACTGCC |
| | | TCTGTTGTG |
| L3 | 675 | GAGCTCGTGATGACGCAGTCTCCAGCCACCCTGTCTTTGTCTCCAGGG |
| | | GAAAGAGCCACCCTCTCCTGCAGGGCCAGTCAGAGTGTTGGCAGCTAC |
| | | TTAGCCTGGTACCAACAGAAACCTGGCCAGGCTCCCAGGCTCCTCATT |
| | | TATGATGCATCCAACAGGGCCACTGGCATCCCAGCCAGGTTCAGTGGC |
| | | AGTGGGTCTGGGACAGACTTCACTCTCACCATCAGCAGCCTAGAGCCT |
| | | GAAGATTTTGCTGTGTATTTTTGTCAGCAGTATGGTAGCTCACCCTTG |
| | | ACGTTCGGCCAAGGGACCAAGGTGGAAATCAAACGAACTGTGGCTGCA |
| | | CCATCTGTCTTCATCTTCCCGCCATCTGATGAGCAGTTGAAATCTGGA |
| | | ACTGCCTCTGTTGTG |
| L4 | 676 | GAGCTCACGCAGTCTCCAGGCACCCTGTCTTTGTCTCCAGGGGAAAGA |
| | | GCCACCCTCTCTTGTAGGGCCGGTCAGACTGTTGCGAGCAATTCCTTA |
| | | GCCTGGTATCAGCACAAACCTGGCCAGGCTCCCAGGCTCCTCATCTAT |
| | | GGTGCCTCCATCAGGGCCAGTGGCATCCCAGACAGGTTCAGTGGCAGT |
| | | GGGTCTGGGACAGACTTCACTCTCACCATCAGCAGACTGGAGCCTGAA |
| | | GATTTTGCAGTGTATTACTGTCAGCAGTATGGTCTTTCTTCCACCTTC |
| | | GGCCAAGGGACACGACTGGAGATTAAACGAACTGTGGCTGCACCATCT |
| | | GTCTTCATCTTCCCGCCATCTGATGAGCAGTTGAAATCTGGAACTGCC |
| | | TCTGTTGTG |
| M | 677 | GAGCTCACACTCACGCAGTCTCCAGGCACCCTGTCTTTGTCTCCAGGG |
| | | GAAAGAGCCACCCTCTCCTGCAGGGCCAGTCAGAGTATTAGGAGCAGT |
| | | TACTTAGCCTGGTACCAGCAGAAACCTGGCCAGGCTCCCAGGCTCCTC |
| | | ATCTATGCTGCAGCCAGCAGGGCCACTGGCATCCCAGACAGGTTCAGT |
| | | GGCAGTGGGTCTGGGACAGACTTCACTCTCACCATCAGCAGACTGGAG |
| | | CCTGAAGATTTTGCAGTGTATTTCTGTCATCACTATGGTGGCTCACCT |
| | | CGGACGTTCGGCCAAGGGACCAAGGTGGAAATCAAACGAACGGTGGCT |
| | | GCACCATCTGTCTTCATCTTCCCGCCATCTGATGAGCAGTTGAAATCT |
| | | GGAACT |
| N1 | 1043 | GAGCTCACACAGTCTCCAGGCACCCTGTCTTTGTCTCCAGGGGAAAGA |
| | | GCCACCCTCTCCTGCAGGGCCAGTCAGAGTGTTAGCAGCAGCTACTTA |
| | | GCCTGGTACCAGCAGAAACCTGGCCAGGCTCCCAGGCTCCTCATCTAT |
| | | GGTGCATCCAACAGGGCCGCTGGCATCCCAGACAGGTTCAGTGGCAGT |
| | | GGGTCTGGGGCAGACTTCACTCTCACCATCAGCAGACTGGAGCCTGAA |
| | | GATTTTGCAGTGTATTACTGTCAGCAGTATGGTAGCTCACTTATCACC |
| | | TTCGGCCAAGGGACACGACTGGAGATTAAACGAACT |
| N2 | 1044 | GAGCTCACGCAGTCTCCAGGCACCCTGTCTTTGTTTCCAGGGGAAAGA |
| | | GCCACCCTCTCCTGCAGGGCCAGTCAGAGTATTCTCGGCAGATACTTA |
| | | GCCTGGTATCAGCAGAAAGGCGGCCGGGCTCCCAGACTCCTCATCTTT |
| | | GGTGCATCCAAGAGGGCCACTGGCATCCCAGACAGGTTCAGTGGCAGT |
| | | GGGTCTGGGACAGACTTCACTCTCACCATCGGCAGACTGGAGCCTGAA |
| | | GATTTTGCAGTGTATTACTGTCAGCACTATGGTAGTTCAATCACCTTC |
| | | GGCCAAGGGACACGGCTGGACATTAAACGAACT |
| N3 | 1045 | GAGCTCACGCAGTCTCCAGGCACCCTGTCTTTGTCTCCAGGGGAAAGA |
| | | GCCACCCTCTCCTGCAGGGCCAGTCAGAGTGTTAGCAGCAGCTACTTA |
| | | GCCTGGTACCAGCAGAAACCTGGCCAGGCTCCCAGGCTCCTCATCTAT |
| | | GGTGCATCCAGCAGGGCCACTGGCATCCCAGACAGGTTCAGTGGCAGT |
| | | GGGTCTGGGACAGACTTCACTCTCACCATCAGCAGACTGGAGCCTGAA |
| | | GATTTTGCAGTGTATTACTGTCAGCAGTATGGTAGCTCCCTCACTTTC |
| | | GGCGGAGGGACCAAGGTGGAGATCAAACGAACT |
| N4 | 1046 | GAGCTCACACTCACGCAGTCTCCAGGCACCCTGTCTTTGTCTCCAGGG |
| | | GAAAGAGCCACCCTCTCCTGCAGGGCCAGTCAGAGTGTTAGCAACAAC |
| | | TACTTAGCCTGGTACCAGCAGAAACCTGGCCAGGCTCCCAGGCTCCTC |
| | | ATCTATGGTGCATCCAGCAGGGCCACTGGCATCCCAGACAGGTTCAGT |
| | | GGCAGTGGGTCTGGGACAGGCTTCACTCTCATCATCAGCAGACTGGAG |
| | | CCTGAAGATTTTGCAGTGTATTACTGTCAGCAGTATGGTAGCTCTTCG |
| | | ATCACCTTCGGCCAAGGGACACGACTGGAGATTAAACGAACT |
| O1 | 1047 | GAGCTCACTCAGTCTCCATCGTCCCTGTCTGCATCGGTAGGAGATAGA |
| | | GTCACCATCACTTGCCGGGCAACTCAGGGCATTGACAACTATTTAAAT |
| | | TGGTATCAGCAAAAACCGGGGAAACCCCCTAGGCTCCTCATCTATGGT |
| | | GCATCCAGTTTGCAAAGTGGGGTCCCATCAAGATTTAGTGGCGGTGGA |
| | | TCTGGGACACATTTCACTCTCACCATCACCAATCTGCAACCTGAAGAT |
| | | TTTGCAACTTACTACTGTCAACAGAGTTACAGTACCCCTCCGGAGACG |
| | | TTCGGCCAGGGGACCAAGGTGGAAATCAAACGAACT |
| P1 | 1048 | GAGCTCACGCAGTCTCCAGGCACCCTGTCTTTGTCTCCAGGGGAAAGA |
| | | GCCACCCTCTCCTGCAGGGCCAGTCAGGTGCGTGGCTTCTTAGCCTGG |
| | | TTCCAGCAGAAACCTGGCCAGGCTCCCAGACTCCTCATCTATGGTGCA |
| | | TCCAACAGGGCCCCTGGAATCCCAGACAGGTTCAGTGGCAGTGGGTCT |
| | | GGGACAGACTTCACTCTCACCATCAGCAGACTGGAGCCTGAAGATTTT |
| | | GCAGTGTATTACTGTCAGCAGTATGGTGACTCACCTCCGATCACCTTC |
| | | GGCCAAGGGACACGACTGGAGATTAAAAGAACT |
| P1a | 1049 | GAGCTCACACAGTCTCCAGGCACCCTGTCTTTGTCTCCAGGGGAAAGA |
| | | GCCACCCTCTCCTGCAGGACCAGTCAGAGTGTTAGCAGCACTTACTTA |
| | | GCCTGGTACCAGCAGAAACCTGGCCAGCCTCCCAGGCTCCTCATCTAT |
| | | GGTGCATCCAATAGGGCCCCTGGAATCCCAGACAGGTTCAGTGGCAGT |
| | | GGGTCTGGGACAGACTTCACTCTCACCATCAGCAGACTGGAGCCTGAA |
| | | GATTTTGCAGTGTATTACTGTCAGCAGTATGGTGACTCACCTCCGATC |
| | | ACCTTCGGCCAAGGGACACGACTGGACATTAAAAGAACT |
| P2 | 1050 | GAGCTCGTGTTGACGCAGTCTCCAGGCACCCTGTCTTTGTCTCCAGGG |
| | | GAAAGAGCCACCCTCTCCTGCAGGGCCAGTCAGAGTGTTAGCAGCAGC |
| | | TACTTAGCCTGGTACCAGCAGAAACCTGGCCAGGCTCCCAGGCTCCTC |
| | | ATCTATGGTGCATCCAGCAGGGCCACTGGCATCCCAGACAGGTTCAGT |
| | | GGCAGTGGGTCTGGGACAGACTTCACTCTCACCATCAGCAGACTGGAG |
| | | CCTGAAGATTTTGCAGTGTATTACTGTCAGCAGTATGGTAGCTCACCC |
| | | TTTTTCGGCCCTGGGACCAAAGTGGATATCAAACGAACT |
| P3 | 1051 | GAGCTCACGCAGTCTCCAGGCACCCTGTCTTTGTCTCCAGGGGAAAGA |
| | | GCCACCCTCTCCTGCAGGGCCAGTCAGAGTGTTAGCAGCACCTACTTA |
| | | GCCTGGTACCAGCAGAAACCTGGCCAGGCTCCCAGGCTCCTCATCTAT |
| | | GGTGCATCCAGCAGGGCCACTGGCATCCCAGACAGGTTTAGTGGCAGT |
| | | GGGTCTGGGACAGACTTCACTCTCACCATCAGCAGACTGGAGCCTGAA |
| | | GATTTTGCAGTGTATTACTGTCAGCAGTATGGTAGCTCACCGCTCACT |
| | | TTCGGCGGAGGGACCAAGGTGGAGATCAAACGAACT |
| P4 | 1052 | GAGCTCACTCAGTCTCCAGGCACCCTGTCTCTGTCTCCAGGGGAAAGA |
| | | GCCACCCTCTCCTGCAGGGCCAGTCAGAGTGTTAGCAGCAGCAACTTA |
| | | GCCTGGTTCCAGCATAAATCTGGCCGGGCTCCCAGGCTCCTCATCTAT |
| | | GGTGCATCCAACAGGGCCCCTGACATCCCAGACAGGTTCAGTGGCAGT |
| | | GGGTCTGGGACAGACTTCACTCTCAGCATCAGCAGACTGGAGCCTGAA |
| | | GATTTTGCAGTGTATTACTGTCAGCGGTATGGTGACTCACCTCCGATC |
| | | ACCTTCGGCCAAGGGACACGACTGGAGATTAAACGAACT |
| P5 | 1053 | GAGCTCACACAGTCTCCAGCCTCCCTGTCTTTGTCTCCAGGGGAAAGG |
| | | GCCACCCTCTCCTGCAGGGCCAGTCAGAGTGTTGGCACCTACTTTGCC |
| | | TGGTACCAACAGAAACCTGGCCAGGCTCCCAGGCTCCTCATCTATGGT |
| | | GCGTCCAACAGGGCCACTGGCATCCCAGACAGGTTCAGTGGCAGTGGG |
| | | TCTGGGACAGACTTCACCCTCACCGTCAGCAGACTGGAACCTGAAGAT |
| | | TTTGCAGTGTATTACTGTCAGCAGTATGGTAGCTCACCGACGTTCGGC |
| | | CAAGGGACCAAGGTGGAAATCAAACGAACT |
| P6 | 1054 | GAGCTCATTCAGTCTCCAGGCACCCTGTTTTTGTCTTCAGGGGAAAGA |
| | | GCCACCCTCTCCTGCAGGGCCAGTCAGAGTGTTAGCAGCAGCAACTTA |
| | | GCCTGGTTCCAGCATAAATCTGGCCGGGCTCCCAGGCTCCTCATCTAT |
| | | GGTGCATCCAACAGGGCCCCTGACATCCCAGACAGGTTCAGTGGCAGT |
| | | GGGTCTGGGACAGACTTCACTATCAGCATCAGCAGACTGGAGCCTGAA |
| | | GATTTTGCAGTGTATTACTGTCAGCGGTATGGTGACTCACCTCCGATC |
| | | ACCTTCGGCCAAGGGACACGACTGGAGATTAAACGAACT |
| Q1 | 1055 | GAGCTCACGCAGTCTCCAGGCACCCTGTCTTTGTCTCCAGGGGAAAGA |
| | | GCCACCCTCTCCTGCAGGGCCAGTCAGAGTGTTAGCAGCAGCTACTTA |
| | | GCCTGGTACCAGCAGAAACCTGGCCAGGCTCCCAGGCTCCTCATCTAT |
| | | GGTGCATCCAGCAGGGCCACTGGCATCCCAGACAGGTTCAGTGGCAGT |
| | | GGGTCTGGGACAGACTTCACTCTCACCATCAGCAGACTGGAGCCTGAA |
| | | GATTTTGCAGTGTATTACTGTCAACAGTATGGTAGCTCACCTCAGACG |
| | | TTCGGCCAAGGGACCAAGGTGGAAATCAAACGAACT |
| Q2 | 1056 | GAGCTCACGCAGTCTCCAGCCTCCCTGTCTCTGTCTCCAGGGGGAAGC |
| | | GCCACCCTCGCCTGCAGGGCCAGCCGGGGTGTTAACAGCAACCTAGCC |
| | | TGGTATCACCAGAGGCCTGGCCAGGCTCCCAGGCTCCTCATTTATAGT |
| | | GCATCTACCAGGGCCACTGGTATCCCAGGCAGGTTCAGCGGCAGTGGG |
| | | TTTGGGACAGAGTTCACTCTCACCATCAACAATCTGCAGTCTGACGAC |
| | | TTTGGAGTTTATTACTGTCAGCAGTATGATGACACGCCTCAGATCACC |
| | | TTCGGCCAGGGAACACGACTGGACATTAAACGGCTGGACATTAAGCGA |
| | | ACT |
| R1 | 1057 | GAGCTCACTCAGTCTCCACTCTCCCTGCCCGTCACCCCTGGAGAGCCG |
| | | GCCTCCATCTCCTGCAGGTCTAGTTACAGCCTCCTCCATATTAATGGA |
| | | TACAAGTATTTGGATTGGTACCTGCAGAGGCCAGGGCAGTCTCCACAG |
| | | CTCCTGATCTATTTGGGTTCTAATCGGGCCCCCGGGGTCCCTGACAGG |
| | | TTCAGTGGCAGTGGATCAGGCACATCTTTTACACTGAAAATCAGCAGA |
| | | GTGGAGGCTGAGGATGTTGGGGTTTATTACTGCATGCAAAGTCTTCAA |
| | | GCTCCGTGGACGTTCGGCCAAGGGACCAAGGTGGAAATGAAACGAACT |
| S1 | 1058 | GAGCTCGTGTTGACACAGTCTCCAGGCACCCTGTCTTTGTCTCCAGGG |
| | | GAAAGAGCCACCCTCTCCTGCAGGGCCAGTCAGAGTGTTAGCAGCAGC |
| | | TACTTAGCCTGGTACCAGCAGAAACCTGGCCAGGCTCCCAGGCTCCTC |
| | | ATCTATGGTGCATCCAGCAGGGCCACTGGCATCCCAGACAGGTTCAGT |
| | | GGCAGTGGGTCTGGGACAGACTTCACTCTCACCATCAGCAGACTGGAG |
| | | CCTGAAGATTTTGCAGTGTATTACTGTCAGCAGTATGGTAGCTCACCT |
| | | CAGACGTTCGGCCAAGGGACCAAGGTGGAAATCAAACGAACT |
| T1 | 1059 | GAGCTCACGCAGTCTCCAGGCACCCTGTCTTTGTCTCCAGGGGAAAGA |
| | | GCCACCCTCTCCTGCAGGGCCAGTCAGAGTGTTAGCAGCAGCTACTTA |
| | | GCCTGGTACCAGCAGAAACCTGGCCAGGCTCCCAGGCTCCTCATCTAT |
| | | GGTGCATCCAGCAGGGCCACTGGCATCCCAGACAGGTTCAGTGGCAGT |
| | | GGGTCTGGGACAGACTTCACTCTCACCATCAGCAGACTGGAGCCTGAA |
| | | GATTTTGCAGTGTATTACTGTCAACAGTATGGTAGCTCACCTCAGACG |
| | | TTCGGCCAAGGGACCAAGGTGGAAATCAAACGAACT |
| U1 | 1060 | GAGCTCACCCAGTCTCCATCCTCCCTGTCTGCATCTGTAGGAGACAGA |
| | | GTCACCATCACTTGCCGGGCAAGTCAGAGCATTAGCAGCTTTTTAAAT |
| | | TGGTATCAGCAGAAACCAGGGAAAGCCCCTAAGCTCCTGATCTATGCT |
| | | GCATCCAGTTTGCAAAGTGGGGTCCCACCAAGGTTCAGTGGCAGTGGA |
| | | TCTGGGACAGATTTCACTCTCACCATCAGCAGTCTGCAACCTGAAGAT |
| | | TTTGCAACTTACTACTGTCAACAGAGTTACAGTACCCCTCGCACTTTT |
| | | GGCCAGGGGACCAAGCTGGAGATCAAACGAACT |
| V1 | 1061 | GAGCTCACCCAGTCTCCATCCTCCCTGTCTGCATCTGTAGGAGACAGA |
| | | GTCACCATCACTTGCCAGGCAAGTCAAGACATTAGCAACTTTTTAAAT |
| | | TGGTATCAACGGAGACCTGGGAAAGCCCCTAATCTCCTGATCTACGAT |
| | | GCAACCCATTTGGAAACAGGGGTCCCATCAAGGTTCAGTGGAAGTGGA |
| | | TTTGGGACACATTTTACTCTCACCATCAACAGCCTGCAGCCTGAAGAT |
| | | ATTGGTACATATTACTGTCAACACTTTGATGACGTCCCCTCTTTCACT |
| | | TTCGGCCCTGGGACCAAAGTGGATCTCAAACGAACT |

Also exemplary of the nucleic acids provided herein are those that encode a CDR1 set forth in SEQ ID NOS: 78-108; 171-205; 725-741, 778-796, 1090, 1091 and 1101-1113; a CDR2 set forth in SEQ ID NOS: 109-139; 206-240; 742-759, 797-815, 1092-1096 and 1114-1121; and a CDR3 set forth in SEQ ID NOS: 140-170; 241-275; 760-777, 816-834, 1097-1100 and 1122-1133. Further, included herein are nucleic acids that encode any of the framework regions set forth in SEQ ID NOS: 309-572, 835-982 and 1134-1205. These nucleic acids can be inserted into an expression cassette or expression vector such that they are operably linked to expression control sequences.

Nucleic acid molecules encoding the polypeptides and anti-HCV antibodies or antigen-binding fragments thereof provided herein can be prepared using well-known recombinant techniques for manipulation of nucleic acid molecules (see, e.g., techniques described in Sambrook et al., (1990) Molecular Cloning, A Laboratory Manual, 2d Ed., Cold Spring Harbor Laboratory, Cold Spring Harbor, N.Y. and Ausubel et al., eds. (1998) Current Protocols in Molecular Biology, John Wiley & Sons, NY). In some examples, methods, such as, but not limited to, recombinant DNA techniques, site directed mutagenesis, and polymerase chain reaction (PCR) can be used to generate modified antibodies or antigen-binding fragments thereof having a different amino acid sequence, for example, to create amino acid substitutions, deletions, and/or insertions.

Polypeptides and antibodies having sequences other than those shown in Tables 3 and 4, e.g. those having other combinations of CDR and/or framework sequences shown in Tables 9, 10, 11, 18, 19, 30 and 31, also can be produced by recombinant expression. First, nucleic acids encoding these polypeptides and antibodies can be constructed by switching the regions of these molecules that encode the CDR and/or framework sequences. In particular, the nucleic acid encoding a first polypeptide can be modified by insertion or replacement of nucleic acid regions encoding, for example, a CDR region, a framework region or a constant region, from another nucleic acid encoding a second polypeptide using known recombinant techniques. Alternatively, nucleic acids encoding each CDR and framework sequences shown in Tables 9, 10, 11, 18, 19, 30 and 31 can be synthesized using conventional methods of oligonucleotide synthesis and/or polymerase chain reaction (PCR).

Nucleic acids encoding each CDR and framework sequence set forth in Tables 9, 10, 11, 18, 19, 30 and 31 can be determined using methods and software known in the art. Examples of coding nucleotide sequences for the exemplary Fab heavy and light chains are shown in Tables 5 and 6. Due to redundancy in the genetic code, other nucleotide sequences also can code for these Fab heavy and light polypeptide sequences. Software that can be used to identify the nucleic acid sequences that code for each CDR and framework sequences are available in the art, see, for example, the software tools Translate, BCM search launcher, Reverse Translate, Reverse Transcription and Translation Tool available at expasy.org/tools/#translate.

Nucleic acids encoding selected CDR and framework sequences can be joined by splicing using overlapping extension PCR, and the resulting nucleic acid inserted into an expression vector for expression in a bacterial or mammalian host cell as described below. See, for example, Horton et al., Biotechniques 8:528-535 (1990). Nucleic acid sequences encoding constant regions of the light and heavy chains of human and other mammalian antibodies are known in the art and can be obtained from the public databases such as Genbank. Examples of nucleic acid sequences encoding constant regions are also described in Kabat et al., Sequences of Proteins of Immunological Interest, 5^{th} Edition, National Institutes of Health Publication No. 91-3242 (1991). See the Cold Spring Harbor Laboratory Manuals cited below for the details involved in DNA sequence engineering. Nucleic acid sequences encoding individual CDR and framework sequences also can be synthesized using known techniques such as, for example, solid phase synthesis. Polypeptides also can be produced through synthetic methods well-known in the art (Merrifield, Science, 85:2149 (1963)).

b. Vectors

Provided herein are vectors that contain nucleic acid encoding the polypeptides and anti-HCV antibodies or antigen-binding fragments thereof. Many expression vectors are available and known to those of skill in the art and can be used for expression of polypeptides. See for example, CURRENT PROTOCOLS IN MOLECULAR BIOLOGY, Ausubel et al. eds., John Willey & Sons, Inc. 1999; see also U.S. Pat. Nos. 5,667,992 and 7,238,356. The choice of expression vector will be influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector in the cells.

Vectors also can contain additional nucleotide sequences operably linked to the ligated nucleic acid molecule, such as, for example, an epitope tag such as for localization, e.g. a his₆ tag or a myc tag, or a tag for purification, for example, a GST fusion, and a sequence for directing protein secretion and/or membrane association.

Expression of the polypeptides and anti-HCV antibodies or antigen-binding fragments thereof can be controlled by any promoter/enhancer known in the art. Suitable bacterial promoters are well known in the art and described herein below. Other suitable promoters for mammalian cells, yeast cells and insect cells are well known in the art and some are exemplified below. Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bernoist and Chambon, Nature 290:304-310 (1981)), the promoter contained in the 3′ long terminal repeat of Rous sarcoma virus (Yamamoto et al. Cell 22:787-797 (1980)), the herpes thymidine kinase promoter (Wagner et al., Proc. Natl. Acad. Sci. USA 78:1441-1445 (1981)), the regulatory sequences of the metallothionein gene (Brinster et al., Nature 296:39-42 (1982)); prokaryotic expression vectors such as the β-lactamase promoter (Jay et al., (1981) Proc. Natl. Acad. Sci. USA 78:5543) or the tac promoter (DeBoer et al., Proc. Natl. Acad. Sci. USA 80:21-25 (1983)); see also “Useful Proteins from Recombinant Bacteria”: in Scientific American 242:79-94 (1980)); plant expression vectors containing the nopaline synthetase promoter (Herrera-Estrella et al., Nature 303:209-213 (1984)) or the cauliflower mosaic virus 35S RNA promoter (Gardner et al., Nucleic Acids Res. 9:2871 (1981)), and the promoter of the photosynthetic enzyme ribulose bisphosphate carboxylase (Herrera-Estrella et al., Nature 310:115-120 (1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 38:639-646 (1984); Ornitz et al., Cold Spring Harbor Symp. Quant. Biol. 50:399-409 (1986); MacDonald, Hepatology 7:425-515 (1987)); insulin gene control region which is active in pancreatic beta cells (Hanahan et al., Nature 315:115-122 (1985)), immunoglobulin gene control region which is active in lymphoid cells (Grosschedl et al., Cell 38:647-658 (1984); Adams et al., Nature 318:533-538 (1985); Alexander et al., Mol. Cell Biol. 7:1436-1444 (1987)), mouse mammary tumor virus control region which is active in testicular, breast, lymphoid and mast cells (Leder et al., Cell 45:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-1648 (1985); Hammer et al., Science 235:53-58 1987)), alpha-1 antitrypsin gene control region which is active in liver (Kelsey et al., Genes and Devel. 1:161-171 (1987)), beta globin gene control region which is active in myeloid cells (Magram et al., Nature 315:338-340 (1985); Kollias et al., Cell 46:89-94 (1986)), myelin basic protein gene control region which is active in oligodendrocyte cells of the brain (Readhead et al., Cell 48:703-712 (1987)), myosin light chain-2 gene control region which is active in skeletal muscle (Shani, Nature 314:283-286 (1985)), and gonadotrophic releasing hormone gene control region which is active in gonadotrophs of the hypothalamus (Mason et al., Science 234:1372-1378 (1986)).

In addition to the promoter, the expression vector typically contains a transcription unit or expression cassette that contains all the additional elements required for the expression of the antibody, or portion thereof, in host cells. A typical expression cassette contains a promoter operably linked to the nucleic acid sequence encoding the germline antibody chain and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a germline antibody chain under the direction of the polyhedron promoter or other strong baculovirus promoter.

Any methods known to those of skill in the art for the insertion of DNA fragments into a vector can be used to construct expression vectors containing a nucleic acid encoding an antibody or antigen-binding fragment thereof provided herein. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

Exemplary plasmid vectors useful to produce the antibodies or antigen-binding fragments provided herein contain a strong promoter, such as the HCMV immediate early enhancer/promoter or the MHC class I promoter, an intron to enhance processing of the transcript, such as the HCMV immediate early gene intron A, and a polyadenylation (polyA) signal, such as the late SV40 polyA signal. The plasmid can be multicistronic to enable expression of the full-length heavy and light chains of the antibody, a single chain Fv fragment or other immunoglobulin fragments.

In particular examples, polypeptides of antigen-binding fragments (e.g. Fabs and F(ab′)₂) can be converted into the IgG, IgM, IgA, IgD or IgE form by cloning into expression vectors that encode the appropriate constant regions. See, for example, the methods and vectors described by Burton et al., Science 266, 1024-27 (1994) and Law et al., Nature Medicine 14:25-27 (2008).

c. Cell Expression Systems

Nucleic acids encoding the anti-HCV antibodies or antigen-binding fragments thereof provided herein can be expressed in a suitable host. Cells containing the vectors and nucleic acids encoding the anti-HCV antibodies or antigen-binding fragments thereof provided herein are provided. Generally, any cell type that can be engineered to express heterologous DNA and has a secretory pathway is suitable. Expression hosts include prokaryotic and eukaryotic organisms, such as bacterial cells (e.g. E. coli), yeast cells, fungal cells, Archae, plant cells, insect cells and animal cells including human cells. Expression hosts can differ in their protein production levels as well as the types of post-translational modifications that are present on the expressed proteins. Further, the choice of expression host is often related to the choice of vector and transcription and translation elements used. For example, the choice of expression host is often, but not always, dependent on the choice of precursor sequence utilized. For example, many heterologous signal sequences can only be expressed in a host cell of the same species (i.e., an insect cell signal sequence is optimally expressed in an insect cell). In contrast, other signal sequences can be used in heterologous hosts such as, for example, the human serum albumin (hHSA) signal sequence which works well in yeast, insect, or mammalian host cells and the tissue plasminogen activator pre/pro sequence which has been demonstrated to be functional in insect and mammalian cells (Tan et al., (2002) Protein Eng. 15:337). The choice of expression host can be made based on these and other factors, such as regulatory and safety considerations, production costs and the need and methods for purification. Thus, the vector system must be compatible with the host cell used.

Expression in eukaryotic hosts can include expression in yeasts such as Saccharomyces cerevisiae and Pichia pastoris, insect cells such as Drosophila cells and lepidopteran cells, plants and plant cells such as tobacco, corn, rice, algae, and lemna. Eukaryotic cells for expression also include mammalian cell lines such as Chinese hamster ovary (CHO) cells or baby hamster kidney (BHK) cells. Eukaryotic expression hosts also include production in transgenic animals, for example, including production in serum, milk and eggs.

Recombinant molecules can be introduced into host cells via, for example, transformation, transfection, infection, electroporation and sonoporation, so that many copies of the gene sequence are generated. Generally, standard transfection methods are used to produce bacterial, mammalian, yeast, or insect cell lines that express a large quantity of antibody chains, which are then purified using standard techniques (see e.g., Colley et al. (1989) J. Biol. Chem., 264:17619-17622; Guide to Protein Purification, in Methods in Enzymology, vol. 182 (Deutscher, ed.), 1990). Transformation of eukaryotic and prokaryotic cells are performed according to standard techniques (see, e.g., Morrison (1977) J. Bact. 132:349-351; Clark-Curtiss and Curtiss (1983) Methods in Enzymology, 101, 347-362). For example, any of the well-known procedures for introducing foreign nucleotide sequences into host cells can be used. These include the use of calcium phosphate transfection, polybrene, protoplast fusion, electroporation, biolistics, liposomes, microinjection, plasma vectors, viral vectors (e.g., baculovirus, vaccinia virus, adenovirus and other viruses), and any other well known method for introducing cloned genomic DNA, cDNA, plasmid DNA, cosmid DNA, synthetic DNA or other foreign genetic material into a host cell.

Exemplary bacterial host cells that can be used to express the polypeptides, including antibodies and fragments thereof, include E. coli XL1-Blue, Top10 and BL21. Exemplary mammalian host cells for expression of the polypeptides, including antibodies and fragments thereof, include Chinese hamster ovary (CHO) cells and HEK293 cells. These host cells are capable of expressing appropriately folded immunoglobulin polypeptide. For example, Fabs and F(ab′)₂ can be produced in bacterial or mammalian cells, and IgGs can be produced in mammalian cells. Thus, the polypeptides provided herein can be produced as assembled Fabs or IgGs in bacteria or mammalian cells, and then purified from bacterial periplasmic extract or cell culture medium using, for example, protein A- or protein G-conjugated agarose.

i. Prokaryotic Expression

Prokaryotes, especially E. coli, provide a system for producing large amounts of proteins and can be used to express the polypeptides and anti-HCV antibodies or antigen-binding fragments thereof provided herein. Typically, E. coli host cells are used for amplification and expression of the provided variant polypeptides. Transformation of E. coli is a simple and rapid technique well known to those of skill in the art. Expression vectors for E. coli can contain inducible promoters, such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated λPL promoter.

Proteins, such as any provided herein, can be expressed in the cytoplasmic environment of E. coli. For some polypeptides, the cytoplasmic environment, can result in the formation of insoluble inclusion bodies containing aggregates of the proteins. Reducing agents such as dithiothreitol and β-mercaptoethanol and denaturants, such as guanidine-HCl and urea can be used to resolubilize the proteins, followed by subsequent refolding of the soluble proteins. An alternative approach is the expression of proteins in the periplasmic space of bacteria which provides an oxidizing environment and chaperonin-like disulfide isomerases and can lead to the production of soluble protein. For example, for phage display of the proteins, the proteins are exported to the periplasm so that they can be assembled into the phage. Typically, a leader sequence is fused to the protein to be expressed which directs the protein to the periplasm. The leader is then removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce non-glycosylated proteins. Thus, if proteins require glycosylation for function, glycosylation can be added in vitro after purification from host cells.

ii. Yeast Cells

Yeasts such as Saccharomyces cerevisae, Schizosaccharomyces pombe, Yarrowia lipolytica, Kluyveromyces lactis and Pichia pastoris are well known yeast expression hosts that can be used to express the anti-HCV antibodies or antigen-binding fragments thereof provided herein. Yeast can be transformed with episomal replicating vectors or by stable chromosomal integration by homologous recombination. Typically, inducible promoters are used to regulate gene expression. Examples of such promoters include GAL1, GAL7 and GAL5 and metallothionein promoters, such as CUP1, AOX1 or other Pichia or other yeast promoter. Expression vectors often include a selectable marker such as LEU2, TRP1, HIS3 and URA3 for selection and maintenance of the transformed DNA. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed polypeptides as they exit the secretion pathway. Yeast also is capable of glycosylation at Asn-X-Ser/Thr motifs.

iii. Insect Cells

Insect cells, particularly using baculovirus expression, can be used to express the polypeptides and anti-HCV antibodies or antigen-binding fragments thereof provided herein. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the bombyx mori nuclear polyhedrosis virus (BmNPV) and an insect cell line such as SD derived from Spodoptera frugiperda, Pseudaletia unipuncta (A7S) and Danaus plexippus (DpN1). For high-level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schnieder 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

iv. Mammalian Cells

Mammalian expression systems can be used to express the polypeptides and anti-HCV antibodies or antigen-binding fragments thereof provided herein. Expression constructs can be transferred to mammalian cells by viral infection, such as, but not limited to adenovirus or vaccinia virus, or by direct DNA transfer such as liposomes, calcium phosphate, DEAE-dextran and by physical means, such as electroporation and microinjection. Expression vectors for mammalian cells typically include an mRNA cap site, a TATA box, a translational initiation sequence (Kozak consensus sequence) and polyadenylation elements. Such vectors often include transcriptional promoter-enhancers for high-level expression, for example the SV40 promoter-enhancer, the human cytomegalovirus (CMV) promoter and the long terminal repeat of Rous sarcoma virus. These promoter-enhancers are active in many cell types. Tissue and cell type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase and thymidine kinase. Fusion with cell surface signaling molecules such as TCR-ξ and Fc_{ε}RI-γ can direct expression of the proteins in an active state on the cell surface.

Many cell lines are available for mammalian expression including mouse, rat human, monkey, chicken and hamster cells. Exemplary cell lines include, but are not limited to, CHO, Huh-7, Balb/3T3, 293, BHK, HeLa, MDCK, MT2, mouse NS0 (nonsecreting) and other myeloma cell lines, hybridoma and heterohybridoma cell lines, lymphocytes, fibroblasts, Sp2/0, COS, NIH3T3, HEK293, W138, BT483, HS578T, HTB2, BT20, T47D, 293S, 2B8, and HKB cells. Cell lines also are available adapted to serum-free media which facilitates purification of secreted proteins from the cell culture media. One such example is the serum free EBNA-1 cell line (Pham et al. (2003) Biotechnol. Bioeng. 84:332-42.)

v. Plants

Transgenic plant cells and plants can be used to express polypeptides such as any described herein. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline syntase promoter, the ribose bisphosphate carboxylase promoter and the ubiquitin and UBQ3 promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce proteases or modified proteases (see for example, Mayfield et al. (2003) PNAS 100:438-442). Because plants have different glycosylation patterns than mammalian cells, this can influence the choice of protein produced in these hosts.

d. Purification

Methods for purification of polypeptides, including the anti-HCV antibodies or antigen-binding fragments thereof provided, from host cells will depend on the chosen host cells and expression systems. For secreted molecules, proteins generally are purified from the culture media after removing the cells. For intracellular expression, cells can be lysed and the proteins purified from the extract. In one example, polypeptides are isolated from the host cells by centrifugation and cell lysis (e.g. by repeated freeze-thaw in a dry ice/ethanol bath), followed by centrifugation and retention of the supernatant containing the polypeptides. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary the proteins can be extracted and further purified using standard methods in the art.

Proteins, such as the anti-HCV antibodies or antigen-binding fragments thereof provided, can be purified, for example, from lysed cell extracts, using standard protein purification techniques known in the art including but not limited to, SDS-PAGE, size fraction and size exclusion chromatography, ammonium sulfate precipitation and ionic exchange chromatography, such as anion exchange. Affinity purification techniques also can be utilized to improve the efficiency and purity of the preparations. For example, antibodies, receptors and other molecules that bind proteases can be used in affinity purification. Expression constructs also can be engineered to add an affinity tag to a protein such as a myc epitope, GST fusion or His₆ and affinity purified with myc antibody, glutathione resin and Ni-resin, respectively. Purity can be assessed by any method known in the art including gel electrophoresis and staining and spectrophotometric techniques.

The isolated polypeptides then can be analyzed, for example, by separation on a gel (e.g. SDS-Page gel), size fractionation (e.g. separation on a Sephacryl™ S-200 HiPrep™ 16×60 size exclusion column (Amersham from GE Healthcare Life Sciences, Piscataway, N.J.). Isolated polypeptides also can be analyzed in binding assays, typically binding assays using a binding partner bound to a solid support, for example, to a plate (e.g. ELISA-based binding assays) or a bead, to determine their ability to bind desired binding partners. The binding assays described in the sections below, which are used to assess binding of precipitated phage displaying the polypeptides, also can be used to assess polypeptides isolated directly from host cell lysates. For example, binding assays can be carried out to determine whether antibody polypeptides bind to one or more antigens, for example, by coating the antigen on a solid support, such as a well of an assay plate and incubating the isolated polypeptides on the solid support, followed by washing and detection with secondary reagents, e.g. enzyme-labeled antibodies and substrates.

e. Additional Methods for Producing Antibodies

Additional methods can be used to produce anti-HCV antibodies. For example, antibodies can be obtained from the blood of a mammal that has been immunized with the E2 glycoprotein or the E1E2 complex of HCV. E2 glycoprotein or E1E2 complex can be obtained using conventional methods. See, for example, Lesniewski et al., J. Med. Virol. 45, 415-422 (1995); Chan-Fook et al., Virology 273, 60-66 (2000); Heile et al., J. Virol. 74: 6885-92 (2000); U.S. Pat. No. 6,274,148; and U.S. Pat. No. 5,667,992. The mammal can be for example, a rabbit, goat, or mouse. At the appropriate time after immunization, antibody molecules can be isolated from the mammal, e.g. from the blood or other fluid of the mammal, and further purified using standard techniques that include, without limitation, precipitation using ammonium sulfate, gel filtration chromatography, ion exchange chromatography or affinity chromatography using protein A. Antibodies that bind to HCV-specific antigens, e.g. antigenic epitopes on E2, can be identified using ELISA. Antibodies that bind to epitopes on the E1E2 complex, but do not bind to E2 epitopes can be identified by a masking technique as described herein.

Polypeptides and antibodies also can be obtained using various methods. Non-limiting examples include: (1) the generation of a polypeptide or antibody from an antibody-producing cell of a mammal that has been immunized or infected with HCV using single human B cell RT-PCR and expression vector cloning; (2) isolation from immortalized antibody-secreting B cells; and (3) isolation from an antibody-producing hybridoma generated by fusion of an antibody-producing cell with a myeloma cell. These techniques are known in the art. See, for example, Kohler & Milstein, Nature 256:495-97 (1975); Kozbor et al. Immunol Today 4: 72 (1983); Tiller et al., J Immunol Methods 329:112-124 (2008) and Traggiai et al., Nat Med 10:871-875 (2004).

Polypeptides or antibodies also can be prepared using other methods known in the art, such as, for example, screening of a recombinant combinatorial immunoglobulin library such as an antibody phage display library using antigenic epitope of the E2 glycoprotein or the E1E2 complex of HCV. See, for example, Barbas, C. F. et al., PHAGE DISPLAY—A LABORATORY MANUAL (2001) Cold Spring Harbor, New York: Cold Spring Harbor Laboratory Press; and Kontermann & Dubel, ANTIBODY ENGINEERING (2001) Berlin, Heidelberg: Springer-Verlag.

Nucleic acids encoding polypeptides, including antibodies and fragments thereof, can be derived from a human patient chronically infected with HCV by generating an expression library using the RNA of the patient's B cells or plasma cells and then screening for antibody-coding sequences. Alternatively, nucleic acids encoding polypeptides, including antibodies and fragments thereof, may be obtained from the cells of a non-human mammal immunized with the E2 glycoprotein or the E1E2 complex as an antigen, or antigenic epitopes of these proteins. See, for example, in Antibodies, A Laboratory Manual by Harlow and Lane, Cold Spring Harbor Laboratory Press, Cold Spring Harbor, N.Y., 1988, and in Molecular Cloning, A Laboratory Manual by Sambrook, et al., Cold Spring Harbor Laboratory Press, Cold Spring Harbor, N.Y. 1989, the disclosures of which are incorporated herein by reference.

Briefly, immune cells sensitized to the specified antigen such as mononuclear cells from the bone marrow of a patient infected with HCV or spleen cells harvested from a non-human mammalian host can be used. The mononuclear cells are processed according to the phage display technology described by Barbas et al. Proc. Natl. Acad. Sci., USA 88, 7978-7982 (1991); and in U.S. Pat. Nos. 5,580,717; 5,972,656; 6,113,898; and 6,140,470. The disclosures of these documents are incorporated herein by reference. Total RNA or RNA that has been processed to obtain the poly A-RNA is isolated from cells. After hybridization of an oligo-d(T) primer, the RNA (mRNA) is reverse transcribed to yield the corresponding cDNA. This cDNA is used to isolate nucleic acids encoding polypeptides. The cDNA can be amplified by polymerase chain reaction (PCR) to obtain nucleic acids encoding polypeptides. Selected primers can be used in PCR to isolate nucleic acids encoding polypeptide fragments such as V_{H}, V_{L}, as well as the constant regions.

The cDNA or PCR products can be inserted into a vector, such as a bacteriophage, a phagemid or a plasmid through use of recombinant DNA techniques well-known in the art. See Sambrook et al. (1989). The vectors containing the cDNA or PCR products can be introduced into bacteria to produce an expression library, the members of which express a polypeptide encoded by the cDNA or PCR product such as a full-length light or heavy chain polypeptide, a single variable chain polypeptide, or a single chain Fab or Fab′.

The procedure also allows for the production of a polypeptide that is fused to a phage coat protein. The library of recombinant phage can be panned as described in the foregoing references and patents to select members of the library that express an antibody that binds specifically to the E2 glycoprotein or the E1E2 complex. The panning may be accomplished by combining the library with an immobilized antigen, removing the phage not bound, and then removing the bound phage.

Host bacterial cells such as E. coli or other suitable bacteria can be transfected with members of the phage library that express an antibody that binds to the immobilized antigen. The resulting bacterial cells can be separated into colonies by serial dilution and plating so that each colony isolated expresses a unique antibody. The phage also can carry a selection marker such as an antibiotic resistance gene so that host bacterial cells expressing an antibody can be identified by cultivating the host cell library in culture medium containing the antibiotic. Cultures from single colonies can be examined by a binding assay using the E2 protein or E1-E2 complex to identify those expressing an antibody having particular immunoreactivity.

Following selection of bacterial colonies that express an antibody having specific immunoreactivities, the nucleic acid sequences coding for the CDR's, framework regions, or variable or constant regions in the selected cultures can be determined using known nucleotide sequencing procedures. Thus, nucleic acids encoding CDR sequences, framework sequences and/or constant regions of an polypeptide can be obtained from a phage library.

The following detailed procedure provides further explanation for production of the polypeptides provided herein.

PCR amplification of Fd and κ regions from the mRNA of the source mononuclear cells may be performed as described by Sastry et al., Proc. Natl. Acad. Sci U.S.A., 86, 5728 (1989) and Barbas et al., PHAGE DISPLAY: A LABORATORY MANUAL. New York: Cold Spring Harbor Laboratory Press (2001). The PCR amplification is performed with cDNA obtained by the reverse transcription of the mRNA with primer specific for amplification of heavy chain sequences or light chain sequences.

The PCR amplification of messenger RNA (mRNA) isolated from the mononuclear cells with oligonucleotides that incorporate restriction sites into the ends of the amplified product may be used to clone and express heavy chain sequences (e.g., the amplification of the Fd fragment) and κ light chain sequences from B cells. The oligonucleotide primers, which are analogous to those that have been successfully used for amplification of V_{H} and V_{L} sequences (see Sastry et al., Proc. Natl. Acad. Sci. U.S.A., 86, 5728 (1989) and Barbas et al., PHAGE DISPLAY: A LABORATORY MANUAL. New York: Cold Spring Harbor Laboratory Press (2001)), may be used for these amplifications. Restriction endonuclease recognition sequences are typically incorporated into these primers to allow for the cloning of the amplified fragment into a suitable vector (i.e. a phagemid or a λ phage) in a predetermined reading frame for expression.

Phage assembly proceeds via an extrusion-like process through the bacterial membrane. For example filamentous phage M13 may be used for this process. This phage has a 406-residue minor phage coat protein (cpIII) which is expressed before extrusion and which accumulates on the inner membrane facing into the periplasm of E. coli. The two functional properties of cpIII, infectivity and normal (nonpolyphage) morphogenesis have been assigned to roughly the first and second half of the gene. The N-terminal domain of cpIII binds to the F′ pili, allowing for infection of E. coli, whereas the membrane-bound C-terminal domain, P198-S406, serves the morphogenic role of capping the trailing end of the filament according to the vectorial polymerization model.

A phagemid vector may be constructed to fuse the antibody fragment chain such as an Fab, Fab′ or preferably an Fd chain with the C-terminal domain of cpIII (see Barbas et al., Proc. Natl. Acad. Sci. USA, 88, 7978 (1991)). A flexible five-amino acid tether (GGGGS), which lacks an ordered secondary structure, may be juxtaposed between the expressed fragment chain and cpIII domains to minimize interaction. The phagemid vector also can be constructed to include a nucleotide coding for the light chain of a Fab fragment. The cpIII/Fd fragment fusion protein and the light chain protein may be placed under control of separate lac promoter/operator sequences and directed to the periplasmic space by pelB leader sequences for functional assembly on the membrane. Inclusion of the phage F1 intergenic region in the vector allows for packaging of single-stranded phagemid with the aid of helper phage. The use of helper phage superinfection may result in expression of two forms of cpIII. Consequently, normal phage morphogenesis may be perturbed by competition between the cpIII/Fd fragment fusion protein and the native cpIII of the helper phage for incorporation into the virion. The resulting packaged phagemid may carry native cpIII, which is necessary for infection, and the fusion protein including the Fab fragment, which may be displayed for interaction with an antigen and used for selection. Fusion at the C-terminal domain of cpIII is necessitated by the phagemid approach because fusion with the infective N-terminal domain would render the host cell resistant to infection. The result is a phage displaying antibody combining sites (“Phabs”). The antibody combining sites, such as Fab fragments, are displayed on the phage coat. This technique may be used to produce Phabs which display recombinantly produced Fab fragments, such as recombinantly produced Fab fragments that immunoreact with an antigen, on the phage coat of a filamentous phage such as M13.

A phagemid vector (i.e. pComb 3 or pComb3H) which allows the display of antibody Fab fragments on the surface of filamentous phage, has been described (see Barbas et al., Proc. Natl. Acad. Sci. USA, 88, 7978 (1991). Xho I and Spe I sites for cloning PCR-amplified heavy-chain Fd sequences are included in pComb 3 and pComb3H. Sac I and Xba I sites are also provided for cloning PCR-amplified antibody light chains. These cloning sites are compatible with known mouse and human PCR primers (see, e.g., Huse et al., Science, 246, 1275-1281 (1989)). The nucleotide sequences of the pelB leader sequences are recruited from the λ HC2 and λ LC2 constructs described in Huse et al, ibid, with reading frames maintained. Digestion of pComb 3 and pComb3H, encoding a selected Fab, with Spe I and Nhe I permit the removal of the gene III fragment, which includes the nucleotide sequences coding for the antibody Fab fragments. Because Spe I and Nhe I produce compatible cohesive ends, the digested vector also can be religated to yield a phagemid that produces soluble Fab.

Fabs may be produced by overnight infection of phagemid containing cells (e.g., infected E. coli XL-1 Blue) yielding typical titers of 10¹¹ cfu/ml. By using phagemids encoding different antibiotic resistances, ratios of clonally distinct phage may easily be determined by titering on selective plates. In single-pass enrichment experiments, clonally mixed phage may be incubated with an antigen-coated plate. Nonspecific phage will be removed by washing, and bound phage may then be eluted with acid and isolated.

The polypeptides provided herein, including antibodies and fragments thereof, can be used to detect the presence, or determine the amount, of HCV in a biological sample as described herein. These polypeptides also can be used for a prophylactic purpose to prevent infection of a mammal or mammalian cell by HCV.

4. Assessing Anti-HCV Antibodies

The anti-HCV antibodies or antigen-binding fragments thereof provided herein can be characterized in a variety of ways well-known to one of skill in the art. For example, the anti-HCV antibodies or antigen-binding fragments thereof provided herein can be assayed for the ability to immunospecifically bind to E1, E2 or E1E2 complex of HCV. Such assays can be performed, for example, in solution (e.g., Houghten (1992) Bio/Techniques 13:412-421), on beads (Lam (1991) Nature 354:82-84), on chips (Fodor (1993) Nature 364:555-556), on bacteria (U.S. Pat. No. 5,223,409), on spores (U.S. Pat. Nos. 5,571,698; 5,403,484; and 5,223,409), on plasmids (Cull et al. (1992) Proc. Natl. Acad. Sci. USA 89:1865-1869) or on phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310). Antibodies or antigen-binding fragments thereof that have been identified to selectively bind to an HCV antigen or a fragment thereof also can be assayed for their specificity and affinity for a HCV antigen. In addition, in vitro assays and in vivo animal models using the anti-HCV antibodies or antigen-binding fragments thereof provided herein can be employed for measuring the level of HCV neutralization effected by contact or administration of the anti-HCV antibodies or antigen-binding fragments thereof.

a. Binding Assays

The anti-HCV antibodies or antigen-binding fragments thereof provided herein can be assessed for their ability to bind a selected target (e.g., HCV virus, E2 polypeptide, or E1E2 complex) and the specificity for such targets by any method known to one of skill in the art. Exemplary assays are provided in the Examples and described herein below. Binding assays can be performed in solution, suspension or on a solid support. For example, target antigens can be immobilized to a solid support (e.g. a carbon or plastic surface, a tissue culture dish or chip) and contacted with antibody or antigen-binding fragment thereof. Unbound antibody or target protein can be washed away and bound complexes can then be detected. Binding assays can be performed under conditions to reduce nonspecific binding, such as by using a high ionic strength buffer (e.g. 0.3-0.4 M NaCl) with nonionic detergent (e.g. 0.1% Triton X-100 or Tween 20) and/or blocking proteins (e.g. bovine serum albumin or gelatin). Negative controls also can be included in such assays as a measure of background binding. Binding affinities can be determined using Scatchard analysis (Munson et al., Anal. Biochem., 107:220 (1980)), surface plasmon resonance, isothermal calorimetry, or other methods known to one of skill in the art.

Exemplary immunoassays that can be used to analyze immunospecific binding and cross-reactivity include, but are not limited to, competitive and non-competitive assay systems using techniques such as, but not limited to, western blots, radioimmunoassays, ELISA (enzyme linked immunosorbent assay), Meso Scale Discovery (MSD, Gaithersburg, Md.), “sandwich” immunoassays, immunoprecipitation assays, ELISPOT, precipitin reactions, gel diffusion precipitin reactions, immunodiffusion assays, agglutination assays, complement-fixation assays, immunoradiometric assays, fluorescent immunoassays, protein A immunoassays. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Other assay formats include liposome immunoassays (LIA), which use liposomes designed to bind specific molecules (e.g., antibodies) and release encapsulated reagents or markers. The released chemicals are then detected according to standard techniques (see Monroe et al., (1986) Amer. Clin. Prod. Rev. 5:34-41). Exemplary immunoassays not intended by way of limitation are described briefly below.

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody or antigen-binding fragment thereof of interest to the cell lysate, incubating for a period of time (e.g., 1 to 4 hours) at 40° C., adding protein A and/or protein G sepharose beads to the cell lysate, incubating for about an hour or more at 40° C., washing the beads in lysis buffer and resuspending the beads in SDS/sample buffer. The ability of the antibody or antigen-binding fragment thereof of interest to immunoprecipitate a particular antigen can be assessed by, e.g., western blot analysis. One of skill in the art is knowledgeable as to the parameters that can be modified to increase the binding of the antibody or antigen-binding fragment thereof to an antigen and decrease the background (e.g., pre-clearing the cell lysate with sepharose beads). For further discussion regarding immunoprecipitation protocols see, e.g., Ausubel et al., eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody or antigen-binding fragment thereof (i.e. the antibody or antigen-binding fragment thereof of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art is knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody or antigen-binding fragment thereof of interest conjugated to a detectable compound such as an enzymatic substrate (e.g., horseradish peroxidase or alkaline phosphatase) to the well and incubating for a period of time, and detecting the presence of the antigen. In ELISAs, the antibody or antigen-binding fragment thereof of interest does not have to be conjugated to a detectable compound; instead, a second antibody (which recognizes the antibody of interest) conjugated to a detectable compound can be added to the well. Further, instead of coating the well with the antigen, the antibody can be coated to the well. In this case, a second antibody conjugated to a detectable compound can be added following the addition of the antigen of interest to the coated well. One of skill in the art is knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody or antigen-binding fragment thereof to an antigen and the off-rate of an antibody-antigen interaction can be determined, for example, by competitive binding assays. One example of a competitive binding assay is a radioimmunoassay comprising the incubation of labeled antigen (e.g., ³H or ¹²⁵I) with the antibody or antigen-binding fragment thereof of interest in the presence of increasing amounts of unlabeled antigen, and the detection of the antibody or antigen-binding fragment thereof bound to the labeled antigen. The affinity of an anti-HCV antibody or antigen-binding fragment thereof provided herein for a HCV antigen and the binding off-rates can be determined from the data by Scatchard plot analysis. Competition with a second antibody can also be determined using radioimmunoassay or assays with other labels for the antibodies, such as biotin (see, e.g., Example 1). In this case, a HCV antigen, such as E1E2 complex, is incubated with an anti-HCV antibody or antigen-binding fragment thereof provided herein conjugated to a labeled compound (e.g., ³H, ¹²⁵I or biotin) in the presence of an unlabeled second antibody. The amount of labeled antibody that is bound to the antigen is then assessed and compared to the binding in the absence of the blocking antibody. Competition is determined by the percentage change in binding signals in the presence of the unlabeled blocking antibody compared to the absence of the blocking antibody. Such competition assays can be used to assess whether two antibodies bind to the same epitope. Typically, competition of 70% or more (i.e. the amount of labeled antibody bound to the antigen in the presence of the unlabeled blocking antibody is 30% or less of the amount of the biotinylated antibody bound to the antigen in the absence of the antibody), such as 70%, 71%, 72%, 73%, 74%, 75%, 80%, 85%, 90%, 95% or more, is indicative that the antibodies bind to the same epitope.

In some examples, surface plasmon resonance (e.g., BiaCore 2000, Biacore AB, Upsala, Sweden and GE Healthcare Life Sciences; Malmqvist (2000) Biochem. Soc. Trans. 27:335) kinetic analysis can be used to determine the binding on and off rates of antibodies or antigen-binding fragments thereof to a HCV antigen. Surface plasmon resonance kinetic analysis comprises analyzing the binding and dissociation of a HCV antigen from chips with immobilized antibodies or fragments thereof on their surface.

b. In Vitro Assays for Analyzing Virus Neutralization Effects of Antibodies

The anti-HCV antibodies or antigen-binding fragments thereof provided herein can be analyzed using neutralization assays. Exemplary neutralization assays for the analysis of anti-HCV antibodies include those that employ HCVcc (cell culture derived HCV) or HCVpp (HCV pseudparticles). HCV typically does not replicate in vitro. However, an HCV genotype 2a clone, designated JFH-1, has been found to replicate in Huh-7 cells (Kato et al., (2003) Gastroenterology 125:1808-1817). Chimeric clones containing the 5′- and 3′-NCRs, as well as the NS3-5B region of JFH-1 and the C-NS2 region of a different genotype 2a isolate designated J6 have even greater replication efficiency (Lindenbach et al. (2005) Science 309, 623-626). Neutralization assays using HCV pseudoparticles (HCVpp), such as those described in Example 1, below, also can be used to characterize the anti-HCV antibodies provided herein. HCVpp are formed by incorporation of the full-length hepatitis C virus glycoproteins E1 and E2 onto lenti- or retroviral core particles. These particles are able to infect and replicate in, for example, 293T cells and Huh-7 cells.

c. In Vivo Animal Models

In vivo studies using animal models can be performed to assess the efficacy of the anti-HCV antibodies or antigen-binding fragments thereof provided herein. In vivo studies using animal models can be performed to assess the ability of the antibodies to prevent or treat HCV infection, and the toxicity of administration of such antibodies or antigen-binding fragments. There are limited animal models for HCV. These include the chimpanzee and the chimeric mouse model. The chimeric mouse model utilizes SCID-beige/Alb-uPA mice, which are immunodeficient SCID mice carrying a urokinase plasminogen activator (uPA) transgene under the control of the albumin promoter (Mercer et al. (2001) Nature Med. 7:927-933). Expression of the uPA transgene in the mouse liver causes a depletion of the mouse hepatocytes. Transplantation of normal human hepatocytes into SCID-beige/Alb-uPA mice results in animals with chimeric human livers which can then be infected with HCV. This model can be used to assess the ability of the anti-HCV antibodies or antigen-binding fragments thereof provided herein to prevent or treat HCV infection, as demonstrated in Examples 1 and 2. The half-life of the antibodies also can be assessed.

5. Uses for Anti-HCV Polypeptides and Antibodies

The polypeptides provided herein, including antibodies and fragments thereof, can be used to detect the presence of HCV in a sample from a source that has been exposed to HCV and is susceptible to HCV infection or capable of sustaining HCV replication. The polypeptide or antibody can be Tabled with a detectable molecule, which can be an enzyme such as alkaline phosphatase, acetylcholinesterase, β-galactosidase or horseradish peroxidase; a prosthetic group such as streptavidin, biotin, or avidin; a fluorescent group such as dansyl chloride, dichlorotriazinylamine, dichlorotriazinylamine fluorescein, fluorescein, fluorescein isothiocyanate, phycoerythrin, rhodamine, umbelliferone; a luminescent group such as luminal; a bioluminescent group such as aequorin, luciferase, and luciferin; or a radioisotope such as ³H, ¹²⁵I, ¹³¹I, ³⁵S.

When combined with a sample from a source that has been exposed to HCV, the presence of HCV can be detected by detecting the formation of a complex between the polypeptide or antibody and HCV particles.

The polypeptides provided herein, including antibodies and fragments thereof, also can be used to prevent or treat infection by an HCV or to prevent or reduce HCV replication. For example, the polypeptides provided herein, including antibodies and fragments thereof, can be administered to a subject that has been exposed to the virus or is likely exposed to the virus, to prevent new or recurring HCV infection and its associated symptoms and/or complications, or prevent or reduce HCV replication. This includes administration to a clinically symptomatic individual to treat new or recurring HCV infection. The polypeptides provided herein, including antibodies and fragments thereof, can be used to prevent or treat infection of a mammalian cell, such as a human cell. It can be used to prevent or treat acute or chronic HCV infection, or prevent or reduce HCV replication, in a mammal such as a human.

The degree of HCV infection or replication can be determined by determining the amount of HCV particles or the amount of HCV RNA in a sample from the mammal using methods described herein.

A subject, such as a mammal, for example a human, that can benefit from the polypeptides provided herein, including antibodies and fragments thereof, includes one who is likely to be or has been exposed to HCV. Such a mammal includes, without limitation, someone present in an area where HCV is prevalent or commonly transmitted, e.g., Africa, Southeast Asia, China, South Asia, Australia, India, the United States, Russia, as well as Central and South American countries. A subject that is likely to be or has been exposed to HCV also includes a recipient of donated body tissues or fluids including, for example, a recipient of blood or one or more of its components such as plasma, platelets, or stem cells and an organ or cell transplant recipient. A subject, such as a mammal, including a human, who is likely to be or has been exposed to HCV also can include medical, clinical or dental personnel handling body tissues and fluids. A mammal who has been exposed to HCV includes, without limitation, someone who has had contact with the body tissue or fluid, e.g. blood, of an infected person or otherwise have come in contact with HCV. A mammal that can benefit from a polypeptide of the invention includes a liver transplantee. Thus, the polypeptides provided herein, including antibodies and fragments thereof, can be used to prevent recurring HCV infection, for example, in an individual who has received a liver transplant.

The polypeptides provided herein, including antibodies and fragments thereof, can be used to prevent or treat HCV infection, or prevent or reduce HCV replication, as well as treat the associated disease condition or clinical symptoms. Thus, the polypeptides provided herein, including antibodies and fragments thereof, can be used to prevent or reduce transmission, to prevent or treat disease progression, and to prevent or reduce HCV replication or reduce viral load. Treatment with the polypeptide includes the alleviation or diminishment of at least one symptom typically associated with the infection. The treatment also includes alleviation or diminishment of more than one symptom. Ideally, the treatment cures, e.g., substantially inhibits viral infection and/or eliminates the symptoms associated with the infection.

Symptoms of HCV exposure or infection include, without limitation, inflammation of the liver, decreased appetite, fatigue, abdominal pain, jaundice, flu-like symptoms, itching, muscle pain, joint pain, intermittent low-grade fevers, sleep disturbances, nausea, dyspepsia, cognitive changes, depression, headaches and mood changes.

Diagnostic and screening techniques useful for identification of patients afflicted with HCV include any that identify antibody-antigen binding. HCV infection can be diagnosed by detecting antibodies to the virus, detecting liver inflammation by biopsy, liver cirrhosis, portal hypertension, thyroiditis, cryoglobulinemia and glomerulonephritis. In addition, diagnosis of exposure or infection or identification of one who is at risk of exposure to HCV could be based on medical history, abnormal liver enzymes or liver function tests during routine blood testing. Generally, infection can be diagnosed using polymerase chain reaction (PCR) for detecting viral nucleic acids, enzyme linked immunosorbent assay (ELISA) for detecting viral antigens or anti-viral antibodies, and immunofluorescence for detecting viral antigens. For example, the polypeptides or antibodies provided herein can be combined with an appropriate sample from the patient to produce a complex. The complex in turn can be detected with a marker reagent for binding with such a complex. Typical marker reagents include secondary antibodies selective for the complex, secondary antibodies selective for certain epitopes of the polypeptide or antibody or a label attached to the polypeptide or antibody itself. In particular, radioimmunoassay (RIA), radioallergosorbent test (RAST), radioimmunosorbent test (RIST), immunoradiometric assay (IRMA), Farr assay, fluorescence immunoassay (FIA), sandwich assay, enzyme linked immunosorbent assay (ELISA), northern or southern blot analysis, and color activation assay may be used following protocols well known for these assays. See for example Immunology, An Illustrated Outline by David Male, C.V. Mosby Company, St Louis, Mo., 1986 and the Cold Spring Harbor Laboratory Manuals cited above. Labels including radioactive labels, chemical labels, fluorescent labels, luciferase and the like also can be directly attached to the polypeptide according to the techniques described in U.S. patent No. (BN patent cite), the disclosure of which is incorporated herein by reference.

Methods of preventing or treating acute or recurring HCV infection include contacting the cell with an effective amount of a polypeptide or antibody or administering to a mammal such as a human a therapeutically effective amount of a polypeptide or antibody of the present invention. Methods of inactivating the virus include contacting the virus with an effective amount of the polypeptide or antibody provided herein.

The polypeptides provided herein, including antibodies and fragments thereof, can be administered in a variety of ways. Routes of administration include, without limitation, oral, parenteral (including subcutaneous, intravenous, intramuscular and intraperitoneal), rectal, vaginal, dermal, transdermal (topical), transmucosal, intrathoracic, intrapulmonary and intranasal (respiratory) routes. The means of administration may be by injection, using a pump or any other appropriate mechanism.

The polypeptides provided herein, including antibodies and fragments thereof, can be administered in a single dose, in multiple doses, in a continuous or intermittent manner, depending, for example, upon the recipient's physiological condition, whether the purpose of the administration is therapeutic or prophylactic, and other factors known to skilled practitioners. The administration of the polypeptides can be essentially continuous over a pre-selected period of time or may be in a series of spaced doses. For example, to prevent recurrent HCV infection in a transplant patient, the polypeptides provided herein, including antibodies and fragments thereof, can be administered during the anhepatic phase, i.e. after removal of the liver and before transplant of a replacement liver, as well as after the transplant, e.g. daily, weekly, biweekly or monthly. Both local and systemic administrations are contemplated.

The dosage to be administered to a mammal may be any amount appropriate to reduce or prevent viral infection or to treat at least one symptom associated with the viral infection. Some factors that determine appropriate dosages are well known to those of ordinary skill in the art and may be addressed with routine experimentation. For example, determination of the physicochemical, toxicological and pharmacokinetic properties may be made using standard chemical and biological assays and through the use of mathematical modeling techniques known in the chemical, pharmacological and toxicological arts. The therapeutic utility and dosing regimen may be extrapolated from the results of such techniques and through the use of appropriate pharmacokinetic and/or pharmacodynamic models. Other factors will depend on individual patient parameters including age, physical condition, size, weight, the condition being treated, the severity of the condition, and any concurrent treatment. The dosage will also depend on the polypeptide or antibody chosen and whether prevention or treatment is to be achieved, and if the polypeptide or antibody is chemically modified. Such factors can be readily determined by the clinician employing viral infection models such as in vitro HCV infection system described herein, or other animal models or test systems that are available in the art.

The precise amount to be administered to a patient will be the responsibility of the attendant physician. The amount useful to establish treatment of HCV can be determined by diagnostic and therapeutic techniques well known to those of ordinary skill in the art. The dosage may be determined by titrating a sample of the patient's blood sera with the polypeptide or antibody to determine the end point beyond which no further immunocomplex is formed. Such titrations may be accomplished by the diagnostic techniques discussed below. Available dosages include administration of from about 1 to about 1 million effective units of antibody per day, wherein a unit is that amount of polypeptide, which will provide at least 1 microgram of antigen-polypeptide complex. Preferably, from about 10 to about 100,000 units of antibody per day can be administered.

To achieve the desired effect(s), one or more polypeptides provided herein, including antibodies and fragments thereof, can be administered as single or divided dosages, for example, of at least about 0.01 mg/kg to about 500, 750 or 1000 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. For post-exposure prophylactic use, the one or more polypeptides or antibodies may be administered immediately, e.g. within 24 hours, after exposure to HCV. To prevent recurrent HCV infection, e.g. in a transplant recipient such as a liver transplant recipient, a polypeptide or antibody of the invention may be administered prior to and after transplantation. For example, the polypeptides, including antibodies, can be administered during the anhepatic phase, as well as during the post-operative phase. The polypeptide can be administered daily, biweekly or monthly after the transplant. The polypeptides provided herein, including antibodies and fragments thereof, can be administered daily for the first week after transplant, weekly for two, three or more weeks after the transplant and then monthly.

The absolute weight of a given polypeptide included in a unit dose can vary widely. For example, about 0.01 to about 2 g, or about 0.1 to about 500 mg, of at least one polypeptide, including an antibody, or a plurality of polypeptides, including a plurality of antibodies can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

The daily dose of the polypeptides provided herein, including antibodies and fragments thereof, can vary as well. Such daily dose can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

The polypeptides provided herein, including antibodies and fragments thereof, can be used alone or in combination with a second medicament. The second medicament can include, but is not limited to, another polypeptide, such as an antibody, provided herein, another polypeptide, such as an antibody, described elsewhere, and a known antiviral agent such as, for example, an interferon-based therapeutic or another type of antiviral medicament such as ribavirin. For example, the anti-HCV antibodies provided herein can be administered in combination with anti-NS3 antibodies having a heavy chain containing the sequence of amino acids set forth in any of SEQ ID NOS: 67-71 and a light chain containing the sequence of amino acids set forth in any of SEQ ID NOS: 72-77, or a CDR and/or framework sequence set forth in any of SEQ ID NOS: 276-308 or 573-606, respectively. In some instances, the second medicament is an anticancer, antibacterial, or another antiviral agent. The antiviral agent may act at any step in the life cycle of the virus from initial attachment and entry to egress. Thus, the second antiviral agent may interfere with attachment, fusion, entry, trafficking, translation, viral polyprotein processing, viral genome replication, viral particle assembly, egress or budding. Stated another way, the antiviral agent may be an attachment inhibitor, entry inhibitor, a fusion inhibitor, a trafficking inhibitor, a replication inhibitor, a translation inhibitor, a protein processing inhibitor, an egress inhibitor, in essence an inhibitor of any viral function. The effective amount of the second medicament will follow the recommendations of the manufacturer of the second medicament, as well as the judgment of the attending physician, and will be guided by the protocols and administrative factors for amounts and dosing as indicated in the PHYSICIAN'S DESK REFERENCE.

To determine the effectiveness of any of the polypeptides, including antibodies or fragments thereof, provided herein, for inhibition and treatment of HCV infection, methods available in the art and those described herein can be used. The effectiveness of the method of treatment can be assessed by monitoring the patient for signs or symptoms of the viral infection as discussed above, as well as determining the presence and/or amount of viral particle or viral RNA present in the blood, e.g. the viral load, using methods known in the art. Viral infection or replication in the presence or absence of the polypeptide can be evaluated, for example, by determining intracellular viral RNA levels or the number of viral foci by immunoassays using antibody against viral proteins as described herein. A polypeptide, including an antibody, is effective for treatment and inhibition of HCV if it can inhibit or reduce viral infection or replication by any amount, for example, by 5%, 10%, 15%, 20%, 25%, 30%, 35%, 40%, 45%, 50%, 55%, 60%, 65%, 70%, 75%, 80%, 85%, 90%, 95% or more. For example, a polypeptide, including an antibody, is effective for treatment and inhibition of HCV if it can inhibit or reduce viral infection or replication by 2 fold or more than 2 fold, such as by 2-5 fold, 5-10 fold, or more than 10 fold.

The polypeptides provided herein, including antibodies and fragments thereof, can be used to increase the safety of blood and blood products, to increase the safety of clinical laboratory samples and to increase the safety of biological tissues as well as articles, devices, or instruments intended for preventative or therapeutic use. For example, a polypeptide, such as an antibody, can be added to blood or blood products such as plasma, platelets, and blood or marrow cells prior to use. The polypeptides provided herein, including antibodies and fragments thereof, can be combined with cells or tissues prior to use or administration. It can be coated on articles, devices or instruments such as, for example, valves, bags and stents.

6. Pharmaceutical Compositions

Provided are pharmaceutical composition that includes at least one or more, including two, three, four, or more than four polypeptides, including antibodies, described above. In some examples, the composition includes a polypeptide, such as an antibody provided herein, in combination with another anti-HCV antibody that recognizes a different or non-overlapping HCV epitope. For example, a pharmaceutical composition can include an antibody that binds an epitope on the E1E2 complex and a second antibody that binds an epitope on the HCV E2 polypeptide. In some examples, the composition includes a cocktail of antibodies, e.g. at least two, three, four, five or more than five anti-HCV antibodies, at least two of which recognize different conformational neutralizing epitopes.

To prepare such a pharmaceutical composition, the polypeptide is obtained by, for example, expression in a host cell, and purified as necessary or desired and then lyophilized and stabilized. The polypeptide can then be adjusted to the appropriate concentration and then combined with other agent(s) or pharmaceutically acceptable carrier(s). A pharmaceutical formulation containing therapeutic amounts of one or more polypeptides, including antibodies, can be prepared by procedures known in the art using well-known and readily available ingredients. For example, one or more polypeptides can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents also can be included such as carboxymethyl cellulose, hydroxymethylcellulose, hydroxypropyl methylcellulose and other cellulose derivatives, alginates, gelatin, and polyvinyl-pyrrolidone.

Moisturizing agents can be included such as glycerol, disintegrating agents such as calcium carbonate and sodium bicarbonate. Agents for retarding dissolution also can be included such as paraffin. Resorption accelerators such as quaternary ammonium compounds also can be included. Surface active agents such as cetyl alcohol and glycerol monostearate can be included. Adsorptive carriers such as kaolin and bentonite can be added. Lubricants such as talc, calcium and magnesium stearate, and solid polyethyl glycols also can be included. Preservatives also can be added. The compositions also can contain thickening agents such as cellulose and/or cellulose derivatives. They also can contain gums such as xanthan, guar or carbo gum or gum arabic, or alternatively polyethylene glycols, bentones and montmorillonites, and the like.

For oral administration, one or more polypeptides, including antibodies, provided herein can be present as a powder, a granular formulation, a solution, a suspension, an emulsion or in a natural or synthetic polymer or resin for ingestion of the active ingredients from a chewing gum. The polypeptide also can be presented as a bolus, electuary or paste. The formulations can, where appropriate, be conveniently presented in discrete unit dosage forms and can be prepared by any of the methods well known to the pharmaceutical arts including the step of mixing the therapeutic agent with liquid carriers, solid matrices, semi-solid carriers, finely divided solid carriers or combinations thereof, and then, if necessary, introducing or shaping the product into the desired delivery system. The total active ingredients in such formulations comprise from 0.1 to 99.9% by weight of the formulation.

The polypeptides, including antibodies, provided herein also can be formulated as elixirs or solutions for convenient oral administration or as solutions appropriate for parenteral administration, for instance by intramuscular, subcutaneous, intraperitoneal or intravenous routes. A pharmaceutical formulation containing one or more polypeptides also can take the form of an aqueous or anhydrous solution or dispersion, or alternatively the form of an emulsion or suspension or salve.

Thus, one or more polypeptides, including antibodies, can be formulated for parenteral administration (e.g., by injection, for example, bolus injection or continuous infusion) and may be presented in unit dose form in ampoules, pre-filled syringes, small volume infusion containers or in multi-dose containers. As noted above, preservatives can be added to help maintain the shelf life of the dosage form. The polypeptides and other ingredients may form suspensions, solutions, or emulsions in oily or aqueous vehicles, and may contain formulatory agents such as suspending, stabilizing and/or dispersing agents. Alternatively, the polypeptides, including antibodies, and other ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable carriers, vehicles and adjuvants that are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol,” polyglycols and polyethylene glycols, C₁-C₄ alkyl esters of short-chain acids, ethyl or isopropyl lactate, fatty acid triglycerides such as the products marketed under the name “Miglyol,” isopropyl myristate, animal, mineral and vegetable oils and polysiloxanes.

It is possible to add, if necessary, an adjuvant chosen from antioxidants, surfactants, other preservatives, film-forming, keratolytic or comedolytic agents, perfumes, flavorings and colorings. Antioxidants such as t-butylhydroquinone, butylated hydroxyanisole, butylated hydroxytoluene and α-tocopherol and its derivatives can be added.

In some examples, the polypeptides provided herein, including antibodies, are formulated as a microbicide, which is administered topically or to mucosal surfaces such as the vagina, the rectum, eyes, nose and the mouth. For topical administration, the polypeptides may be formulated as is known in the art for direct application to a target area. Forms chiefly conditioned for topical application take the form, for example, of creams, milks, gels, dispersion or microemulsions, lotions thickened to a greater or lesser extent, impregnated pads, ointments or sticks, aerosol formulations (e.g., sprays or foams), soaps, detergents, lotions or cakes of soap. Thus, in one example, a polypeptide provided herein can be formulated as a vaginal cream or a microbicide to be applied topically. Other conventional forms for this purpose include wound dressings, coated bandages or other polymer coverings, ointments, creams, lotions, pastes, jellies, sprays, and aerosols. Thus, one or more polypeptides can be delivered via patches or bandages for dermal administration. Alternatively, the polypeptides can be formulated to be part of an adhesive polymer, such as polyacrylate or acrylate/vinyl acetate copolymer. For long-term applications it might be desirable to use microporous and/or breathable backing laminates, so hydration or maceration of the skin can be minimized. The backing layer can be any appropriate thickness that will provide the desired protective and support functions. A suitable thickness will generally be from about 10 to about 200 microns.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active peptides also can be delivered via iontophoresis, e.g., as disclosed in U.S. Pat. No. 4,140,122; 4,383,529; or 4,051,842. The percent by weight of one or more polypeptides or antibodies present in a topical formulation will depend on various factors, but generally will be from 0.01% to 95% of the total weight of the formulation, and typically 0.1-85% by weight.

Drops, such as eye drops or nose drops, can be formulated with one or more of the polypeptides, including antibodies, provided herein in an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The polypeptides can further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; and mouthwashes comprising the composition in a suitable liquid carrier.

The pharmaceutical formulations containing the anti-HCV polypeptides provided herein include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations include water and physiologically acceptable buffered saline solutions such as phosphate buffered saline solutions pH 7.0-8.0.

The polypeptides provided herein, including or antibodies or fragments thereof also can be administered to the respiratory tract. Thus, also provided are aerosol pharmaceutical formulations and dosage forms for use in the methods herein. In general, such dosage forms comprise an amount of at least one of the polypeptides or antibodies effective to treat or prevent the clinical symptoms of the viral infection. Any statistically significant attenuation of one or more symptoms of the infection that has been treated pursuant to the method herein is considered to be a treatment of such infection.

Alternatively, for administration by inhalation or insufflation, the composition can take the form of a dry powder, for example, a powder mix of one or more polypeptides or antibodies and a suitable powder base such as lactose or starch. The powder composition may be presented in unit dosage form in, for example, capsules or cartridges, or, e.g., gelatin or blister packs from which the powder may be administered with the aid of an inhalator, insufflator, or a metered-dose inhaler (see, for example, the pressurized metered dose inhaler (MDI) and the dry powder inhaler disclosed in Newman, S. P. in Aerosols and the Lung, Clarke, S. W. and Davia, D. eds., pp. 197-224, Butterworths, London, England, 1984).

The anti-HCV polypeptides, including antibodies or fragments thereof, also can be administered in an aqueous solution when administered in an aerosol or inhaled form. Thus, other aerosol pharmaceutical formulations can contain, for example, a physiologically acceptable buffered saline solution containing between about 0.1 mg/mL and about 100 mg/mL of one or more of the polypeptides or antibodies specific for the indication or disease to be treated. Dry aerosol in the form of finely divided solid polypeptide or antibody particles that are not dissolved or suspended in a liquid also can be useful. Polypeptides can be formulated as dusting powders and comprise finely divided particles having an average particle size of between about 1 and 5 μm, alternatively between 2 and 3 μm. Finely divided particles may be prepared by pulverization and screen filtration using techniques well known in the art. The particles may be administered by inhaling a predetermined quantity of the finely divided material, which can be in the form of a powder. It will be appreciated that the unit content of active ingredient or ingredients contained in an individual aerosol dose of each dosage form need not in itself constitute an effective amount for treating the particular infection, indication or disease since the necessary effective amount can be reached by administration of a plurality of dosage units. Moreover, the effective amount may be achieved using less than the dose in the dosage form, either individually, or in a series of administrations.

For administration to the upper (nasal) or lower respiratory tract by inhalation, the polypeptides can be delivered from a nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Nebulizers include, but are not limited to, those described in U.S. Pat. Nos. 4,624,251; 3,703,173; 3,561,444; and 4,635,627. Aerosol delivery systems of the type disclosed herein are available from numerous commercial sources including Fisons Corporation (Bedford, Mass.), Schering Corp. (Kenilworth, N.J.) and American Pharmoseal Co., (Valencia, Calif.). For intra-nasal administration, the therapeutic agent also can be administered via nose drops, a liquid spray, such as via a plastic bottle atomizer or metered-dose inhaler. Typical of atomizers are the Mistometer (Wintrop) and the Medihaler (Riker).

An exemplary formulation involves lyophilized polypeptides, including antibodies, and a separate pharmaceutical carrier. Immediately prior to administration, the formulation is constituted by combining the lyophilized polypeptides and pharmaceutical carrier. Administration by a parenteral or oral regimen will deliver the polypeptides or antibodies to the desired site of action. Pharmaceutical formulations of the polypeptides can be prepared as liquids, gels and suspensions. The formulations typically are suitable for injection, insertion or inhalation. Injection can be accomplished by needle, cannula, catheter and the like. Insertion can be accomplished by lavage, trochar, spiking, surgical placement and the like. Inhalation can be accomplished by aerosol, spray or mist formulation. The polypeptides, including antibodies, also can be administered topically such as to the epidermis, the buccal cavity and instillation into the ear, eye and nose. The polypeptides can be present in the pharmaceutical formulation at concentrations ranging from about 1 percent to about 50 percent, preferably about 1 percent to about 20 percent, more preferably about 2 percent to about 10 percent by weight relative to the total weight of the formulation.

The anti-HCV polypeptides provided herein, including antibodies and fragments thereof, also can be used in combination with one or more known therapeutic agents, for example, a pain reliever; an antiviral agent such as an anti-HBV, other anti-HCV (HCV inhibitor, HCV protease inhibitor) or an anti-herpetic agent; an antibacterial agent; an anti-cancer agent; an anti-inflammatory agent; an antihistamine; a bronchodilator and appropriate combinations thereof, whether for the conditions described or some other condition.

7. Miscellaneous Compositions and Articles of Manufacture

Provided are articles of manufacture that include a pharmaceutical composition containing one or more polypeptides or antibodies for controlling microbial infections. The articles can contain a therapeutically effective amount of a pharmaceutical composition for controlling or preventing HCV infection. The device can be packaged in a kit along with instructions for using the pharmaceutical composition for control of the infection. The pharmaceutical composition includes at least one polypeptide or antibody, in a therapeutically effective amount such that viral infection is controlled.

An article of manufacture can be a vessel or filtration unit that can be used for collection, processing or storage of a biological sample containing a polypeptide or antibody. The vessel may be evacuated. Vessels include, without limitation, a capillary tube, a vacutainer, a collection bag for blood or other body fluids, a cannula, a catheter. The filtration unit can be part of another device, for example, a catheter for collection of biological fluids. The polypeptides can be adsorbed onto or covalently attached to the article of manufacture, for example, a vessel or filtration unit. Thus, when material in the article is decanted therefrom or passed through, the material will not retain substantial amounts of the polypeptides. However, adsorption or covalent attachment of the polypeptides, including antibodies, to the article kills viruses or prevents their transmission, thereby helping to control viral infection. Thus, for example, the one or more polypeptides can be in filtration units integrated into biological collection catheters and vials, or added to collection vessels to remove or inactivate viral particles that may be present in the biological samples collected, thereby preventing transmission of the disease.

Provided are compositions containing one or more polypeptides provided herein, including one or more antibodies, and one or more clinically useful agents such as a biological stabilizer. Biological stabilizer includes, without limitation, an anticoagulant, a preservative and a protease inhibitor. Anticoagulants include, without limitation, oxalate, ethylene diamine tetraacetic acid, citrate and heparin. Preservatives include, without limitation, boric acid, sodium formate and sodium borate. Protease inhibitors include inhibitors of dipeptidyl peptidase IV. Compositions comprising a polypeptide and a biological stabilizer may be included in a collection vessel such as a capillary tube, a vacutainer, a collection bag for blood or other body fluids, a cannula, a catheter or any other container or vessel used for the collection, processing or storage of biological samples.

Also provided are compositions containing one or more polypeptides provided herein, including one or more antibodies, and a biological sample such as blood, semen or other body fluids that is to be analyzed in a laboratory or introduced into a recipient mammal. For example, the polypeptides can be mixed with blood prior to laboratory processing and/or transfusions. The polypeptides can be present in at least about 0.15 mg/mL of the sample, e.g. 0.16 mg/mL, 0.17 mg/mL, 0.18 mg/mL, 0.19 mg/mL, 0.2 mg/mL, 0.22 mg/mL, 0.24 mg/mL, 0.25 mg/mL, 0.27 mg/mL, 0.3 mg/mL, 0.35 mg/mL, 0.4 mg/mL or more than 0.4 mg/mL of the sample.

In some examples, polypeptides provided herein, including antibodies, can be included in physiological media used to store and transport biological tissues, including transplantation tissues. Thus, for example, liver, heart, kidney and other tissues can be bathed in media containing the polypeptides to inhibit viral transmission to transplant recipients. In this case, the one or more polypeptides or antibodies is present in at least about 1.5 mg/kg of the sample, e.g. 1.6 mg/kg, 1.7 mg/kg, 1.8 mg/kg, 1.9 mg/kg, 2 mg/kg, 2.2 mg/kg, 2.4 mg/kg, 2.5 mg/kg, 2.7 mg/kg, 2.8 mg/kg, 2.9 mg/kg, 3 mg/kg, 3.5 mg/kg, 4 mg/kg or more than 4 mg/kg.

### D. MODIFIED E1 AND E2 POLYPEPTIDES

Provided are modified E1 and E2 polypeptides. In some instances, the modified E1 and E2 polypeptides, when expressed as an E1E2 complex on the surface of HCV or a virus-like particle, can result in increased infectivity of the virus or virus-like particle compared to one expressing unmodified E1 or E2 polypeptides. Few HCV isolates, including the JFH-1 isolate and chimeric viruses constructed using JFH-1 as a backbone and structural proteins from other isolates, (also known as HCVcc) or HCV E1E2 envelope glycoprotein complexes cloned from human samples for the generation of HCV pseudotype particles (also known as HCVpp) can produce infectious virions for in vitro assays, such as antibody neutralization assays and anti-viral drug screening. Mutations that can enhance viral infectivity can, therefore, be of particular use for the construction of recombinant HCVcc or HCVpp to increase their infectivity in vitro and possibly in vivo. Thus, the modified E1 and E2 polypeptides provided herein can be used to generate genetically diverse, infectious HCV, including HCVcc and HCVpp, which can be used, for example, in infectivity assays, such as for screening anti-HCV antibodies or other anti-viral agents. The modified E1 and E2 polypeptides provided herein also can be used to generate functional recombinant E1E2 complexes, which can be used, for example, for screening anti-HCV antibodies and other anti-HCV agents, including polypeptides, peptides and drugs.

Modifications in an E1 or E2 polypeptide can be made to any form of an E1 or E2 polypeptide known in the art, including any E1 or E2 polypeptide from any HCV isolate. For example, the modifications provided herein can be made in the E1 or E2 regions within the HCV polyprotein, or in E1 or E2 polypeptides that are not part of the HCV polyprotein, such as following posttranslational cleavage or by recombinant expression of the E1 or E2 polypeptides alone or as part of the E1E2 complex. For example, modifications in an E1 polypeptide can be made in any polypeptide that contains an E1 polypeptide from any HCV isolate, variant or fragment thereof that has 40%, 50%, 60%, 70%, 80%, 90%, 91%, 92%, 93%, 94%, 95%, 96%, 97%, 98%, or 99% sequence identity to the E1 polypeptide set forth in SEQ ID NO: 1337. Modifications in an E2 polypeptide can be made in any polypeptide that contains an E2 polypeptide from any HCV isolate, variant or fragment thereof that has 40%, 50%, 60%, 70%, 80%, 90%, 91%, 92%, 93%, 94%, 95%, 96%, 97%, 98%, or 99% sequence identity to the E2 polypeptide set forth in SEQ ID NO: 1312. Modifications in an E1 or E2 polypeptide can be made to an E1 or E2 polypeptide that also contains other modifications, such as those described in the art, including modifications of the primary sequence and modifications not in the primary sequence of the polypeptide. For example, modifications can be made in truncated E1 or E2 polypeptides, such as those that lack the C-terminal transmembrane domain. Thus, provided herein are soluble modified E1 and E2 polypeptides. In another example, the modifications to an E2 polypeptide are made in an E2 polypeptide that lacks all or a portion of the N-terminal hypervariable region, such as all or a portion of amino acid residues 384 to 411.

Modifications provided herein of a starting, unmodified reference polypeptide include amino acid replacements or substitution, additions or deletions of amino acids, or any combination thereof. For example, modified E1 and modified E2 polypeptides include those with 1, 2, 3, 4, 5, 6, 7, 8, 9, 10, 11, 12, 13, 14, 15, 16, 17, 18, 19, 20, 30, 40, 50 or more modified positions. Any modification provided herein can be combined with any other modification known to one of skill in the art. Typically, the resulting modified E1 or E2 polypeptide retains the ability to form an E1E2 complex. When expressed as part of HCV or a virus-like particle, the virus or virus like particle typically retains its ability to infect a host cell. In some instances, the virus or virus like particle containing the modified E1 and/or E2 polypeptide exhibits increased infectivity compared to a virus or virus like particle containing an unmodified E1 or E2 polypeptide.

The modifications provided herein can be made by standard recombinant DNA techniques such as are routine to one of skill in the art. Any method known in the art to effect mutation of any one or more amino acids in a target protein can be employed. Methods include standard site-directed mutagenesis (using e.g., a kit, such as QuikChange available from Stratagene) of encoding nucleic acid molecules, or by solid phase polypeptide synthesis methods. Other modifications that are or are not in the primary sequence of the polypeptide also can be included in a modified E1 or E2 polypeptide, or conjugate thereof, including, but not limited to, the addition of a carbohydrate moiety, the addition of a polyethylene glycol (PEG) moiety, the addition of an Fc domain, etc. For example, such additional modifications can be made to increase the stability or half-life of the protein.

1. Exemplary E1 Modifications

Provided are modified E1 polypeptides, including modified E1 polypeptides that form an E1E2 complex with an E2 polypeptide. In some examples, the modified E1 polypeptides are expressed on the surface of a virus, such as HCV, or a virus-like particle. The resulting virus or virus-like particle can exhibit altered infectivity compared to a virus or virus-like particle that expresses an unmodified E1 polypeptide. In some examples, the virus or virus-like particle that expresses a modified E1 polypeptide exhibits increased infectivity compared to a virus or virus-like particle that expresses an unmodified E1 polypeptide. The increased infectivity of the virus or virus-like particle that expresses a modified E1 polypeptide can be increased by at least or about 1%, 2%, 3%, 4%, 5%, 6%, 7%, 8%, 9%, 10%, 20%, 30%, 40%, 50%, 60%, 70%, 80%, 90%, 100%, 110%, 120%, 130%, 140%, 150%, 160%, 170%, 180%, 190%, 200%, 300%, 400%, 500%, or more compared to the infectivity of the virus or virus-like particle that expresses an unmodified E1 polypeptide. In some examples, the virus or virus-like particle that expresses a modified E1 polypeptide exhibits decreased infectivity compared to a virus or virus-like particle that expresses an unmodified E1 polypeptide. The decreased infectivity of the virus or virus-like particle that expresses the modified E1 polypeptide can be decreased by at least or about 1%, 2%, 3%, 4%, 5%, 6%, 7%, 8%, 9%, 10%, 20%, 30%, 40%, 50%, 60%, 70%, 80%, 90%, or more compared to the infectivity of the virus or virus-like particle that expresses an unmodified E1 polypeptide.

Exemplary of the modified E1 polypeptides are those E1 polypeptides that contain a modification at amino acid position 192, 193, 194, 195, 196, 198, 201, 204, 205, 206, 209, 211, 212, 221, 222, 224, 225, 227, 228, 234, 236, 239, 244, 246, 255, 258, 259, 260, 261, 263, 266, 269, 273, 274, 276, 278, 279, 282, 289, 291, 292, 293, 294, 295, 296, 298, 302, 305, 307, 309, 311, 314, 315, 316, 317, 318, 319, 320, 321, 322, 323, 324, 326, 327, 328, 329, 339, 341, 346, 350, 352, 353 or 354, with numbers corresponding to the HCV polypeptide set forth in SEQ ID NO:607.

As is customary, amino acid positions of mature HCV proteins, such as E1, are numbered relative to the HCV polypeptide, such as that set forth in SEQ ID NO:607, not the mature polypeptide. Thus, for example, above-referenced amino acid position 192 corresponds to position 1 of the mature E1 polypeptide from HCV genotype 1a H77 with a sequence set forth in SEQ ID NO:1337. Similarly, above-referenced position 193, 194, 195, 196, 198, 201, 204, 205, 206, 209, 211, 212, 221, 222, 224, 225, 227, 228, 234, 236, 239, 244, 246, 255, 258, 259, 260, 261, 263, 266, 269, 273, 274, 276, 278, 279, 282, 289, 291, 292, 293, 294, 295, 296, 298, 302, 305, 307, 309, 311, 314, 315, 316, 317, 318, 319, 320, 321, 322, 323, 324, 326, 327, 328, 329, 339, 341, 346, 350, 352, 353 and 354 correspond to positions 2, 3, 4, 7, 10, 13, 14, 15, 18, 20, 21, 30, 31, 33, 34, 36, 43, 44, 48, 53, 55, 64, 67, 68, 69, 70, 72, 75, 78, 82, 83, 85, 87, 88, 91, 98, 100, 101, 102, 103, 104, 105, 107, 111, 114, 115, 118, 120, 123, 124, 125, 126, 127, 128, 129, 130, 131, 132, 133, 135, 136, 137, 138, 148, 150, 155, 159, 161, 162, and 163 of the mature E1 polypeptide from HCV genotype 1a H77 with a sequence set forth in SEQ ID NO:1337.

These amino acid positions can be modified such as by amino acid replacement, deletion or substitution. The residues at these positions can be replaced or substituted with any another amino acid, such as, for example, tyrosine (Tyr, Y), glycine (Gly, G), phenylalanine (Phe, F), methionine (Met, M), alanine (Ala, A), serine (Ser, S), isoleucine (Ile, I), leucine (Leu, L), threonine (Thr, T), valine (Val, V), proline (Pro, P), lysine (Lys, K), histidine (His, H), glutamine (Gln, Q), glutamic acid (Glu, E), tryptophan (Tip, W), arginine (Arg, R), aspartic acid (Asp, D), asparagine (Asn, N) or cysteine (Cys, C). In one example, the residue at amino acid position 192, 193, 194, 195, 196, 198, 201, 204, 205, 206, 209, 211, 212, 221, 222, 224, 225, 227, 228, 234, 236, 239, 244, 246, 255, 258, 259, 260, 261, 263, 266, 273, 276, 278, 279, 282, 289, 291, 292, 293, 294, 295, 296, 298, 302, 305, 307, 309, 311, 314, 315, 316, 317, 318, 320, 321, 322, 323, 324, 326, 327, 328, 329, 339, 341, 346, 350, 352, 353 or 354 (with numbering relative to the polyprotein set forth in SEQ ID NO:607) is replaced with an alanine (Ala, A). In another example, the amino acid residue at position 269, 274 or 319 is replaced with a glycine (Gly, G). Combination mutants in which amino acid replacements are made at more than one of the above-identified positions in an E1 polypeptide also can be generated. The modified E1 polypeptide can contain 1, 2, 3, 4, 5, 6, 7, 8, 9, 10 or more modifications. Included among such combination mutants are those having two or more mutations at amino acid positions 196, 209, 234, 305 and 325. The modifications can be made in any E1 polypeptide, including, but not limited to, the E1 polypeptides having a sequence of amino acids set forth in any of SEQ ID NOS:1337-1361. Table 7 provides non-limiting examples of exemplary amino acid replacements at the identified positions, and provides the sequence identifier that sets forth the amino acid sequence of the resulting modified E1 polypeptide.

TABLE 7
E1 mutants

| Mutation | SEQ ID NO |
|---|---|
| xN196A/xN209A | 1206 |
| xN196A/xN234A | 1207 |
| xN196A/xN305A | 1208 |
| xN196A/xN325A | 1209 |
| xN305A/xN209A | 1210 |
| xN305A/xN234A | 1211 |
| xN305A/xN325A | 1212 |
| Y192A | 1213 |
| Q193A | 1214 |
| V194A | 1215 |
| R195A | 1216 |
| xN196A | 1217 |
| S198A | 1218 |
| Y201A | 1219 |
| T204A | 1220 |
| N205A | 1221 |
| D206A | 1222 |
| xN209A | 1223 |
| S211A | 1224 |
| I212A | 1225 |
| L221A | 1226 |
| H222A | 1227 |
| P224A | 1228 |
| G225A | 1229 |
| V227A | 1230 |
| P228A | 1231 |
| xN234A | 1232 |
| S236A | 1233 |
| W239A | 1234 |
| P244A | 1235 |
| V246A | 1236 |
| T255A | 1237 |
| L258A | 1238 |
| R259A | 1239 |
| H261A | 1240 |
| D263A | 1241 |
| V266A | 1242 |
| A269G | 1243 |
| S273A | 1244 |
| A274G | 1245 |
| Y276A | 1246 |
| G278A | 1247 |
| D279A | 1248 |
| G282A | 1249 |
| Q289A | 1250 |
| F291A | 1251 |
| T292A | 1252 |
| F293A | 1253 |
| S294A | 1254 |
| P295A | 1255 |
| R296A | 1256 |
| H298A | 1257 |
| Q302A | 1258 |
| xN305A | 1259 |
| S307A | 1260 |
| Y309A | 1261 |
| G311A | 1262 |
| T314A | 1263 |
| G315A | 1264 |
| H316A | 1265 |
| R317A | 1266 |
| M318A | 1267 |
| A319G | 1268 |
| W320A | 1269 |
| D321A | 1270 |
| M322A | 1271 |
| M323A | 1272 |
| M324A | 1273 |
| xN325A | 1274 |
| W326A | 1275 |
| S327A | 1276 |
| P328A | 1277 |
| T329A | 1278 |
| R339A | 1279 |
| P341A | 1280 |
| D346A | 1281 |
| G350A | 1282 |
| H352A | 1283 |
| W353A | 1284 |
| G354A | 1285 |

In some examples, a virus or virus-like particle, such as HCVcc or HCVpp, that expresses a modified E1 polypeptide exhibits increased infectivity compared to a virus or virus-like particle that expresses an unmodified E1 polypeptide. The increased infectivity of the virus or virus-like particle that expresses a modified E1 polypeptide can be increased by at least or about 1%, 2%, 3%, 4%, 5%, 6%, 7%, 8%, 9%, 10%, 20%, 30%, 40%, 50%, 60%, 70%, 80%, 90%, 100%, 110%, 120%, 130%, 140%, 150%, 160%, 170%, 180%, 190%, 200%, 300%, 400%, 500%, or more compared to the infectivity of the virus or virus-like particle that expresses an unmodified E1 polypeptide. Exemplary of modified E1 polypeptides that can be expressed by a virion, resulting in increased infectivity of the virion compared to one that expresses an unmodified E1 polypeptide, are polypeptides containing a modification at an amino acid position corresponding to amino acid positions 192, 194, 195, 224, 227, 255, 293, 294, 295, 314, 316, 317, 324, or 325 (with amino acid numbering relative to the HCV polyprotein set forth in SEQ ID NO:607). Exemplary modified E1 polypeptides include those with a modification corresponding to Y192A, V194A, R195A, P224A, V227A, T255A, F293A, S294A, P295A, T314A, H316A, R317A, M324A or N325A, including a modified E1 polypeptide with a sequence set forth in any of SEQ ID NOS:1213, 1215, 1216, 1228, 1230, 1237, 1253, 1254, 1255, 1263, 1265, 1266, 1273 and 1274, respectively. The modified E1 polypeptides provided herein can be used to generate recombinant HCVcc or HCVpp that infect host cells at higher infectivity than recombinant HCVcc or HCVpp that contain an unmodified E1 polypeptide. (see, e.g. Examples 4 and 6). In some examples, the virus or virus-like particles containing the modified E1 polypeptides also can exhibit increased resistance to antibody neutralization.

2. Exemplary E2 Modifications

Provided are modified E2 polypeptides, including modified E2 polypeptides that form an E1E2 complex with an E1 polypeptide. In some examples, the modified E2 polypeptides are expressed on the surface of a virus, such as HCV, or a virus-like particle. The resulting virus or virus-like particle can exhibit altered infectivity compared to a virus or virus-like particle that expresses an unmodified E2 polypeptide. In some examples, the virus or virus-like particle that expresses a modified E2 polypeptide exhibits increased infectivity compared to a virus or virus-like particle that expresses an unmodified E2 polypeptide. In other examples, the virus or virus-like particle that expresses a modified E2 polypeptide exhibits decreased infectivity compared to a virus or virus-like particle that expresses an unmodified E2 polypeptide.

Exemplary of the modified E2 polypeptides are those that contain a modification at an amino acid position corresponding to amino acid position 425, 427, 428, 435, 436, 439, 441, 442, 443, 447, 448, 614, 617, 618, 619, 621, 623, 624, 630, 634, 635, 637, 638, 639, 642 or 643, with numbering relative to the HCV polyprotein set forth in SEQ ID NO:607. As noted above, amino acid positions of mature HCV proteins, such as E2, are numbered relative to the HCV polypeptide, such as that set forth in SEQ ID NO:607, not the mature polypeptide. Thus, for example, above-referenced amino acid position 425 corresponds to position 42 of the mature E2 polypeptide from HCV genotype 1a H77 with a sequence set forth in SEQ ID NO:1312.

These amino acid positions can be modified such as by amino acid replacement, deletion or substitution. The residues at these positions can be replaced or substituted with any other amino acid, such as, for example, tyrosine (Tyr, Y), glycine (Gly, G), phenylalanine (Phe, F), methionine (Met, M), alanine (Ala, A), serine (Ser, S), isoleucine (Ile, I), leucine (Leu, L), threonine (Thr, T), valine (Val, V), proline (Pro, P), lysine (Lys, K), histidine (His, H), glutamine (Gln, Q), glutamic acid (Glu, E), tryptophan (Trp, W), arginine (Arg, R), aspartic acid (Asp, D), asparagine (Asn, N) or cysteine (Cys, C). In one example, the residue at amino acid position 425, 427, 428, 435, 436, 441, 442, 443, 447, 448, 614, 617, 618, 619, 621, 623, 624, 630, 634, 635, 637, 638 or 639 is replaced with an alanine (Ala, A). In another example, the amino acid residue at position 439, 642 or 643 is replaced with a glycine (Gly, G). Combination mutants in which amino acid replacements are made at more than one of the above-identified positions in an E2 polypeptide also can be generated. The modified E2 polypeptide can contain 1, 2, 3, 4, 5, 6, 7, 8, 9, 10 or more modifications. The modifications can be made in any E2 polypeptide, including, but not limited to, the E2 polypeptides having a sequence of amino acids set forth in any of SEQ ID NOS:1312-1336. Table 8 provides non-limiting examples of exemplary amino acid replacements at the identified positions, and provides the sequence identifier that sets forth the amino acid sequence of the resulting modified E2 polypeptide.

TABLE 8
Additional E2 mutants

| Mutation | SEQ ID NO |
|---|---|
| T425A | 1286 |
| L427A | 1287 |
| N428A | 1288 |
| T435A | 1289 |
| G436A | 1290 |
| A439A | 1291 |
| L441A | 1292 |
| F442A | 1293 |
| Y443A | 1294 |
| F447A | 1295 |
| N448A | 1296 |
| R614A | 1297 |
| R617A | 1298 |
| Y618A | 1299 |
| P619A | 1300 |
| T621A | 1301 |
| N623A | 1302 |
| Y624A | 1303 |
| R630A | 1304 |
| G634A | 1305 |
| G635A | 1306 |
| E637A | 1307 |
| H638A | 1308 |
| R639A | 1309 |
| A642G | 1310 |
| A643G | 1311 |

3. Production of the Modified E1 and E2 Polypeptides

The modified E1 and E2 polypeptides provided herein can be obtained by methods well known in the art for protein purification and recombinant protein expression. Any method known to those of skill in the art for identification of nucleic acids that encode desired genes can be used to obtain the nucleic acids encoding E1 and E2. Any method available in the art can be used to obtain a nucleic acid encoding an E1 or E2 polypeptide. Modified E1 and E2 polypeptides can be engineered as described herein, such as by site-directed mutagenesis. The modified E1 and E2 polypeptide can be expressed alone, as part of an E1E2 complex, or on a hepatitis C virus, such as HCVcc or HCVpp.

E1 and E2 polypeptides can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Methods for amplification of nucleic acids can be used to isolate nucleic acid molecules encoding a polypeptide, including for example, polymerase chain reaction (PCR) methods. A nucleic acid containing material can be used as a starting material from which an E1- or E2-encoding nucleic acid molecule can be isolated. For example, tissue extracts (e.g. from liver) or fluid samples (e.g. blood, serum, saliva) from a subject infected with hepatitis C virus can be used in amplification methods. Nucleic acid libraries also can be used as a source of starting material. Primers can be designed to amplify an E1- or E2-encoding molecule. For example, primers can be designed based on expressed sequences from which an E1 or E2 polypeptide is generated. Primers can be designed based on back-translation of an E1 or E2 amino acid sequence. Nucleic acid molecules generated by amplification can be sequenced and confirmed to encode the E1 or E2 polypeptide. Additional nucleotide sequences can be joined to an E1- or E2-encoding nucleic acid molecule, including linker sequences containing restriction endonuclease sites for the purpose of cloning the synthetic gene into a vector, for example, a protein expression vector or a vector designed for the amplification of the DNA sequences.

The identified and isolated nucleic acids can then be inserted into an appropriate cloning vector. Vectors and compatible expression systems are discussed above and can be used to produce the modified E1 and E2 polypeptides provided herein. Methods of polypeptide purification also are discussed above, and can be similarly used to purify the modified E1 or modified E2 polypeptides. The modified E1 and E2 polypeptide can be expressed and purified alone (i.e. as recombinant E1 or E2 polypeptides), as part of an E1E2 complex, or can be expressed on a hepatitis C virus, such as HCVcc or HCVpp. The modified E1 and E2 polypeptides also can contain additional modifications, as described in detail above, such as pegylation. While the methods discussed above for producing and purifying polypeptides can be used to produce the modified E1 or modified E2 polypeptides provided herein, the discussion below includes particular exemplary methods for producing these polypeptides.

a. Nucleic Acids Encoding Modified E1 and/or E2

Provided herein are nucleic acids encoding modified E1 polypeptides or modified E2 polypeptides. Nucleic acids encoding modified E1 polypeptides or modified E2 polypeptides can be generated from nucleic acids encoding the naturally-occurring HCV polyprotein using methods known to those of skill in the art. For example, nucleic acids encoding modified E1 polypeptides or modified E2 polypeptides containing various amino acid substitutions can be produced by site-specific mutagenesis and polymerase chain reaction (PCR) amplification from the nucleic acids encoding the naturally-occurring HCV polyprotein. Nucleic acid sequences encoding the naturally-occurring HCV polyproteins are well known in the art, and include, for example, those set forth in Genbank Accession AF009606; D10749; U01214; AY051292; AY746460; AY232731; D50409; DQ155561; AB031663; DQ437509; D49374; D63821; Y11604; DQ516083; EF589160; AF064490; AY859526; NC009827; EF420130; DQ314805; DQ835764; D63822; D84264; DQ835763; and DQ278894.

Methods for isolating nucleic acids encoding the naturally-occurring HCV polyprotein, as well as technologies for generation of nucleic acids encoding E1 or E2 polypeptides are known to those of skill in the art. See for example, Current Protocols in Molecular Biology, Ausubel et al. edts. (John Wiley & Sons, Inc., 1999) or Molecular Cloning: A Laboratory Manual, Sambrook et al. (Cold Spring Harbor Laboratory Press, New York, 1989).

In some examples, nucleic acid encoding only E1 or E2 are used to produce the modified E1 or E2 polypeptides provided herein. In other examples, nucleic acid molecules encoding both E1 and E2 are used, such that an E1E2 complex is produced upon expression. Exemplary of a nucleic acid molecule encoding unmodified E1E2 is set forth in SEQ ID NO:985. Example 1, below, describes the cloning of nucleic acid encoding E1E2 from HCV-infected human serum. Such nucleic acid can then be modified using methods well known in the art, such as site directed mutagenesis, to produce nucleic acid encoding modified E1 and/or modified E2. In some example, only the nucleic acid encoding E1 is modified. In other examples, only the nucleic acid encoding E2 is modified. In further example, the nucleic acids encoding both E1 and E2 are modified.

b. Vectors and Expression Systems

Nucleic acid encoding a the modified E1 and/or modified E2 polypeptides can be operably-linked to an expression control sequence in an expression vector, which can be introduced into a host cell for expression of the encoded polypeptide or administered to a mammal to elicit an immune response against the polypeptide. The expression vector also can include other sequences such as enhancer sequences, synthetic introns, adenovirus tripartite leader (TPL) sequences and modified polyadenylation and transcriptional termination sequences, e.g. bovine growth hormone or rabbit beta-globulin polyadenylation sequences, to improve expression of the nucleic acid encoding the polypeptides.

Nucleic acids encoding modified E1 and/or modified E2 polypeptides can be incorporated into viral, bacterial, insect, yeast or mammalian expression vectors. As such, nucleic acids encoding modified E1 and/or modified E2 polypeptides can be operably-linked to expression control sequences such as viral, bacterial, insect, yeast or mammalian promoters and enhancers. Examples of expression control sequences such as enhancers and promoters include viral promoters such as SV 40 promoter, Rous Sarcoma Virus (RSV) promoter, and cytomegalovirus (CMV) immediate early promoter. Examples of viral vectors include retrovirus-based vectors, e.g. Lentiviruses, Adenoviruses and Adeno-associated viruses.

In a particular example, nucleic acid encoding E1E2 is cloned into the pcDNA3.1/V5-His TOPO TA vector, which facilitates both bacterial and mammalian expression. DNA sequencing of nucleic acid from bacterial clones can be performed to confirm the correct nucleic acid sequence, then the same vectors can be transfected into a suitable mammalian cell, such as 293T cells, for expression. Because E1 and E2 are both glycoproteins, expression typically is performed in cells that facilitate correct glycosylation of the polypeptides, which itself aides correct folding of the polypeptides. Exemplary host cells that facilitate glycosylation of recombinant polypeptides are provided above, and include, for example, mammalian cells, such as 293T cells and Huh7 cells. In some examples, the nucleic acid encoding the E1, E2 or E1E2 is introduced into a viral vector, such as, for example, a vaccinia vector. Such vectors can be introduced into mammalian cells for expression of the encoded polypeptides (see, e.g. Example 1).

The nucleic acid encoding modified E1 and/or modified E2 polypeptides also can be linked to nucleic acid sequences that code for unrelated amino acid sequences such as N-terminal ubiquitin signals to improve antigen targeting, a poly-histidine sequence, a FLAG (DYKDDDDK) sequence (SEQ ID NO:685), an HA sequence (SEQ ID NO:687), a myc sequence (SEQ ID NO:688), a V5 sequence (SEQ ID NO:984), a chitin binding protein sequence, a maltose binding protein sequence or a glutathione-S-transferase sequence.

Expression vectors containing nucleic acids encoding modified E1 and/or modified E2 polypeptides can be introduced into bacterial, insect, yeast or mammalian host cells for expression using conventional methods including, without limitation, transformation, transduction and transfection.

c. Purification and Assessment

When expressed in bacterial, yeast, insect or mammalian host cells, modified E1 and/or modified E2 polypeptides can be purified using a variety of methods, including those discussed above in section C.3.d. In some examples, modified E1, E2 or E1E2 complexes are purified by affinity chromatography using appropriate antibodies that bind to these polypeptides, such as, for example, anti-HCV antibodies AR3A, AR3B, AR3C, AR3D, N4 or V1 in combination with size exclusion chromatography. For example, modified E1 and/or modified E2 polypeptides can be separated from unrelated proteins by affinity chromatography using an appropriate binding antibody. The polypeptide can be eluted at acidic, neutral or basic pH using: (1) 0.2M glycine pH 2.2, (2) 2M sodium thiocyanate (pH adjusted to pH 7.4 with 50 mM Tris-HCl); or (3) 0.2M glycine pH 11.5, and then further purified by size-exclusion chromatography (see e.g., U.S. patent application Ser. No. 12/290,017). Because the above described antibodies recognize conformational epitopes, E1, E2 and/or E1E2 that are properly folded can be isolated.

Similar methods can be used to determine whether the purified, modified polypeptide or E1E2 complex is correctly folded. Included among the anti-HCV antibodies provided herein are those that recognize conformational epitopes on E1 and E2 and on the E1E2 complex. As described in Example 4, modified E1 and E2 polypeptides expressed as an E1E2 complex can be characterized using the conformational antibodies. If an antibody that binds a conformational epitope on an unmodified E1E2 does not bind the modified E1E2 complex, then the modification is one that affects the correct folding of the polypeptide. In another example, the modified E1 or E2 can be expressed on a hepatitis C virus, such as HCVcc or HCVpp, and the infectivity of this virus can be compared to the infectivity of a virus that contains an unmodified E1 or E2, respectively (see e.g., Example 4). The effect of the modification on the ability of the virus to infect and replicate in host cells, such as Huh7 or 293 cells, can then be assessed.

4. Uses for the Modified E1 and E2 Polypeptides.

The modified E1 and E2 polypeptides provided herein can be used, for example, to make genetically diverse infectious HCV for different infectivity assays and also to make highly functional recombinant HCV E1E2 complexes for antibody and drug screening. The modified E1 or E2 polypeptides provided herein can be used to generate HCVcc or HCVpp. Thus, provided herein are hepatitis C viruses that express the modified E1 and/or E2 polypeptides provided herein. In some examples, the hepatitis C virus expressing the modified E1 or E2 polypeptides exhibit increased infectivity compared to the hepatitis C virus expressing the unmodified E1 or E2 polypeptides, respectively. These HCV can be of particular use, for example, in assays, such as neutralization assays, to assess anti-viral agents. The anti-viral agents include, but are not limited to, polypeptides, including anti-HCV antibodies, interferons, cytokines and toxins, small molecules and drugs, and antisense molecules. Methods for performing these assays are well known in the art, and typically include contacting the virus with the anti-viral agent, then infecting a suitable host cell, such as Huh7 or 293T cells. In some examples, the cells are infected with the HCV before being contacted with the anti-viral agent. In other examples, the cells are infected after the virus is incubated with the anti-viral agent. In some examples, a combination of anti-viral agents are tested. For example, any one or more of the anti E2 or E1E2 antibodies provided herein can be assessed using neutralization assays that utilize HCV containing the modified E1 or E2 polypeptides provided herein. In other examples, other anti-HCV antibodies also are included, such as in combination with the anti E2 or E1E2 antibodies provided herein.

5. Articles of Manufacture and Kits

Modified E1 or E2 polypeptides, E1E2 complexes containing the modified E1 or E2 polypeptides, HCV containing the modified E1 or E2 polypeptides, or encoding nucleic acids thereof, can be packaged as articles of manufacture. The articles of manufacture provided herein can contain packaging materials. Packaging materials for use in packaging pharmaceutical products are well known to those of skill in the art. See, for example, U.S. Pat. Nos. 5,323,907, 5,052,558 and 5,033,352, each of which is incorporated herein in its entirety. Examples of packaging materials include, but are not limited to, blister packs, bottles, tubes, inhalers, pumps, bags, vials, containers, syringes, bottles, and any packaging material suitable for a selected formulation and intended mode of administration and treatment.

Modified E1 or E2 polypeptides, E1E2 complexes containing the modified E1 or E2 polypeptides, HCV containing the modified E1 or E2 polypeptides, or encoding nucleic acids thereof, also can be provided as kits. Kits can include one or more modified E1 or E2 polypeptides, E1E2 complexes containing the modified E1 or E2 polypeptides or HCV virions containing the modified E1 or E2 polypeptides. The kit can, optionally, include instructions for performing an assay, such as a neutralization assay. Also included in the kits can be, optionally, buffers, labels, dyes, tubes or microwell plates.

### E. EXAMPLES

The following examples are included for illustrative purposes only and are not intended to limit the scope of the invention.

### Example 1

### Materials and Methods

This Example describes some of the procedures and materials used in developing the invention.

Cells, Antibodies and Viruses.

Huh-7 (Zhong, J. et al. Proc. Natl. Acad. Sci. U.S.A. 102, 9294-9299 (2005)) and 293T cells were grown in Dulbecco's Modified Eagle Medium (D-MEM) supplemented with 10% fetal calf serum (FCS) (Invitrogen). The human MAbs CBH-2, CBH-5, CBH-4B and CBH-7, Mouse MAbs A4, H53, AP33, AP320 and ALP98, and rat MAbs 7/59, 9/27, 3/11, 1/39, 2/69A, 7/16B, 11/20, 9/75 and 6/53 have been described elsewhere. Keck, Z. Y. et al. J. Virol. 78, 9224-9232 (2004); Keck, Z. Y. et al. J. Virol. 81, 1043-1047 (2007); Keck, Z. Y. et al. J. Virol. 79, 13199-13208 (2005); Dubuisson, J. et al. J. Virol. 68, 6147-6160 (1994); Clayton, R. F. et al. J. Virol. 76, 7672-7682 (2002); Deleersnyder, V. et al. J. Virol, 71, 697-704 (1997); Owsianka, A. et al. J. Virol. 79, 11095-11104 (2005); Tarr, A. W. et al. Hepatology 43, 592-601 (2006); Flint, M. et al. J. Virol. 73, 6235-6244 (1999); Hsu, M. et al. Proc. Natl. Acad. Sci. U.S.A. 100, 7271-7276 (2003); Maruyama, T. et al. Am. J. Pathol. 165, 53-61 (2004). The panel of linear epitope-specific MAbs covers known linear regions. The generation of HCVpp has been described below.

Phage Display Antibody Library Construction.

In a study of autoantibodies in patients with Sjögren's syndrome, bone marrow mononuclear cell RNA from a 35-year-old female patient with Sjögren's syndrome and chronic HCV infection was used as source material for an IgG1 Fab phage display library (Maruyama, T. et al. Am. J. Pathol. 165, 53-61 (2004)). The donor was diagnosed with HCV in 1991 and developed mixed cryoglobulinemia, symptoms of Sjögren's syndrome and tested positive for antinuclear antibody in 1994. The donor was treated with interferon-α with initial decrease in viral load but the treatment was stopped due to severe drop in platelet count (idiopathic thrombocytopenic purpura). Bone marrow samples were collected for the evaluation of neutropenia as an outpatient clinical procedure at Scripps Clinic. After meeting the needs of clinical pathology, a fraction of the biopsy was used to construct the antibody library. The human subjects protocol was approved by the Human Subjects Committee for General Clinical Research Center of Scripps Clinic and informed consent was obtained from the donor. Due to subsequent relapse of HCV, the donor underwent a liver transplant in 2000 and has been maintained on anti-rejection medications since. The viral genotype in this donor was not determined at the time of tissue donation but was found to be genotype 1a seven years later. The bone marrow (˜2 ml) was separated by Histopaque-1077 gradient (Sigma-Aldrich) and RNA was extracted from mononuclear cells (7×10⁷ cells) homogenized in 10 mL of TRI reagent (Sigma-Aldrich). First-strand cDNA was synthesized using SuperScript First-Strand Synthesis Kit (Invitrogen), and the light chain and IgG1 heavy chain fragments were amplified by PCR using gene-specific primers and were sequentially cloned into the SacI/XbaI and XhoI/SpeI sites of a phagemid vector, pComb3H, as described previously (Maruyama, T. et al. J. Infect. Dis. 179 Suppl 1, S235-239 (1999)). The Fab heavy chains were expressed as a fusion protein with the phage gene III surface protein for display. The library was amplified in XL-1 Blue cells (Stratagene) using 0.3% SeaPrep agarose (BioWhittaker) in SuperBroth (SB) Medium by a semi-solid phase amplification method.

Library Panning on HCV E2 Glycoprotein

The phagemid library was transformed into E. coli (XL-1 Blue) (Stratagene) by electroporation and the phage was propagated overnight with VCS-M13 helper phage (Stratagene). Recombinant E2 glycoprotein (genotype 1a, amino acids 388-644; Lesniewski, R. et al. J. Med. Virol. 45, 415-422 (1995)) was coated directly onto a microtiter plate overnight at 4° C. (Costar). The wells were washed and then blocked with 4% non-fat dry milk in phosphate-buffered saline (PBS). The phage library was added to the wells and incubated for 1-2 hours at 37° C. and unbound phage washed away with PBS. Bound phage were eluted and used to infect freshly grown E. coli (XL1-Blue) (Stratagene) for titration on LB agar plates with carbenicillin. The phage libraries were panned for four consecutive rounds with increasing washing stringency.

Library Panning by an Epitope Masking Strategy.

In order to broaden the diversity of antibody specificities selected, library panning was repeated using recombinant E1E2 fused to glutathione S transferase (GST-E1E2; Chan-Fook, C. et al. Virology 273, 60-66 (2000)) pre-incubated with Fabs obtained above. GST-E1E2 was first captured with goat anti-GST antibody (Amersham Biosciences) and the wells were washed and blocked with 4% non-fat dry milk in PBS. Fabs obtained from the panning using E2 antigen above were added to the captured antigens to mask corresponding specific epitopes. The epitope-masked GST-E1E2 was used to pan the phage library as described above. It is important to note that, highly isolate-specific antibodies, e.g. those against HVR1, were not selected due to the use of heterologous antigens in panning.

Screening of Fab Displayed Phage.

Single individual colonies were isolated from titration plates after the 2nd, the 3rd, and the 4th round. The colonies were grown in SB medium with carbenicillin and tetracycline and Fab-phage production was induced with the addition of helper phage overnight at 30° C. The specificities of the Fab-phage were assessed by ELISA and the DNA sequences of the Fab-phage that bound with high specificity were determined. To produce soluble Fabs, the phage gene III surface protein in fusion with the Fab heavy chain was excised by restriction enzymes SpeI and NheI. The cut phagemids were self-ligated and transformed into XL1-Blue cells for the production of soluble Fabs by standard protocols. Barbas III, C. F., Burton, D. R., Scott, J. K. & Silverman, G. J. Phage Display: A Laboratory Manual, (Cold Spring Harbor Laboratory Press, New York, 2001).

Conversion of Fab into IgG1

The vectors pDR12 (Burton, D. R. et al. Science 266, 1024-1027 (1994)) and pIgG1 encoding the leader sequence and constant region of human IgG1 gene were used for the cloning and expression of full length IgG1. Vector pIgG1 is a derivative of pDR12 in which heavy and light chain cloning sites were altered to XhoI/BstEII and SacI/XbaI sites to facilitate direct cloning of the antibody gene fragments. For pDR12, the heavy and light chain genes of Fab C1 were amplified by PCR then inserted sequentially into the SacI/XbaI and HindIII/EcoRI sites of the vector (Burton, D. R. et al. Science 266, 1024-1027 (1994)). For pIgG1, the heavy and light chain gene fragments were excised from the phagemids and inserted sequentially into the XhoI/BstEII and SacI/XbaI sites of the vector. The recombinant plasmids were transfected into Chinese hamster ovarian (CHO) cells. Stable cell clones were established by selection with L-methionine sulfoxide (MSX) and by limiting dilution. Cell clones expressing high IgG levels were amplified and the IgGs were purified using a protein A-agarose column (Pharmacia).

ELISA. (i) To study the relative reactivity of Fabs to GST-E1E2 and E2, soluble Fabs were added to ELISA wells coated with soluble E2 (4 μg/mL), with GST-E1E2 (8 μg/mL) captured by pre-coated goat anti-GST-antibody (10 μg/mL), or with ovalbumin (4 μg/mL). Specific binding was detected by alkaline phosphatase (AP)-conjugated goat anti-human IgG F(ab′)₂ antibody (Pierce) (1:500) in 1% BSA/PBS and disodium p-nitrophenyl phosphate (Sigma). (ii) To study the relationship of different ARs to the mouse MAb epitope H53 (Cocquerel et al., J. J. Virol. 72, 2183-2191 (1998)), a saturating concentration of MAb H53 was added to vaccinia-expressed E1E2 (isolate HCV-1, obtained through the NIH AIDS Research and Reference Reagent Program: rVV E12 C/B from Chiron Corporation; Cooper, S. et al. Immunity 10, 439-449 (1999); Selby, M. et al. J. Immunol. 162, 669-676 (1999)) captured by pre-coated Galanthus nivalis lectin (5 μg/mL, Sigma) for 30 min before the addition of soluble Fabs (2 μg/mL). Non-fat milk (4%, BioRad) in PBS was used as a blocker in assays using lectin-captured antigens. The ELISA plates were washed after a 1 hour incubation and binding of human Fabs was detected by peroxidase (HRP)-conjugated goat anti-human IgG F(ab′)₂ antibody (1:2000) (Pierce) and TMB substrate (Pierce). The level of inhibition by MAb H53 was calculated as the % reduction of optical density signals produced by the human Fabs in the presence of H53. (iii) To study whether the MAbs recognized continuous or discontinuous epitopes, vaccinia-expressed E1E2 was either captured directly onto ELISA wells pre-coated with lectin (folded protein), or unfolded with 0.1% SDS, 50 mM DTT and incubated at 100° C. for 5 minutes before capture onto ELISA wells (unfolded protein). Binding of the MAbs to folded and unfolded proteins was detected using the peroxidase system. Mouse MAb A4 (Dubuisson, J. et al. J. Virol. 68, 6147-6160 (1994)), specific for a linear epitope in E1, was used as a positive control. (iv) To study the ability of MAb in inhibiting E1E2 binding to CD81, serially diluted MAbs (4-fold dilution from 10 μg/mL) were incubated with E1E2 (isolate H77) for 30 min before adding to ELISA wells pre-coated with the large extracellular loop of CD81 (CD81-LEL). After 1 hour incubation, the plates were washed and binding of E1E2 to CD81-LEL was detected with an anti-E1 mouse MAb A4 (Dubuisson, J. et al. J. Virol. 68, 6147-6160 (1994)), HRP-conjugated goat anti-mouse Fc antibody (Pierce) (1:2000) and TMB substrate. Two forms of recombinant CD81-LEL, either in fusion with glutathione S-transferase (GST) (Owsianka, A. M. et al. J. Virol. 80, 8695-8704 (2006)) or maltose binding protein (MBP) (Chan-Fook, C. et al. Virology 273, 60-66 (2000)), were used and the results were equivalent. (v) To study the apparent affinity of the MAbs, serially diluted MAbs (2-fold dilution from 20 μg/mL) were added to lectin-captured E1E2 antigens for 1 hour. E1E2 antigens were prepared from cell lysates from vaccinia-expressed HCV-1 E1E2, 293T cells transfected with H77 E1E2-expression plasmid (McKeating, J. A. et al. J. Virol. 78, 8496-8505 (2004)). The binding of human MAbs was detected by HRP-conjugated goat anti-human IgG F(ab′)₂ antibody as above. Non-infected/non-transfected cell lysate were used as negative controls to determine background for each MAb. Apparent affinity was defined by the concentration of MAbs that produced half of the maximal specific binding in the titration curves. (vi) To construct the MAb competition matrix, saturating concentrations of blocking MAbs (typically at 20 μg/mL or undiluted hybridoma supernatants) were added to lectin-captured vaccinia-expressed HCV-1 E1E2 for 30 minutes before the addition of an equal volume of biotinylated human MAbs (2 μg/mL). The E1E2 antigens were titrated to ensure that saturating concentrations of the blocking MAbs were used in the assays. It is important to note that, MAbs recognizing linear epitopes bind to both folded and unfolded proteins but the biotinylated human MAbs bind conformational epitopes on folded E2. Consequently, competition is performed with the MAbs to linear epitopes as blocking MAbs to eliminate potential non-specific signals caused by misfolded proteins in the system. After incubation for 1 h, the ELISA plates were washed and binding of biotinylated MAbs was detected with HRP-conjugated streptavidin (1:2000, Sigma-Aldrich) in PBS with 1% BSA and TMB substrate (Pierce). Competition was determined by the % change in binding signals in the presence of the blocking antibodies. (vii) To study the effect of alanine substitution in E2 on MAb binding, E1E2 mutant antigens were produced by transfection of 293T cells with the corresponding expression plasmids and the antigens in clarified cell lysate were captured by lectin as above. A panel of 38 H77 E1E2 mutants having the conserved residues in the putative CD81 binding pocket substituted with alanine was used in this study (Owsianka, A. M. et al. J. Virol. 80, 8695-8704 (2006)). The binding signals of the human MAbs to the alanine mutants were compared to the wildtype H77 E1E2 to determine the importance of the residues in the antibody-antigen interaction. (viii) To quantify human IgG in mouse serum, diluted mouse sera in triplicate were added to ELISA wells coated with human goat anti-human IgG F(ab)′₂ (10 μg/mL, Pierce) for 1 hour and bound human IgG was detected with AP-conjugated goat anti-human F(ab)′₂ (10 μg/mL) (Pierce). Serially diluted MAb AR3A (2-fold dilution starting from 4 g/mL) was used to construct a standard curve in each ELISA plate. The concentration of human IgG in each serum sample was calculated from the 4-parameter fitted standard curve using SOFTmax Pro Software (Molecular Devices).

HCV neutralization assays. The neutralization assays were performed in Dulbecco's Modified Eagle Medium (D-MEM) supplemented with 10% fetal calf serum (FCS) (Invitrogen). For HCVpp neutralization, HCVpp was generated by co-transfection of 293T cells with pNL4-3.lucR-E- (Connor et al., Virology 206, 935-944 (1995); He, J. et al. J. Virol. 69, 6705-6711 (1995)) and the corresponding expression plasmids encoding the E1E2 genes at 4:1 ratio by polyethylenimine (Boussif, O. et al. Proc. Natl. Acad. Sci. U.S.A. 92, 7297-7301 (1995)). Virus infectivity was detected using the firefly luciferase assay system (Promega). Background infectivity of the pseudotype virus was determined using cells transfected with pNL4-3.lucR-E-only. The E1E2 expression plasmids for the isolates H77, H, CH35, OH8, UKN1B12.16, J6, UKN2A1.2, UKN2B1.1, UKN3A13.6, UKN3A1.28c, UKN4.21.16, UKN5.15.7 and UKN6.5.8 have been described previously (Owsianka, A. et al. J. Virol. 79, 11095-11104 (2005); McKeating, J. A. et al. J. Virol. 78, 8496-8505 (2004); Lavillette, D. et al. Hepatology 41, 265-274 (2005)). The expression plasmids encoding E1E2 of the virus in an infected human serum (KP) used in the protection experiment are described below. The majority of HCV Envs, except H77, H and OH8, produce only low levels of HCVpp (<5,000 Relative Light Unit, RLU). To ensure the quality of data for determining virus neutralization, 1 HCVpp of low infectivity was concentrated 10-20 fold by centrifugation at 38,000×g for 1 hour. Serially diluted antibodies were first incubated with the virus for 1 hour at 37° C. before adding to Huh-7 cells in triplicate and the cells were incubated for 3 days. After background subtraction, virus neutralization was determined by the % change of RLU in the presence of antibodies. Virus concentration did not affect the neutralization of the prototype HCVpp-H77 by the MAbs in comparison to unconcentrated virus (data not shown). Although virus concentration improved the signals of several HCV Envs, consistent signals were not obtained with HCVpp displaying CH35, UKN3A1.28c, UKN6.5.8 or KP E1E2 in repeated experiments and these Envs were excluded in the analysis.

Cloning of E1E2 from an Infected Human Serum.

Total RNA in the HCV GT1a-infected human serum KP (140 μl) was purified using a QIAamp Viral RNA Mini Kit (Qiagen). First strand cDNA was generated using either a reverse primer specific to HCV1a (HCV1aOuterR, GGGATGCTGCATTGAGTA, (SEQ ID NO: 697); Lavillette, D. et al. Hepatology 41, 265-274 (2005)) or random hexamer using the SuperScript III reverse transcriptase (Invitrogen). The GT1a E1 E2 genes were amplified by a nested PCR as described previously (Lavillette, D. et al. Hepatology 41, 265-274 (2005)) and the PCR products were cloned using the pcDNA3.1/V5-His TOPO TA Expression Kit (Invitrogen). An HIV-positive human serum was used as a negative control throughout the experiments and no non-specific product was generated. The sequences of 40 clones were determined by DNA sequencing and analyzed using VectorNTI software (Invitrogen). Expression of E1E2 proteins was confirmed by the presence of folded E2 proteins in cell lysates, prepared from 293T cells transfected with the corresponding DNA plasmids, by ELISA using MAb AR3A.

Antibody Protection Studies.

Human liver-chimeric mice were prepared as described previously. Mercer, D. F. et al. Nat. Med. 7, 927-933 (2001); Kneteman, N. M. et al. Hepatology 43, 1346-1353 (2006). The animal experiments were approved by the University of Alberta Animal Care and Use Committee for Health Sciences. All human liver biopsies and sera were collected under informed consent and the human subjects protocols were approved by the University of Alberta Health Research Ethics Board (Biomedical Panel). Colonization of human hepatocytes in the livers of Alb-uPA/SCID mice was confirmed by the presence of human alpha-1-anti-trypsin (hAAT) in the mice. Only mice with serum levels of hAAT greater than 60 μg/mL at 6 weeks and 100 μg/mL at 8 weeks, an indication for successful transplantation, were used in the protection study (˜50% of transplanted mice). Mice with low level of human liver chimerism were used in preliminary experiments to measure the toxicity and kinetics of MAbs in Alb-uPA/SCID mice, and the level of human IgG present in mice injected with a genotype 1a HCV-infected human serum KP. This serum, serially diluted from 1:50 to 1:4050, did not neutralize HCVpp-H77 (data not shown). Different doses of MAbs, at 10 mg/mL, were injected into the mice via the intraperitoneal route. For virus challenge, the experiments were conducted in blinded fashion; the identity of the MAbs was not provided to the technicians performing the animal procedures and HCV RNA tests. Human liver-chimeric mice were given MAbs by intraperitoneal injection (200 mg/kg) 24 hours before virus challenge. Mice were anaesthetized and injected intrajugularly with 100 μL of infected serum KP (2.3×10⁶ IU/mL). Blood was sampled by tail bleed and sera were prepared by centrifugation of clotted blood for ELISA and viral load measurement.

HCV RNA Quantification.

HCV RNA in mouse serum was quantified by a real-time TaqMan PCR assay. The two primers in the real-time PCR system were designed to produce a 194 bp PCR fragment corresponding to the 5′ non-coding region with maximum specificity to all HCV genotypes. The forward primer (T-149-F, 5′-TGCGGAACCGGTGAGTACA, (SEQ ID NO: 698) and reverse primer (T-342-R, 5′-AGGTTTAGGATTCGTGCTCAT, (SEQ ID NO: 699) were designed with the aid of software Primer Express (PE biosystems) and were purchased from PE Applied Biosystems. To quantify HCV RNA, total RNA in serum was isolated by the guanidinium thiocyanate (GuSCN) and silico method (Boom, R. et al. J. Clin. Microbiol. 28, 495-503 (1990)). Briefly, 30 μL of serum was mixed with 500 μl GuSCN lysis buffer and 20 μl, size-fractionated silica particles for 15 minutes. The silica particles were pelleted and washed twice with 500 μL washing buffer, twice with 70% ethanol and once with acetone. The pellet was dried for 10 min on a heat block, and RNA was eluted in 20 μL distilled water and quantified by optical density. SuperScript II First-Strand Synthesis Kit (Invitrogen) was used to synthesize first-strand cDNA for PCR. Five μL of the serum RNA was mixed with 100 μM of SuperScript II reverse transcriptase, 20 μM of RNAseOut and 14 μL reaction cocktail (which includes 1× first-strand buffer, 5 μM DTT, 375 nM dNTP, 1.25 μM T-342-R primer) and incubated at 42° C. for 60 min then 70° C. for 15 minutes. For quantitative PCR, a 50 μL mixture contained 9 μL of template HCV cDNA, 1×TaqMan Universal PCR Master Mixture (Applied Biosystems Inc.), 375 nM dNTP, 400 nM of T-149-F and T-342-R primers and 200 nM TaqMan probe (6-FAM18 CACCCTATCAGGCAGTACCACAAGGCC-TAMRA, (SEQ ID NO: 700). Thermocycling was performed on a Taqman 7300 (Applied Biosystems Inc.) using the default setting program recommended by the manufacturer: 50° C. for 2 min, 95° C. for 10 min, and 45 cycles of 95° C. for 15 s and 60° C. for 60 s. A serial dilution of HCV cDNA, including 1.5×10⁶, 1.5×10⁵, 1.5×10⁴, 1.5×10³, 1.5×10², 1.5×10¹, 1.5×10⁰ IU, was used to generate a standard curve for calculation of HCV RNA copy number. The dynamic range of HCV RNA detection for the two step RT-PCR procedure is 6.0×10² IU/ml to 3.0×10⁸ IU/mL. Each assay run incorporates in duplicate a negative control and an HCV RNA positive control. The positive control is the OptiQual HCV RNA 1 Control purchased from AcroMetrix which has been calibrated to the WHO first International Standard for HCV RNA.

Statistical Analysis.

GraphPad Prism 4 software was used for statistical analysis of the antibody protection experiment. Animals seropositive for HCV RNA by the quantitative PCR assay at or after day 7 post-infection were scored as “infected” subjects and animals seronegative up to week 6 were scored as “censored” subjects. The scores were used to construct the Kaplan-Meier survival (infection in this case) curves to calculate statistical significance between the neutralizing antibody-treated and isotype antibody control groups by a two-tailed log rank test within the experimental period. Motulsky, H. Survival curves. in GraphPad Prism4 Statistics Guide: Statistical analyses for laboratory and clinical researchers 107-117 (GraphPad Software, San Diego, 2005).

### Example 2

### Anti-HCV Neutralizing Antibodies

This Example describes the identification of human monoclonal antibodies (mAbs) that neutralize genetically diverse HCV isolates and protect against heterologous HCV quasispecies challenge in a human liver-chimeric mouse model. The results provide evidence that broadly neutralizing antibodies to HCV protect against heterologous viral infection and suggest that a prophylactic vaccine against HCV may be achievable.

A total of 115 clones that exhibit specific binding to HCV E2 glycoprotein were isolated from an antibody antigen-binding fragment (Fab) phage display library generated from a donor chronically infected with HCV (see Example 1). DNA sequence analysis identified 36 distinct Fabs with 13 unique heavy chain sequences. The sequences of the 36 distinct Fabs belonging to 13 groups based on the heavy chain sequences are also shown in Table 10 below. Table 9 sets forth the sequence of the HCDR3. Fabs with the same designation and * or ** have the same heavy chain but distinct light chains, e.g. H1, H1* and H1** have the same heavy chain, but 3 different light chains.

TABLE 9
Fab HCDR3 Sequences

| | | Isolated | |
|---|---|---|---|
| | | by | |
| | | masking | |
| | Fab | with Fab | HCDR3 sequence |
| 1 | A | | ENKFRYCRGGSCYSGAFDM |
| | | | (SEQ ID NO: 140) |
| 2 | B1 | | DPYVYAGDDVWSLSR (SEQ ID NO: 141) |
| 3 | B2 | | DPYVYAGDDVRSLSR (SEQ ID NO: 142) |
| 4 | B3 | | DPYVYAGDDVWSLSR (SEQ ID NO: 143) |
| 5 | C1 | | PETPRYCSGGFCYGEFDN |
| | | | (SEQ ID NO: 144) |
| 6 | C1* | B1 | PETPRYCSGGFCYGEFDN |
| | | | (SEQ ID NO: 144) |
| 7 | C2 | B1 | PETPRYCRGGFCYGEFDN |
| | | | (SEQ ID NO: 145) |
| 8 | C2* | B1 | PETPRYCRGGFCYGEFDN |
| | | | (SEQ ID NO: 145) |
| 9 | C3 | B1 | PETPRYCSGGVCYGEFDN |
| | | | (SEQ ID NO: 146) |
| 10 | C4 | B1 | PETPRYCSGGFCYGEFDN |
| | | | (SEQ ID NO: 147) |
| 11 | C5 | B1 | PETPRYCSGGFCYGEFDN |
| | | | (SEQ ID NO: 148) |
| 12 | C6 | B1 | PETPRYCSGGFCYGEFDN |
| | | | (SEQ ID NO: 149) |
| 13 | D1 | C1 | DPLLFAGGPNWFDH (SEQ ID NO: 150) |
| 14 | D2 | C1 | DPLLFAGGPNWFDH (SEQ ID NO: 151) |
| 15 | D3 | C1, B1 & | DPLLFAGGPNWFDH (SEQ ID NO: 152) |
| | | C1 | |
| 16 | D4 | B1 & C1 | DPLLFAGGPNWFDH (SEQ ID NO: 153) |
| 17 | E | C1 | GPYVGLGEGFSE (SEQ ID NO: 154) |
| 18 | F | B1 & C1 | GGGTE (SEQ ID NO: 155) |
| 19 | G | B1 & C1 | DRGLAINGVVFPYFGLDV |
| | | | (SEQ ID NO: 156) |
| 20 | H1 | B1 | SVTPRHCGGGFCYGEFDY |
| | | | (SEQ ID NO: 157) |
| 21 | H1* | B1 | SVTPRHCGGGFCYGEFDY |
| | | | (SEQ ID NO: 157) |
| 22 | H1** | B1 | SVTPRHCGGGFCYGEFDY |
| | | | (SEQ ID NO: 157) |
| 23 | H2 | B1 | SVTPRHCGGGFCYGEFDY |
| | | | (SEQ ID NO: 158) |
| 24 | H3 | B1 | SVTPRYCGGGFCYGEFDY |
| | | | (SEQ ID NO: 159) |
| 25 | I | B1 | PHGPGLSLGIYSAEYFDE |
| | | | (SEQ ID NO: 160) |
| 26 | J1 | B1 | VGVRGIILVGGLAMNWLDP |
| | | | (SEQ ID NO: 161) |
| 27 | J2 | B1 | VGLRGIVMVGGLAMNWLDP |
| | | | (SEQ ID NO: 162) |
| 28 | J3 | B1 | VGLRGITLVGGLAMNWLDP |
| | | | (SEQ ID NO: 163) |
| 29 | J3* | B1 | VGLRGITLVGGLAMNWLDP |
| | | | (SEQ ID NO: 163) |
| 30 | J4 | B1 | VGLRGINMVGGLAMNWFDP |
| | | | (SEQ ID NO: 164) |
| 31 | K | B1 & C1 | DFYIGPTRDVYYGMDV (SEQ ID NO: 165) |
| 32 | L1 | B1 | AGDLSVGGVLAGGVPHLRHFDP |
| | | | (SEQ ID NO: 166) |
| 33 | L2 | B1 | AGDLSVGGVLAGGVPHLRHFDP |
| | | | (SEQ ID NO: 167) |
| 34 | L3 | B1 | AGDLSVGGVLAGGVPHLRHFDP |
| | | | (SEQ ID NO: 168) |
| 35 | L4 | B1 | AGDLSVGGVLAGGVPHLRHFDP |
| | | | (SEQ ID NO: 169) |
| 36 | M | B1 | ESLYMIAFGRVIWPPLDY |
| | | | (SEQ ID NO: 170) |

TABLE 10A
Anti-HCV E2 Fabs (IgGκ, heavy chain)

| | | |
|---|---|---|
| Fab | FRAMEWORK 1 | CDR1 |

| | | | |
|---|---|---|---|
| 1 | A | LEQSGAEVKKPGSSVKVSCKASGGTFS (SEQ ID NO: 309) | SFVIN (SEQ ID NO: 78) |
| 2 | B1 | LEQSGAEVKKPGSSVKVSCRASGSPFS (SEQ ID NO: 310) | SYTIT (SEQ ID NO: 79) |
| 3 | B2 | LEQSGAEVKKPGSSVKVSCRASGSPYS (SEQ ID NO: 311) | SYTIT (SEQ ID NO: 80) |
| 4 | B3 | LEQSGAEVKKPGSSVKVSCRASGSPYS (SEQ ID NO: 312) | SYTIT (SEQ ID NO: 81) |
| 5 | C1 | LEQSGAEVKTPGSSVRVSCRPPGGNFN (SEQ ID NO: 313) | SYSIN (SEQ ID NO: 82) |
| 6 | C1* | LEQSGAEVKTPGSSVRVSCRPPGGNFN (SEQ ID NO: 313) | SYSIN (SEQ ID NO: 82) |
| 7 | C2 | LEQSGAEVKKPGSSVRVSCRAPGGTFN (SEQ ID NO: 314) | SYSVN (SEQ ID NO: 83) |
| 8 | C2* | LEQSGAEVKKPGSSVRVSCRAPGGTFN (SEQ ID NO: 314) | SYSVN (SEQ ID NO: 83) |
| 9 | C3 | LEQSGAEVKEPGSSVRVSCRAPGGTFN (SEQ ID NO: 315) | SYSIN (SEQ ID NO: 84) |
| 10 | C4 | LEQSGAEVKKPGSSVRVSCRPPGGTFN (SEQ ID NO: 316) | SYSIN (SEQ ID NO: 85) |
| 11 | C5 | LEQSGAEVKKPGSSVRVSCRAPGGTLN (SEQ ID NO: 317) | SYSIN (SEQ ID NO: 86) |
| 12 | C6 | LEQSGAEVKKPGSSVRVSCRPPGGTFN (SEQ ID NO: 318) | SYSIN (SEQ ID NO: 87) |
| 13 | D1 | LE SGGGLVQPGGSLRLSCEASGYYFS (SEQ ID NO: 319) | SFAMS (SEQ ID NO: 88) |
| 14 | D2 | LEQSGGGLVQPGGSLRLSCEASGYYFS (SEQ ID NO: 320) | SFAMS (SEQ ID NO: 89) |
| 15 | D3 | LE SGGGLVQPGGSLRLSCEASGYYFS (SEQ ID NO: 321) | SFAMS (SEQ ID NO: 90) |
| 16 | D4 | LE SGGGLVQPGGSLRLSCEASGYYFS (SEQ ID NO: 322) | SFAMS (SEQ ID NO: 91) |
| 17 | E | LEQSGAELKKPGSSVKVSCKPSDGTFR (SEQ ID NO: 323) | AYTLS (SEQ ID NO: 92) |
| 18 | F | LEQSGNEVKKPGASVKVSCRAYGYNFG (SEQ ID NO: 324) | SERLS (SEQ ID NO: 93) |
| 19 | G | LEQSGAEMKKPGASLKVSCKTSGYTFD (SEQ ID NO: 325) | DYGVT (SEQ ID NO: 94) |
| 20 | H1 | LEQSGAEVKKPGSSVKVSCEASGGTFD (SEQ ID NO 326) | NYSLN (SEQ ID NO 95) |
| 21 | H1* | LEQSGAEVKKPGSSVKVSCEASGGTFD (SEQ ID NO: 326) | NYSLN (SEQ ID NO: 95) |
| 22 | H1** | LEQSGAEVKKPGSSVKVSCEASGGTFD (SEQ ID NO: 326) | NYSLN (SEQ ID NO: 95) |
| 23 | H2 | LEQSGAEVKKPGSSVKVSCETSGGTFD (SEQ ID NO: 327) | NYALN (SEQ ID NO: 96) |
| 24 | H3 | LEQSGAEVKKPGSSVKVSCETSGGTLD (SEQ ID NO: 328) | NYALN (SEQ ID NO: 97) |
| 25 | I | LE SGGGLVQPGRSLRLSCKASGFNFA (SEQ ID NO: 329) | QYTMN (SEQ ID NO: 98) |
| 26 | J1 | LEQSGPEVKKPGSSVKVSCKGSGDRFN (SEQ ID NO: 330) | DPVT (SEQ ID NO: 99) |
| 27 | J2 | LEQSGPEVKKPGSSVKVSCKDSGDTFN (SEQ ID NO: 331) | EPVT (SEQ ID NO: 100) |
| 28 | J3 | LEQSGPEVKKPGSSVKVSCKGSGDTFN (SEQ ID NO: 332) | DPVT (SEQ ID NO: 101) |
| 29 | J3* | LEQSGPEVKKPGSSVKVSCKGSGDTFN (SEQ ID NO: 332) | DPVT (SEQ ID NO: 101) |
| 30 | J4 | LEQSGAEVKKPGSSVRVSCEVSGDTFR (SEQ ID NO: 333) | EPVS (SEQ ID NO: 102) |
| 31 | K | LEQSGPGLVKPGRPFSLTCAISGDSVS (SEQ ID NO: 334) | SDSAAWN (SEQ ID NO: 103) |
| 32 | L1 | LEQSGAEVKKPGSSVKVSCKASGDTFR (SEQ ID NO: 335) | SYVIT (SEQ ID NO: 104) |
| 33 | L2 | LEQSGAEVKMPGSSVKVSCKASGDTFR (SEQ ID NO: 336) | SSVIT (SEQ ID NO: 105) |
| 34 | L3 | LEQSGAEVKKPGSSVKVSCKASGDTFR (SEQ ID NO: 337) | SYVIT (SEQ ID NO: 106) |
| 35 | L4 | LEQSGAEVKKPGSSVKVSCKASGDTFR (SEQ ID NO: 338) | SYVIT (SEQ ID NO: 107) |
| 36 | M | LEQSGAEVKKPGASVKVSCKASGYTFT (SEQ ID NO: 339) | NYAIT (SEQ ID NO: 108) |

TABLE 10B
Anti-HCV E2 Fabs (IgGκ, heavy chain)

| | | |
|---|---|---|
| Fab | FRAMEWORK 2 | CDR2 |

| | | |
|---|---|---|
| A | WVRQAPGQGLEWVG (SEQ ID NO: 340) | GIFQAPGPEREWLRDINPISGTINYAQRFQG (SEQ ID |
| | | NO: 109) |
| B1 | WVRQAPGQGLEWMG (SEQ ID NO: 341) | GIILMTGKANYAQKFQG (SEQ ID NO: 110) |
| B2 | WVRQAPGQGLEWMG (SEQ ID NO: 342) | GIILMTGKANYAQKFQG (SEQ ID NO: 111) |
| B3 | WVRQAPGQGLEWMG (SEQ ID NO: 343) | GIILMTGKANYAQKFQG (SEQ ID NO: 112) |
| C1 | WVRQAPGHGLEWVG (SEQ ID NO: 344) | TFIPMFGTSKYAQKFQG (SEQ ID NO: 113) |
| C1* | WVRQAPGHGLEWVG (SEQ ID NO: 344) | TFIPMFGTSKYAQKFQG (SEQ ID NO: 113) |
| C2 | WVRQAPGHGLEWVG (SEQ ID NO: 345) | TLIPMFGTSSYAQKFQG (SEQ ID NO: 114) |
| C2* | WVRQAPGHGLEWVG (SEQ ID NO: 345) | TLIPMFGTSSYAQKFQG (SEQ ID NO: 114) |
| C3 | WVRQAPGHGLEWVG (SEQ ID NO: 346) | TLIPMFGTSNYAQKFQG (SEQ ID NO: 115) |
| C4 | WVRQAPGHGLEWVG (SEQ ID NO: 347) | TLIPMFGTSKYAQKLQG (SEQ ID NO: 116) |
| C5 | WVRQAPGHGLEWVG (SEQ ID NO: 348) | TLIPMFGTSNYAQKFQG (SEQ ID NO: 117) |
| C6 | WVRQAPGHGLEWVG (SEQ ID NO: 349) | TIIPMFGTSKYAQKLQG (SEQ ID NO: 118) |
| D1 | WVRQTPGKGLEWVS (SEQ ID NO: 350) | SIAGGTLGRTSYRDSVKG (SEQ ID NO: 119) |
| D2 | WVRQTPGKGLEWVS (SEQ ID NO: 351) | SIAGGTLGRTSYRDSVKG (SEQ ID NO: 120) |
| D3 | WVRQTPGKGLEWVS (SEQ ID NO: 352) | SIAGGTLGRTSYRDSVKG (SEQ ID NO: 121) |
| D4 | WVRQTPGKGLEWVS (SEQ ID NO: 353) | SIAGGTLGRTSYRDSVKG (SEQ ID NO: 122) |
| E | WVRQAPGQTLEWMG (SEQ ID NO: 354) | RIMPTVGITNYAQKFQG (SEQ ID NO: 123) |
| F | WVRQAPGQGLEWMG (SEQ ID NO: 355) | WISAYNGGINYSQKFQG (SEQ ID NO: 124) |
| G | WVRQAPGQGLEWMG (SEQ ID NO: 356) | WISAYSGNTFYARKFQG (SEQ ID NO: 125) |
| H1 | WVRQAPGQGLEWIG (SEQ ID NO: 357) | GVVPLFGTTKYAQKFQG (SEQ ID NO: 126) |
| H1* | WVRQAPGQGLEWIG (SEQ ID NO: 357) | GVVPLFGTTKYAQKFQG (SEQ ID NO: 126) |
| H1** | WVRQAPGQGLEWIG (SEQ ID NO: 357) | GVVPLFGTTKYAQKFQG (SEQ ID NO: 126) |
| H2 | WVRQAPGQGLEWIG (SEQ ID NO: 358) | GVVPLFGTTKYAQKFQG (SEQ ID NO: 127) |
| H3 | WVRQAPGQGLEWIG (SEQ ID NO: 359) | GVVPLFGTTRNAQKFQG (SEQ ID NO: 128) |
| I | WVRQAPGKGLEWIG (SEQ ID NO: 360) | LIRTTAYDAATHYAASVEG (SEQ ID NO: 129) |
| J1 | WVRQAPGQGLEWIG (SEQ ID NO: 361) | GIIPAFGATKYAQKFQG (SEQ ID NO: 130) |
| J2 | WVRQAPGQGLEWIG (SEQ ID NO: 362) | GIIPAFGVTKYAQKFQG (SEQ ID NO: 131) |
| J3 | WVRQAPGQGLEWIG (SEQ ID NO: 363) | GIIPLFGAAKYAQKFQG (SEQ ID NO: 132) |
| J3* | WVRQAPGQGLEWIG (SEQ ID NO: 363) | GIIPLFGAAKYAQKFQG (SEQ ID NO: 132) |
| J4 | WVRQAPGQGFEWIG (SEQ ID NO: 364) | GIIPMFGATHYAQKLQG (SEQ ID NO: 133) |
| K | WVRQSPSRGLEWLG (SEQ ID NO: 365) | RTFYRSKWYYDYTVSVKS (SEQ ID NO: 134) |
| L1 | WARQAPGQGLEWMG (SEQ ID NO: 366) | AIIPFFGTTNLAQKFQG (SEQ ID NO: 135) |
| L2 | WARQAPGQGLEWMG (SEQ ID NO: 367) | AIIPFFGTTNLAQKFQG (SEQ ID NO: 136) |
| L3 | WARQAPGQGLEWMG (SEQ ID NO: 368) | AIIPFFGTTNLAQKFQG (SEQ ID NO: 137) |
| L4 | WARQAPGQGLEWMG (SEQ ID NO: 369) | AIIPFFGTTNLAQKFQG (SEQ ID NO: 138) |
| M | WVRQAPGQGLEWMG (SEQ ID NO: 370) | WISGDSTNTYYGQKFQG (SEQ ID NO: 139) |

TABLE 10C
Anti-HCV E2 Fabs (IgGκ, heavy chain)

| | | | |
|---|---|---|---|
| Fab | FRAMEWORK3 | CDR3 | FRAMEWORK4 |

| | | | |
|---|---|---|---|
| A | RVTMTADESMTTVYMELSSLRSEDTAMYYCAR | ENKFRYCRGGSCYSGAFDM | WGQGTMVTVSSAS |
| | (SEQ ID NO: 371) | (SEQ ID NO: 140) | (SEQ ID NO: 402) |
| B1 | RVTITADRSTTTAYMEMSSLTSDDTAIYYCAR | DPYVYAGDDVWSLSR | WGQGTLVIVSSAS |
| | (SEQ ID NO: 372) | (SEQ ID NO: 141) | (SEQ ID NO: 403) |
| B2 | RVTITADRATATAYMEMSSLTSDDTAIYYCAR | DPYVYAGDDVRSLSR | WGQGTPVIVSSAS |
| | (SEQ ID NO: 373) | (SEQ ID NO: 142) | (SEQ ID NO: 404) |
| B3 | RVTITADRATATAYMEMSSLTSDDTAIYYCAR | DPYVYAGDDVWSLSR | WGQGTPVIVSSAS |
| | (SEQ ID NO: 374) | (SEQ ID NO: 143) | (SEQ ID NO: 405) |
| C1 | RVTITADGSSGTAYMDLNSLRSDDTAFYYCVR | PETPRYCSGGFCYGEFDN | WGQGTLVTVSSAS |
| | (SEQ ID NO: 375) | (SEQ ID NO: 144) | (SEQ ID NO: 406) |
| C1* | RVTITADGSSGTAYMDLNSLRSDDTAFYYCVR | PETPRYCSGGFCYGEFDN | WGQGTLVTVSSAS |
| | (SEQ ID NO: 375) | (SEQ ID NO: 144) | (SEQ ID NO: 406) |
| C2 | RVTITADGSSGTAYMELNSLRSDDTAVYYCVR | PETPRYCRGGFCYGEFDN | WGQGTLVTVSSAS |
| | (SEQ ID NO: 376) | (SEQ ID NO: 145) | (SEQ ID NO: 407) |
| C2* | RVTITADGSSGTAYMELNSLRSDDTAVYYCVR | PETPRYCRGGFCYGEFDN | WGQGTLVTVSSAS |
| | (SEQ ID NO: 376) | (SEQ ID NO: 145) | (SEQ ID NO: 407) |
| C3 | RVTITADGSSGTAYMELNSLRSDDTAVYYCVR | PETPRYCSGGVCYGEFDN | WGQGTLVTVSSAS |
| | (SEQ ID NO: 377) | (SEQ ID NO: 146) | (SEQ ID NO: 408) |
| C4 | RVTITADGSSGTAYMELNSLRSDDTAVYYCVR | PETPRYCSGGFCYGEFDN | WGQGTLVTVSSAS |
| | (SEQ ID NO: 378) | (SEQ ID NO: 147) | (SEQ ID NO: 409) |
| C5 | RVTITADGSSGTAYMELNSLRSDDTAVYYCVR | PETPRYCSGGFCYGEFDN | WGQGTLVTVSSAS |
| | (SEQ ID NO: 379) | (SEQ ID NO: 148) | (SEQ ID NO: 410) |
| C6 | RVTITADGSSGTAYMELNSLRSDDTAVYYCVR | PETPRYCSGGFCYGEFDN | WGQGTLVTVSSAS |
| | (SEQ ID NO: 380) | (SEQ ID NO: 149) | (SEQ ID NO: 411) |
| D1 | RFTISRDNSKNTVFLHMNNLRPEDTAVYYCAK | DPLLFAGGPNWFDH | WGQGTLVTVSSAS |
| | (SEQ ID NO: 381) | (SEQ ID NO: 150) | (SEQ ID NO: 412) |
| D2 | RFTISRDNSKNTVFLHMNNLRPEDTAVYYCAK | DPLLFAGGPNWFDH | WGQGTLVTVSSAS |
| | (SEQ ID NO: 382) | (SEQ ID NO: 151) | (SEQ ID NO: 413) |
| D3 | RFTISRDNSKNTMFLHMNNLRPEDTAVYYCAK | DPLLFAGGPNWFDH | WGQGTLVTVSSAS |
| | (SEQ ID NO: 383) | (SEQ ID NO: 152) | (SEQ ID NO: 414) |
| D4 | RFTISRDNSKNTVFLHMSNLRPEDTAVYYCAK | DPLLFAGGPNWFDH | WGQGTLVTVSSAS |
| | (SEQ ID NO: 384) | (SEQ ID NO: 153) | (SEQ ID NO: 415) |
| E | RVTISADMSTATAYMELSSLRSDDTAIYYCAK | GPYVGLGEGFSE | WGQGTLVTVSSAS |
| | (SEQ ID NO: 385) | (SEQ ID NO: 154) | (SEQ ID NO: 416) |
| F | RFTMTTDTSTRTGYMELRNLRSDDTAVYYCAR | GGGTE | WGQGTLVIVSSDE |
| | (SEQ ID NO: 386) | (SEQ ID NO: 155) | (SEQ ID NO: 417) |
| G | RVTMTTDPSTRTAYMELRSLRSDDTAVYFCAR | DRGLAINGVVFPYFGLDV | WGQGTTVTVSSAS |
| | (SEQ ID NO: 387) | (SEQ ID NO: 156) | (SEQ ID NO: 418) |
| H1 | RVTISDDKSTGTGHMELRSLRSEDTAVYYCVR | SVTPRHCGGGFCYGEFDY | WGQGTLVTVSSAS |
| | (SEQ ID NO: 388) | (SEQ ID NO: 157) | (SEQ ID NO: 419) |
| H1* | RVTISDDKSTGTGHMELRSLRSEDTAVYYCVR | SVTPRHCGGGFCYGEFDY | WGQGTLVTVSSAS |
| | (SEQ ID NO: 388) | (SEQ ID NO: 157) | (SEQ ID NO: 419) |
| H1** | RVTISDDKSTGTGHMELRSLRSEDTAVYYCVR | SVTPRHCGGGFCYGEFDY | WGQGTLVTVSSAS |
| | (SEQ ID NO: 388) | (SEQ ID NO: 157) | (SEQ ID NO: 419) |
| H2 | RVTISDDKSTGTGHMELRSLRSEDTAVYYCVR | SVTPRHCGGGFCYGEFDY | WGQGTLVTVSSAS |
| | (SEQ ID NO: 389) | (SEQ ID NO: 158) | (SEQ ID NO: 420) |
| H3 | RVTISDDKSTGTGHMELRSLRSEDTAVYYCVR | SVTPRYCGGGFCYGEFDY | WGQGTLVTVSSAS |
| | (SEQ ID NO: 390) | (SEQ ID NO: 159) | (SEQ ID NO: 421) |
| I | RFTISRDDSKSTAYLQINGLKTEDTAVYYCAR | PHGPGLSLGIYSAEYFDE | WGQGTLVTVSSAS |
| | (SEQ ID NO: 391) | (SEQ ID NO: 160) | (SEQ ID NO: 422) |
| J1 | RVVISADASTDTAYMELSSLRSEDTAVYYCAK | VGVRGIILVGGLAMNWLDP | WGQGTLVTVSAAS |
| | (SEQ ID NO: 392) | (SEQ ID NO: 161) | (SEQ ID NO: 423) |
| J2 | RVIISADASTATAYLELSSLRSEDTAVYYCAK | VGLRGIVMVGGLAMNWLDP | WGQGTQVTVSSAS |
| | (SEQ ID NO: 393) | (SEQ ID NO: 162) | (SEQ ID NO: 424) |
| J3 | RVTISADASALTTYMELSSLRPEDTAVYYCAK | VGLRGITLVGGLAMNWLDP | WGQGTLITVSSAS |
| | (SEQ ID NO: 394) | (SEQ ID NO: 163) | (SEQ ID NO: 425) |
| J3* | RVTISADASALTTYMELSSLRPEDTAVYYCAK | VGLRGITLVGGLAMNWLDP | WGQGTLITVSSAS |
| | (SEQ ID NO: 394) | (SEQ ID NO: 163) | (SEQ ID NO: 425) |
| J4 | RITISADQSTNTVYMELSSLRSDDTAVYYCAK | VGLRGINMVGGLAMNWFDP | WGQGTLVTVSSAS |
| | (SEQ ID NO: 395) | (SEQ ID NO: 164) | (SEQ ID NO: 426) |
| K | RITINSDTSKNQFSLHLNSVTPEDTAVYYCVR | DFYIGPTRDVYYGMDV | WGQGTTVTVSSAS |
| | (SEQ ID NO: 396) | (SEQ ID NO: 165) | (SEQ ID NO: 427) |
| L1 | RVTITADESTQTVYMDLSSLRSDDTAVYYCAK | AGDLSVGGVLAGGVPHLRHFDP | WGQGTLVTVSSAS |
| | (SEQ ID NO: 397) | (SEQ ID NO: 166) | (SEQ ID NO: 428) |
| L2 | RVTITADESTKTVYMDLSSLRSDDTAVYYCAK | AGDLSVGGVLAGGVPHLRHFDP | WGQGTLVTVSSAS |
| | (SEQ ID NO: 398) | (SEQ ID NO: 167) | (SEQ ID NO: 429) |
| L3 | RVTITADESTKTVYMDLSSLTSDDTAVYYCAK | AGDLSVGGVLAGGVPHLRHFDP | WGQGTLVTVSSAS |
| | (SEQ ID NO: 399) | (SEQ ID NO: 168) | (SEQ ID NO: 430) |
| L4 | RVTITADESTKTVYMDLSSLRSDDTAVYYCAK | AGDLSVGGVLAGGVPHLRHFDP | WGQGTLVTVSSAS |
| | (SEQ ID NO: 400) | (SEQ ID NO: 169) | (SEQ ID NO: 431) |
| M | RVTMTTDTSTSTAYMELTSLTSEDTAVYYCAR | ESLYMIAFGRVIWPPLDY | WGQGTLVTISSAS |
| | (SEQ ID NO: 401) | (SEQ ID NO: 170) | (SEQ ID NO: 432) |

TABLE 11A
Anti-HCV E2 Fabs (IgGκ, Light chain)

| | | |
|---|---|---|
| Fab | FRAMEWORK1 | CDR1 |

| | | |
|---|---|---|
| A | EL TQSPATLSVSPGESATLSC (SEQ ID NO: 433) | RASQSVSDN LA (SEQ ID NO: 171) |
| B1 | ELTLTQSPGTLSLSPGERATLSC (SEQ ID NO: 434) | RASQSVSNS YLA (SEQ ID NO: 172) |
| B2 | TLTQSPDSLAVSLGERATINC (SEQ ID NO: 435) | KSSQSVLYSSNNKNVLA (SEQ ID NO: 173) |
| B3 | ELVMTQSPGTLSLSPGERATLSC (SEQ ID NO: 436) | RASQRVGSS YLA (SEQ ID NO: 174) |
| C1 | ELTLTQSPGTLSLSPGKRATLSC (SEQ ID NO: 437) | RASQSVSGN YLA (SEQ ID NO: 175) |
| C1* | EL TQSPSTLSLSPGEGATLSC (SEQ ID NO: 438) | RPSQSVSRN YLA (SEQ ID NO: 176) |
| C2 | EL TQSPGTLSLSPGERAALSC (SEQ ID NO: 439) | RASQSISTN YLA (SEQ ID NO: 177) |
| C2* | EL TQSPGTLSLSPGERATLSC (SEQ ID NO: 440) | RASQSVSSS YLA (SEQ ID NO: 178) |
| C3 | EL TQSPGTLSLSPGERATLSC (SEQ ID NO: 441) | RASQSVSSS YLA (SEQ ID NO: 179) |
| C4 | EL TQSPGTLSLSPGERATLSC (SEQ ID NO: 442) | RASQSVSSS YLA (SEQ ID NO: 180) |
| C5 | EL TQSPATLYVSPGERATLSC (SEQ ID NO: 443) | RASQSVPDN HLA (SEQ ID NO: 181) |
| C6 | EL TQSPATLSVSPGESATLSC (SEQ ID NO: 444) | RASQSVSSN LA (SEQ ID NO: 182) |
| D1 | EL TQSPGTLSLSPGERATLSC (SEQ ID NO: 445) | RASQSVSSS YLA (SEQ ID NO: 183) |
| D2 | EL TQSPATLSVSPGERATLSC (SEQ ID NO: 446) | RASQTISDN LA (SEQ ID NO: 184) |
| D3 | ELTLTQSPGTLSLSPGERATLSC (SEQ ID NO: 447) | RASQTVSSS YLA (SEQ ID NO: 185) |
| D4 | ELVMTQSPGTLSLSPGERATLSC (SEQ ID NO: 448) | RASQSVSSS YLA (SEQ ID NO: 186) |
| E | ELVLTQSPLSLPVTLGQPASISC (SEQ ID NO: 449) | RSTQSLVYSDGNT YLN (SEQ ID NO: 187) |
| F | ELQMTQSPSFLSASVGDRVTITC (SEQ ID NO: 450) | RASQGISS YLA (SEQ ID NO: 188) |
| G | EL TQSPVSLPVTPGEPASISC (SEQ ID NO: 451) | RSSQSLLHSNGNH YLD (SEQ ID NO: 189) |
| H1 | ELTLTQSPGTLSLSPGERATLSC (SEQ ID NO: 452) | RASQSISSS YLA (SEQ ID NO: 190) |
| H1* | EL TQSPATLSVSPGERATLSC (SEQ ID NO: 453) | RASRGISSN LA (SEQ ID NO: 191) |
| H1** | ELTLTQSPGTLSLSPGERATLSC (SEQ ID NO: 454) | RASQSVSSDS LA (SEQ ID NO: 192) |
| H2 | ELTLTQSPGTLSLSPGERGTLSC (SEQ ID NO: 455) | RASQSVSSS YLA (SEQ ID NO: 193) |
| H3 | EL TQSPATLSVSPGERATLSC (SEQ ID NO: 456) | RASQSVSSN LA (SEQ ID NO: 194) |
| I | ELTLTQSPATLSVSPGERATLFC (SEQ ID NO: 457) | RANQSVGRN LA (SEQ ID NO: 195) |
| J1 | ELVLTQSPGTLSLSPGERATLSC (SEQ ID NO: 458) | RASQSVSSS YLA (SEQ ID NO: 196) |
| J2 | EL TQSPGTLSLSPGERATLSC (SEQ ID NO: 459) | RASQSVSSS YLA (SEQ ID NO: 197) |
| J3 | EL TQSPGTLSLSPGERATLSC (SEQ ID NO: 460) | RASQSVSSS YLA (SEQ ID NO: 198) |
| J3* | EFTLTQSPGTLSLSPGERGTLSC (SEQ ID NO: 461) | RASQSVSSS YLA (SEQ ID NO: 199) |
| J4 | ELTLTQSPGTLSLSPGERATLSC (SEQ ID NO: 462) | RASQSVSSS YLA (SEQ ID NO: 200) |
| K | EL TQSPGTLSLSPGERATLSC (SEQ ID NO: 463) | RASQSVSSNS LA (SEQ ID NO: 201) |
| L1 | EL TQSPGTLSLSPGERATLSC (SEQ ID NO: 464) | RASQSITSR YLA (SEQ ID NO: 202) |
| L2 | EL TQSPGTLSLSPGERATLSC (SEQ ID NO: 464) | RASQSITSR YLA (SEQ ID NO: 202) |
| L3 | ELVMTQSPATLSLSPGERATLSC (SEQ ID NO: 465) | RASQSVGS YLA (SEQ ID NO: 203) |
| L4 | EL TQSPGTLSLSPGERATLSC (SEQ ID NO: 466) | RAGQTVASNS LA (SEQ ID NO: 204) |
| M | ELTLTQSPGTLSLSPGERATLSC (SEQ ID NO: 467) | RASQSIRSS YLA (SEQ ID NO: 205) |

TABLE 11B
Anti-HCV E2 Fabs (IgGκ, Light chain)

| | | | |
|---|---|---|---|
| Fab | FRAMEWORK2 | CDR2 | FRAMEWORK 3 |

| | | | |
|---|---|---|---|
| A | WYQQKPGQAPRLLIY | GASSRAP (SEQ ID NO: 206) | AIPGRFSGSGSGTDFTLTISRLEPEDLAVYHC |
| | (SEQ ID NO: 468) | | (SEQ ID NO: 503) |
| B1 | WYQQKPGQAPRLLIY | GASSRAT (SEQ ID NO: 207) | GIPDRFSGSGSGTDFTLTISRLEPEDFAVYYC |
| | (SEQ ID NO: 469) | | (SEQ ID NO: 504) |
| B2 | WYQQKPGQPPQLLIY | WASTRES (SEQ ID NO: 208) | GVPDRFSGSGSGTDFTLTISSLQAEDVAVYFC |
| | (SEQ ID NO: 470) | | (SEQ ID NO: 505) |
| B3 | WYQQKPGQAPRLLVY | GASSRAT (SEQ ID NO: 209) | GIPDRFSGSGSGTDFTLTISRLQPEDFAVYYC |
| | SEQ ID NO: 471) | | (SEQ ID NO: 506) |
| C1 | WYQQKPGQAPRLLIY | GASNRAT (SEQ ID NO: 210) | GIPHRFSGSGSGTDFTLTISRLEPEDFAVYYC |
| | (SEQ ID NO: 472) | | (SEQ ID NO: 507) |
| C1* | WYQQKPGQAPRLLIY | GASTRAT (SEQ ID NO: 211) | GIPDRFSGSGSGTNFTLTISRLEPEDFAVYFC |
| | (SEQ ID NO: 473) | | (SEQ ID NO: 508) |
| C2 | WYQQKPGQAPRLLIY | GTSNRAT (SEQ ID NO: 212) | GIPDRFSGTGSGTDFSLTISRLEPEDSAVYYC |
| | (SEQ ID NO: 474) | | (SEQ ID NO: 509) |
| C2* | WYQQKPGQAPRLLIY | GASSRAT (SEQ ID NO: 213) | GIPDRFSGSGSGTDFTLTISRLEPEDFAVYYC |
| | (SEQ ID NO: 475) | | (SEQ ID NO: 510) |
| C3 | WYQQKPGQAPRLLIY | GASSRAT (SEQ ID NO: 214) | GIPDRFSGSGSGTDFTLTISGLEPEDFAVYYC |
| | (SEQ ID NO: 476) | | (SEQ ID NO: 511) |
| C4 | WYQQKPGQAPRLLIY | GASSRAT (SEQ ID NO: 215) | GIPDRFSGSGSGTDFTLTISRLEPEDFAVYYC |
| | (SEQ ID NO: 477) | | (SEQ ID NO: 512) |
| C5 | WYQQKPGQTPRLLIY | GASKRAT (SEQ ID NO: 216) | GIPDRFSGSGSGTDFTLTISRLEPEDFAVYYC |
| | (SEQ ID NO: 478) | | (SEQ ID NO: 513) |
| C6 | WYQQKPGQAPRLLIY | GASTRAT (SEQ ID NO: 217) | GIPARFSGSGSGTDFTLTISRLEPEDFAVYYC |
| | (SEQ ID NO: 479) | | (SEQ ID NO: 514) |
| D1 | WYQQKPGQAPRLLIY | GASSRAT (SEQ ID NO: 218) | GIPDRFSGSGSGTDFTLTISRLEPEDFAVYYC |
| | (SEQ ID NO: 480) | | (SEQ ID NO: 515) |
| D2 | WYQQKPGQAPRLLIY | GASSRAT (SEQ ID NO: 219) | GIPDRFSGSGSGTDFTLTISRLEPEDFAVYYC |
| | (SEQ ID NO: 481) | | (SEQ ID NO: 516) |
| D3 | WYQQKPGQAPRLLIY | GASSRAT (SEQ ID NO: 220) | GIPDRFSGSGSGTDFTLTISRLEPEDFAVYYC |
| | (SEQ ID NO: 482) | | (SEQ ID NO: 517) |
| D4 | WYQQKPGQAPRLLIY | GASSRAT (SEQ ID NO: 221) | GIPDRFSGSGSGTDFTLTITRLEPEDFAVYYC |
| | (SEQ ID NO: 483) | | (SEQ ID NO: 518) |
| E | WFHQRAGQPPRRLIY | KVSNRDS (SEQ ID NO: 222) | GVPERFSGSGSGTDFTLKISRVEAEDVGIYYC |
| | (SEQ ID NO: 484) | | (SEQ ID NO: 519) |
| F | WYQQKPGKAPKLLIS | SVSTLQS (SEQ ID NO: 223) | GVSSRFSGSGSGTGFTLTISSLQSEDSATYYC |
| | (SEQ ID NO: 485) | | (SEQ ID NO: 520) |
| G | WYLQKPGQSPQLLMY | LGSNRAS (SEQ ID NO: 224) | GVPDRFSGSGSGTDFTLKISRVEAEDVGVYYC |
| | (SEQ ID NO: 486) | | (SEQ ID NO: 521) |
| H1 | WYQQKPGQAPRLLIY | GASRRAT (SEQ ID NO: 225) | GIPDRFSGSGSGTDFTLTISRLEPEDFAVYYC |
| | (SEQ ID NO: 487) | | (SEQ ID NO: 522) |
| H1* | WYQQKPGQAPRLLIY | GASSRAT (SEQ ID NO: 226) | GIPDRFSGSGSGTDFTLTISRLEPEDFAVYYC |
| | (SEQ ID NO: 488) | | (SEQ ID NO: 523) |
| H1** | WYQQKPGQAPRLLIY | GASSRAT (SEQ ID NO: 227) | GIPDRFSGSGSGTDFTLTISRLEPEDLGVYYC |
| | (SEQ ID NO: 489) | | (SEQ ID NO: 524) |
| H2 | WYQQKPGQAPRLLIY | GASSRAT (SEQ ID NO: 228) | GIPDRFSGSGSGTDFTLTISRLEPEDFAVYYC |
| | (SEQ ID NO: 490) | | (SEQ ID NO: 525) |
| H3 | WYQQKPGQAPRLLIY | GASTRAT (SEQ ID NO: 229) | GIPARFSGSGSGTDFTLTVSRLEPEDSAVYFC |
| | (SEQ ID NO: 491) | | (SEQ ID NO: 526) |
| I | WYQQKPGQAPRLLIY | GISTRTT (SEQ ID NO: 230) | TTPTRFSGSGSGTDFTLTISRLQSEDFAVYYC |
| | (SEQ ID NO: 492) | | (SEQ ID NO: 527) |
| J1 | WYQQKPGQAPRLLIY | GASSRAT (SEQ ID NO: 231) | GIPDRFSGSGSGTDFALTITRLEPEDFAVYYC |
| | (SEQ ID NO: 493) | | (SEQ ID NO: 528) |
| J2 | WYQQKPGQAPRLLIY | GASSRAT (SEQ ID NO: 232) | GIPDRFSGSGSGTDFTLTISRLEPEDFAVYYC |
| | (SEQ ID NO: 494) | | (SEQ ID NO: 529) |
| J3 | WYQQKPGQAPRLLIY | GASSRAT (SEQ ID NO: 233) | GIPDRFSGSGSGTDFTLTISGLEPEDFAVYYC |
| | (SEQ ID NO: 495) | | (SEQ ID NO: 530) |
| J3* | WYQQKPGQAPRLLIY | GASSRAT (SEQ ID NO: 234) | GIPDRFSGSGSGTDFTLTISRLEPEDFAVYYC |
| | (SEQ ID NO: 496) | | (SEQ ID NO: 531) |
| J4 | WYQQKPGQAPRLLIY | GASSRAT (SEQ ID NO: 235) | GIPDRFSGSGSGTDFTLTISRLEPEDFAVYYC |
| | (SEQ ID NO: 497) | | (SEQ ID NO: 532) |
| K | WYQQKPGLAPRLLIY | GASSRAT (SEQ ID NO: 236) | GIPDRFSGSGSGTGFTLTISTLEPEDFAIYYC |
| | (SEQ ID NO: 498) | | (SEQ ID NO: 533) |
| L1 | WYQQKPGQAPRLLIY | GASSRAT (SEQ ID NO: 237) | GIPDRFSGSGSGTDFTLTISRLEPEDFAVYYC |
| | (SEQ ID NO: 499) | | (SEQ ID NO: 534) |
| L2 | WYQQKPGQAPRLLIY | GASSRAT (SEQ ID NO: 237) | GIPDRFSGSGSGTDFTLTISRLEPEDFAVYYC |
| | (SEQ ID NO: 499) | | (SEQ ID NO: 534) |
| L3 | WYQQKPGQAPRLLIY | DASNRAT (SEQ ID NO: 238) | GIPARFSGSGSGTDFTLTISSLEPEDFAVYFC |
| | (SEQ ID NO: 500) | | (SEQ ID NO: 535) |
| L4 | WYQHKPGQAPRLLIY | GASIRAS (SEQ ID NO: 239) | GIPDRFSGSGSGTDFTLTISRLEPEDFAVYYC |
| | (SEQ ID NO: 501) | | (SEQ ID NO: 536) |
| M | WYQQKPGQAPRLLIY | AAASRAT (SEQ ID NO: 240) | GIPDRFSGSGSGTDFTLTISRLEPEDFAVYFC |
| | (SEQ ID NO: 502) | | (SEQ ID NO: 537) |

TABLE E11C
Anti-HCV E2 Fabs (IgGκ, Light chain)

| | | |
|---|---|---|
| Fab | CDR3 | FRAMEWORK4 |

| | | |
|---|---|---|
| A | QQYGAS PWT (SEQ ID NO: 241) | FGQGTKVEIKRTVAAPSVFIFPPSDEQLKSGTASVV (SEQ ID NO: |
| | | 538) |
| B1 | QQYGSS PQT (SEQ ID NO: 242) | FGQGTKVEIKRTVAAPSVFIFPPSDEQLKSGTASVV (SEQ ID NO: |
| | | 539) |
| B2 | QQYYST PFT (SEQ ID NO: 243) | FGPGTKVEIKRTVAAPSVFIFPPSDEQLKSGTASVV (SEQ ID NO: |
| | | 540) |
| B3 | QQYGTT (SEQ ID NO: 244) | FGQGTRVDIKRTVAAPSVSIFPPSDEQLKSGTASVV (SEQ ID NO: |
| | | 541) |
| C1 | QQYGSS PT (SEQ ID NO: 245) | FGQGTRVDIKRTVAAPSVFIFPPSDEQLKSGTASV (SEQ ID NO: 542) |
| C1* | QHYGNS PPYT (SEQ ID NO: 246) | FGQGTKLEIKRTVAAPSVFIFPP (SEQ ID NO: 543) |
| C2 | QQYGTS PFT (SEQ ID NO: 247) | FGPGTKVDIKRTVAAPSVFIFPPS (SEQ ID NO: 544) |
| C2* | QQYGSS PQT (SEQ ID NO: 248) | FGQGTKVEIKRTVAAPSVFIFPPSDEQLKSGTASVV (SEQ ID NO: |
| | | 545) |
| C3 | QQYGSS PLT (SEQ ID NO: 249) | FGGGTKVEIKRTVAAPSVFIFPPSD (SEQ ID NO: 546) |
| C4 | QHYGSS SYT (SEQ ID NO: 250) | FGQGTRVEIKRTVAAPSVFIFPPSDEQLKSGTASVV (SEQ ID NO: |
| | | 547) |
| C5 | QQYGSS PQT (SEQ ID NO: 251) | FGQGTKVEIKRTVAAPSVFIFPPSDEQLKSGTASVV (SEQ ID NO: |
| | | 548) |
| C6 | QQYGGSPPYT (SEQ ID NO: 252) | FGQGTKLEIKRTVAAPSVFIFPPSDEQLKSGTASVV (SEQ ID NO: |
| | | 549) |
| D1 | QQYGSS PQT (SEQ ID NO: 253) | FGQGTKVEIKRTVAAPSVFIFPPSDEQLKSGTASVV (SEQ ID NO: |
| | | 550) |
| D2 | QQYGSS PQT (SEQ ID NO: 254) | FGQGTKVEIKRTVAAPSVFIFPPSDEQLKSGTASVV (SEQ ID NO: |
| | | 551) |
| D3 | QQYGSS PQT (SEQ ID NO: 255) | FGQGTKVEIKRTVAAPSVFIFPPSDEQLKSGTASVV (SEQ ID NO: |
| | | 552) |
| D4 | QQYGSS PQT (SEQ ID NO: 256) | FGQGTKVQIKRTVAAPSVFIFPPSDEQLKSGTASVV (SEQ ID NO: |
| | | 553) |
| E | MQGAHW PPT (SEQ ID NO: 257) | FGGGTKVEINRTVAAPSVFIFPPSDEQLKSGTA (SEQ ID NO: 554) |
| F | EQLNSF PYT (SEQ ID NO: 258) | FGQGTKLEIKRTVAAPSVFIFPPSD (SEQ ID NO: 555) |
| G | MQGLQT PWT (SEQ ID NO: 259) | FGQGTKVEIKRTVAAPSVFIFPPSD (SEQ ID NO: 556) |
| H1 | QQYGSS PLT (SEQ ID NO: 260) | FGGGTKVEIKRTVAAPSVFIFPPSD (SEQ ID NO: 557) |
| H1* | QQYGSS PQT (SEQ ID NO: 261) | FGQGTEVEIKRTVAAPSVFIFPPSDEQ (SEQ ID NO: 558) |
| H1** | QQYGPS PPGYT (SEQ ID NO: 262) | FGQGTKLEIKRTVAAPSVFIFPPSDEQLKSGTASVV (SEQ ID NO: |
| | | 559) |
| H2 | QQYGSS PQT (SEQ ID NO: 263) | FGQGTKVEIKRTVAAPSVFIFPPSDEQLKSGTASVV (SEQ ID NO: |
| | | 560) |
| H3 | QQYYRS PLT (SEQ ID NO: 264) | FGGGTKVEIKRTVAAPSVFIFPPSDEQLKSGTASVV (SEQ ID NO: |
| | | 561) |
| I | QQYNKWPPWT (SEQ ID NO: 265) | FGQGTKLEIKRTVAAPSVFVFPPS (SEQ ID NO: 562) |
| J1 | QQYGSS PQT (SEQ ID NO: 266) | FGQGTKVEIKRTVAAPSVFIFPPSDEQLKSGTASVV (SEQ ID NO.: |
| | | 563) |
| J2 | QQYGSS PQT (SEQ ID NO: 267) | FGQGTKVEIKRTVAAPSVFIFPPSDEQLKSGTASVV (SEQ ID NO: |
| | | 564) |
| J3 | QQYGSS PQT (SEQ ID NO: 268) | FGQGTKVEIKRTVAAPSVFIFPPSDEQLKSGTASVV (SEQ ID NO: |
| | | 565) |
| J3* | QQYGSS PQT (SEQ ID NO: 269) | FGQGTKVEIKRTVAAPSVFIFPPSDEQLKSGTASVV (SEQ ID NO: |
| | | 566) |
| J4 | QQYGSS PQT (SEQ ID NO: 270) | FGQGTEVEIKRTVAAPSVFIFPPSDEQLKSGTASVV (SEQ ID NO: |
| | | 567) |
| K | QQYGGS PPRFT (SEQ ID NO: 271) | FGPGTKVDIRRTVAAPSVFIFPPSDEQLKSGTASVV (SEQ ID NO: |
| | | 568) |
| L1 | QQYGDS VG (SEQ ID NO: 272) | FGPGTKVEIKRTVAAPSVFIFPPSDEQLKSGTASVV (SEQ ID NO: |
| | | 569) |
| L2 | QQYGDS VG (SEQ ID NO: 272) | FGPGTKVEIKRTVAAPSVFIFPPSDEQLKSGTASVV (SEQ ID NO: |
| | | 569) |
| L3 | QQYGSS PLT (SEQ ID NO: 273) | FGQGTKVEIKRTVAAPSVFIFPPSDEQLKSGTASVV (SEQ ID NO: |
| | | 570) |
| L4 | QQYGLS ST (SEQ ID NO: 274) | FGQGTRLEIKRTVAAPSVFIFPPSDEQLKSGTASVV (SEQ ID NO: |
| | | 571) |
| M | HHYGGS PRT (SEQ ID NO: 275) | FGQGTKVEIKRTVAAPSVFIFPPSDEQLKSGTASVV (SEQ ID NO: |
| | | 572) |

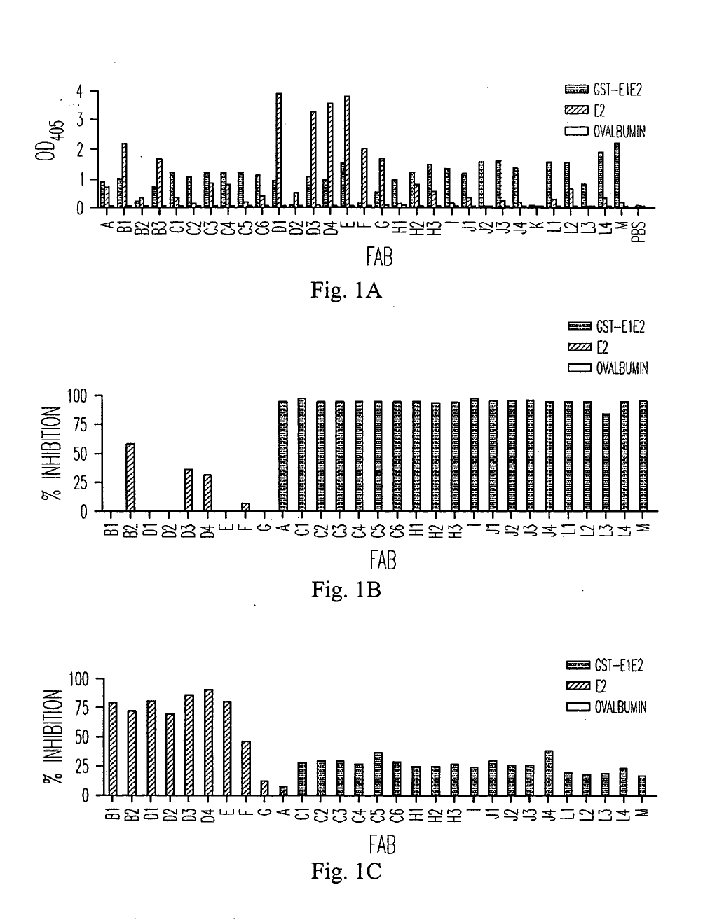

The binding properties of soluble Fabs prepared from the phage-Fab clones were characterized (FIG. 1). This allowed the Fabs to be sorted into three groups recognizing three antigenic regions (AR) of HCV E2 as shown in the table below.

TABLE 12
Three Distinct Antigenic Regions Defined by the Fab Panel

| | | | Competition | Competition |
|---|---|---|---|---|
| | | Epitope | with MAb | with MAb |
| AR | AR Recognized by Fab | Presentation | AR3A | H53 |
| 1 | B1, B2, B3, D1, D2, | E2 > E1E2 | No/Partial | Strong |
| | D3, D4, E (63%) | | | |
| 2 | F, G (2%) | E2 > E1E2 | No | Partial |
| 3 | A, C1, C2, C3, C4, | E1E2 > E2 | E1E2 > E2 | Partial |
| | C5, C6, H1, H2, H3, | | | |
| | I, J1, J2, J3, J4, L1, L2, | | | |
| | L3, L4, M (35%) | | | |

The numbers in parenthesis denote the percentage of clones recognizing each AR in the phage-display panning. It is important to note that highly isolate-specific antibodies, e.g. those against HVR1, would unlikely be selected in this study due to the use of heterologous antigens in the panning. Fab K was excluded in this table due to its poor signal in FIG. 1.

A total of seven Fabs from different heavy-chain groups recognizing the three different antigenic regions were converted into full-length IgG1s and their binding properties were evaluated (Table 13). In addition, the neutralizing activities of the mAbs were studied using a panel of HCV pseudotype virus particles (HCVpp) displaying E1E2 from diverse genotypes (Table 14). See Wakita et al. Nat. Med. 11: 791-96 (2005); Bartosch et al., J. Exp. Med. 197: 633-42 (2003); Hsu et al., Proc. Natl. Acad. Sci. USA 100: 7271-7276 (2003)).

TABLE 13
Binding properties of E2-specific IgGs

| | | | | | Apparent affinity for E1- | |
|---|---|---|---|---|---|---|
| | | | | | E2 (nM)^{d} | |
| | Derived from | | | IC50 E1-E2 binding to | (GT1a) | (GT1a) |
| IgG1 | Fab | Specificity | Epitope | CD81-LEL^{a} | HCV-1^{b} | H77^{c} |
| AR1A | B2 | AR1 | Discontinuous | 5.7 | 2.6 | 3.8 |
| AR1B | D1 | AR1 | Discontinuous | — | 0.4 | 0.6 |
| AR2A | G | AR2 | Discontinuous | — | 3.1 | 1.6 |
| AR3A | C1 | AR3 | Discontinuous | 0.5 | 1.3 | 3.7 |
| AR3B | J2 | AR3 | Discontinuous | 1.6 | 2.0 | 6.0 |
| AR3C | H3 | AR3 | Discontinuous | 2.0 | 1.4 | 2.3 |
| AR3D | L4 | AR3 | Discontinuous | 1.0 | 2.4 | 4.0 |

^{a}Antibody concentration (nM) to inhibit 50% of E1-E2 (isolate H77) binding to immobilized recombinant large extracellular loop of CD81 (CD81-LEL).
^{b}Vaccinia-expressed E1-E2.
^{c}E1-E2 produced by transfected 293T cells.
^{d}Apparent affinity is defined as the antibody concentration required to achieve half-maximal binding in an ELISA. Data shown are the means of at least two independent experiments. All mAbs bind natively folded, but not reduced and denatured, E2. GT1a indicates genotype 1a, GT2a indicates genotype 2a and dashes indicate that no significant inhibition or binding was observed with the highest mAb concentration tested.

TABLE 14
Neutralizing activity (IC50) of E2-specific IgGs

| | HCVpp^{b} | | | | | | | | | |
|---|---|---|---|---|---|---|---|---|---|---|
| | 1a | | 1b | | 2a | | 2b | 4 | 5 | Control |
| IgG^{a} | H77 | H | OH8 | UKN1B12.16 | J6 | UKN2A1.2 | UKN2B1.1 | UKN4.21.16 | UKN5.15.7 | VSV |
| AR1A | — | | — | — | — | — | — | — | — | — |
| AR1B | — | | — | — | — | — | — | — | — | — |
| AR2A | 1 | 5 | — | — | — | 5 | 10 | 1 | 10 | — |
| AR3A | 1 | 1 | 5 | 1 | 10 | 10 | 10 | 1 | 1 | — |
| AR3B | 1 | 1 | 5 | 1 | 10 | 5 | 10 | 1 | 1 | — |
| AR3C | 1 | 1 | 5 | 1 | 10 | 10 | 10 | 1 | 1 | — |
| AR3D | 1 | 1 | 5 | 1 | 50 | 25 | 25 | 1 | 10 | — |

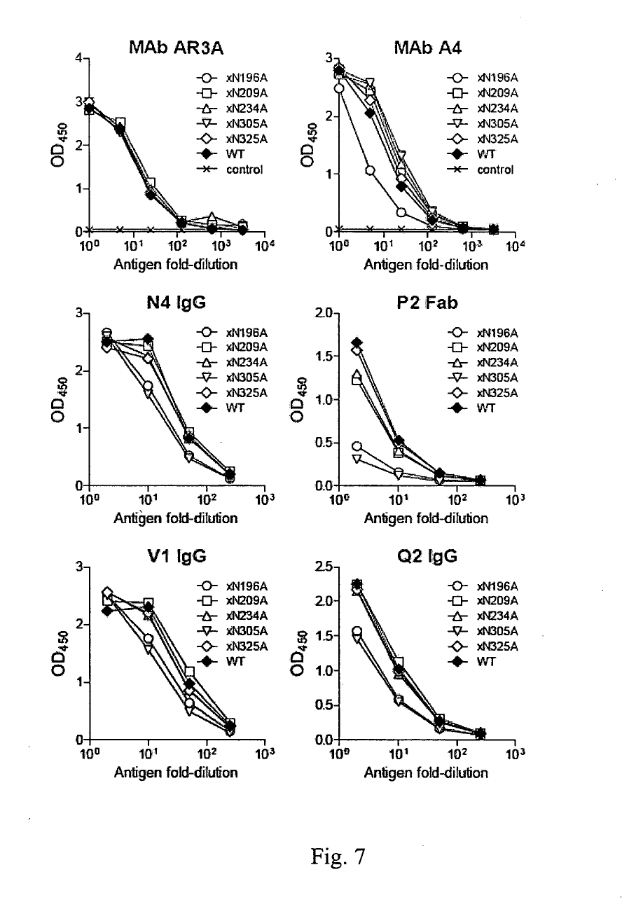

^{a}mAbs at 50, 25, 10, 5 or 1 μg/mL were tested for virus neutralization, and the lowest antibody concentrations that reduced >50% of virus infectivity are shown. Dashes indicate no or <50% virus neutralization with 50 μg/mL mAb. Data shown are the means of at least two experiments.
^{b}Neutralization of HCVpp was determined by the reduction in luciferase activity in Huh-7 cells infected with HCVpp displaying Env from different HCV isolates. The panel of HCVpps shown includes HCV Env proteins that produce a signal at least tenfold higher than the background signal induced by the control pseudotype virus generated without HCV Env cDNA. Many HCV Env proteins, including CH35 (genotype 1b), UKN3A1.28c (genotype 3a), UKN6.5.8 (genotype 6) and 13 different KP Env clones (genotype 1a, see FIG. 7B), did not produce a consistent signal tenfold higher than background and were excluded from this analysis.

The above results indicate that all recombinant mAbs bound the E1-E2 complex from HCV genotype 1a with approximately similar apparent affinities, in the range of 0.4-6 nM, but only antigenic region 3 (AR3)-specific mAbs reacted with genotype 2a HCV, suggesting that epitopes in AR3 are highly conserved. Monoclonal antibodies ARIA and AR3A-D inhibited the binding of E1-E2 to the virus co-receptor CD81 (Pileri et al. Science 282, 938-41 (1998); Cocquerel et al., J. Virol. 77, 10677-83 (2003)) at nanomolar concentrations, suggesting that these antibodies could potentially block HCV interaction with CD81 and thereby inhibit infection.

In addition, these experiments indicate the following.

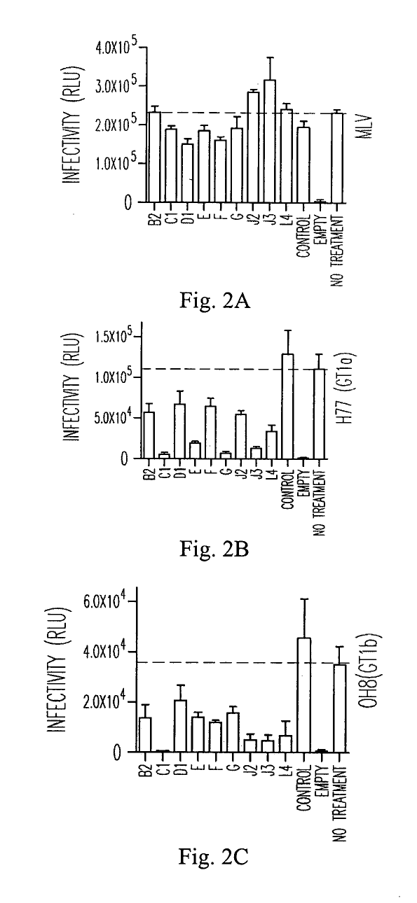

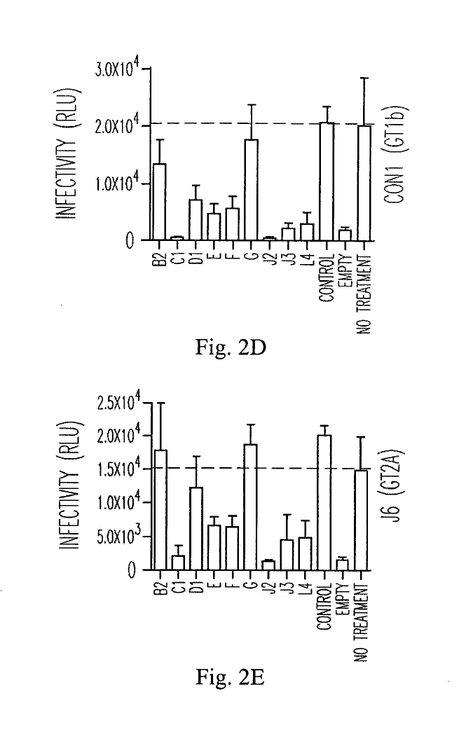

First, antibodies that bind E2 in an ELISA did not necessarily neutralize the corresponding virus. The AR1-specific antibodies bound recombinant E1-E2 from genotype 1a HCV isolate H77 with a similar or higher affinity than AR3-specific antibodies, but they did not neutralize the virus, suggesting that the AR1 epitopes are available on isolated envelope proteins but not on infectious virions. Of note, the Fab fragments of antibodies ARIA and AR1B (that is, B2 and D1, Table 13) did neutralize HCVpp-H77 (FIG. 2B), indicating that steric hindrance, possibly by E1, prevents virus neutralization by whole AR1-specific antibodies.

Second, the ability of the antibodies to inhibit E1-E2 binding to CD81 in the neutralization of binding (NOB) assay (Rosa et al., Proc. Natl. Acad. Sci. USA 93: 1759-63 (1996)) did not fully predict virus neutralization.

Third, and most notably, the AR3-specific antibodies bound E1-E2 from both genotypes 1a and 2a at nanomolar affinities and cross-neutralized many HCVpps tested. These results show that AR3 is a relatively conserved neutralizing site on HCV E2.

The specificity, affinity and neutralizing activities of the E2-specific human monoclonal antibodies were evaluated by mapping the antigenic regions using competition ELISA and alanine-mutagenesis scanning. Results are shown in the following Tables 15 and 16.

TABLE 15
Antibody Competition

| | | Blocking MAb | | | | | | | | | | |
|---|---|---|---|---|---|---|---|---|---|---|---|---|
| | origin | h | h | h | h | h | h | h | h | h | h | h |
| | specificity | AR1 | AR1 | AR2 | AR3 | AR3 | AR3 | AR3 | Dom A | Dom B | Dom B | Dom C |
| | MAb | AR1A | AR1B | AR2A | AR3A | AR3B | AR3C | AR3D | CBH-4B | CBH-2 | CBH-5 | CBH-7 |
| Binding MAb | AR1A | 15 | 15 | 100 | 44 | 53 | 37 | 54 | 102 | 83 | 54 | 16 |
| (biotinylated) | AR1B | 15 | 6 | 89 | 76 | 80 | 66 | 84 | 106 | 98 | 89 | 20 |
| | AR2A | 76 | 57 | 15 | 76 | 92 | 86 | 75 | 87 | 93 | 91 | 17 |
| | AR3A | 30 | 96 | 72 | 10 | 12 | 10 | 12 | 93 | 48 | 17 | 33 |
| | AR3B | 35 | 104 | 87 | 18 | 21 | 19 | 20 | 93 | 46 | 21 | 35 |
| | AR3C | 34 | 91 | 83 | 9 | 12 | 8 | 12 | 99 | 65 | 19 | 39 |
| | AR3D | 43 | 92 | 75 | 13 | 15 | 13 | 14 | 97 | 51 | 18 | 33 |

| | | Blocking MAb | | | | | | |
|---|---|---|---|---|---|---|---|---|
| | origin | m | r | r | m | r | r | r |
| | specificity | dis-cont. | 384-391 | 396-407 | 412-424 | 412-423 | 432-443 | 436-443 |
| | MAb | H53 | 7/59 | 9/27 | AP33* | 3/11* | 1/39 | 2/69A |
| Binding MAb | AR1A | 20 | 70 | 74 | 102 | 66 | 84 | 86 |
| (biotinylated) | AR1B | 12 | 77 | 70 | 69 | 61 | 89 | 85 |
| | AR2A | 107 | 99 | 67 | 63 | 94 | 90 | 82 |
| | AR3A | 75 | 84 | 48 | 26 | 36 | 76 | 55 |
| | AR3B | 73 | 86 | 49 | 32 | 41 | 79 | 58 |
| | AR3C | 77 | 95 | 62 | 33 | 44 | 83 | 68 |
| | AR3D | 74 | 86 | 49 | 27 | 37 | 80 | 56 |

| | | Blocking MAb | | | | | | |
|---|---|---|---|---|---|---|---|---|
| | origin | r | r | m | r | r | m | m |
| | specificity | 436-446 | 436-447 | 464-471 | 524-531 | 544-551 | 640-654 | 197-207 |
| | MAb | 7/16B | 11/20 | AP320 | 9/75 | 6/53 | ALP98 | A4 |
| Binding MAb | AR1A | 86 | 84 | 84 | 24 | 89 | 92 | 97 |
| (biotinylated) | AR1B | 91 | 79 | 83 | 38 | 92 | 92 | 103 |
| | AR2A | 95 | 84 | 70 | 82 | 92 | 94 | 92 |
| | AR3A | 101 | 47 | 77 | 39 | 104 | 96 | 94 |
| | AR3B | 92 | 49 | 79 | 44 | 108 | 102 | 91 |
| | AR3C | 99 | 62 | 81 | 51 | 105 | 99 | 93 |
| | AR3D | 94 | 47 | 77 | 53 | 103 | 97 | 90 |

Numbers indicate percentage of residual binding signals of biotinylated human mAbs in the presence of blocking mAbs. Origin: h, human; m, mouse; r, rat.

TABLE 16
Alanine-scanning Mutagenesis

| | | Q412A | L413A | I414A | N415A | T416A | N417A | G418A | S419A | W420A | H421A | I422A | N423A | S424A |
|---|---|---|---|---|---|---|---|---|---|---|---|---|---|---|
| Human IgG | AR1A | 145 | 103 | 128 | 106 | 23 | 17 | 51 | 84 | 141 | 167 | 106 | 79 | 103 |
| | AR1B | 48 | 109 | 147 | 131 | 99 | 54 | 143 | 118 | 41 | 72 | 58 | 38 | 103 |
| | AR2A | 95 | 92 | 180 | 122 | 99 | 58 | 83 | 93 | 80 | 84 | 95 | 69 | 91 |
| | AR3A | 53 | 88 | 132 | 103 | 82 | 64 | 86 | 92 | 78 | 88 | 113 | 60 | 5 |
| | AR3B | 37 | 90 | 116 | 89 | 41 | 65 | 47 | 83 | 65 | 53 | 78 | 35 | 7 |
| | AR3C | 85 | 111 | 207 | 138 | 152 | 73 | 188 | 102 | 78 | 91 | 111 | 84 | 7 |
| | AR3D | 44 | 83 | 126 | 131 | 90 | 74 | 60 | 73 | 68 | 87 | 113 | 65 | 6 |

| | | R483A | P484A | Y485A | W487A | H488A | Y489A | P491A | G523A | P525A | T526A | Y527A | W529A |
|---|---|---|---|---|---|---|---|---|---|---|---|---|---|
| Human IgG | AR1A | 64 | 38 | 17 | 89 | 91 | 189 | 119 | 112 | 101 | 81 | 61 | 367 |
| | AR1B | 32 | 45 | 30 | 70 | 63 | 52 | 118 | 33 | 33 | 48 | 83 | 94 |
| | AR2A | 68 | 77 | 67 | 93 | 72 | 138 | 123 | 129 | 109 | 101 | 149 | 132 |
| | AR3A | 114 | 62 | 124 | 109 | 90 | 111 | 161 | 19 | 0 | 111 | 163 | 194 |
| | AR3B | 97 | 58 | 125 | 113 | 90 | 53 | 178 | 49 | 26 | 82 | 119 | 80 |
| | AR3C | 96 | 75 | 112 | 64 | 48 | 98 | 173 | 38 | 16 | 123 | 54 | 51 |
| | AR3D | 151 | 90 | 145 | 119 | 123 | 113 | 204 | 48 | 53 | 135 | 119 | 88 |

| | | G530A | N532A | D533A | T534A | D535A | V538A | N540A | R543A | P544A | P545A | G547A | W549A | F550A |
|---|---|---|---|---|---|---|---|---|---|---|---|---|---|---|
| Human IgG | AR1A | 227 | 82 | 101 | 100 | 235 | 17 | 0 | 239 | 69 | 74 | 34 | 0 | 379 |
| | AR1B | 50 | 73 | 90 | 49 | 121 | 52 | 25 | 135 | 16 | 20 | 0 | 0 | 257 |
| | AR2A | 53 | 58 | 60 | 84 | 133 | 62 | 30 | 125 | 126 | 106 | 83 | 151 | 156 |
| | AR3A | 9 | 121 | 89 | 126 | 0 | 47 | 22 | 195 | 241 | 212 | 115 | 207 | 218 |
| | AR3B | 14 | 132 | 101 | 113 | 20 | 67 | 29 | 265 | 303 | 321 | 177 | 228 | 259 |
| | AR3C | 0 | 129 | 66 | 119 | 0 | 34 | 0 | 111 | 158 | 159 | 64 | 155 | 134 |
| | AR3D | 24 | 159 | 146 | 181 | 32 | 95 | 56 | 276 | 318 | 307 | 202 | 274 | 320 |

The panel of variants (top row) includes substitutions at conserved residues in the putative CD81-binding regions of E2. Substitutions important for CD81 binding are shaded and include L413A, W420A, H421A, I422A, N423A, S424A, G523A, T526A, Y527A, W529A, G530A, D535A, V538A, N540A and F550A. (Owsianka, A. M. et al., J. Virol. 80, 8695-8704 (2006)).

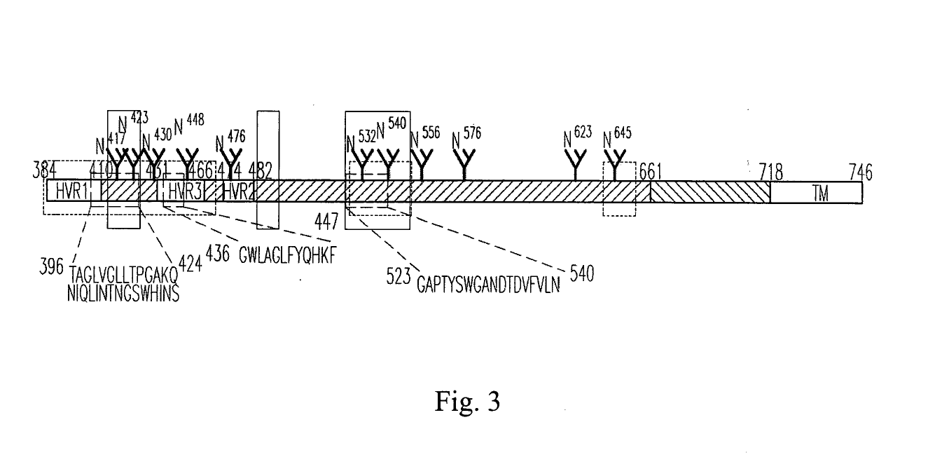

The antibody competition study shows that mAbs AP33 and 3/11 (*) recognize epitopes partially dependent on proper protein folding (Tarr, A. W. et al. Hepatology 43, 592-601 (2006)). The results confirm the broad designation of the antigenic regions and suggest that the discontinuous epitopes in AR3 are formed by at least three segments between amino acids 396-424, 436-447 and 523-540; the first and third segments also contribute to the CD81-binding domain of E2 (Owsianka, A. M. et al. J. Virol. 80, 8695-8704 (2006)), and the conserved residues Ser424, Gly523, Pro525, Gly530, Asp535, Val538 and Asn540 (Owsianka, A. M. et al. J. Virol. 80, 8695-8704 (2006)) are probably involved in the binding of the AR3-specific antibodies (FIG. 3).

A key question is whether broadly neutralizing AR3-specific antibodies can protect against infection by heterologous HCV quasispecies. As a first step to evaluate the mAbs and establish the essential parameters for passive antibody protection, the human liver-chimeric Alb-uPA/SCID mouse model was used (Kneteman, N. M. et al. Hepatology 43, 1346-1353 (2006); Lindenbach, B. D. et al. Proc. Natl. Acad. Sci. USA 103, 3805-3809 (2006)). Although this animal model is not suitable for studying virus pathogenesis, owing to its lack of a functional adaptive immune system, the question of whether antibodies can protect against HCV challenge is appropriate.

The kinetics and tolerability were first established in the animal model for the antibodies AR3A, AR3B and a human isotype control IgG1 to HIV-1, b6. Previous passive antibody studies in animal models have reported that relatively high antibody concentrations are needed for protection. For instance, to achieve sterilizing immunity by single mAB treatment against HIV in hu-PBL/SCID mice (Gauduin et al. Nat. Med. 3, 1389-1393 (1997) (and against chimeric simian/human immunodeficiency virus (SHIV) in macaques (Parren et al. J. Virol. 75, 8340-8347 (2001)), serum concentrations in the animals of the order of 100-fold in vitro 90% neutralization titers (IC₉₀) have been required. To study the kinetics of the human MAbs AR3A, AR3B and b6 in control Alb-uPA/SCID mice, transplanted Alb-uPA/SCID mice with a low level of human liver chimerism were injected intraperitoneally with 100, 150 or 200 mg/kg MAb, and blood samples were collected by tail bleed, Human antibody in the murine sera was measured by a quantitative sandwich ELISA using conjugated and unconjugated goat anti-human F(ab)′₂ antibody. The IC₉₀ titers (against HCVpp-H77) for MAb AR3A and AR3B are 11 and 20 g/mL, respectively, suggesting that relatively large doses of antibody may be required for protection. A dose of 200 mg/kg given through intraperitoneal injection was required to achieve mean serum titers approximately 100× higher than in vitro neutralization titers. No specific weight loss or signs of illness associated with the administration of the MAbs were noted in the mice during the experiment. One mouse was euthanized due to unrelated morbidity at Day 7.

The neutralizing activity in mouse sera collected ten days after injection was determined by HCVpp-H77 neutralization assay. Mouse sera containing anti-HCV MAbs AR3A and AR3B neutralized 50% of HCVpp infectivity (IC₅₀) in the range of 1:200 to 1:1000. Control mouse sera from mice injected with an isotype MAb DEN3 or PBS (neutralized non-specifically ˜50% virus infectivity at 10-fold dilution. Non-specific neutralization was not observed when the control sera were diluted 100-fold. The conservation of virus neutralizing activity of anti-HCV MAbs also was measured and normalized to the level of human IgG in the mouse sera. In this experiment, MAbs AR3A and AR3B were titrated alongside with the mouse sera to construct the standard curves and the IC₅₀ titers of MAbs AR3A and AR3B were 0.4 and 1 μg/mL, respectively. The IC₅₀ titers of mouse sera containing anti-HCV MAbs AR3A and AR3B were in the range of 0.4-1.1 (mean 0.8±s.d. 0.3) and 0.5-3 (mean 1.2±s.d. 0.9) μg/mL, respectively. Isotype control MAbs b6 & DEN3 did not neutralize HCVpp.

The observed half-lives of mAbs AR3A, AR3B and b6 were found to be 6.0±2.2 d, 9.0±1.3 d and 7.3±1.8 d (mean±s.d.), respectively, and their specific neutralizing activities (that is, neutralizing activity relative to serum mAb concentration) were stable for at least 10 days in the mice.

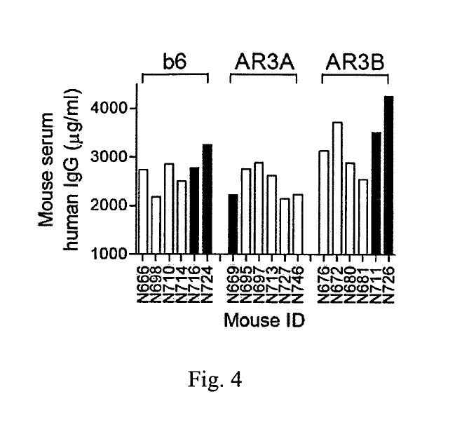

The mAbs were administered intraperitoneally in passive transfer experiments to mice with high levels of human liver chimerism (see Example 1), and the mean serum titers of mAbs AR3A, AR3B and the control mAb b6, at 24 hours after injection were ˜2.5±0.3 mg/mL, 3.1±0.5 mg/mL and 2.6±0.3 mg/mL, respectively (FIG. 4). To simulate a natural human exposure to virus, we inoculated genotype 1a HCV-infected human serum intravenously into the mice. The partial amino acid sequences (residues 384-622) of forty HCVs found in the viral quasispecies population in the HCV genotype 1a-infected human serum are shown below.

TABLE 17
Cloned Variant Sequences of E2 Amino Acid Residues 384-622

| | |
|---|---|
| Name | Sequence |

| | |
|---|---|
| KP S9 | ETHVTGGATAHGASVLASLLTTGAKQNIQLINTNGSWHINRTALNCNDSLHT |
| | GFIAGLFYYNKFNSSGCPERLASCRRLDDFAQGWGPISHVNVSGPGERPYCW |
| | HYPPRPCGIVPARDVCGPVYCFTPSPVVVGTTDRAGAPTYNWGANETDVFVL |
| | NNTRPPLGNWFGCTWMNSTGFTKVCGAPPCVIGGVGNNTLRCPTDCFRKHPD |
| | ATYSRCGSGPWITPRCLVDYPYRLWHYPCTI (SEQ ID NO: 701) |
| KP R14 | ETHVTGGATAHGASVLASLLTTGAKQNIQLINTNGSWHINRTALNCNDSLHT |
| | GFIAGLFYYNKFNSSGCPERLAGCRRLDDFAQGWGPISHVNVSGPGERPYCW |
| | HYPPRPCGIVPARDVCGPVYCFTPSPVVVGTTDRAGAPTYNWGANETDVFVL |
| | NNTRPPLGNWFGCTWMNSTGFTKVCGAPPCVIGGVGNNTLRCPTDCFRKHPD |
| | ATYSRCGSGPWITPRCLVDYPYRLWHYPCTI (SEQ ID NO: 702) |
| KP S6 | ETHVTGGATAHGASVLASLLTTGAKQNIQLINTNGSWHINRTALNCNDSLHT |
| | GFIAGLFYYNKFNSSGCPERLASCRRLDDFAQGWGPISHVNVSGPGERPYCW |
| | HYPPRPCGIVPARDVCGPVYCFTPSPVVVGTTDRAGAPSYNWGANETDVFVL |
| | NNTRPPLGNWFGCTWMNSTGFTKVCGAPPCVIGGVGNNTLRCPTDCFRKHPD |
| | ATYSRCGSGPWITPRCLVDYPYRLWHYPCTI (SEQ ID NO: 703) |
| KP S18 | ETHVTGGATAHGASVLASLLTTGAKQNIQLINTNGSWHINRTALNCNDSLHT |
| | GFIAGLFYYNKFNSSGCPERLASCRRLDDFAQGWGPISHVNVSGPGERPYCW |
| | HYPPRPCGIVPARDVCGPVYCFTPSPVVVGTTDRAGAPTYNWGANETDVFVL |
| | NNTRPPLGNWFGCTWMDSTGFTKVCGAPPCVIGGVGNNTLRCPTDCFRKHPD |
| | ATYSRCGSGPWITPRCLVDYPYRLWHYPCTI (SEQ ID NO: 704) |
| KP S16 | ETHVTGGATAHGASVLASLLTTGAKQNIQLINTNGSWHINRTALNCNDSLHT |
| | GFIAGLFYYNKFNSSGCPERLASCRRLDDFAQGWGPISHVNVSGPGERPYCW |
| | HYPPRPCGIVPARDVCGPVYCFTPSPVVVGTTDRAGAPTYNWGANETDVFVL |
| | NNTRPPLGNWFGCTWMNSTGFTKVCGALPCVIGGVGNNTLRCPTDCFRKHPD |
| | ATYSRCGSGPWITPRCLVDYPYRLWHYPCTI (SEQ ID NO: 705) |
| KP R8 | ETHVTGGATAHGASVLASLLTTGAKQNIQLINTNGSWHINRTALNCNDSLHT |
| | GFIAGLFYYNKFNSSGCPERLASCRRLDDFAQGWGPISHVNVSGPGERPYCW |
| | HYPPRPCGIVPARDVCGPVYCFTPSPVVVGTTDRAGAPTYNWGANETDVFVL |
| | NNTRPPLGNWFGCTWMNSTGFTKVCGAPPCVIGGVGSNTLRCPTDCFRKHPD |
| | ATYSRCGSGPWITPRCLVDYPYRLWHYPCTI (SEQ ID NO: 706) |
| KP S20 | ETHVTGGATAHGASVLASLLTTGAKQNIQLINTNGSWHVNRTALNCNDSLHT |
| | GFIAGLFYYNKFNSSGCPERLASCRRLDDFAQGWGPISHVNVSGPGERPYCW |
| | HYPPRPCGIVPARDVCGPVHCFTPSPVVVGTTDRAGAPTYNWGANETDVFVL |
| | NNTRPPLGNWFGCTWMNSTGFTKVCGAPPCVIGGVGNNTLRCPTDCFRKHPD |
| | ATYSRCGSGPWITPRCLVDYPYRLWHYPCTI (SEQ ID NO: 707) |
| KP S4 | ETHVTGGATAHGASVLASLLTTGAKQNIQLINTNGSWHINRTALNCNDSLHT |
| | GFIVGLFYYNKFNSSGCPERLASCRRLDDFAQGWGPISYVNVSGPGERPYCW |
| | HYPPRPCGIVPARDVCGPVYCFTPSPVVVGTTDRAGAPTYNWGANETDVFVL |
| | NNTRPPLGNWFGCTWMNSTGFTKVCGAPPCVIGGVGNNTLRCPTDCFRKHPD |
| | ATYSRCGSGPWITPRCLVDYPYRLWHYPCTI (SEQ ID NO: 708) |
| KP R3 | ETHVTGGATAHGASVLASLLTTGAKQNIQLINTNGSWHINRTALNCNDSLHT |
| | GFIAGLFYYNKFDSSGCPERLASCRRLDDFAQGWGPISYVNVSGPGERPYCW |
| | HYPPRPCGIVPARDVCGPVYCFTPSPVVVGTTDRAGAPTYNWGANETDVFVL |
| | NNTRPPLGNWFGCTWMNSTGFTKVCGAPPCVIGGVGNNTLRCPTDCFRKHPD |
| | ATYSRCGSGPWITPRCLVDYPYRLWHYPCTI (SEQ ID NO: 709) |
| KP S3 | ETHVTGGATAHGASVLASLLTTGAKQNIQLINTNGSWHINRTALNCNDSLHT |
| | GFIAGLFYYNKFDSSGCPERLASCRRLDDFAQGWGPISYVNVSGPGERPYCW |
| | HYPPRPCGIVPARDVCGPVYCFTPSPVVVGTTDRAGAPTYNWGANETDVFVL |
| | NNTRPPLGNWFGCTWMNSTGFTKVCGAPPCDIGGVGNNTLRCPTDCFRKHPD |
| | ATYSRCGSGPWITPRCLVDYPYRLWHYPCTI (SEQ ID NO: 710) |
| KP S12 | ETHVTGGATAHGASVLASLLTPGAKQHVQLINTNGSWHINRTALNCNDSLHT |
| | GFIAGLFYYNKFDSSGCPERLASCRRLDDFAQGWGPISYVNVSGPGERPYCW |
| | HYPPRPCGIVPARDVCGPVYCFTPSPVVVGTTDRAGAPTYNWGANETDVFVL |
| | NNTRPPLGNWFGCTWMNSTGFTKVCGAPPCVIGGVGNNTLRCPTDCFRKHPD |
| | ATYSRCGSGPWITPRCLVDYPYRLWHYPCTI (SEQ ID NO: 711) |
| KP S15 | ETHVTGGATAHGASVLASLLTPGAKQNIQLINTNGSWHINRTALNCNDSLHT |
| | GFVAGLFYYNKFDSSGCPERLASCRRLDDFAQGWGPISHVNVSGPGERPYCW |
| | HYPPRPCGIVPARDVCGPVYCFTPSPVVVGTTDRAGAPTYNWGANETDVFVL |
| | NNTRPPLGNWFGCTWMNSTGFTKVCGAPPCVIGGVGNNTLRCPTDCFRKHPD |
| | ATYSRCGSGPWITPRCLVDYPYRLWHYPCTI (SEQ ID NO: 712) |
| KP S5 | ETHVTGGATAHGASVLASLLTPGAKQNIQLINTNGSWHINRTALNCNDSLHT |
| | GFIAGLFYYNKFDSSGCLERLASCRRLDDFAQGWGPISHVNVSGPGERPYCW |
| | HYPPRPCGIVPARDVCGPVYCFTPSPVVVGTTDRAGAPTYNWGANETDVFVL |
| | NNTRPPLGNWFGCTWMNSTGFTKVCGAPPCAIGGVGNNTLRCPTDCFRKHPD |
| | ATYSRCGSGPWITPRCLVDYPYRLWHYPCTI (SEQ ID NO: 713) |
| KP R7 | ETHVTGGATAHGASVLASLLTPGAKQNIQLINTNGSWHINRTALNCNDSLHT |
| | GFIAGLFYYNKFNSSGCPERLASCRRLDDFAQGWGPISHVNVSGPGERPYCW |
| | HYPPRPCGIVPARDVCGPVYCFTPSPVVVGTTDRAGAPTYNWGANETDVFVL |
| | NNTRPPLGNWFGCTWMNSTGFTKVCGAPPCAIGGVGNNTLRCPTDCFRKHPD |
| | ATYSRCGSGPWITPRCLVDYPYRLWHYPCTI (SEQ ID NO: 714) |
| KP R11 | ETHVTGGATAHGASVLASLLTPGAKQNIQLINTNGSWHINRTALNCNDSLHT |
| | GFIAGLFYYNKFNSSGCPERLASCRRLDDFAQGWGPISHVNVSGPGERPYCW |
| | HYPPRPCGIVPARDVCGPVYCFTPSPVVVGTTDRAGAPTYNWGANETDVFVL |
| | NNTRPPLGNWFGCTWMNTTGFTKVCGAPSCVIGGVGNNTLRCPTDCFRKHPD |
| | ATYSRCGSGPWITPRCLVDYPYRLWHYPCTI (SEQ ID NO: 715) |
| KP R1 | ETHVTGGATAHGASVLASLLTPGAKQNIQLINTNGSWHINRTALNCNDSLHT |
| | GFIAGLFYYNKFNSSGCPERLASCRRLDDFAQGWGPISHVNVSGPGERPYCW |
| | HYPPRPCGIVPARDVCGPVYCFTPSPVVVGTTDRAGAPTYNWGANETDVFVL |
| | NNTRPPLGNWFGCTWMNTTGFTKVCGAPPCVIGGVGNNTLRCPTDCFRKHPD |
| | ATYSRCGSGPWITPRCLVDYPYRLWHYPCTI (SEQ ID NO: 716) |
| KP R12 | ETHVTGGATAHGASVLASLLTPGAKQNIQLINTNGSWHINRTALNCNDSLHT |
| | GFIAGLFYYNKFNSSGCPERLASCRRLDDFAQGWGPISHVNVSGPGERPYCW |
| | HYPPRPCGIVPARDVCGPVYCFTPSPVVVGTTDRAGAPTYNWGANETDVFVL |
| | NNTRPPLGNWFGCTWMNSTGFTKVCGAPPCVIGGVGNNTLRCPTDCFRKHPD |
| | ATYSRCGSGPWITPRCLVDYPYRLWHYPCTI (SEQ ID NO: 717) |
| KP S7 | ETHVTGGATAHGASVLASLLTPGAKQNIQLINTNGSWHINRTALNCNDSLHT |
| | GFIAGLFYYNKFNSSGCPERLASCRRLDDFAQGWGPISHVNVSGPGERPYCW |
| | HYPPRPCGIVPARDVCGPVYCFTPSPVVVGTTDRAGAPTYNWGANETDVFVL |
| | NNTRPPLGNWFGCTWMNSTGFTKVCGAPPCVIGGVGNNTLRCPTDCFRKHPD |
| | ATYSRCGSGPCITPRCLVDYPYRLWHYPCTI (SEQ ID NO: 718) |
| KP R15 | ETHVTGGATAHGASVLTSLLTTGAKQNIQLINTNGSWHINRTALNCNDSLHT |
| | GFIAGLFYYNKFNSSGCPERLASCRRLDDFAQGWGPISHVNVSGPGERPYCW |
| | HYPPRPCGIVPARDVCGPVYCFTPSPVVVGTTDRAGAPTYNWGANETDIFVL |
| | NNTRPPLGNWFGCTWMNSTGFTKVCGAPPCVIGGVGNNTLRCPTDCFRKHPD |
| | ATYSRCGSGPWITPRCLVDYPYRLWHYPCTI (SEQ ID NO: 719) |
| KP R18 | ETYVTGGATAHGASVLASLLTTGAKQNIQLINTNGSWHINRTALNCNDSLHT |
| | GFIAGLFYYNKFNSSGCPERLASCRRLDDFAQGWGPISHVNVSGPGERPYCW |
| | HYPPRPCGIVPARDVCGPVYCFTPSPVVVGTTDRAGAPTYNWGANETDVFVL |
| | NNTRPPLGNWFGCTWMNSTGFTKVCGAPPCVIGGVGNNTLRCPTDCFRKHPD |
| | ATYSRCGSGPWITPRCLVDYPYRLWHYPCTI (SEQ ID NO: 720) |
| KP S11 | ETHVTGGATAHGASVFASLLTTGAKQNIQLINTNGSWHINRTALNCNDSLHT |
| | GFIAGLFYYNKFNSSGCPERLASCRRLDDFAQGWGPISHVNVSGPGERPYCW |
| | HYPPRPCGIVPARDVCGPVYCFTPSPVVVGTTDRAGAPTYNWGANETDVFVL |
| | NNTRPPLGNWFGCTWMNSTGFTKVCGAPPCVIGGVGNNTLRCPTDCFRKHPD |
| | ATYSRCGSGPWITPRCLVDYPYRLWHYPCTI (SEQ ID NO: 721) |
| KP R20 | ETHVTGGATAHGASVFASLLTTGAKQNIQLINTNGSWHINRTALNCNDSLHT |
| | GFIAGLFYYNKFDSSGCPERLASCRRLDDFAQGWGPISHVNVSGPGERPYCW |
| | HYPPRPCGIVPARDVCGPVYCFTPSPVVVGTTDRAGAPTYNWGANETDVFVL |
| | NNTRPPLGNWFGCTWMNSTGFTKVCGAPPCVIGGVGNNTLRCPTDCFRKHPD |
| | ATYSRCGSGPWITPRCLVDYPYRLWHYPCTI (SEQ ID NO: 722) |
| H77 | ETHVTGGSAGRTTAGLVGLLTPGAKQNIQLINTNGSWHINSTALNCNESLNT |
| | GWLAGLFYQHKFNSSGCPERLASCRRLTDFAQGWGPISYANGSGLDERPYCW |
| | HYPPRPCGIVPAKSVCGPVYCFTPSPVVVGTTDRSGAPTYSWGANDTDVFVL |
| | NNTRPPLGNWFGCTWMNSTGFTKVCGAPPCVIGGVGNNTLLCPTDCFRKHPE |
| | ATYSRCGSGPWITPRCMVDYPYRLWHYPCTI (SEQ ID NO: 723) |
| UKN1b12.16 | RTRTTGGSAAQTTYGLTSLFRSGPSQKIQLVNTNGSWHINRTALNCNDSLNT |
| | GFLAALFYVRNFNSSGCPERMASCRPIDTFDQGWGPITYTEPHSLDQRPYCW |
| | HYAPQPCGIVPAAQVCGPVYCFTPSPVVVGTTDRFGAPTYTWGENETDVLIL |
| | NNTRPPQGNWFGCTWMNGTGFTKTCGGPPCNIGGAGNNTLICPTDCFRKHPE |
| | ATYTRCGSGPWLTPRCMVDYPYRLWHYPCTV (SEQ ID NO: 724) |

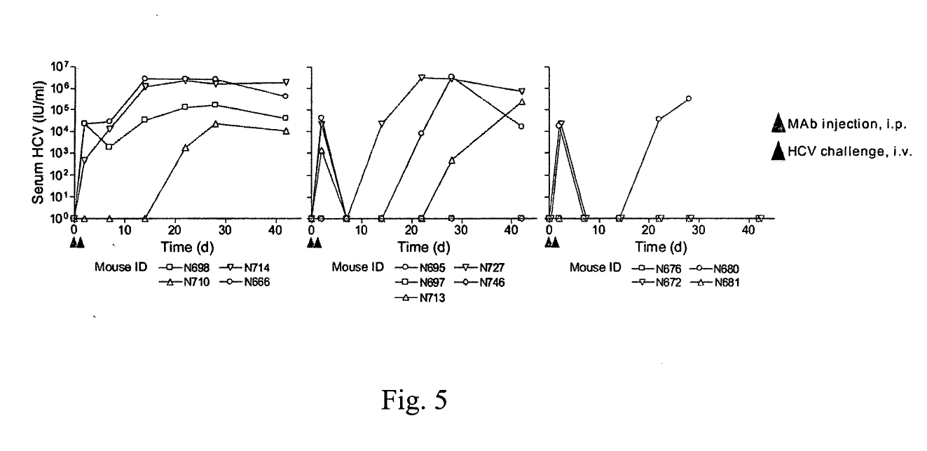

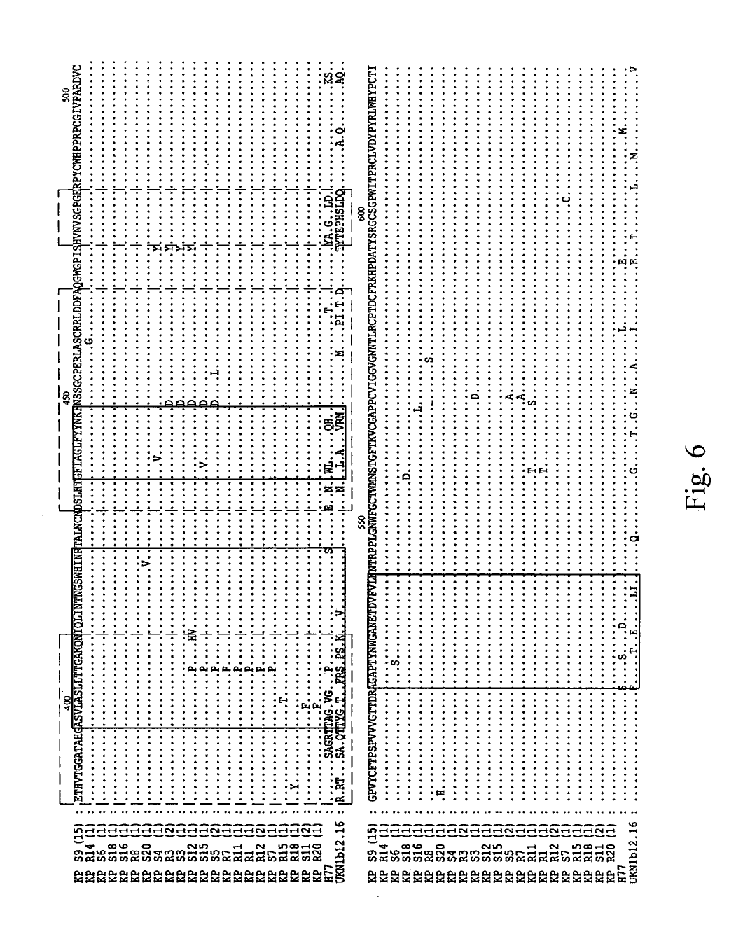

The alignment of these sequences are shown in FIG. 6. Infection was monitored by assessing serum viral load up to 6 weeks after inoculation (see FIG. 5). Protection in this mouse model is defined as the absence of serum HCV RNA as detected by quantitative PCR at or after 6 days post virus challenge. All mice in the control group (n=4) were infected, and serum viral load was maintained at >10,000 RNA copies/mL until the completion of the study. In mice that received mAb AR3A (n=5) or AR3B (n=4), HCV was detected the day after challenge in five of nine mice, but was cleared 6 days after virus challenge. High levels of HCV RNA were detected in four mice between weeks 2 and 4, indicating virus replication concurrent with the decay of antibody in these mice. By week 6, when the mAbs would have decayed to <10% of the initial serum level, two of five mice receiving mAb AR3A and three of four mice receiving mAb AR3B were still protected. The protection was highly significant compared to the isotype control antibody group (two-tailed log-rank test: AR3A, P=0.0298; AR3B, P=0.0171). The experiments ended at week 6 because two mice became morbid and were killed on day 41 and day 45, respectively, but the remaining mice were monitored to week 8, and a signal below the sensitivity of the quantitative PCR assay (6.0×10² international units/mL) was noted in one additional mouse in each neutralizing antibody-treated group (mice N681 and N697).

In summary, (i) it is possible to use mAbs against AR3 to protect against challenge with a heterologous HCV quasispecies swarm, consistent with the notion that AR3 is the principal conserved neutralizing antibody determinant of HCV; (ii) high concentrations of the mAbs were required for protection, suggesting that more potent antibody preparations will likely be required in immunotherapy, but that the mAbs described will be useful for comparative in vitro studies with newly identified mAbs and combinations of mAbs; and (iii) considering that one-third of the 115 phage-Fab clones isolated in this study are AR3 specific and are diverse in their heavy-chain sequences, and similar mAbs were isolated from different HCV-infected donors elsewhere (Keck, Z. Y. et al. J. Virol. 78, 9224-9232 (2004)), AR3 seems to be relatively immunogenic in humans and thus a favorable target for vaccine design. So, despite the enormous diversity of HCV, the prospects for developing a vaccine against this virus, that may target both conserved B and T cell epitopes (Elmowalid, G. A. et al. Proc. Natl. Acad. Sci. USA 104, 8427-8432 (2007); Folgori, A. et al. Nat. Med. 12, 190-197 (2006)), seem favorable.

### Example 3

### Isolation and Characterization of Additional Monoclonal Antibodies

Using epitope-masking/panning of phage-display. Fab library as described in Example 1, about twenty new monoclonal antibodies with unique properties were isolated. In this experiment, E1E2 complex masked with AR1- and AR3-antibodies was used as a panning antigen. First, E1E2 complex from isolate H77 produced by transient transfection of 293T cells was captured by MAb AR3A onto microwells. MAb AR3A binds only folded E2 and neutralizes HCVpp. Second, a saturating concentration of MAb AR1B antibody was added to the captured E1E2 to block all AR1 sites on the complex. The AR3-captured and AR1-masked E1E2 complex was used in library panning and ˜200 Fab clones obtained from the 3rd and 4th rounds of panning were analyzed for specific reactivity to unmasked E1E2 and a total of 29 clones were found to bind E1E2 of isolate H77 (genotype 1a) and 7 of those also cross-reacted with isolate J6 (genotype 2a) (data not shown). DNA sequence analysis revealed 19 unique antibody sequences, none of which has the same sequence found in the antibody panel we isolated previously. According to heavy chain sequences, the Fabs were sorted into 9 groups. The heavy and light chain amino acid sequences of these nineteen antibodies are shown in the tables below.

TABLE 18A
Fab Heavy Chain Sequences

| Fab | FRAMEWORK 1 | CDR1 |
|---|---|---|
| N1 | EVQLLEQSGPEVKKPGDSLRISCKMSGDSLV | TTWIG |
| | (SEQ ID NO: 835) | (SEQ ID NO: 725) |
| N2 | EVQLLEQSGPEVKKPGDSLRISCKMSGDSLV | TTWIG |
| | (SEQ ID NO: 836) | (SEQ ID NO: 726) |
| N3 | EVQLLEQSGAEVKKPGDSLRISCKMSGDSLV | WIG |
| | (SEQ ID NO: 837) | (SEQ ID NO: 983) |
| N4 | EVQLLEQSGPEVKKPGDSLRISCKMSGDSLV | TTWIG |
| | (SEQ ID NO: 838) | (SEQ ID NO: 727) |
| O1 | EVQLLEQSGAEVKKAGESVRLSCKASGYRFG | DYWIA |
| | (SEQ ID NO: 839) | (SEQ ID NO: 728) |
| P1 | EVQLLE SGGGVVQPGGSLRLSCAASGFTFT | SFTMH |
| | (SEQ ID NO: 840) | (SEQ ID NO: 729) |
| P2 | EVQLLE SGGGVVQPGRSLRLSCAASGFTLD | TFTMH |
| | (SEQ ID NO: 841) | (SEQ ID NO: 730) |
| P3 | EVQLLE SGGGVVQPGKSLRLSCAVSGFTLN | TFAMH |
| | (SEQ ID NO: 842) | (SEQ ID NO: 731) |
| P4 | EVQLLE SGGGVVQPGKSLTVSCAASGFTFS | TFTMH |
| | (SEQ ID NO: 843) | (SEQ ID NO: 732) |
| P5 | EVQLLE SGGGVVQPGKSLTVSCAASGFTFS | TFTMH |
| | (SEQ ID NO: 844) | (SEQ ID NO: 733) |
| P6 | EVQLLE SGGGVVQPGKSLTVSCAASGFTFS | TFTMH |
| | (SEQ ID NO: 845) | (SEQ ID NO: 734) |
| Q1 | EVQLLEQSGAEVRKPGASVKVSCKASGYTFT | NNGLN |
| | (SEQ ID NO: 846) | (SEQ ID NO: 735) |
| Q2 | EVQLLEQSGAEVKKPGASVKVSCKASGYTFT | NNGLN |
| | (SEQ ID NO: 847) | (SEQ ID NO: 736) |
| R1 | EVQLLEQSGAEVKKPGASVKVSCKASGYTFS | IYGVA |
| | (SEQ ID NO: 848) | (SEQ ID NO: 737) |
| S1 | EVQLLEQSGAEVKKPGTSVKVSCTASGYIFT | SFGIS |
| | (SEQ ID NO: 849) | (SEQ ID NO: 738) |
| T1 | EVQLLE SGGGLVQPGGSLRLSCAASGFTFS | RFWMH |
| | (SEQ ID NO: 850) | (SEQ ID NO: 739) |
| U1 | EVQLLEQSGPEVKRPGTSVKMSCKISGGASI | TQAMS |
| | (SEQ ID NO: 851) | (SEQ ID NO: 740) |
| V1 | EVQLLEQSGAEVQKPGASVKVSCKPSGYIFT | NFGIS |
| | (SEQ ID NO: 852) | (SEQ ID NO: 741) |

TABLE 18B
Fab Heavy Chain Sequences - Con't

| | | |
|---|---|---|
| Fab | FRAMEWORK 2 | CDR2 |

| | | |
|---|---|---|
| N1 | WVRQKPGQGLEWMG (SEQ ID NO: 853) | IINPGDSSTNIYPGDSATRYGPSFQG (SEQ ID NO: 742) |
| N2 | WVRQKPGQGLEWMG (SEQ ID NO: 854) | IINPGDSSTNIYPGDSATRYGPSFQG (SEQ ID NO: 743) |
| N3 | WVRQKPGQGLEWMG (SEQ ID NO: 855) | IINPGDSATNIYPGDSDTRYGPSFQG (SEQ ID NO: 744) |
| N4 | WVRQKPGQGLEWMG (SEQ ID NO: 856) | IINPGDSSTNIYPGDSATRYGPSFQG (SEQ ID NO: 745) |
| O1 | WVRQLPGRAPEWMG (SEQ ID NO: 857) | IIYPDDSDTKYSPSFQGQVTISADKSI (SEQ ID NO: 746) |
| P1 | WVRQAPGKGLEWVA (SEQ ID NO: 858) | LISHDGSNKDYADSVRG (SEQ ID NO: 747) |
| P2 | WVRQSPGKGLEWVA (SEQ ID NO: 859) | LISHDANNKDYADSVKG (SEQ ID NO: 748) |
| P3 | WVRQVPGKGLEWVA (SEQ ID NO: 860) | LTSHDGSRQDYADSVRG (SEQ ID NO: 749) |
| P4 | WVRQAPGKWLEWVA (SEQ ID NO: 861) | VISHDGGTEHYADSVTG (SEQ ID NO: 750) |
| P5 | WVRQAPGKGLEWVA (SEQ ID NO: 862) | VISHDGGTEHYADSVTG (SEQ ID NO: 751) |
| P6 | WVRQAPGKWLEWVA (SEQ ID NO: 863) | VISHDGGTEHYADSVTG (SEQ ID NO: 752) |
| Q1 | WVRQAPGQGLEWMG (SEQ ID NO: 864) | WISPYNGDTDFAHKFQG (SEQ ID NO: 753) |
| Q2 | WVRQAPGQGLEWMG (SEQ ID NO: 865) | WISPYNGDTDFAHKFQG (SEQ ID NO: 754) |
| R1 | WVRQAPGQGLEWMG (SEQ ID NO: 866) | WISPQNGDTHSPQKFQG (SEQ ID NO: 755) |
| S1 | WVRQAPGQGLEWMG (SEQ ID NO: 867) | RIDTYNGKTNYAQKLQG (SEQ ID NO: 756) |
| T1 | WVRQAPGKGLVWVA (SEQ ID NO: 868) | RINSDGSSTTYADSVKG (SEQ ID NO: 757) |
| U1 | WVRQAPGQGLEWMG (SEQ ID NO: 869) | GITPIFGTVNYAQKILG (SEQ ID NO: 758) |
| V1 | WVRQAPGQGLEWMA (SEQ ID NO: 870) | WINTYNGKTTYAQSLQG (SEQ ID NO: 759) |

TABLE 18C
Fab Heavy Chain Sequences - Con't

| | | | |
|---|---|---|---|
| Fab | FRAMEWORK 3 | CDR3 | FRAMEWORK 4 |

| | | | |
|---|---|---|---|
| N1 | QVTISIDKSTSTAYLQWNAVKPSDTGIYYCAR | HVPVPISGTFLWREREMHDFGYFDD | WGQGTLVIVSS |
| | (SEQ ID NO: 871) | (SEQ ID NO: 760) | (SEQ ID NO: 889) |
| N2 | QVTISIDKSTSTAYLQWNTVKPSDTGIYYCAR | HVPVPISGTFLWREREMHDFGYFDD | WGQGTLVIVSS |
| | (SEQ ID NO: 872) | (SEQ ID NO: 761) | (SEQ ID NO: 890) |
| N3 | QVTISIDKSTSTAYLQWNAVKASDTGIYYCAR | HVPVPISGTFLWREREMHDLGYFDD | WGQGTLVIVSS |
| | (SEQ ID NO: 873) | (SEQ ID NO: 762) | (SEQ ID NO: 891) |
| N4 | QVTISIDKSTSTAYLQWNNVKASDTGIYYCAR | HVPVPISGTFLWREREMHDFGYFDD | WGQGTLVIVSS |
| | (SEQ ID NO: 874) | (SEQ ID NO: 763) | (SEQ ID NO: 892) |
| O1 | RTT FLD WGSLKASDTAIYYCAR | GCLGAKCYYPHYYYGL DV | WGQGTTVIVSS |
| | (SEQ ID NO: 875) | (SEQ ID NO: 764) | (SEQ ID NO: 893) |
| P1 | RFTVSRDNSKKMVYLQMSSLRPDDAAVYYCAR | GGPAYYTYSDTLTGYHNVVG DY | WGQGTLVTVSS |
| | (SEQ ID NO: 876) | (SEQ ID NO: 765) | (SEQ ID NO: 894) |
| P2 | RFTISRDNSKKLVYLQMDSLRSEDTAVYYCAR | GGPAYYLYSDVLTGFHNVVG DY | WGQGTLVTVSA |
| | (SEQ ID NO: 877) | (SEQ ID NO: 766) | (SEQ ID NO: 895) |
| P3 | RFTISRDNSKSMVFLLMNSLRAEDTAVYYCVR | GGPAYYTYNDVLTGYAYVVG DF | WGQGTLVTVSS |
| | (SEQ ID NO: 878) | (SEQ ID NO: 767) | (SEQ ID NO: 896) |
| P4 | RFTISRDNSKNTLHLQMNSLRPEDTAVYFCAR | GGPAYYLYNDVLTGYYNVVG DF | WGQGTLVTVSS |
| | (SEQ ID NO: 879) | (SEQ ID NO: 768) | (SEQ ID NO: 897) |
| P5 | RFTISRDNSKNTLHLQMNSLRPEDTAVYFCAR | GGPAYYLYNDVLTGYYNVVG DF | WGQGTLVTVSS |
| | (SEQ ID NO: 880) | (SEQ ID NO: 769) | (SEQ ID NO: 898) |
| P6 | RFTISRDNSKNTLHMQMNSLRLEDTAVYFCAR | GGPAYYLYNDVLTGYYNVVG DF | WGQGTLVTVSS |
| | (SEQ ID NO: 881) | (SEQ ID NO: 770) | (SEQ ID NO: 899) |
| Q1 | RISMTTDTSTNTAYMELRSLRSDDTAVYYCAR | DRNSAGGTWLFRDPPPGSTFF DS | WGQGSLVTVSS |
| | (SEQ ID NO: 882) | (SEQ ID NO: 771) | (SEQ ID NO: 900) |
| Q2 | RISMTTDTSTNTAYMELRSLRSDDTAVYYCAR | DRNSAGGTWLFRDPPPGSTFF DS | WGQGSLVTVSS |
| | (SEQ ID NO: 883) | (SEQ ID NO: 772) | (SEQ ID NO: 901) |
| R1 | RLTMTTDTSTSTAYMELRSLRSDDTAVYFCAR | DYGVNFGGGSEHNL DY | WGRGTRVTVSS |
| | (SEQ ID NO: 884) | (SEQ ID NO: 773) | (SEQ ID NO: 902) |
| S1 | RVTMTTDTYTSTAYMELRSLTSDDTAVYYCAR | DDCRSSTCYLAQHNWQAYYH DS | WGQGTLVTVSS |
| | (SEQ ID NO: 885) | (SEQ ID NO: 774) | (SEQ ID NO: 903) |
| T1 | RFTISRDNAKNTLYLQMNSLRDEDTAVYFCAR | GGDSSSPYYYPM DV | WGQGTTVAVSS |
| | (SEQ ID NO: 886) | (SEQ ID NO: 775) | (SEQ ID NO: 904) |
| U1 | RVTITADED TVSLELSSLKSEDTAVYYCAR | EVNLKTWNLAHPNVF DV | WGQGTMLTVSS |
| | (SEQ ID NO: 887) | (SEQ ID NO: 776) | (SEQ ID NO: 905) |
| V1 | RVTLTTDPYTNTVFMELRSLRSDDTAVYYCAR | ENEGEYVWGHFRS DY | WGQGTLVTVSS |
| | (SEQ ID NO: 888) | (SEQ ID NO: 777) | (SEQ ID NO: 906) |

TABLE 19A
Fab Light Chain Sequences

| | | |
|---|---|---|
| Fab | FRAMEWORK 1 | CDR1 |

| | | |
|---|---|---|
| N1 | EL TQSPGTLSLSPGERATLSC (SEQ ID NO: 907) | RASQS VSSSYLA (SEQ ID NO: 778) |
| N2 | EL TQSPGTLSLFPGERATLSC (SEQ ID NO: 908) | RASQS ILGRYLA (SEQ ID NO: 779) |
| N3 | EL TQSPGTLSLSPGERATLSC (SEQ ID NO: 909) | RASQS VSSSYLA (SEQ ID NO: 780) |
| N4 | ELTLTQSPGTLSLSPGERATLSC (SEQ ID NO: 910) | RASQS VSNNYLA (SEQ ID NO: 781) |
| O1 | EL TQSPSSLSASVGDRVTITC (SEQ ID NO: 911) | RATQ GIDNYLN (SEQ ID NO: 782) |
| P1 | EL TQSPGTLSLSPGERATLSC (SEQ ID NO: 912) | RASQ VRG FLA (SEQ ID NO: 783) |
| P1a | EL TQSPGTLSLSPGERATLSC (SEQ ID NO: 913) | RTSQS VSSTYLA (SEQ ID NO: 784) |
| P2 | ELVLTQSPGTLSLSPGERATLSC (SEQ ID NO: 914) | RASQS VSSSYLA (SEQ ID NO: 785) |
| P3 | EL TQSPGTLSLSPGERATLSC (SEQ ID NO: 915) | RASQS VSSTYLA (SEQ ID NO: 786) |
| P4 | EL TQSPGTLSLSPGERATLSC (SEQ ID NO: 916) | RASQS VSSSNLA (SEQ ID NO: 787) |
| P5 | EL TQSPASLSLSPGERATLSC (SEQ ID NO: 917) | RASQS V GTYFA (SEQ ID NO: 788) |
| P6 | EL IQSPGTLFLSSGERATLSC (SEQ ID NO: 918) | RASQS VSSSNLA (SEQ ID NO: 789) |
| Q1 | EL TQSPGTLSLSPGERATLSC (SEQ ID NO: 919) | RASQS VSSSYLA (SEQ ID NO: 790) |
| Q2 | EL TQSPASLSLSPGGSATLAC (SEQ ID NO: 920) | RASRG VNS NLA (SEQ ID NO: 791) |
| R1 | EL TQSPLSLPVTPGEPASISC (SEQ ID NO: 921) | RSSYSLLHINGYKYLD (SEQ ID NO: 792) |
| S1 | ELVLTQSPGTLSLSPGERATLSC (SEQ ID NO: 922) | RASQS VSSSYLA (SEQ ID NO: 793) |
| T1 | EL TQSPGTLSLSPGERATLSC (SEQ ID NO: 923) | RASQS VSSSYLA (SEQ ID NO: 794) |
| U1 | EL TQSPSSLSASVGDRVTITC (SEQ ID NO: 924) | RASQS ISSFLN (SEQ ID NO: 795) |
| V1 | EL TQSPSSLSASVGDRVTITC (SEQ ID NO: 925) | QASQD ISNFLN (SEQ ID NO: 796) |

TABLE 19B
Fab Light Chain Sequences - Con't

| Fab | FRAMEWORK 2 | CDR2 |
|---|---|---|
| N1 | WYQQKPGQAPRLLIY | GASNRAA (SEQ ID NO: 797) |
| | (SEQ ID NO: 926) | |
| N2 | WYQQKGGRAPRLLIF | GASKRAT (SEQ ID NO: 798) |
| | (SEQ ID NO: 927) | |
| N3 | WYQQKPGQAPRLLIY | GASSRAT (SEQ ID NO: 799) |
| | (SEQ ID NO: 928) | |
| N4 | WYQQKPGQAPRLLIY | GASSRAT (SEQ ID NO: 800) |
| | (SEQ ID NO: 929) | |
| O1 | WYQQKPGKPPRLLIY | GASSLQS (SEQ ID NO: 801) |
| | (SEQ ID NO: 930) | |
| P1 | WFQQKPGQAPRLLIY | GASNRAP (SEQ ID NO: 802) |
| | (SEQ ID NO: 931) | |
| P1a | WYQQKPGQPPRLLIY | GASNRAP (SEQ ID NO: 803) |
| | (SEQ ID NO: 932) | |
| P2 | WYQQKPGQAPRLLIY | GASSRAT (SEQ ID NO: 804) |
| | (SEQ ID NO: 933) | |
| P3 | WYQQKPGQAPRLLIY | GASSRAT (SEQ ID NO: 805) |
| | (SEQ ID NO: 934) | |
| P4 | WFQHKSGRAPRLLIY | GASNRAP (SEQ ID NO: 806) |
| | (SEQ ID NO: 935) | |
| P5 | WYQQKPGQAPRLLIY | GASNRAT (SEQ ID NO: 807) |
| | (SEQ ID NO: 936) | |
| P6 | WFQHKSGRAPRLLIY | GASNRAP (SEQ ID NO: 808) |
| | (SEQ ID NO: 937) | |
| Q1 | WYQQKPGQAPRLLIY | GASSRAT (SEQ ID NO: 809) |
| | (SEQ ID NO: 938) | |
| Q2 | WYHQKPGQAPRLLIY | SASTRAT (SEQ ID NO: 810) |
| | (SEQ ID NO: 939) | |
| R1 | WYLQRPGQSPQLLIY | LGSNRAP (SEQ ID NO: 811) |
| | (SEQ ID NO: 940) | |
| S1 | WYQQKPGQAPRLLIY | GASSRAT (SEQ ID NO: 812) |
| | (SEQ ID NO: 941) | |
| T1 | WYQQKPGQAPRLLIY | GASSRAT (SEQ ID NO: 813) |
| | (SEQ ID NO: 942) | |
| U1 | WYQQKPGKAPKLLIY | AASSLQS (SEQ ID NO: 814) |
| | (SEQ ID NO: 943) | |
| V1 | WYQRRPGKAPNLLIY | DATHLET (SEQ ID NO: 815) |
| | (SEQ ID NO: 944) | |

TABLE 19C
Fab Light Chain Sequences - Con't

| Fab | FRAMEWORK 3 |
|---|---|
| N1 | GIPDRFSGSGSGADFTLTISRLEPEDFAVYYC |
| | (SEQ ID NO: 945) |
| N2 | GIPDRFSGSGSGTDFTLTIGRLEPEDFAVYYC |
| | (SEQ ID NO: 946) |
| N3 | GIPDRFSGSGSGTDFTLTISRLEPEDFAVYYC |
| | (SEQ ID NO: 947) |
| N4 | GIPDRFSGSGSGTGFTLIISRLEPEDFAVYYC |
| | (SEQ ID NO: 948) |
| O1 | GVPSRFSGGGSGTHFTLTITNLQPEDFATYYC |
| | (SEQ ID NO: 949) |
| P1 | GIPDRFSGSGSGTDFTLTISRLEPEDFAVYYC |
| | (SEQ ID NO: 950) |
| P1a | GIPDRFSGSGSGTDFTLTISRLEPEDFAVYYC |
| | (SEQ ID NO: 951) |
| P2 | GIPDRFSGSGSGTDFTLTISRLEPEDFAVYYC |
| | (SEQ ID NO: 952) |
| P3 | GIPDRFSGSGSGTDFTLTISRLEPEDFAVYYC |
| | (SEQ ID NO: 953) |
| P4 | DIPDRFSGSGSGTDFTLSISRLEPEDFAVYYC |
| | (SEQ ID NO: 954) |
| P5 | GIPDRFSGSGSGTDFTLTVSRLEPEDFAVYYC |
| | (SEQ ID NO: 955) |
| P6 | DIPDRFSGSGSGTDFTISISRLEPEDFAVYYC |
| | (SEQ ID NO: 956) |
| Q1 | GIPDRFSGSGSGTDFTLTISRLEPEDFAVYYC |
| | (SEQ ID NO: 957) |
| Q2 | GIPGRFSGSGFGTEFTLTINNLQSDDFGVYYC |
| | (SEQ ID NO: 958) |
| R1 | GVPDRFSGSGSGTSFTLKISRVEAEDVGVYYC |
| | (SEQ ID NO: 959) |
| S1 | GIPDRFSGSGSGTDFTLTISRLEPEDFAVYYC |
| | (SEQ ID NO: 960) |
| T1 | GIPDRFSGSGSGTDFTLTISRLEPEDFAVYYC |
| | (SEQ ID NO: 961) |
| U1 | GVPPRFSGSGSGTDFTLTISSLQPEDFATYYC |
| | (SEQ ID NO: 962) |
| V1 | GVPSRFSGSGFGTHFTLTINSLQPEDIGTYYC |
| | (SEQ ID NO: 963) |

TABLE 19D
Fab Light Chain Sequences - Con't

| | | |
|---|---|---|
| Fab | CDR3 | FRAMEWORK 4 |

| | | |
|---|---|---|
| N1 | QQYGSSL IT (SEQ ID NO: 816) | FGQGTRLEIKRT (SEQ ID NO: 964) |
| N2 | QHYGSS IT (SEQ ID NO: 817) | FGQGTRLDIKRT (SEQ ID NO: 965) |
| N3 | QQYGSSL T (SEQ ID NO: 818) | FGGGTKVEIKRT (SEQ ID NO: 966) |
| N4 | QQYGSSS IT (SEQ ID NO: 819) | FGQGTRLEIKRT (SEQ ID NO: 967) |
| O1 | QQSYSTPPET (SEQ ID NO: 820) | FGQGTKVEIKRT (SEQ ID NO: 968) |
| P1 | QQYGDSPPIT (SEQ ID NO: 821) | FGQGTRLEIKRT (SEQ ID NO: 969) |
| P1a | QQYGDSPPIT (SEQ ID NO: 822) | FGQGTRLDIKRT (SEQ ID NO: 970) |
| P2 | QQYGDSPP F (SEQ ID NO: 823) | FGPGTKVDIKRT (SEQ ID NO: 971) |
| P3 | QQYGSSP LT (SEQ ID NO: 824) | FGGGTKVEIKRT (SEQ ID NO: 972) |
| P4 | QRYGDSPPIT (SEQ ID NO: 825) | FGQGTRLEIKRT (SEQ ID NO: 973) |
| P5 | QQYGSSP T (SEQ ID NO: 826) | FGQGTKVEIKRT (SEQ ID NO: 974) |
| P6 | QRYGDSPPIT (SEQ ID NO: 827) | FGQGTRLEIKRT (SEQ ID NO: 975) |
| Q1 | QQYGSSP QT (SEQ ID NO: 828) | FGQGTKVEIKRT (SEQ ID NO: 976) |
| Q2 | QQYDDTPQIT (SEQ ID NO: 829) | FGQGTRLDIKRL (SEQ ID NO: 977) |
| R1 | MQSLQAP WT (SEQ ID NO: 830) | FGQGTKVEMKRT (SEQ ID NO: 978) |
| S1 | QQYGSSP QT (SEQ ID NO: 831) | FGQGTKVEIKRT (SEQ ID NO: 979) |
| T1 | QQYGSSP QT (SEQ ID NO: 832) | FGQGTKVEIKRT (SEQ ID NO: 980) |
| U1 | QQSYSTP RT (SEQ ID NO: 833) | FGQGTKLEIKRT (SEQ ID NO: 981) |
| V1 | QHFDDVPSFT (SEQ ID NO: 834) | FGPGTKVDLKRT (SEQ ID NO: 982) |

The relation of the new Fabs to AR1-, AR2- and AR3-MAbs was determined by competition ELISA. Briefly, E1E2 antigens (H77) were captured by lectin onto microwells, and then blocked with undiluted soluble Fabs for 30 minutes before the addition of AR1-, AR2-, and AR3-specific MAbs, or the murine MAb A4, which is specific for a linear E1 epitope (residues 197-207). Bound IgG was detected either with HRP-conjugated anti-human Fcγ or anti-mouse Fcγ. The change of binding signals of the MAbs comparing a control Fab was calculated and tabulated. Fab P 1a has the same heavy chain but a different light chain compared to Fab P1. Tables 20A and B, set forth the results. Residual binding levels of between 0-25% are indicated in bold italic font; residual binding levels of between 51-75% are indicated in bold font; residual binding levels of between 76-150% are indicated in normal font; and residual binding levels of greater than 151% are indicated in italic font.

TABLE 20A

| Binding | Blocking Fab | | | | | | | | | |
|---|---|---|---|---|---|---|---|---|---|---|
| MAb | N1 | N2 | N3 | N4 | O1 | P1 | P1a | P2 | P3 | P4 |
| A4 | 100 | 101 | 96 | 100 | 96 | 101 | 100 | 102 | 100 | 98 |
| AR1A | 83 | 82 | 71 | 94 | 76 | 86 | 88 | 91 | 85 | 85 |
| AR1B | 95 | 100 | 92 | 95 | 95 | 97 | 100 | 99 | 93 | 95 |
| AR2A | 85 | 86 | 69 | 74 | 88 | 82 | 82 | 82 | 72 | 79 |
| AR3A | 118 | 76 | 60 | 111 | 92 | 79 | 113 | 133 | 133 | 128 |
| AR3B | 178 | 122 | 136 | 116 | 89 | 187 | 175 | 172 | 141 | 177 |
| AR3C | 146 | 116 | 92 | 112 | 77 | 131 | 145 | 140 | 124 | 155 |
| AR3D | 131 | 98 | 107 | 92 | 83 | 145 | 138 | 138 | 118 | 145 |

TABLE 20B

| Binding | Blocking Fab | | | | | | | | | | |
|---|---|---|---|---|---|---|---|---|---|---|---|
| MAb | P5 | P6 | Q1 | Q2 | R1 | S1 | T1 | U1 | V1 | AR1B | AR2A |
| A4 | 100 | 99 | 95 | 98 | 99 | 100 | 99 | 101 | 99 | 85 | 87 |
| AR1A | 87 | 82 | 79 | 78 | 100 | 69 | 80 | 55 | 58 | [symbol] | 113 |
| AR1B | 95 | 95 | 97 | 96 | 101 | 101 | 102 | 103 | 102 | [symbol] | 105 |
| AR2A | 73 | 76 | 75 | 72 | 75 | 79 | 93 | 132 | 79 | 59 | [symbol] |
| AR3A | 119 | 101 | 113 | 89 | 236 | 203 | 72 | [symbol] | 94 | 202 | 158 |
| AR3B | 143 | 116 | 117 | 97 | 242 | 247 | 124 | [symbol] | 113 | 141 | 115 |
| AR3C | 117 | 103 | 97 | 75 | 163 | 149 | 105 | [symbol] | 82 | 95 | 97 |
| AR3D | 132 | 106 | 86 | 71 | 133 | 100 | 107 | [symbol] | 70 | 65 | 58 |

Except Fab U1, which competed strongly with AR3MAbs and partially with MAb AR1A, significant competition was not observed for the new Fab panel. Table 21 illustrates the cross-reactivity of the antibodies to diverse HCV isolates. Specific binding of Fabs to E1E2 antigens from a diverse HCV panel also was studied by ELISA. MAb AR3A was included as a positive control (1 μg/mL). Table 21 sets forth the results, which indicate that many of the new Fabs cross-react with E1E2 from diverse HCV genotypes. For example, N1, N2, N3, N4, P2, P5, Q1, S1 and V1 bind well to the E1E2 complexes of many of the HCV isolates tested.

TABLE 21

| | Binding of Fab to E1E2 Ag from different isolates (OD 450 nm) | | | | | | | | | | |
|---|---|---|---|---|---|---|---|---|---|---|---|
| | | UKN | | | | | UKN | UKN | UKN | UKN | UKN |
| Fab | H77 | 1B12.16 | OH8 | CH35 | J6E3 | JFH | 2B1.1 | 3A1.28c | 4.21.16 | 5.15.7 | 6.5.8 |
| N1 | 1.13 | 0.65 | 1.13 | 0.59 | 0.64 | 0.99 | 0.33 | 1.08 | 0.93 | 0.43 | 0.29 |
| N2 | 1.45 | 0.81 | 1.44 | 0.82 | 0.92 | 1.34 | 0.44 | 1.36 | 1.09 | 0.46 | 0.40 |
| N3 | 1.54 | 0.62 | 1.32 | 0.59 | 0.66 | 1.20 | 0.29 | 1.27 | 0.96 | 0.37 | 0.23 |
| N4 | 1.71 | 1.06 | 1.75 | 1.03 | 0.99 | 1.57 | 0.48 | 1.73 | 1.49 | 0.71 | 0.44 |
| O1 | 1.37 | 0.17 | 0.44 | 0.27 | 0.05 | 0 | 0.02 | 0.48 | 0.57 | 0.10 | 0.02 |
| P1 | 1.44 | 0.26 | 0.80 | 0.27 | 0.08 | 0.12 | 0.03 | 0.98 | 0.62 | 0.17 | 0.06 |
| P1a | 0.61 | 0.15 | 0.24 | 0.08 | 0.03 | 0 | 0.00 | 0.18 | 0.04 | −0.04 | 0.05 |
| P2 | 1.42 | 0.59 | 1.00 | 0.47 | 0.60 | 0.88 | 0.18 | 1.05 | 0.78 | 0.25 | 0.22 |
| P3 | 0.58 | 0.20 | 0.36 | 0.11 | 0.11 | 0 | 0 | 0.33 | 0.15 | 0.06 | 0.03 |
| P4 | 1.60 | 0.59 | 1.15 | 0.52 | 0.10 | 0.45 | 0.09 | 1.07 | 0.99 | 0.31 | 0.19 |
| P5 | 1.70 | 0.33 | 1.14 | 0.41 | 0.01 | 0.06 | 0 | 1.22 | 1.00 | 0.19 | 0.10 |
| P6 | 0.61 | 0.26 | 0.46 | 0.21 | 0.02 | 0.00 | 0.13 | 0.37 | 0.23 | 0.08 | 0.12 |
| Q1 | 1.34 | 0.38 | 0.96 | 0.48 | 0.19 | 0.43 | 0.03 | 1.07 | 0.85 | 0.30 | 0.10 |
| Q2 | 0.45 | 0.21 | 0.43 | 0.19 | 0.25 | 0.18 | 0.15 | 0.35 | 0.17 | 0.05 | 0.16 |
| R1 | 1.24 | 0.03 | 0.50 | 0.15 | 0 | 0 | 0.02 | 0 | 0.01 | 0.00 | 0.05 |
| S1 | 1.73 | 0.26 | 1.32 | 0.79 | 0.07 | 0.21 | 0.03 | 0.55 | 1.12 | 0.18 | 0.08 |
| T1 | 0.49 | 0 | 0.15 | 0.00 | 0 | 0 | 0 | 0 | 0.08 | 0.07 | 0.01 |
| U1 | 0.53 | 0.11 | 0.26 | 0.07 | 0.08 | 0.07 | 0.00 | 0.11 | 0.00 | 0.06 | 0.06 |
| V1 | 1.74 | 0.16 | 1.35 | 0.77 | 0.73 | 1.27 | 0.30 | 0.06 | 1.02 | 0.30 | 0.05 |
| AR3A | 1.83 | 1.81 | 1.83 | 1.48 | 1.82 | 1.80 | 1.09 | 1.31 | 1.77 | 0.87 | 1.33 |
| IgG | | | | | | | | | | | |

Two of the Fabs, N4 and P2, were produced at a larger scale and purified for the study of virus neutralization. Results show that both Fabs neutralized HCVpp-H77, and Fab N4 also cross-neutralized HCVpp-J6. Of note, the neutralizing activity of Fab N4 against HCVpp-H77 is significantly higher than the Fab fragment of MAb AR3A. For example, at a concentration of 0.4 μg/mL, approximately 60% neutralization was observed using Fab N4, compared to about 10% neutralization observed using AR3A or P2. At a higher concentration (2 μg/mL), approximately 85% neutralization was observed using Fab N4; approximately 40% neutralization observed using Fab P2; and approximately 20% neutralization observed using AR3A.

The properties of N4, Q2 and V1 anti-HCV E1-E2 antibodies presented as full-length IgG molecules were also analyzed in an antibody competition study. In the antibody competition study, the IgG N4, Q2 and V1 antibodies were expressed by transient transfection of 293Freestyle cells. The antibody concentrations in the transfected cell supernatants (5-11 μg/mL) were determined by a sandwich ELISA as described herein, e.g., Example 1. Undiluted cell supernatants containing the new antibodies were incubated with lectin-captured E1E2 antigens before the addition of biotinylated human MAbs or Fab N4. Results are shown in the table below.

TABLE 22

| Binding MAb | Blocking Ab | | |
|---|---|---|---|
| (biotinylated) | IgG N4 | IgG Q2 | IgG V1 |
| MAb AR1A | 92 | 96 | 62 |
| MAb AR1B | 95 | 103 | 100 |
| MAb AR2A | 91 | 100 | 92 |
| MAb AR3A | 114 | 119 | 72 |
| Fab N4 | 18 | 64 | 86 |

The numbers indicate the % residual binding signals of biotinylated antibodies in the presence of blocking MAbs and the extent of competition is highlighted.

These results indicate that two of the new MAbs, i.e. IgG N4 and Q2, recognize epitopes outside the three antigenic regions 1 to 3 of E2 as determined in the antibody competition study. Slight competition with MAbs ARIA and AR3A was observed with MAb IgG V1.

For the antibody reactivity studies, ELISA-based assays were used to evaluate the binding of the new antibodies to different formats of E1 and E2. E1E2 was prepared from cell lysate of 293T cells transfected with H77 E1E2 cDNA. Soluble E1 and E2 were produced by Drosophila S2 cells. Soluble E2ΔTM was produced by transfection of 293T cells. MAbs AR1-3 and MAb A4 (Dubuisson et al. J. Virol. 68, 6147-60 (1994)) are positive controls for E2 and E1 (linear epitope) binding, respectively. Results of these antibody reactivity studies are summarized in the following table.

TABLE 23

| | | Denatured/ | | Soluble |
|---|---|---|---|---|
| | Folded | reduced | Soluble E1 by | E2ΔTM by |
| Antibody | E1E2 | E1E2 | Drosophila S2 cells | 293T cells |
| MAb AR1A | + | − | − | + |
| MAb AR2A | + | − | − | + |
| MAb AR3A | + | − | − | + |
| N4 | + | − | ? | − |
| V1 | + | − | ? | − |
| MAb A4 | + | + | + | N.D. |

+ and − indicate positive and negative binding, respectively
? indicates same background signal obtained using S2-cell expressed E2
N.D. means not determined

These data indicate that the new MAbs bind folded E1E2 complex, but not denatured/reduced E1E2, misfolded E1 expressed by insect cell-expressed sE2, or soluble E2ΔTM expressed by mammalian cells.

Antibodies to discontinuous neutralizing epitopes on E1 are not widely known. To confirm these novel HCV epitopes are present on E1, the five N-glycans on E1 of the prototypic HCV isolate H77 were removed individually and the effect of these changes in E1 on antibody binding was investigated. The removal of a N-glycan in a glycoprotein can disturb the local conformation hence changing the binding property of an antibody to its nearby regions. The N-glycosylation motifs in E1 at N196, N209, N234, N305 and N325 were eliminated by replacing the specified asparagines with alanines. Wildtype E1E2 (WT) and the corresponding alanine mutants (xN196A, xN209A, xN234A, xN305A and xN325A) were expressed in 293T cells by transient transfection. These E1E2 antigens were extracted from the transfected cells using Tris buffer (pH 7.4, 140 mM NaCl) supplemented with non-denaturing detergents (Triton X-100 and NP-40). The antigens were diluted serially and then captured onto microwells pre-coated with lectin as described above. Captured E1E2 antigens were then probed with the different antibodies (FIG. 7).

The results in FIG. 7 show that none of the E1 mutations (xN196A, xN209A, xN234A, xN305A and xN325A) affected the binding of the anti-E2 MAb AR3A to the antigens, confirming that these mutations did not affect the overall expression level of the E1E2 complex and the proper folding of E2 in the transfected cells. Second, the N196A mutation reduced binding of the anti-E1 MAb A4 which recognizes a linear epitope (residues 197-207) next to N196, demonstrating that the removal of the nearby N-glycan adversely affected the binding of MAb A4. Third, reduced binding of antibodies N4, P2, V1 and Q2 was observed for the E1 N-glycan mutants xN196A and xN305A, suggesting that the regions nearby these two N-glycans are involved in forming the discontinuous neutralizing epitopes. The fact that IgG N4 neutralizes two different genotypic HCV strains indicate that it is recognizing a relatively conserved epitope on the E1E2 complex. Therefore, the N4 antibody is a good candidate to use with AR3-specific antibodies in an antibody cocktail to simultaneously target two non-overlapping, conserved neutralizing epitopes on HCV.

### Example 4

### Additional Characterization of N4 IgG and V1 IgG

To determine the nature of the epitopes recognized by N4 IgG and V1 IgG, a series of E1 and E2 mutants were prepared and used in binding studies.

### A. Generation and Expression of E1 Mutants

A series of E1 or E2 mutants, set forth in Tables 24 and 25, respectively, was prepared by site directed mutagenesis. Amino acid residues that are relatively conserved among a panel of HCV isolates from diverse genotypes (H77, H, Con1, OH8.1, Ch35.1, UKN1b12.16, JFH-1, J6, UKN2a1.2, UKN2b1.1, UKN3a1.28, UKN4.21.16, UKN5.14.4, UKN5.15.7, UKN6.5.340 and UKN6.5.8) were selected for site-directed mutagenesis to alanine, or from alanine to glycine. Briefly, specific mutations were introduced to the E1E2 expression plasmid pH77.20 with oligonucleotides encoding the corresponding mutations using the QuikChange Lightning Site-Directed Mutagenesis Kit with the following PCR conditions: 95° C., 2 minutes; 18 cycles of 95° C. (20 seconds), 60° C. (10 seconds) and 70° C. (3.5 minutes); and 70° C., 5 min. After removal of the template DNA by Dpn I-digestion, the mutated DNA samples were transformed into chemically-competent E. coli. Mutant DNA plasmids were purified from the bacteria and the desired mutations confirmed by DNA sequencing of the corresponding regions for mutagenesis.

TABLE 24
E1 mutants

| Mutation | SEQ ID NO |
|---|---|
| xN196A/xN209A | 1206 |
| xN196A/xN234A | 1207 |
| xN196A/xN305A | 1208 |
| xN196A/xN325A | 1209 |
| xN305A/xN209A | 1210 |
| xN305A/xN234A | 1211 |
| xN305A/xN325A | 1212 |
| Y192A | 1213 |
| Q193A | 1214 |
| V194A | 1215 |
| R195A | 1216 |
| xN196A | 1217 |
| S198A | 1218 |
| Y201A | 1219 |
| T204A | 1220 |
| N205A | 1221 |
| D206A | 1222 |
| xN209A | 1223 |
| S211A | 1224 |
| I212A | 1225 |
| L221A | 1226 |
| H222A | 1227 |
| P224A | 1228 |
| G225A | 1229 |
| V227A | 1230 |
| P228A | 1231 |
| xN234A | 1232 |
| S236A | 1233 |
| W239A | 1234 |
| P244A | 1235 |
| V246A | 1236 |
| T255A | 1237 |
| L258A | 1238 |
| R259A | 1239 |
| H261A | 1240 |
| D263A | 1241 |
| V266A | 1242 |
| A269G | 1243 |
| S273A | 1244 |
| A274G | 1245 |
| Y276A | 1246 |
| G278A | 1247 |
| D279A | 1248 |
| G282A | 1249 |
| Q289A | 1250 |
| F291A | 1251 |
| T292A | 1252 |
| F293A | 1253 |
| S294A | 1254 |
| P295A | 1255 |
| R296A | 1256 |
| H298A | 1257 |
| Q302A | 1258 |
| xN305A | 1259 |
| S307A | 1260 |
| Y309A | 1261 |
| G311A | 1262 |
| T314A | 1263 |
| G315A | 1264 |
| H316A | 1265 |
| R317A | 1266 |
| M318A | 1267 |
| A319G | 1268 |
| W320A | 1269 |
| D321A | 1270 |
| M322A | 1271 |
| M323A | 1272 |
| M324A | 1273 |
| xN325A | 1274 |
| W326A | 1275 |
| S327A | 1276 |
| P328A | 1277 |
| T329A | 1278 |
| R339A | 1279 |
| P341A | 1280 |
| D346A | 1281 |
| G350A | 1282 |
| H352A | 1283 |
| W353A | 1284 |
| G354A | 1285 |

TABLE 25
Additional E2 mutants

| Mutation | SEQ ID NO |
|---|---|
| T425A | 1286 |
| L427A | 1287 |
| N428A | 1288 |
| T435A | 1289 |
| G436A | 1290 |
| A439A | 1291 |
| L441A | 1292 |
| F442A | 1293 |
| Y443A | 1294 |
| F447A | 1295 |
| N448A | 1296 |
| R614A | 1297 |
| R617A | 1298 |
| Y618A | 1299 |
| P619A | 1300 |
| T621A | 1301 |
| N623A | 1302 |
| Y624A | 1303 |
| R630A | 1304 |
| G634A | 1305 |
| G635A | 1306 |
| E637A | 1307 |
| H638A | 1308 |
| R639A | 1309 |
| A642G | 1310 |
| A643G | 1311 |

### B. Infectivity of the E1 and E2 Mutants

To investigate the effect of the mutations on the folding and function of the E1E2 complex, the mutant plasmids were co-transfected into 293T cells with the pNL4-3.lucR-E-plasmid to generate HCV pseudotype virus particles (HCVpp). A wildtype HCVpp H77.20 control also was generated. The pNL4-3.lucR-E-plasmid encodes a replication-defective HIV-1 genome for pseudotype virus particle packaging and a luciferase reporter gene for virus infectivity determination (Connor et al. (1195) Virology 206, 935-44). To determine the infectivity of the mutants, the supernatants from the transfected cells were harvested 3 days post-transfection and added to Huh7 cell monolayers to allow for a single-round infection. The infected Huh7 cells were lysed and the viral infectivity determined using a firefly luciferase assay system (Promega), according to the manufacturer's instructions. Tables 26 and 27 show the infectivity of the HCVpp containing modified E1 and modified E2 polypeptides. In many instances, infectivity was significantly reduced, suggesting that the mutation affected correct folding of the E1/E2 complex. In some instances, infectivity was significantly increased. For example, HCVpp containing the E1 mutants F293A, P295A, T314A or H316A showed increased infectivity compared to the HCVpp H77.20 wild type control. These mutants were tested in a separate experiment, which indicated that HCVpp containing F293A or P295A exhibited an approximate 5-fold increase in infectivity compared to HCVpp H77.20, and HCVpp containing T314A or H316A exhibited an approximate 3-fold increase in infectivity compared to HCVpp H77.20.

TABLE 26
Infectivity of HCVpp containing E1 mutants

| | Infectivity | | Infectivity | | Infectivity | | Infectivity |
|---|---|---|---|---|---|---|---|
| Mutant | (% of wt) | Mutant | (% of wt) | Mutant | (% of wt) | Mutant | (% of wt) |
| N196A/ | 3 | L221A | 10 | Y276A | 140 | R317A | 169 |
| N209A | | | | | | | |
| N196A/ | 7 | H222A | 8 | G278A | 5 | M318A | 3 |
| N234A | | | | | | | |
| N196A/ | 4 | P224A | 158 | D279A | 5 | A319G | 90 |
| N305A | | | | | | | |
| N196A/ | 11 | G225A | 119 | G282A | 13 | W320A | 35 |
| N325A | | | | | | | |
| N305A/ | 3 | V227A | 175 | Q289A | 18 | D321A | 11 |
| N209A | | | | | | | |
| N305A/ | 7 | P228A | 12 | F291A | 26 | M322A | 21 |
| N234A | | | | | | | |
| N305A/ | 38 | N234A | 148 | T292A | 106 | M323A | 7 |
| N325A | | | | | | | |
| Y192A | 260 | S236A | 25 | F293A | 794 | M324A | 156 |
| Q193A | 43 | W239A | 6 | S294A | 269 | xN325A | 257 |
| V194A | 184 | P244A | 40 | P295A | 1291 | W326A | 4 |
| R195A | 177 | V246A | 66 | R296A | 8 | xS327A | 94 |
| N196A | 18 | T255A | 278 | H298A | 4 | P328A | 6 |
| S198A | 39 | L258A | 103 | Q302A | 4 | T329A | 11 |
| Y201A | 5 | R259A | 2 | N305A | 16 | R339A | 13 |
| T204A | 11 | H261A | 12 | S307A | 15 | P341A | 16 |
| N205A | 8 | D263A | 163 | Y309A | 4 | D346A | 129 |
| D206A | 17 | V266A | 45 | G311A | 5 | G350A | 44 |
| N209A | 14 | A269G | 70 | T314A | 508 | H352A | 4 |
| S211A | 24 | S273A | 22 | G315A | 5 | W353A | 21 |
| I212A | 5 | A274G | 60 | H316A | 941 | G354A | 57 |

TABLE 27
Infectivity of HCVpp containing E2 mutants

| | Infectivity | | Infectivity | | Infectivity | | Infectivity |
|---|---|---|---|---|---|---|---|
| Mutant | (% of wt) | Mutant | (% of wt) | Mutant | (% of wt) | Mutant | (% of wt) |
| T425A | 3 | F442A | 4 | Y618A | 3 | G634A | 3 |
| L427A | 7 | Y443A | 4 | P619A | 3 | G635A | 3 |
| N428A | 14 | F447A | 6 | T621A | 2 | E637A | 3 |
| T435A | 104 | xN448A | 6 | xN623A | 1 | H638A | 6 |
| G436A | 12 | R614A | 4 | Y624A | 2 | R639A | 4 |
| A439G | 35 | H617A | 14 | R630A | 2 | A642G | 4 |
| L441A | 33 | | | | | A643G | 4 |

### C. Binding of the Antibodies to the E1 and E2 Mutants

To determine which residues are important for binding of N4 IgG and V1 IgG to the E1/E2 complex, the ability of these antibodies to bind to the mutants described above was assessed. The anti-E2 antibody, MAb AR3A, and the mouse anti-E1 A4 antibody, which binds a linear epitope from residues 197-207, were included as controls.

The transfected 293T cells from Example 4B, above, were solubilized in a non-denaturing lysis buffer (25 mM Tris-HCl, 1% Triton X-100, 0.5% Nonidet-P40 and 140 mM NaCl, pH 7.6) to prepare the mutant E1E2 complex antigens for antibody binding analysis. The dissolved E1E2 antigens were first captured to microwells by Galanthus nivalis lectin pre-coated onto the surface of ELISA plates. After blocking with 1% non-fat milk in phosphate-buffered saline, test antibodies at 1 μg/ml were added to the captured E1E2, followed by the horse radish peroxidase (HRP)-conjugated secondary antibodies and the TMB colorimetric substrate (ThermoScientific) for detecting bound antibodies.

Tables 28 and 29 set forth the binding of the antibodies to the E1E2 complexes containing the E1 or E2 mutants, respectively. Mutation of some residues in E1 appeared to significantly reduce the ability of N4 IgG and V1 IgG to bind. For example, the binding of both antibodies to E1/E2 complexes containing the N196A/N209A, N196A1N234A, N196A/N305A, N196A/N325A, N305A/N209A, or N305A/N234A double mutation, or Y201A, T204A, N205A, D206A or Q302A single mutation in the E1 polypeptide was significantly reduced compared to the binding of these antibodies to E1/E2 complexes containing wild-type E1. Furthermore, mutation of A439G, L441A, F442A, Y443A, R614A, H617A or H638A, and A439G, L441A, F442A, Y443A, F447A, N448A, R614A, H617A, R630A, or R639A, in the E2 polypeptide also significantly reduced the ability of N4 IgG and V1 IgG, respectively, to bind to E1/E2 complex. The data show that the folding and function of the E1/E2 complex is highly complicated and inter-dependent. It is unclear however, whether these residues are directly involved in forming the two non-overlapping discontinuous epitopes recognized by N4 and V1, or if they are just important for correct folding of the E1 and E2 polypeptides in the context of the E1/E2 complex. Correct folding of the E1/E2 are found to be critical for the binding of both N4 and V1, as many of the mutations in the E2 polypeptide also significantly reduced binding of these antibodies to the E1/E2 complex. The relatively conserved residues described above are found to be important for the formation of fully functional, natively folded E1/E2 complex. The N4 IgG and V1 IgG recognize two previous undescribed discontinuous epitopes on the E1/E2 complex, that are different to the epitopes located at antigenic regions (AR) 1, 2 and 3 described herein (see Example 1) and elsewhere (Law et al., (2008) Nature Med. 14:25-27).

TABLE 28
Binding of Na4 IgG and V1 IgG to E1E2 complexes containing E1 mutants

| | Binding relative to wt (%) | | | | | Binding relative to wt (%) | | | |
|---|---|---|---|---|---|---|---|---|---|
| | Mab | A4 | N4 | V1 | | Mab | A4 | N4 | V1 |
| | AR3A | mouse | IgG | IgG | | AR3A | mouse | IgG | IgG |
| N196A/ | 103 | 51 | 21 | 13 | Y276A | 79 | 82 | 103 | 97 |
| N209A | | | | | | | | | |
| N196A/ | 84 | 71 | 32 | 23 | G278A | 67 | 58 | 68 | 88 |
| N234A | | | | | | | | | |
| N196A/ | 78 | 69 | 4 | 6 | D279A | 43 | 28 | 31 | 36 |
| N305A | | | | | | | | | |
| N196A/ | 116 | 64 | 54 | 39 | G282A | 83 | 50 | 128 | 102 |
| N325A | | | | | | | | | |
| N305A/ | 107 | 117 | 17 | 13 | Q289A | 65 | 49 | 94 | 98 |
| N209A | | | | | | | | | |
| N305A/ | 81 | 109 | 30 | 21 | F291A | 120 | 67 | 131 | 109 |
| N234A | | | | | | | | | |
| N305A/ | 87 | 114 | 74 | 95 | T292A | 92 | 90 | 104 | 96 |
| N325A | | | | | | | | | |
| Y192A | 111 | 134 | 122 | 108 | F293A | 52 | 87 | 95 | 102 |
| Q193A | 126 | 81 | 117 | 109 | S294A | 57 | 99 | 71 | 155 |
| V194A | 127 | 160 | 109 | 100 | P295A | 65 | 132 | 82 | 115 |
| R195A | 88 | 197 | 121 | 115 | R296A | 52 | 108 | 138 | 114 |
| N196A | 77 | 45 | 52 | 75 | H298A | 46 | 164 | 62 | 94 |
| S198A | 90 | 61 | 78 | 52 | Q302A | 8 | 215 | 6 | 5 |
| Y201A | 81 | 5 | 7 | 7 | N305A | 46 | 123 | 42 | 80 |
| T204A | 82 | 102 | 24 | 23 | S307A | 69 | 157 | 58 | 84 |
| N205A | 113 | 96 | 3 | 6 | Y309A | 90 | 78 | 32 | 31 |
| D206A | 96 | 81 | 17 | 13 | G311A | 90 | 89 | 87 | 107 |
| N209A | 76 | 119 | 117 | 113 | T314A | 93 | 99 | 83 | 93 |
| S211A | 94 | 106 | 93 | 100 | G315A | 122 | 91 | 87 | 125 |
| I212A | 101 | 125 | 51 | 82 | H316A | 91 | 95 | 70 | 81 |
| L221A | 100 | 104 | 100 | 103 | R317A | 112 | 95 | 98 | 108 |
| H222A | 79 | 132 | 44 | 74 | M318A | 97 | 97 | 92 | 80 |
| P224A | 118 | 99 | 104 | 104 | A319G | 119 | 101 | 113 | 111 |
| G225A | 90 | 106 | 79 | 85 | W320A | 140 | 105 | 121 | 112 |
| V227A | 107 | 150 | 98 | 106 | D321A | 102 | 91 | 92 | 107 |
| P228A | 59 | 171 | 38 | 55 | M322A | 65 | 115 | 143 | 98 |
| N234A | 83 | 105 | 114 | 99 | M323A | 78 | 69 | 99 | 85 |
| S236A | 71 | 88 | 81 | 87 | M324A | 74 | 80 | 89 | 92 |
| W239A | 86 | 103 | 67 | 86 | N325A | 109 | 83 | 150 | 96 |
| P244A | 78 | 85 | 95 | 95 | W326A | 91 | 72 | 125 | 96 |
| V246A | 91 | 96 | 97 | 99 | S327A | 89 | 85 | 69 | 121 |
| T255A | 100 | 106 | 96 | 95 | P328A | 97 | 123 | 127 | 106 |
| L258A | 96 | 99 | 89 | 97 | T329A | 123 | 89 | 114 | 96 |
| R259A | 93 | 97 | 102 | 100 | R339A | 233 | 87 | 119 | 120 |
| H261A | 100 | 96 | 106 | 103 | P341A | 129 | 101 | 105 | 104 |
| D263A | 99 | 96 | 85 | 91 | D346A | 85 | 87 | 83 | 73 |
| V266A | 96 | 96 | 92 | 95 | G350A | 77 | 79 | 68 | 57 |
| A269G | 38 | 47 | 55 | 74 | H352A | 101 | 85 | 60 | 48 |
| S273A | 41 | 38 | 70 | 79 | W353A | 80 | 75 | 70 | 75 |
| A274G | 81 | 53 | 71 | 85 | G354A | 111 | 105 | 90 | 91 |

TABLE 29
Binding of Na4 IgG and V1 IgG to E1E2 complexes containing E2 mutants

| | Binding relative to wt (%) | | | | | Binding relative to wt (%) | | | |
|---|---|---|---|---|---|---|---|---|---|
| | Mab | A4 | N4 | V1 | | Mab | A4 | N4 | V1 |
| | AR3A | mouse | IgG | IgG | | AR3A | mouse | IgG | IgG |
| T425A | 19 | 104 | 100 | 93 | Y618A | 20 | 157 | 33 | 51 |
| L427A | 18 | 102 | 125 | 112 | P619A | 29 | 108 | 41 | 61 |
| N428A | 22 | 127 | 98 | 83 | T621A | 21 | 114 | 34 | 36 |
| T435A | 80 | 117 | 122 | 89 | xN623A | 30 | 116 | 54 | 60 |
| G436A | 30 | 96 | 107 | 97 | Y624A | 32 | 121 | 59 | 59 |
| A439G | 12 | 119 | 12 | 12 | R630A | 89 | 106 | 115 | 20 |
| L441A | 9 | 111 | 18 | 11 | G634A | 89 | 112 | 117 | 101 |
| F442A | 7 | 90 | 22 | 14 | G635A | 76 | 109 | 80 | 86 |
| Y443A | 7 | 28 | 5 | 3 | E637A | 55 | 122 | 67 | 101 |
| F447A | 27 | 98 | 28 | 19 | H638A | 23 | 104 | 12 | 55 |
| xN448A | 29 | 97 | 48 | 19 | R639A | 64 | 162 | 114 | 9 |
| R614A | 12 | 133 | 16 | 12 | A642G | 20 | 137 | 65 | 74 |
| H617A | 11 | 140 | 21 | 21 | A643G | 17 | 131 | 34 | 47 |

### Example 5

### Sensitivity of HCVpp Containing Modified E1 with the F293A, P295A, T314A or H316A Mutation to Antibodies

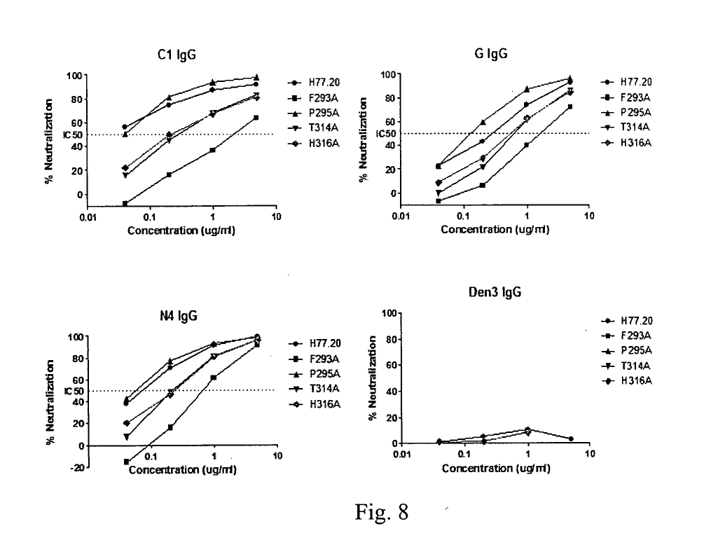

As demonstrated above, HCVpp containing F293A or P295A exhibited an approximate 5-fold increase in infectivity compared to HCVpp 1177.20, and HCVpp containing T314A or H316A exhibited an approximate 3-fold increase in infectivity compared to HCVpp 1177.20. Due to their increased infectivity compared to wild-type HCVpp, these virions, and others containing these E1 mutants, could be of particular use in screening anti-HCV antibodies and other anti-virals, as very few HCV isolates (including HCVcc and HCVpp) can produce infectious virions for in vitro assays. A key issue is whether the mutations also increase virus sensitivity to antibody neutralization, thus rendering them insuitable for the in vitro assays. Therefore, to determine whether the E1 mutations result in increased sensitivity to antibody neutralization, a neutralization assay was performed with C1 IgG (recognizes AR3), G IgG (recognizes AR2), N4 IgG (recognizes conformational E1/E2 epitope) and a negative control antibody, Den3 IgG (recognizes Dengue virus NS1 protein). The assay was performed essentially as described in Example 1. None of the HCVpp tested were neutralized by the negative control antibody, Den3 IgG (FIG. 8). HCVpp containing E1 with the P295A mutation exhibited similar sensitivity to the C1 IgG and N4 IgG as wildtype H77.20, with perhaps slightly increased sensitivity to G IgG. HCV containing E1 with the T314A or H316A mutation exhibited increased resistance to antibody neutralization compared to wildtype HCV. HCV containing E1 with the F293A mutation exhibited the greatest resistance to neutralization by all three anti-HCV antibodies compared to wild-type H77.20.

### Example 6

### Isolation of Further Monoclonal Antibodies

Additional rounds of epitope-masking/panning of the phage-display Fab library, essentially as described in Example 1, were performed and fourteen new monoclonal antibodies were isolated. The E1E2 complex from isolate H77 produced by transient transfection of 293T cells was used as a panning antigen. The antigens were presented in two different formats for two additional panning experiments. The formats were (1) E1E2 complex captured by Galanthus nivalis lectin, and (2) E1E2 complex captured by MAb AR3A then blocked with a cocktail of MAbs: AR1B, N4 and V1. The unmasked E1E2 complex was used in library panning for 4 rounds. The heavy chain of approximately 96 clones from rounds 3 and 4 of panning were analyzed by DNA sequencing and 11 new clones with specific reactivity to unmasked E1E2 were obtained. The antibody-masked E1E2 complex was used in library panning for a total of 6 rounds because no specific binder was obtained in rounds 3 and 4. The heavy chain of approximately 48 clones from round 6 were analyzed by DNA sequencing and 3 more clones with specific reactivity to unmasked E1E2 were obtained. DNA sequence analysis revealed 14 unique antibody sequences compared to those antibodies identified in the antibody panels described in Examples 1 and 3. The Fabs were sorted into groups based on heavy chain sequence. Four Fabs, labeled B4, B5, B6a, B6b, B6c and B7, were found to be heavy chain somatic variants of the previously identified Fabs B1 and B2. Three Fabs, labeled E2, E3 and E4, were found to be heavy chain somatic variants of the previously identified Fab E. One Fab, labeled J5, was found to be a heavy chain somatic variant of the previously identified Fabs J1, J2, J3 and J4. Three Fabs, L5, L6 and L7 were found to be heavy chain somatic variants of the previously identified Fabs L1, L2, L3 and L4. One Fab, labeled S2, was found to be a heavy chain somatic variant of the previously identified Fab S. Two Fabs, labeled U2 and U3, were found to be heavy chain somatic variants of the previously identified Fab U. The heavy and light chain amino acid sequences of these fourteen antibodies are shown in Tables 30 and 31, respectively. Fabs B7, E4 and L7 were identified by using the antibody-masked E1E2 panning strategy, while the remaining Fabs were identified using the unmasked E1E2 panning strategy. Also shown in the Tables are previously isolated Fabs B1, E1, J1, L1 S1 and U1, for comparison.

TABLE 30A
Heavy chain Framework 1 and CDR1 sequences

| | Framework 1 | | CDR1 | |
|---|---|---|---|---|
| Fab | Sequence | SEQ ID NO | Sequence | SEQ ID NO |

| | | | | |
|---|---|---|---|---|
| B1 | LEQSGAEVKKPGSSVKVSCRASGSPFS | 310 | SYTIT | 79 |
| B4 | EVQLLEQSGAEVKKPGSSVKVSCRASGSPFS | 1134 | SYTIT | 79 |
| B5 | EVQLLE SGAEVKKPGSSVKVSCRASGSPYS | 1135 | SYTIT | 79 |
| B6 | EVQLLEQSGAEVKKPGSSVKVSCRASGSPYS | 1136 | SYTIT | 79 |
| B7 | EVQLLEQSGAEVKKPGSSVKVSCRASGSPYS | 1136 | SYTIT | 79 |
| E | LEQSGAELKKPGSSVKVSCKPSDGTFR | 323 | AYTLS | 92 |
| E2 | EVGLLEQSGAELKKPGSSVKVSCKPSDGTFR | 1137 | AYTLS | 92 |
| E3 | EVGLLEQSGAELKKPGSSVKVSCKPSDGTFR | 1137 | AYTLS | 92 |
| E4 | EVGLLEQSGAELKKPGSSVKVSCKPSDGTFR | 1137 | AYTLS | 92 |
| J1 | LEQSGPEVKKPGSSVKVSCKGSGDRFN | 330 | DPVT | 99 |
| J5 | EVQLLEQSGAEVKKPGSSVTVSCKASGGTVS | 1138 | SYPIT | 1090 |
| L1 | LEQSGAEVKKPGSSVKVSCKASGDTFR | 335 | SYVIT | 104 |
| L5 | EVQLLEQSGTEVKKPGSSVKVSCKVPGDTFR | 1139 | SYVIT | 104 |
| L6 | EVQLLEQSGTEVKKPGSSVKVSCKVPGDTFR | 1139 | SYVIT | 104 |
| L7 | EVQLLEQSGAEVKKPGSSVKVTCKVPGDTFR | 1140 | SYVIT | 104 |
| S1 | EVQLLEQSGAEVKKPGTSVKVSCTASGYIFT | 849 | SFGIS | 738 |
| S2 | EVQLLEQSGAEVKKPGASVKVSCTASGYIFT | 1141 | SFGIS | 738 |
| U1 | EVQLLEQSGPEVKRPGTSVKMSCKISGGASI | 851 | TQAMS | 740 |
| U2 | EVQLLEQSGAEVKRPGSSVKLSCKNSGGTFI | 1142 | TQAMT | 1091 |
| U3 | EVQLLEQSGAEVKRPGSSVKISCKNSGGTFI | 1143 | TQAMS | 740 |

TABLE 30B
Heavy chain Framework 2 and CDR2 sequences

| | Framework 2 | | CDR2 | |
|---|---|---|---|---|
| | | SEQ ID | | SEQ ID |
| Fab | Sequence | NO | Sequence | NO |
| B1 | WVRQAPGQGLEWMG | 341 | GIILMTGKANYAQKFQG | 110 |
| B4 | WVRQAPGQGLEWMG | 341 | GIILMTGKANYAQKFQG | 110 |
| B5 | WVRQAPGQGLEWMG | 341 | GIILMTGKANYAQKFQG | 110 |
| B6 | WVRQAPGQGLEWMG | 341 | GIILMTGKANYAQKFQG | 110 |
| B7 | WVRQAPGQGLEWMG | 341 | GIILMTGKANYAQKFQG | 110 |
| E | WVRQAPGQTLEWMG | 354 | RIMPTVGITNYAQKFQG | 123 |
| E2 | WVRQAPGQTLEWLG | 1144 | RIMPTVGITNYAQKFQG | 123 |
| E3 | WVRQAPGQALEWMG | 1145 | RIMPTVGITNYARKFQG | 1092 |
| E4 | WVRQAPGQTLEWMG | 354 | RIMPTVGITNYAQKFQG | 123 |
| J1 | WVRQAPGQGLEWIG | 361 | GIIPAFGATKYAQKFQG | 130 |
| J5 | WVRQAPGQGLEWMG | 341 | GTIPVFGAPKYAPKFQG | 1093 |
| L1 | WARQAPGQGLEWMG | 366 | AIIPFFGTTNLAQKFQG | 135 |
| L5 | WVRQAPGQGLEWLG | 1146 | GILPFFGTTNLAQKFQG | 1094 |
| L6 | WVRQSPGQGLEWLG | 1147 | GILPFFGTTNLAQKFQG | 1094 |
| L7 | WVRQAPGQGLEWLG | 1146 | GILPFFGTTNLAQKFQG | 1094 |
| S1 | WVRQAPGQGLEWMG | 867 | RIDTYNGKTNYAQKLQG | 756 |
| S2 | WVRQAPGQGLEWMG | 341 | RIDTYNGKTNYAQKLQG | 756 |
| U1 | WVRQAPGQGLEWMG | 869 | GITPIFGTVNYAQKILG | 758 |
| U2 | WVRQAPGQGLEWMG | 341 | GIIPVFGTVNYAQNILD | 1095 |
| U3 | WVRQAPGQGLEWMG | 341 | GIIPIFGTVNYAQKILG | 1096 |

TABLE 30C
Heavy chain Framework 3 and CDR3 sequences

| | |
|---|---|
| Framework 3 | CDR3 |

| | | | | |
|---|---|---|---|---|
| | | SEQ | | SEQ |
| | | ID | | ID |
| Fab | Sequence | NO | Sequence | NO |

| | | | | |
|---|---|---|---|---|
| B1 | RVTITADRSTTTAYMEMSSLTSDDTAIYYCAR | 372 | DPYVYAGDDVWSLSR | 141 |
| B4 | RVTITADRSTTTAYMEMSSPTSDDTAIYYCAR | 1148 | DPYVYAGDDVWSLSR | 141 |
| B5 | RVTITADRATATAYMEMSSLTSDDTAIYYCAR | 1149 | DPYVYAGDDVWSLSR | 141 |
| B6 | RVTITADRATATAYMEMSSLTSDDTAIYYCAR | 1149 | DPYVYAGDDVWSLSR | 141 |
| B7 | RVTITADRATATAYMEMSSLTSDDTAIYYCAR | 1149 | DPYVYAGDDVWSLSR | 141 |
| E | RVTISADMSTATAYMELSSLRSDDTAIYYCAK | 385 | GPYVGLGEGFSE | 154 |
| E2 | RVTISADMSTATAYMQLSSLRPDDTAIYYCAK | 1150 | GPYVGLGEGFSE | 154 |
| E3 | RVTISADMSTATAYMELSSLRSDDTAIYYCAK | 385 | GPYVGLGEGFSE | 154 |
| E4 | RVTISADMSTATAYMELSSLRSDDTAIYYCGK | 1151 | GPYVGLGEGFSE | 154 |
| J1 | RVVISADASTDTAYMELSSLRSEDTAVYYCAK | 392 | VGVRGIILVGGLAMNWLDP | 161 |
| J5 | RVTITADESTSTAYMELRSLRSDDTAMYYCAI | 1152 | VGMRGITLVGGLAMNWLDP | 1097 |
| L1 | RVTITADESTQTVYMDLSSLRSDDTAVYYCAK | 397 | AGDLSVGGVLAGGVPHLRHFDP | 166 |
| L5 | RVTLTADESTTTAYMELSSLRAEDTAVYYCAK | 1153 | AGDLAFGGVIAGGVPHLSHFDP | 1098 |
| L6 | RVTLTADESTTTAYMELSSLRSEDTAVYYCAK | 1154 | AGDLAFGGVIAGGVPHLSHFDP | 1098 |
| L7 | RVTLTADESTTTAYMELSSLRAEDTAVYYCAK | 1153 | AGDLAFGGVIAGGVPHLSHFDP | 1098 |
| S1 | RVTMTTDTYTSTAYMELRSLTSDDTAVYYCAR | 885 | DDCRSSTCYLAQHNWQAYYH DS | 774 |
| S2 | RVTMTTDTYTSTAYMELRSLTSDDTAVYYCAR | 885 | DDCRSSTCYLAQHNWQAYYH DS | 774 |
| U1 | RVTITADED TVSLELSSLKSEDTAVYYCAR | 887 | EVNLKTWNLAHPNVF DV | 776 |
| U2 | RATATADED TFSLELRGLRLEDTAVYYCAR | 1155 | EVNLKTWNLARPDVF DI | 1099 |
| U3 | RVTITADED TVSMELSSLGFEDTAVYYCAR | 1156 | EVNLKSWNLAHPDVF DI | 1100 |

TABLE 30D
Heavy chain Framework 4

Framework 4

| | | SEQ |
|---|---|---|
| | | ID |
| Fab | Sequence | NO |
| B1 | WGQGTLVIVSSAS | 403 |
| B4 | WGQGTLVIVSP | 1157 |
| B5 | WGQGTPVIVSS | 1158 |
| B6 | WGQGTPVTVSS | 1159 |
| B7 | WGQGTPVIVSS | 1158 |
| E | WGQGTLVTVSSAS | 416 |
| E2 | WGQGTLVTVSS | 1160 |
| E3 | WGQGTLVTVSS | 1160 |
| E4 | WGQGTLVTVSS | 1160 |
| J1 | WGQGTLVTVSAAS | 423 |
| J5 | WGQGTLVIVSS | 1161 |
| L1 | WGQGTLVTVSSAS | 428 |
| L5 | WGQGTLVTVSS | 1160 |
| L6 | WGQGTLVTVSA | 1162 |
| L7 | WGQGTLVTVSS | 1160 |
| S1 | WGQGTLVTVSS | 903 |
| S2 | WGQGTLVTVSS | 1160 |
| U1 | WGQGTMLTVSS | 905 |
| U2 | WGPGTLVTVSS | 1163 |
| U3 | WGQGTLVTVSS | 1164 |

TABLE 31A
Light chain Framework 1 and CDR1 sequences

| | Framework 1 | | | |
|---|---|---|---|---|
| | | SEQ ID | CDR1 | |
| Fab | Sequence | NO | Sequence | SEQ ID NO |

| | | | | |
|---|---|---|---|---|
| B1 | ELTLTQSPGTLSLSPGERATLSC | 434 | RASQS VSNSYLA | 172 |
| B4 | ELTLTQSPGTLSLSPGKRATLSC | 1165 | RASQS VSGSYLA | 1101 |
| B5 | ELTQSPLSLPVIIGQPASISC | 1166 | SSSESLVDSDGNTYLH | 1102 |
| B6a | LTQSPGTLSLSPGERATLSC | 1167 | RASQS VASNYVA | 1103 |
| B6b | ELTQSPGTLSLSPGERATLSC | 1171 | RASQS VSRGNLA | 1107 |
| B6c | ELTQSPGTLSLSPGERATLSC | 1171 | RASQS VSSSYLA | 196 |
| B7 | EL TQSPLSLPVTLGQPASISC | 1168 | RSSQSLVYSDGNTYLN | 1104 |
| E | ELVLTQSPLSLPVTLGQPASISC | 449 | RSTQSLVYSDGNTYLN | 187 |
| E2 | ELTQSPLSLPVTLGQPASISC | 1169 | RSSQSLVYSDGNTYLI | 1105 |
| E3 | ELVLTQSPLSLPVTLGQPASISC | 1170 | RSTQSLVYSDGNTYLN | 1106 |
| J1 | ELVLTQSPGTLSLSPGERATLSC | 485 | RASQS VSSSYLA | 196 |
| J5 | EL TQSPGTLSLSPGERATLSC | 464 | RASQS VSSNYLA | 1108 |
| L1 | EL TQSPGTLSLSPGERATLSC | 464 | RASQS ITSRYLA | 202 |
| L5 | EL TQSPGTLSLSPGERATLSC | 464 | RASQS VRSNYLA | 1109 |
| L6 | EL TQSPGTLSLSPGERATLSC | 464 | RASQS VSSSYLA | 196 |
| L7 | EL TQSPGTLSLSPGERATLSC | 464 | RASQS VPSSYLG | 1110 |
| S1 | ELVLTQSPGTLSLSPGERATLSC | 922 | RASQS VSSSYLA | 196 |
| S2 | EL TQSPATLSLSPGERATLSC | 1172 | RASQS VSSRFLA | 1111 |
| U1 | EL TQSPSSLSASVGDRVTITC | 924 | RASQS ISSFLN | 795 |
| U2 | EL TQSPSSLSASIGDRVTIPC | 1173 | RASQS ILNHLN | 1112 |
| U3 | EL TQSPSSLSASVGDRVTITC | 924 | RASQS IRSYLN | 1113 |

TABLE 31B
Light chain Framework 2 and CDR2 sequences

| | Framework 2 | | | |
|---|---|---|---|---|
| | | SEQ ID | CDR2 | |
| Fab | Sequence | NO | Sequence | SEQ ID NO |
| B1 | WYQQKPGQAPRLLIY | 469 | GASSRAT | 207 |
| B4 | WYQQKPGQAPRLLIY | 469 | GASNRAT | 1114 |
| B5 | WFQQRPGQSPRRLIY | 1174 | KVSNRDA | 1115 |
| B6a | WYQQKPGQAPRLLIY | 1175 | GTSYRAT | 1116 |
| B6b | WYQQKRGQPPRLLIY | 1179 | GASYRAT | 1119 |
| B6c | WYQQKPGQAPRLLIY | 1175 | GASSRAT | 207 |
| B7 | WFQQRPGQSPRRLIY | 1176 | KVSNRDS | 222 |
| E | WFHQRAGQPPRRLIY | 484 | KVSNRDS | 222 |
| E2 | WFQQRPGQSPRRLIY | 1177 | NVSNRDS | 1117 |
| E3 | WFHQRPGQPPRRLIY | 1178 | ELVLTQS | 1118 |
| J1 | WYQQKPGQAPRLLIY | 493 | GASSRAT | 231 |
| J5 | WYQQKPGQAPRLLIY | 469 | GASSRAT | 231 |
| L1 | WYQQKPGQAPRLLIY | 499 | GASSRAT | 237 |
| L5 | WYQQKPGQAPRLLIY | 469 | GASSRAT | 207 |
| L6 | WYQQKPGQAPRLLIY | 469 | GASSRAT | 207 |
| L7 | WYQQKPGQAPRLLIY | 469 | GASSRAT | 207 |
| S1 | WYQQKPGQAPRLLIY | 941 | GASSRAT | 812 |
| S2 | WYQQKPGQAPRLLIY | 469 | GASSRAT | 207 |
| U1 | WYQQKPGKAPKLLIY | 943 | AASSLQS | 814 |
| U2 | WYQQKPGQPPKLLIF | 1180 | AASNLQS | 1120 |
| U3 | WYQQKPGKAPNLLIY | 1181 | SASNLQS | 1121 |

TABLE 31C
Light chain Framework 3 and CDR3 sequences

| | |
|---|---|
| Framework 3 | CDR3 |

| | | | | |
|---|---|---|---|---|
| | | SEQ ID | | SEQ ID |
| Fab | Sequence | NO | Sequence | NO |

| | | | | |
|---|---|---|---|---|
| B1 | GIPDRFSGSGSGTDFTLTISRLEPEDFAVYYC | 504 | QQYGSSPQT | 242 |
| B4 | GIPHRFSGSGSGTDFTLTISRLEPEDFAVYYC | 1182 | QQYGSSPPT | 1122 |
| B5 | GVPDRFSGSGSGTDFTLKISRVEAEDVAVYYC | 1183 | MQATHWPPIT | 1123 |
| B6a | GIPGRFSGSGSGTDFTLTISGLEPEDFAVYYC | 1184 | QQYGSSPQT | 1124 |
| B6b | GIPDRFSGSGSGTDFTLTITKLEPEDFAVYYC | 1188 | QQYGHSLA | 1128 |
| B6c | GIPDRFSGSGSGTDFTLTISRLEPEDFAVYYC | 504 | QQYGSSPQT | 1124 |
| B7 | GVPDRFSGSGSGTDFTLKISRVEAEDVGVYYC | 1185 | MQGTHWPPT | 1125 |
| E | GVPERFSGSGSGTDFTLKISRVEAEDVGIYYC | 519 | MQGAHWPPT | 257 |
| E2 | GVPDRFSGSGSGTDFTLKISRVEAEDLGVYYC | 1186 | MQGAHWPPT | 1126 |
| E3 | GVPERFSGSGSGTDFALKISRVEADDVGIYYC | 1187 | MQGAHWPPT | 1127 |
| J1 | GIPDRFSGSGSGTDFALTITRLEPEDFAVYYC | 528 | QQYGSSPQT | 266 |
| J5 | GIPDRFSGSGSGTDFTLSISRLEPEDFAVYYC | 1189 | QQYGSSPLT | 1129 |
| L1 | GIPDRFSGSGSGTDFTLTISRLEPEDFAVYYC | 534 | QQYGDSVG | 272 |
| L5 | GIPDRFSGSGSGTDFTLTISRLEPEDFAMYYC | 1190 | QQYGSSMCS | 1130 |
| L6 | GIPDRFSGSGSGTDFTLTISRLEPEDFAVYYC | 534 | QQYGSSVT | 1131 |
| L7 | GIPERFSGSGSGTDFTLTISRLEPEDFAVYYC | 1191 | QQYGSSLS | 1132 |
| S1 | GIPDRFSGSGSGTDFTLTISRLEPEDFAVYYC | 960 | QQYGSSPQT | 831 |
| S2 | GIPDRFSGSGSGTDFTLTISRLEPEDFAVYYC | 534 | QQYGSSPQT | 831 |
| U1 | GVPPRFSGSGSGTDFTLTISSLQPEDFATYYC | 962 | QQSYSTPRT | 833 |
| U2 | GVPSRFSGSGSGTDFTLTISSLQTEDFATYYC | 1192 | QQSYSTPRT | 833 |
| U3 | GVPSRFRGSGSGTDFTLTISSLQPEDFATYYC | 1193 | QQSYRTPRT | 1133 |

TABLE 31D
Light chain Framework 4

Framework 4

| | | SEQ |
|---|---|---|
| | | ID |
| Fab | Sequence | NO |
| B1 | FGQGTKVEIKRTVAAPSVFIFPPSDEQLKSGTASVV | 539 |
| B4 | FGQGTRVDIKRT | 1194 |
| B5 | FGPGTRLEVKRT | 1195 |
| B6a | FGQGTKVEIKRT | 1196 |
| B6b | FGQGTKVEIKGT | 1200 |
| B6c | FGQGTKVEIKRT | 1196 |
| B7 | FGQGTKVDIKRT | 1197 |
| E | FGGGTKVEINRTVAAPSVFIFPPSDEQLKSGTA | 554 |
| E2 | FGQGTRLEIKGT | 1198 |
| E3 | FGGGTKVEISRT | 1199 |
| J1 | FGQGTKVEIKRTVAAPSVFIFPPSDEQLKSGTASVV | 563 |
| J5 | FGGGTKVEIKRT | 1201 |
| L1 | FGPGTKVEIKRTVAAPSVFIFPPSDEQLKSGTASVV | 569 |
| L5 | FGQGTKVEIKRT | 979 |
| L6 | FGPGTKVDIKRT | 1202 |
| L7 | FGQGTKVDLKRT | 1203 |
| S1 | FGQGTKVEIKRT | 979 |
| S2 | FGQGTKVEIKRT | 979 |
| U1 | FGQGTKLEIKRT | 981 |
| U2 | FGQGTKVEVKRT | 1204 |
| U3 | FGQGTKVESKRT | 1205 |

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the statements. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the statements. As used herein and in the appended statements, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as statemented. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended statements.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

The following statements of the invention are intended to characterize possible elements of the invention according to the foregoing description given in the specification. Because this application is a provisional application, these statements may become changed upon preparation and filing of a nonprovisional application. Such changes are not intended to affect the scope of equivalents according to the statements issuing from the nonprovisional application, if such changes occur. According to 35 U.S.C. §111(b), statements are not required for a provisional application. Consequently, the statements of the invention cannot be interpreted to be statements pursuant to 35 U.S.C. §112.

Since modifications will be apparent to those of skill in this art, it is intended that this invention be limited only by the scope of the appended claims.

1-164. (canceled)

165. An antibody or antigen-binding fragment thereof, wherein:
the antibody or antigen-binding fragment thereof selectively binds to the hepatitis C virus (HCV) E1E2 complex;
the antibody or antigen-binding fragment thereof does not bind to a purified E2 polypeptide that is not in an E1E2 complex;
the antibody or antigen-binding fragment thereof does not bind to a linear epitope on the HCV E1 polypeptide; and
the antibody or antigen-binding fragment thereof neutralizes at least 2 isolates of hepatitis C virus.

166. The antibody or antigen-binding fragment thereof of claim 165, comprising
a variable heavy chain (V_{H}) complementary determining region (CDR) CDR1 comprising a sequence of amino acids set forth in any of SEQ ID NOS: 725-741, 983 and 1090-1091 or a sequence having at least 70%, 80%, 90%, 91%, 92%, 93%, 94%, 95%, 96%, 97%, 98%, 99% or more sequence identity thereto;
a V_{H} CDR2 having a sequence of amino acids set forth in any of SEQ ID NOS: 742-759 and 1092-1096 or a sequence having at least 70%, 80%, 90%, 91%, 92%, 93%, 94%, 95%, 96%, 97%, 98%, 99% or more sequence identity thereto; and
a V_{H} CDR3 comprising a sequence of amino acids set forth in any of SEQ ID NOS: 760-777 and 1097-1100 or a sequence having at least 70%, 80%, 90%, 91%, 92%, 93%, 94%, 95%, 96%, 97%, 98%, 99% or more sequence identity thereto.

167. The antibody or antigen-binding fragment thereof of claim 165, comprising
a V_{H} CDR1 comprising a sequence of amino acids set forth in SEQ ID NO:727 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:745 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:763 or a sequence having at least 70%, 80%, 90%, 91%, 92%, 93%, 94%, 95%, 96%, 97%, 98%, 99% or more sequence identity thereto; or
a V_{H} CDR1 comprising a sequence of amino acids set forth in SEQ ID NO:725 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:742 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:760 or a sequence having at least 70%, 80%, 90%, 91%, 92%, 93%, 94%, 95%, 96%, 97%, 98%, 99% or more sequence identity thereto; or
a V_{H} CDR1 comprising a sequence of amino acids set forth in SEQ ID NO:730 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:748 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:766 or a sequence having at least 70%, 80%, 90%, 91%, 92%, 93%, 94%, 95%, 96%, 97%, 98%, 99% or more sequence identity thereto; or
a V_{H} CDR1 comprising a sequence of amino acids set forth in SEQ ID NO:741 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:759 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:777 or a sequence having at least 70%, 80%, 90%, 91%, 92%, 93%, 94%, 95%, 96%, 97%, 98%, 99% or more sequence identity thereto; or
a V_{H} CDR1 comprising a sequence of amino acids set forth in SEQ ID NO:726 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:743 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:761 or a sequence having at least 70%, 80%, 90%, 91%, 92%, 93%, 94%, 95%, 96%, 97%, 98%, 99% or more sequence identity thereto; or
a V_{H} CDR1 comprising a sequence of amino acids set forth in SEQ ID NO:983 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:744 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:762 or a sequence having at least 70%, 80%, 90%, 91%, 92%, 93%, 94%, 95%, 96%, 97%, 98%, 99% or more sequence identity thereto; or
a V_{H} CDR1 comprising a sequence of amino acids set forth in SEQ ID NO:733 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:751 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:769 or a sequence having at least 70%, 80%, 90%, 91%, 92%, 93%, 94%, 95%, 96%, 97%, 98%, 99% or more sequence identity thereto; or
a V_{H} CDR1 comprising a sequence of amino acids set forth in SEQ ID NO:735 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:753 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:771 or a sequence having at least 70%, 80%, 90%, 91%, 92%, 93%, 94%, 95%, 96%, 97%, 98%, 99% or more sequence identity thereto; or
a V_{H} CDR1 comprising a sequence of amino acids set forth in SEQ ID NO:738 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:756 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:774 or a sequence having at least 70%, 80%, 90%, 91%, 92%, 93%, 94%, 95%, 96%, 97%, 98%, 99% or more sequence identity thereto; or
a V_{H} CDR1 comprising a sequence of amino acids set forth in SEQ ID NO:728 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:746 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:764 or a sequence having at least 70%, 80%, 90%, 91%, 92%, 93%, 94%, 95%, 96%, 97%, 98%, 99% or more sequence identity thereto; or
a V_{H} CDR1 comprising a sequence of amino acids set forth in SEQ ID NO:729 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:747 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:765 or a sequence having at least 70%, 80%, 90%, 91%, 92%, 93%, 94%, 95%, 96%, 97%, 98%, 99% or more sequence identity thereto; or
a V_{H} CDR1 comprising a sequence of amino acids set forth in SEQ ID NO:731 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:749 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:767 or a sequence having at least 70%, 80%, 90%, 91%, 92%, 93%, 94%, 95%, 96%, 97%, 98%, 99% or more sequence identity thereto; or
a V_{H} CDR1 comprising a sequence of amino acids set forth in SEQ ID NO:732 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:750 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:768 or a sequence having at least 70%, 80%, 90%, 91%, 92%, 93%, 94%, 95%, 96%, 97%, 98%, 99% or more sequence identity thereto; or
a V_{H} CDR1 comprising a sequence of amino acids set forth in SEQ ID NO:734 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:752 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:770 or a sequence having at least 70%, 80%, 90%, 91%, 92%, 93%, 94%, 95%, 96%, 97%, 98%, 99% or more sequence identity thereto; or
a V_{H} CDR1 comprising a sequence of amino acids set forth in SEQ ID NO:736 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:754 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:772 or a sequence having at least 70%, 80%, 90%, 91%, 92%, 93%, 94%, 95%, 96%, 97%, 98%, 99% or more sequence identity thereto; or
a V_{H} CDR1 comprising a sequence of amino acids set forth in SEQ ID NO:737 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:755 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:773 or a sequence having at least 70%, 80%, 90%, 91%, 92%, 93%, 94%, 95%, 96%, 97%, 98%, 99% or more sequence identity thereto; or
a V_{H} CDR1 comprising a sequence of amino acids set forth in SEQ ID NO:739 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:757 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:775 or a sequence having at least 70%, 80%, 90%, 91%, 92%, 93%, 94%, 95%, 96%, 97%, 98%, 99% or more sequence identity thereto; or
a V_{H} CDR1 comprising a sequence of amino acids set forth in SEQ ID NO:740 or a sequence having at least 70%, 80%, 90%, 91%, 92%, 93%, 94%, 95%, 96%, 97%, 98%, 99% or more sequence identity thereto, a V_{H} CDR2 comprising a sequence of amino acids set forth in SEQ ID NO:758 or a sequence having at least 70%, 80%, 90%, 91%, 92%, 93%, 94%, 95%, 96%, 97%, 98%, 99% or more sequence identity thereto, and a V_{H} CDR3 comprising a sequence of amino acids set forth in SEQ ID NO:776 or a sequence having at least 70%, 80%, 90%, 91%, 92%, 93%, 94%, 95%, 96%, 97%, 98%, 99% or more sequence identity thereto.

168. The antibody or antigen-binding fragment thereof of claim 165 that is an antigen-binding fragment selected from among a single-chain Fv (scFv), Fab, Fab′, F(ab′)₂, Fv, dsFv, diabody, Fd and a Fd′ fragment.

169. The antibody or antigen-binding fragment thereof of claim 165, comprising (1) a variable light chain (V_{L}) CDR1 with a sequence of amino acids set forth in any of SEQ ID NOS:778-796 and 1101-1113 or a sequence having at least 70%, 80%, 90%, 91%, 92%, 93%, 94%, 95%, 96%, 97%, 98%, 99% or more sequence identity thereto; (2) a V_{L} CDR2 with a sequence of amino acids set forth in any of SEQ ID NOS:797-815 and 1114-1121 or a sequence having at least 70%, 80%, 90%, 91%, 92%, 93%, 94%, 95%, 96%, 97%, 98%, 99% or more sequence identity thereto; and (3) a V_{L} CDR3 with a sequence of amino acids set forth in any of SEQ ID NOS:816-834 and 1122-1133 or a sequence having at least 70%, 80%, 90%, 91%, 92%, 93%, 94%, 95%, 96%, 97%, 98%, 99% or more sequence identity thereto.

170. The antibody or antigen-binding fragment thereof of claim 165, comprising a heavy chain, wherein the heavy chain comprises a sequence of amino acids set forth in any of SEQ ID NOS:986-1004 or 1062-1075.

171. The antibody or antigen-binding fragment thereof of claim 165, comprising a light chain, wherein the light chain comprises a sequence of amino acids set forth in any of SEQ ID NOS:1005-1023 or 1076-1089.

172. The antibody or antigen-binding fragment thereof of claim 165, comprising:
a V_{H} CDR1 having a sequence of amino acids set forth in SEQ ID NO:727;
a V_{H} CDR2 having a sequence of amino acids set forth in SEQ ID NO:745;
a V_{H} CDR3 having a sequence of amino acids set forth in SEQ ID NO:763;
a V_{L} CDR1 having a sequence of amino acids set forth in SEQ ID NO:781;
a V_{L} CDR2 having a sequence of amino acids set forth in SEQ ID NO:800; and
a V_{L} CDR3 having a sequence of amino acids set forth in SEQ ID NO:819.

173. The antibody or antigen-binding fragment thereof of claim 165, comprising
a heavy chain having a sequence of amino acids set forth SEQ ID NO:989; and
a light chain having a sequence of amino acids set forth SEQ ID NO:1008.

174. An antibody or antigen-binding fragment thereof that selectively binds to the same epitope as that which is selectively bound by the antibody or antigen-binding fragment thereof of claim 165.

175. A method for detecting hepatitis C virus in a sample, comprising:
contacting the sample with an antibody or antigen-binding fragment of claim 165; and
determining whether the antibody binds specifically to the sample, wherein the binding of the antibody to the sample indicates that the sample contains a hepatitis C virus.

176. A method for treating or preventing hepatitis C viral infection in a mammal susceptible to infection by a hepatitis C virus, comprising administering to the mammal an effective amount of an antibody or antigen-binding fragment of claim 165.

177. An isolated nucleic acid molecule that encodes the antibody or antigen-binding fragment of claim 165.

178. An expression vector, comprising the nucleic acid molecule of claim 177.

179. A cell comprising the expression vector of claim 178.

180. A polypeptide, comprising at least one CDR sequence that contains a sequence of amino acids selected from among any set forth in any of SEQ ID NOS: 725-834, 985 and 1090-1133; or a sequence having at least 70%, 80%, 90%, 91%, 92%, 93%, 94%, 95%, 96%, 97%, 98%, 99% or more sequence identity with a sequence of amino acids set forth in any of SEQ ID NOS: 725-834 and 1090-1133, and that, when in an antibody or antigen-binding fragment thereof, the antibody or antigen-binding fragment selectively binds to E1, E1/E2 complex and/or to an HCV virion.

181. The polypeptide of claim 180 that has two CDR sequences, wherein the first CDR (CDR1) sequence has an amino acid sequence set forth in any of SEQ ID NOS: 725-741, 778-796, 985, 1090-1091 and 1101-1113; and the second CDR (CDR2) sequence has an amino acid sequence set forth in any of SEQ ID NOS: 742-759, 797-815, 1092-1096 and 1114-1121.

182. The polypeptide of claim 180 that has three CDR sequences, wherein the first CDR1 sequence has an amino acid sequence set forth in any of SEQ ID NOS: 725-741, 778-796, 985, 1090-1091 or 1101-1113; the second CDR2 sequence has an amino acid sequence set forth in any of SEQ ID NOS: 742-759, 797-815, 1092-1096 and 1114-1121; and the third CDR (CDR3) sequence has an amino acid sequence set forth in any of SEQ ID NOS: 760-777, 816-834, 1097-1100 and 1122-1133.

183. The polypeptide of claim 180, further comprising a framework sequence, wherein the framework sequence has a sequence of amino acids set forth in any of SEQ ID NOS:835-982 and 1134-1205.

184. The polypeptide of claim 180, comprising two framework sequences, wherein:
the first framework (framework-1) sequence, from the N-terminus of the polypeptide, has a sequence of amino acids set forth in any of SEQ ID NOS: 835-852, 907-925, 1134-1143 and 1165-1173; and
the second framework (framework-2) sequence, from the N-terminus of the polypeptide, has a sequence of amino acids set forth in any of SEQ ID NOS: 853-870, 926-944, 1144-1147, and 1174-1181.

185. The polypeptide of claim 180, comprising three framework sequences, wherein:
the framework-1 sequence, from the N-terminus of the polypeptide, has a sequence of amino acids set forth in any of SEQ ID NOS: 835-852, 907-925, 1134-1143 and 1165-1173;
the framework-2 sequence, from the N-terminus of the polypeptide, has a sequence of amino acids set forth in any of SEQ ID NOS: 853-870, 926-944, 1144-1147, and 1174-1181; and
the third framework sequence (framework-3), from the N-terminus of the polypeptide, has a sequence of amino acids set forth in any of SEQ ID NOS: 871-888, 945-963, 1148-1156 and 1182-1193.

186. The polypeptide of claim 180, comprising four framework sequences, wherein:
the framework-1 sequence, from the N-terminus of the polypeptide, has a sequence of amino acids set forth in any of SEQ ID NOS: 835-852, 907-925, 1134-1143 and 1165-1173;
the framework-2 sequence, from the N-terminus of the polypeptide, has a sequence of amino acids set forth in any of SEQ ID NOS: 853-870, 926-944, 1144-1147, and 1174-1181;
the framework-3 sequence, from the N-terminus of the polypeptide, has a sequence of amino acids set forth in any of SEQ ID NOS: 871-888, 945-963, 1148-1156 and 1182-1193; and
the fourth framework (framework-4) sequence, from the N-terminus of the polypeptide, has a sequence of amino acids set forth in any of SEQ ID NOS: 889-906, 964-982, 1157-1164, and 1194-1205.

187. The polypeptide of claim 180 that has a sequence of amino acids set forth in any of SEQ ID NOS: 986-1023, 1062-1089 and 1365.
